SEQUENCE LISTING (865 sequences)

SEQ ID NO: 1 - DNA, 235 bp, DIDYMIUM IRIDIS
ttttggttgggttgggaagtatcatggctaatcaccatgatgcaatcgggttgaacacttaattgggttaaaacggtgggggacgatcccgtaacatccgtcctaacggcgacagactgcacggccctgcctcttaggtgtgttcaatgaacagtcgttccgaaaggaagcatccggtatcccaagacaatcaaatctaaggataccaatctgtgcacttcaacaacaatggtga

SEQ ID NO: 2 - DNA, 180 bp, NAEGLERIA JAMIESONI
ccgttgttgtgcgatggggttcataccttaatctgccaaaacgggacctctgttgaggtataaccaatattccgtactaaggatttcgatccagaacgtctagagactacacggtagaccaattttggtggtatgaatggatagtccctagtaaccatctaggcatcccatacaaaatgg

SEQ ID NO: 3 - RNA, 22 nt, HOMO SAPIENS
aauccuuugucccugggugaga

SEQ ID NO: 4 - RNA, 21 nt, HOMO SAPIENS
aaacuacugaaaaucaaagau

SEQ ID NO: 5 - RNA, 21 nt, HOMO SAPIENS
aauauaacacagauggccugu

SEQ ID NO: 6 - RNA, 22 nt, HOMO SAPIENS
ugagaacugaauuccauaggcu

SEQ ID NO: 7 - RNA, 23 nt, HOMO SAPIENS
aucaacagacauuaauugggcgc

SEQ ID NO: 8 - RNA, 24 nt, HOMO SAPIENS
aaagacauaggauagagucaccuc

SEQ ID NO: 9 - RNA, 22 nt, HOMO SAPIENS
aacgcacuucccuuuagagugu

SEQ ID NO: 10 - RNA, 21 nt, HOMO SAPIENS
uaacagucuccagucacggcc

SEQ ID NO: 11 - RNA, 22 nt, HOMO SAPIENS
cuuucagucggauguuugcagc

SEQ ID NO: 12 - RNA, 21 nt, HOMO SAPIENS
uagauaaaauauugguaccug

SEQ ID NO: 13 - RNA, 22 nt, HOMO SAPIENS
uggaauguaaggaagugugugg

SEQ ID NO: 14 - RNA, 19 nt, HOMO SAPIENS
aagcagcugccucugaggc

SEQ ID NO: 15 - RNA, 22 nt, HOMO SAPIENS
aacacaccugguuaaccucuuu

SEQ ID NO: 16 - RNA, 23 nt, HOMO SAPIENS
gugccuacugagcugauaucagu

SEQ ID NO: 17 - RNA, 20 nt, HOMO SAPIENS
uugcauagucacaaaaguga

SEQ ID NO: 18 - RNA, 21 nt, HOMO SAPIENS
uagguaguuucauguuguugg

SEQ ID NO: 19 - RNA, 24 nt, HOMO SAPIENS
ugaaacauacacgggaaaccucuu

SEQ ID NO: 20 - RNA, 21 nt, HOMO SAPIENS
gaaggcgcuucccuuuagagc

SEQ ID NO: 21 - RNA, 23 nt, HOMO SAPIENS
ugugcaaaucuaugcaaaacuga

SEQ ID NO: 22 - RNA, 23 nt, HOMO SAPIENS
agcagcauuguacagggcuauca

SEQ ID NO: 23 - RNA, 22 nt, HOMO SAPIENS
ucacagugaaccggucucuuuc

SEQ ID NO: 24 - RNA, 21 nt, HOMO SAPIENS
aaagcgcuucccuuugcugga

SEQ ID NO: 25 - RNA, 22 nt, HOMO SAPIENS
uagcagcacauaaugguuugug

SEQ ID NO: 26 - RNA, 21 nt, HOMO SAPIENS
uaguagaccguauagcguacg

SEQ ID NO: 27 - RNA, 22 nt, HOMO SAPIENS
gucauacacggcucuccucucu

SEQ ID NO: 28 - RNA, 19 nt, HOMO SAPIENS
gugucugcuuccuguggga

SEQ ID NO: 29 - RNA, 23 nt, HOMO SAPIENS
aauggcgccacuaggguugugca

SEQ ID NO: 30 - RNA, 22 nt, HOMO SAPIENS
gcacauuacacggucgaccucu

SEQ ID NO: 31 - RNA, 20 nt, HOMO SAPIENS
gcgugcgccggccggccgcc

SEQ ID NO: 32 - RNA, 21 nt, HOMO SAPIENS
cuuuuugcggucugggcuugc

SEQ ID NO: 33 - RNA, 21 nt, HOMO SAPIENS
aacuggccuacaaagucccag

SEQ ID NO: 34 - RNA, 23 nt, HOMO SAPIENS
agugacaucacauauacggcagc

SEQ ID NO: 35 - RNA, 19 nt, HOMO SAPIENS
aggcacggugucagcaggc

SEQ ID NO: 36 - RNA, 23 nt, HOMO SAPIENS
ucccugagacccuuuaaccugug

SEQ ID NO: 37 - RNA, 22 nt, HOMO SAPIENS
ucacagugaaccggucucuuuu

SEQ ID NO: 38 - RNA, 22 nt, HOMO SAPIENS
uggucuaggauuguuggaggag

SEQ ID NO: 39 - RNA, 23 nt, HOMO SAPIENS
uaaagugcuuauagugcagguag

SEQ ID NO: 40 - RNA, 23 nt, HOMO SAPIENS
aaagugcuuccuuuuagaggguu

SEQ ID NO: 41 - RNA, 22 nt, HOMO SAPIENS
gugagucucuaagaaaagagga

SEQ ID NO: 42 - RNA, 21 nt, HOMO SAPIENS
acagucugcugagguuggagc

SEQ ID NO: 43 - RNA, 22 nt, HOMO SAPIENS
ccacugccccaggugcugcugg

SEQ ID NO: 44 - RNA, 23 nt, HOMO SAPIENS
cgaauguugcucggugaaccccu

SEQ ID NO: 45 - RNA, 23 nt, HOMO SAPIENS
uauggcacugguagaauucacug

SEQ ID NO: 46 - RNA, 21 nt, HOMO SAPIENS
uggaauguaaagaaguaugua

SEQ ID NO: 47 - RNA, 22 nt, HOMO SAPIENS
caaccuggaggacuccaugcug

SEQ ID NO: 48 - RNA, 21 nt, HOMO SAPIENS
aucauagaggaaaauccacgu

SEQ ID NO: 49 - RNA, 23 nt, HOMO SAPIENS
gaagugcuucgauuuuggggugu

SEQ ID NO: 50 - RNA, 22 nt, HOMO SAPIENS
uaugugccuuuggacuacaucg

SEQ ID NO: 51 - RNA, 22 nt, HOMO SAPIENS
uaauacugccggguaaugaugg

SEQ ID NO: 52 - RNA, 22 nt, HOMO SAPIENS
acauagaggaaauuccacguuu

SEQ ID NO: 53 - RNA, 23 nt, HOMO SAPIENS
aaugacacgaucacucccguuga

SEQ ID NO: 54 - RNA, 21 nt, HOMO SAPIENS
ucuuguguucucuagaucagu

SEQ ID NO: 55 - RNA, 21 nt, HOMO SAPIENS
cauuauuacuuuugguacgcg

SEQ ID NO: 56 - RNA, 21 nt, HOMO SAPIENS
uagguaguuuccuguuguugg

SEQ ID NO: 57 - RNA, 23 nt, HOMO SAPIENS
uccagcuccuauaugaugccuuu

SEQ ID NO: 58 - RNA, 22 nt, HOMO SAPIENS
aauugcacgguauccaucugua

SEQ ID NO: 59 - RNA, 24 nt, HOMO SAPIENS
guccaguuuucccaggaaucccuu

SEQ ID NO: 60 - RNA, 22 nt, HOMO SAPIENS
cagugcaauguuaaaagggcau

SEQ ID NO: 61 - RNA, 21 nt, HOMO SAPIENS
aacaggugacugguuagacaa

SEQ ID NO: 62 - RNA, 22 nt, HOMO SAPIENS
cuacaaagggaagcacuuucuc

SEQ ID NO: 63 - RNA, 22 nt, HOMO SAPIENS
ucagugcaucacagaacuuugu

SEQ ID NO: 64 - RNA, 22 nt, HOMO SAPIENS
uuuuugcgauguguuccuaaua

SEQ ID NO: 65 - RNA, 23 nt, HOMO SAPIENS
uccgucucaguuacuuuauagcc

SEQ ID NO: 66 - RNA, 22 nt, HOMO SAPIENS
acucaaaaugggggcgcuuucc

SEQ ID NO: 67 - RNA, 21 nt, HOMO SAPIENS
gagugccuucuuuuggagcgu

SEQ ID NO: 68 - RNA, 22 nt, HOMO SAPIENS
uucaaguaauucaggauagguu

SEQ ID NO: 69 - RNA, 21 nt, HOMO SAPIENS
aguuaaugaauccuggaaagu

SEQ ID NO: 70 - RNA, 21 nt, HOMO SAPIENS
uaaagugcugacagugcagau

SEQ ID NO: 71 - RNA, 22 nt, HOMO SAPIENS
caaagcgcuccccuuuagaggu

SEQ ID NO: 72 - RNA, 21 nt, HOMO SAPIENS
cagcagcacacugugguuugu

SEQ ID NO: 73 - RNA, 22 nt, HOMO SAPIENS
ucaggcucaguccccucccgau

SEQ ID NO: 74 - RNA, 22 nt, HOMO SAPIENS
ugagguaguagauuguauaguu

SEQ ID NO: 75 - RNA, 21 nt, HOMO SAPIENS
aauaauacaugguugaucuuu

SEQ ID NO: 76 - RNA, 24 nt, HOMO SAPIENS
cugaagugauguguaacugaucag

SEQ ID NO: 77 - RNA, 21 nt, HOMO SAPIENS
gaaggcgcuucccuuuggagu

SEQ ID NO: 78 - RNA, 23 nt, HOMO SAPIENS
ugauugguacgucuguggguaga

SEQ ID NO: 79 - RNA, 23 nt, HOMO SAPIENS
uaagugcuuccauguuugagugu

SEQ ID NO: 80 - RNA, 22 nt, HOMO SAPIENS
aucgugcauccuuuuagagugu

SEQ ID NO: 81 - RNA, 22 nt, HOMO SAPIENS
uaguaccaguaccuuguguuca

SEQ ID NO: 82 - RNA, 23 nt, HOMO SAPIENS
ugucugcccgcaugccugccucu

SEQ ID NO: 83 - RNA, 23 nt, HOMO SAPIENS
aucgcugcgguugcgagcgcugu

SEQ ID NO: 84 - RNA, 21 nt, HOMO SAPIENS
uugugcuugaucuaaccaugu

SEQ ID NO: 85 - RNA, 23 nt, HOMO SAPIENS
acuuuaacauggaagugcuuucu

SEQ ID NO: 86 - RNA, 21 nt, HOMO SAPIENS
gcgacccauacuugguuucag

SEQ ID NO: 87 - RNA, 22 nt, HOMO SAPIENS
aaaccuguguuguucaagaguc

SEQ ID NO: 88 - RNA, 23 nt, HOMO SAPIENS
aaagugcugcgacauuugagcgu

SEQ ID NO: 89 - RNA, 20 nt, HOMO SAPIENS
ccucugggcccuuccuccag

SEQ ID NO: 90 - RNA, 24 nt, HOMO SAPIENS
aauccuuggaaccuaggugugagu

SEQ ID NO: 91 - RNA, 22 nt, HOMO SAPIENS
uagcagcacaucaugguuuaca

SEQ ID NO: 92 - RNA, 22 nt, HOMO SAPIENS
gucaacacuugcugguuuccuc

SEQ ID NO: 93 - RNA, 22 nt, HOMO SAPIENS
aucaugaugggcuccucggugu

SEQ ID NO: 94 - RNA, 21 nt, HOMO SAPIENS
gaagugugccguggugugucu

SEQ ID NO: 95 - RNA, 22 nt, HOMO SAPIENS
uagguuauccguguugccuucg

SEQ ID NO: 96 - RNA, 23 nt, HOMO SAPIENS
gacacgggcgacagcugcggccc

SEQ ID NO: 97 - RNA, 21 nt, HOMO SAPIENS
ucccacguuguggcccagcag

SEQ ID NO: 98 - RNA, 22 nt, HOMO SAPIENS
agacuucccauuugaagguggc

SEQ ID NO: 99 - RNA, 20 nt, HOMO SAPIENS
guugugucaguuuaucaaac

SEQ ID NO: 100 - RNA, 21 nt, HOMO SAPIENS
uucaaguaauccaggauaggc

SEQ ID NO: 101 - RNA, 23 nt, HOMO SAPIENS
uguaguguuuccuacuuuaugga

SEQ ID NO: 102 - RNA, 23 nt, HOMO SAPIENS
uggagugugacaaugguguuugu

SEQ ID NO: 103 - RNA, 23 nt, HOMO SAPIENS
aaacaaacauggugcacuucuuu

SEQ ID NO: 104 - RNA, 22 nt, HOMO SAPIENS
caucuuaccggacagugcugga

SEQ ID NO: 105 - RNA, 20 nt, HOMO SAPIENS
uagcaccauuugaaaucggu

SEQ ID NO: 106 - RNA, 22 nt, HOMO SAPIENS
uaacagucuacagccauggucg

SEQ ID NO: 107 - RNA, 20 nt, HOMO SAPIENS
agaccauggguucucauugu

SEQ ID NO: 108 - RNA, 19 nt, HOMO SAPIENS
ugagcugcuguaccaaaau

SEQ ID NO: 109 - RNA, 20 nt, HOMO SAPIENS
aggaauguuccuucuuugcc

SEQ ID NO: 110 - RNA, 24 nt, HOMO SAPIENS
ucagaacaaaugccgguucccaga

SEQ ID NO: 111 - RNA, 21 nt, HOMO SAPIENS
augaccuaugaauugacagac

SEQ ID NO: 112 - RNA, 23 nt, HOMO SAPIENS
acuugggcacugaaacaaugucc

SEQ ID NO: 113 - RNA, 22 nt, HOMO SAPIENS
uucaacggguauuuauugagca

SEQ ID NO: 114 - RNA, 22 nt, HOMO SAPIENS
aaucauacacgguugaccuauu

SEQ ID NO: 115 - RNA, 23 nt, HOMO SAPIENS
cacucagccuugagggcacuuuc

SEQ ID NO: 116 - RNA, 23 nt, HOMO SAPIENS
uggcagugucuuagcugguuguu

SEQ ID NO: 117 - RNA, 22 nt, HOMO SAPIENS
acuuguaugcuagcucagguag

SEQ ID NO: 118 - RNA, 22 nt, HOMO SAPIENS
uuaugguuugccugggacugag

SEQ ID NO: 119 - RNA, 23 nt, HOMO SAPIENS
uauggcuuuuuauuccuauguga

SEQ ID NO: 120 - RNA, 23 nt, HOMO SAPIENS
uagcaccauuugaaaucaguguu

SEQ ID NO: 121 - RNA, 85 nt, HOMO SAPIENS
cucaggcugugacccucuagagggaagcacuuucuguugcuugaaagaagagaaagcgcuuccuuuuagaggauuacucuuugag

SEQ ID NO: 122 - RNA, 110 nt, HOMO SAPIENS
aguauaauuauuacauaguuuuugaugucgcagauacugcaucaggaacugauuggauaagaaucagucaccaucaguuccuaaugcauugccuucagcaucuaaacaag

SEQ ID NO: 123 - RNA, 100 nt, HOMO SAPIENS
ugugucucucucuguguccugccagugguuuuacccuaugguagguuacgucaugcuguucuaccacaggguagaaccacggacaggauaccggggcacc

SEQ ID NO: 124 - RNA, 88 nt, HOMO SAPIENS
ucccaugcugugacccucuagaggaagcacuuucuguuuguugucugagaaaaaacaaagugcuucccuuuagaguuacuguuuggga

SEQ ID NO: 125 - RNA, 124 nt, HOMO SAPIENS
aacccuccuugggaagugaagcucaggcugugauuucaagccagggggcguuuuucuauaacuggaugaaaagcaccuccagagcuugaagcucacaguuugagagcaaucgucuaaggaaguu

SEQ ID NO: 126 - RNA, 89 nt, HOMO SAPIENS
gccggcgcccgagcucuggcuccgugucuucacucccgugcuuguccgaggagggagggagggacgggggcugugcuggggcagcugga

SEQ ID NO: 127 - RNA, 77 nt, HOMO SAPIENS
agucuaguuacuaggcaguguaguuagcugauugcuaauaguaccaaucacuaaccacacggccagguaaaaagauu

SEQ ID NO: 128 - RNA, 93 nt, HOMO SAPIENS
ccggggagaaguacggugagccugucauuauucagagaggcuagauccucuguguugagaaggaucaugaugggcuccucgguguucuccagg

SEQ ID NO: 129 - RNA, 85 nt, HOMO SAPIENS
ggcugagccgcaguaguucuucaguggcaagcuuuauguccugacccagcuaaagcugccaguugaagaacuguugcccucugcc

SEQ ID NO: 130 - RNA, 100 nt, HOMO SAPIENS
ucccaucuggacccugcugggcagggcuucugagcuccuuagcacuagcaggaggggcuccaggggcccucccuccauggcagccaggacaggacucuca

SEQ ID NO: 131 - RNA, 67 nt, HOMO SAPIENS
agagaugguagacuauggaacguaggcguuaugauuucugaccuauguaacaugguccacuaacucu

SEQ ID NO: 132 - RNA, 86 nt, HOMO SAPIENS
ugcuucccgaggccacaugcuucuuuauauccccauauggauuacuuugcuauggaauguaaggaagugugugguuucggcaagug

SEQ ID NO: 133 - RNA, 85 nt, HOMO SAPIENS
aggccucucucuccguguucacagcggaccuugauuuaaauguccauacaauuaaggcacgcggugaaugccaagaauggggcug

SEQ ID NO: 134 - RNA, 97 nt, HOMO SAPIENS
cuagauaaguuauuaggugggugcaaagguaauugcaguuuuucccauuauuuuaauugcgaaaacagcaauuaccuuugcaccaaccugauggagu

SEQ ID NO: 135 - RNA, 96 nt, HOMO SAPIENS
guuaugugaagguauucuuguguucucuagaucagugcuuuuagaaaauuugugugaucuaaagaacacaaagaauaccuacacagaaccaccugc

SEQ ID NO: 136 - RNA, 94 nt, HOMO SAPIENS
gcuaggcgugguggcgggcgccugugaucccaacuacucaggaggcuggggcagcagaaucgcuugaacccgggaggcgaagguugcagugagc

SEQ ID NO: 137 - RNA, 76 nt, HOMO SAPIENS
uacuugaagagaaguuguucgugguggauucgcuuuacuuaugacgaaucauucacggacaacacuuuuuucagua

SEQ ID NO: 138 - RNA, 83 nt, HOMO SAPIENS
guggucucagaaucgggguuuugagggcgagaugaguuuauguuuuauccaacuggcccucaaagucccgcuuuuggggucau

SEQ ID NO: 139 - RNA, 86 nt, HOMO SAPIENS
ugcucccucucucacaucccuugcaugguggagggugagcuuucugaaaaccccucccacaugcaggguuugcaggauggcgagcc

SEQ ID NO: 140 - RNA, 87 nt, HOMO SAPIENS
gaaagcgcuuuggaaugacacgaucacucccguugagugggcacccgagaagccaucgggaaugucguguccgcccagugcucuuuc

SEQ ID NO: 141 - RNA, 71 nt, HOMO SAPIENS
ggagaggaggcaagaugcuggcauagcuguugaacugggaaccugcuaugccaacauauugccaucuuucc

SEQ ID NO: 142 - RNA, 71 nt, HOMO SAPIENS
ugcccuagcagcgggaacaguucugcagugagcgaucggugcucugggguauuguuuccgcugccagggua

SEQ ID NO: 143 - RNA, 78 nt, HOMO SAPIENS
uggccgauuuuggcacuagcacauuuuugcuugugucucuccgcucugagcaaucaugugcagugccaauaugggaaa

SEQ ID NO: 144 - RNA, 88 nt, HOMO SAPIENS
accaaguuucaguucauguaaacauccuacacucagcuguaauacauggauuggcugggagguggauguuuacuucagcugacuugga

SEQ ID NO: 145 - RNA, 137 nt, HOMO SAPIENS
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 146 - RNA, 110 nt, HOMO SAPIENS
ccugugcagagauuauuuuuuaaaaggucacaaucaacauucauugcugucgguggguugaacuguguggacaagcucacugaacaaugaaugcaacuguggccccgcuu

SEQ ID NO: 147 - RNA, 87 nt, HOMO SAPIENS
ucucaggcagugacccucuagauggaagcacugucuguuguauaaaagaaaagaucgugcaucccuuuagaguguuacuguuugaga

SEQ ID NO: 148 - RNA, 122 nt, HOMO SAPIENS
gcccuguccccugugccuugggcgggcggcuguuaagacuugcagugauguuuaacuccucuccacgugaacaucacagcaagucugugcugcuucccgucccuacgcugccugggcagggu

SEQ ID NO: 149 - RNA, 95 nt, HOMO SAPIENS
cggccggcccuggguccaucuuccaguacaguguuggauggucuaauugugaagcuccuaacacugucugguaaagauggcucccggguggguuc

SEQ ID NO: 150 - RNA, 97 nt, HOMO SAPIENS
aaucuaucacugcuuuuuaggauaagcuugacuuuuguucaaauaaaaaugcaaaaggaaaguguauccuaaaaggcaaugacaguuuaauguguuu

SEQ ID NO: 151 - RNA, 99 nt, HOMO SAPIENS
gaaacugggcucaaggugaggggugcuaucugugauugagggacaugguuaauggaauugucucacacagaaaucgcacccgucaccuuggccuacuua

SEQ ID NO: 152 - RNA, 97 nt, HOMO SAPIENS
uacuuacucuacgugugugucacucgaugaccacugugaagacaguaaaauguacagugguucucuuguggcucaagcguaauguagaguacugguc

SEQ ID NO: 153 - RNA, 72 nt, HOMO SAPIENS
ggaucuuuuugcggucugggcuugcuguuccucucaacaguagucaggaagcccuuaccccaaaaaguaucu

SEQ ID NO: 154 - RNA, 78 nt, HOMO SAPIENS
uacugcccucggcuucuuuacagugcugccuuguugcauauggaucaagcagcauuguacagggcuaugaaggcauug

SEQ ID NO: 155 - RNA, 97 nt, HOMO SAPIENS
ugugaucacugucuccagccugcugaagcucagagggcucugauucagaaagaucaucggauccgucugagcuuggcuggucggaagucucaucauc

SEQ ID NO: 156 - RNA, 98 nt, HOMO SAPIENS
auacagugcuugguuccuaguagguguccaguaaguguuugugacauaauuuguuuauugaggaccuccuaucaaucaagcacugugcuaggcucugg

SEQ ID NO: 157 - RNA, 87 nt, HOMO SAPIENS
ucucaggcugugucccucuagagggaagcgcuuucuguugucugaaagaaaagaaaaugguucccuuuagaguguuacgcuuugaga

SEQ ID NO: 158 - RNA, 87 nt, HOMO SAPIENS
ucucagccugugacccucuagagggaagcgcuuucuguugucugaaagaaaagaaagugcaucuuuuuagaggauuacaguuugaga

SEQ ID NO: 159 - RNA, 71 nt, HOMO SAPIENS
guagcacuaaagugcuuauagugcagguaguguuuaguuaucuacugcauuaugagcacuuaaaguacugc

SEQ ID NO: 160 - RNA, 100 nt, HOMO SAPIENS
caguccuucuuugguauuuaaaacguggauauuccuucuauguuuacgugauuccugguuaaucauagaggaaaauccauguuuucaguaucaaaugcug

SEQ ID NO: 161 - RNA, 94 nt, HOMO SAPIENS
gaguuugguuuuguuuggguuuguucuagguauggucccagggaucccagaucaaaccaggccccugggccuauccuagaaccaaccuaagcuc

SEQ ID NO: 162 - RNA, 98 nt, HOMO SAPIENS
aagucacgugcuguggcuccagcuucauaggaaggcucuugucugucaggcaguggaguuacuuacagacaagagccuugcucaggccagcccugccc

SEQ ID NO: 163 - RNA, 81 nt, HOMO SAPIENS
gggcuuucaagucacuagugguuccguuuaguagaugauugugcauuguuucaaaauggugcccuagugacuacaaagccc

SEQ ID NO: 164 - RNA, 77 nt, HOMO SAPIENS
ccgggacccaguucaaguaauucaggauagguugugugcuguccagccuguucuccauuacuuggcucggggaccgg

SEQ ID NO: 165 - RNA, 97 nt, HOMO SAPIENS
ugaugcuuugcuggcuggugcagugccugagggaguaagagcccuguuguuguaagauagugucuuacucccucaggcacaucuccaacaagucucu

SEQ ID NO: 166 - RNA, 97 nt, HOMO SAPIENS
ugaccugaaucagguaggcaguguauuguuagcuggcugcuugggucaagucagcagccacaacuacccugccacuugcuucuggauaaauucuucu

SEQ ID NO: 167 - RNA, 97 nt, HOMO SAPIENS
accaagugauauucauugucuaccugagcuagaauacaaguaguuggcgucuucagagacacuuguaugcuagcucagguagauauugaaugaaaaa

SEQ ID NO: 168 - RNA, 68 nt, HOMO SAPIENS
cuucaauuuuauuuuaaaacggugagauuuuguuuugucugagaaaaucucgcuguuuuagacugagg

SEQ ID NO: 169 - RNA, 95 nt, HOMO SAPIENS
uccccucuggcggcugcgcacgggccgugugagcuauugcggugggcuggggcagaugacgcgugcgccggccggccgccgaggggcuaccguuc

SEQ ID NO: 170 - RNA, 82 nt, HOMO SAPIENS
gcuucgcuccccuccgccuucucuucccgguucuucccggagucgggaaaagcuggguugagagggcgaaaaaggaugaggu

SEQ ID NO: 171 - RNA, 87 nt, HOMO SAPIENS
ucucgggcugugacucuccaaagggaagaauuuucucuugucuaaaagaaaagaacgcacuucccuuuagaguguuaccgugugaga

SEQ ID NO: 172 - RNA, 71 nt, HOMO SAPIENS
uguucuaaggugcaucuagugcagauagugaaguagauuagcaucuacugcccuaagugcuccuucuggca

SEQ ID NO: 173 - RNA, 97 nt, HOMO SAPIENS
ucccuuucccaggggaggggcuggguuuacguugggagaacuuuuacggugaaccaggagguucucccaacguaagcccagccccuccccucugccu

SEQ ID NO: 174 - RNA, 75 nt, HOMO SAPIENS
uguugucggguggaucacgaugcaauuuugaugaguaucauaggagaaaaauugcacgguauccaucuguaaacc

SEQ ID NO: 175 - RNA, 96 nt, HOMO SAPIENS
agagaagcuggacaaguacuggucucagcagauugaggagagcaccacaguggucaucacacagucugcugagguuggagcugcugagaugacacu

SEQ ID NO: 176 - RNA, 97 nt, HOMO SAPIENS
uagccagucagaaaugagcuuauucauaaaagugcaguauggugaagucaaucuguaauuuuauguauaagcuagucucugauugaaacaugcagca

SEQ ID NO: 177 - RNA, 96 nt, HOMO SAPIENS
ucccuggcgugaggguaugugccuuuggacuacaucguggaagccagcaccaugcaguccaugggcauauacacuugccucaaggccuaugucauc

SEQ ID NO: 178 - RNA, 82 nt, HOMO SAPIENS
ugguaccugaaaagaaguugcccauguuauuuucgcuuuauaugugacgaaacaaacauggugcacuucuuuuucgguauca

SEQ ID NO: 179 - RNA, 84 nt, HOMO SAPIENS
guggcagcuugguggucguaugugugacgccauuuacuugaaccuuuaggagugacaucacauauacggcagcuaaacugcuac

SEQ ID NO: 180 - RNA, 89 nt, HOMO SAPIENS
accagacuuuuccuagucccugagacccuaacuugugagguauuuuaguaacaucacaagucaggcucuugggaccuaggcggagggga

SEQ ID NO: 181 - RNA, 75 nt, HOMO SAPIENS
ucaucccuggguggggauuuguugcauuacuuguguucuauauaaaguauugcacuugucccggccuguggaaga

SEQ ID NO: 182 - RNA, 83 nt, HOMO SAPIENS
ucaugcuguggcccuccagagggaagcgcuuucuguugucugaaagaaaacaaagcgcuccccuuuagagguuuacgguuuga

SEQ ID NO: 183 - RNA, 87 nt, HOMO SAPIENS
ucucaggcugucguccucuagagggaagcacuuucuguugucugaaagaaaagaaagugcuuccuuuuagaggguuaccguuugaga

SEQ ID NO: 184 - RNA, 87 nt, HOMO SAPIENS
ccuaggaagagguaguagguugcauaguuuuagggcagggauuuugcccacaaggagguaacuauacgaccugcugccuuucuuagg

SEQ ID NO: 185 - RNA, 72 nt, HOMO SAPIENS
agguugagguaguagguuguauaguuuagaauuacaucaagggagauaacuguacagccuccuagcuuuccu

SEQ ID NO: 186 - RNA, 87 nt, HOMO SAPIENS
ucucaugcugugacccucuagagggaagcgcuuucuguugucugaaagaaaagaacgcgcuucccuauagaggguuacccuuugaga

SEQ ID NO: 187 - RNA, 97 nt, HOMO SAPIENS
cugcuccuucucccauacccauugcauaucggaguugugaauucucaaaacaccuccugugugcauggauuacaggagggugagccuugucaucgug

SEQ ID NO: 188 - RNA, 97 nt, HOMO SAPIENS
cuucauccaccaguccuccaggaacaucaaggaucuuaaacuuugccagagcuacaaaggcaaaguuuaagauccuugaaguuccugggggaaccau

SEQ ID NO: 189 - RNA, 87 nt, HOMO SAPIENS
ucucaggcugugucccucuacagggaagcgcuuucuguugucugaaagaaaggaaagugcauccuuuuagaguguuacuguuugaga

SEQ ID NO: 190 - RNA, 95 nt, HOMO SAPIENS
ccagcucgggcagccguggccaucuuacugggcagcauuggauggagucaggucucuaauacugccugguaaugaugacggcggagcccugcacg

SEQ ID NO: 191 - RNA, 75 nt, HOMO SAPIENS
ggcugugccggguagagagggcagugggagguaagagcucuucacccuucaccaccuucuccacccagcauggcc

SEQ ID NO: 192 - RNA, 110 nt, HOMO SAPIENS
agaagggcuaucaggccagccuucagaggacuccaaggaacauucaacgcugucggugaguuugggauuugaaaaaaccacugaccguugacuguaccuugggguccuua

SEQ ID NO: 193 - RNA, 84 nt, HOMO SAPIENS
uugacuuagcuggguaguggggaacccuuccaugaggaguagaacacuccuuaugcaagauucccuucuaccuggcuggguugg

SEQ ID NO: 194 - RNA, 84 nt, HOMO SAPIENS
aggcugagguaguaguuuguacaguuugagggucuaugauaccacccgguacaggagauaacuguacaggccacugccuugcca

SEQ ID NO: 195 - RNA, 64 nt, HOMO SAPIENS
augacugauuucuuuugguguucagagucaauauaauuuucuagcaccaucugaaaucgguuau

SEQ ID NO: 196 - RNA, 98 nt, HOMO SAPIENS
uacaauccaacgaggauucuaauuucuccacgucuuugguaauaagguuuggcaaagauguggaaaaauuggaauccucauucgauugguuauaacca

SEQ ID NO: 197 - RNA, 110 nt, HOMO SAPIENS
guguuggggacucgcgcgcuggguccagugguucuuaacaguucaacaguucuguagcgcaauugugaaauguuuaggaccacuagacccggcgggcgcggcgacagcga

SEQ ID NO: 198 - RNA, 68 nt, HOMO SAPIENS
guguauucuacagugcacgugucuccaguguggcucggaggcuggagacgcggcccuguuggaguaac

SEQ ID NO: 199 - RNA, 96 nt, HOMO SAPIENS
aggaaguguuggccuguggcugcacucacuuccuucagccccaggaagccuuggucgggggcaggagggagggucaggcagggcugggggccugac

SEQ ID NO: 200 - RNA, 98 nt, HOMO SAPIENS
acgaauggcuaugcacugcacaacccuaggagagggugccauucacauagacuauaauugaauggcgccacuaggguugugcagugcacaaccuacac

SEQ ID NO: 201 - RNA, 85 nt, HOMO SAPIENS
ugcaggccucugugugauauguuugauauauuagguuguuauuuaauccaacuauauaucaaacauauuccuacagugucuugcc

SEQ ID NO: 202 - RNA, 85 nt, HOMO SAPIENS
gcacauuguaggccucauuaaauguuuguugaaugaaaaaaugaaucaucaacagacauuaauugggcgccugcucugugaucuc

SEQ ID NO: 203 - RNA, 99 nt, HOMO SAPIENS
uccagccugugcccagcagccccugagaaccacgucugcucugagcuggguacugccuguucagaacaaaugccgguucccagacgcugccagcuggcc

SEQ ID NO: 204 - RNA, 110 nt, HOMO SAPIENS
ugaacauccaggucuggggcaugaaccuggcauacaauguagauuucuguguucguuaggcaacagcuacauugucugcuggguuucaggcuaccuggaaacauguucuc

SEQ ID NO: 205 - RNA, 100 nt, HOMO SAPIENS
gugagcgggcgcggcagggaucgcgggcggguggcggccuagggcgcggagggcggaccgggaauggcgcgccgugcgccgccggcguaacugcggcgcu

SEQ ID NO: 206 - RNA, 97 nt, HOMO SAPIENS
cauuggcaucuauuagguuggugcaaaaguaauugcgguuuuugccauuacuuucaguagcaaaaaucucaauuacuuuugcaccaacuuaauacuu

SEQ ID NO: 207 - RNA, 101 nt, HOMO SAPIENS
ccgcccccgcgucuccagggcaaccguggcuuucgauuguuacugugggaacuggagguaacagucuacagccauggucgccccgcagcacgcccacgcgc

SEQ ID NO: 208 - RNA, 70 nt, HOMO SAPIENS
ggcacccacccguagaaccgaccuugcggggccuucgccgcacacaagcucgugucuguggguccguguc

SEQ ID NO: 209 - RNA, 96 nt, HOMO SAPIENS
gggaccugcgugggugcgggcgugugagugugugugugugagugugugucgcuccggguccacgcucaugcacacacccacacgcccacacucagg

SEQ ID NO: 210 - RNA, 82 nt, HOMO SAPIENS
gcaguccucuguuaguuuugcauaguugcacuacaagaagaauguaguugugcaaaucuaugcaaaacugaugguggccugc

SEQ ID NO: 211 - RNA, 90 nt, HOMO SAPIENS
ucucaggcugugaccaucuggagguaagaagcacuuucuguuuugugaaagaaaagaaagugcuuccuuucagaggguuacucuuugaga

SEQ ID NO: 212 - RNA, 91 nt, HOMO SAPIENS
cugggguacggggauggauggucgaccaguuggaaaguaauuguuucuaauguacuucaccugguccacuagccguccguauccgcugcag

SEQ ID NO: 213 - RNA, 68 nt, HOMO SAPIENS
ccucuacuuuaacauggaggcacuugcugugacaugacaaaaauaagugcuuccauguuugagugugg

SEQ ID NO: 214 - RNA, 90 nt, HOMO SAPIENS
cucacagcugccagugucauuuuugugaucugcagcuaguauucucacuccaguugcauagucacaaaagugaucauuggcagguguggc

SEQ ID NO: 215 - RNA, 116 nt, HOMO SAPIENS
caacuacagccacuacuacaggaccaucgaggaccugcgggacaagauucuuggugccaccauugagaacgccaggauuguccugcagaucaacaaugcucaacuggcugcagaug

SEQ ID NO: 216 - RNA, 109 nt, HOMO SAPIENS
aucaagauuagaggcucugcucuccguguucacagcggaccuugauuuaaugucauacaauuaaggcacgcggugaaugccaagagcggagccuacggcugcacuugaa

SEQ ID NO: 217 - RNA, 84 nt, HOMO SAPIENS
ucucagucuguggcacucagccuugagggcacuuucuggugccagaaugaaagugcugucauagcugagguccaaugacugagg

SEQ ID NO: 218 - RNA, 65 nt, HOMO SAPIENS
cuguuaaugcuaaucgugauagggguuuuugccuccaacugacuccuacauauuagcauuaacag

SEQ ID NO: 219 - RNA, 69 nt, HOMO SAPIENS
gggauacucaaaaugggggcgcuuuccuuuuugucuguacugggaagugcuucgauuuugggguguccc

SEQ ID NO: 220 - RNA, 96 nt, HOMO SAPIENS
aaccauucaaauauaccacaguuuguuuaaccuuuugccuguugguugaagaugccuuucaacaggugacugguuagacaaacugugguauauaca

SEQ ID NO: 221 - RNA, 84 nt, HOMO SAPIENS
ggcuguggcuggauucaaguaauccaggauaggcuguuuccaucugugaggccuauucuugauuacuuguuucuggaggcagcu

SEQ ID NO: 222 - RNA, 110 nt, HOMO SAPIENS
gauggcugugaguuggcuuaaucucagcuggcaacugugagauguucauacaaucccucacaguggucucugggauuaugcuaaacagagcaauuuccuagcccucacga

SEQ ID NO: 223 - RNA, 100 nt, HOMO SAPIENS
cccccagaaucugucaggcaccagccaggcauugcucagcccguuucccucugggggagcaaggaguggugcuggguuugucucugcugggguuucuccu

SEQ ID NO: 224 - RNA, 85 nt, HOMO SAPIENS
cucaagcugugacucuccagagggaugcacuuucucuuaugugaaaaaaaagaaggcgcuucccuuuagagcguuacgguuuggg

SEQ ID NO: 225 - RNA, 90 nt, HOMO SAPIENS
aggccucgcuguucucuauggcuuuuuauuccuaugugauucuacugcucacucauauagggauuggagccguggcgcacggcggggaca

SEQ ID NO: 226 - RNA, 96 nt, HOMO SAPIENS
uagauugaggaaggggcugagugguaggcggugcugcugugcucugaugaagacccauguggcuagcaacagcgcuuaccuuuugucucugggucc

SEQ ID NO: 227 - RNA, 97 nt, HOMO SAPIENS
ugugauguguauuagguuugugcaaaaguaauugggguuuuuugccguuaaaaguaauggcaaaacuggcaauuacuuuugcaccaaacuaauauaa

SEQ ID NO: 228 - RNA, 84 nt, HOMO SAPIENS
ggccaguguugagaggcggagacuugggcaauugcuggacgcugcccugggcauugcacuugucucggucugacagugccggcc

SEQ ID NO: 229 - RNA, 72 nt, HOMO SAPIENS
cuugggaauggcaaggaaaccguuaccauuacugaguuuaguaaugguaaugguucucuugcuauacccaga

SEQ ID NO: 230 - RNA, 88 nt, HOMO SAPIENS
caccuuguccucacgguccaguuuucccaggaaucccuuagaugcuaagauggggauuccuggaaauacuguucuugaggucaugguu

SEQ ID NO: 231 - RNA, 69 nt, HOMO SAPIENS
ccaccacuuaaacguggauguacuugcuuugaaacuaaagaaguaagugcuuccauguuuuggugaugg

SEQ ID NO: 232 - RNA, 97 nt, HOMO SAPIENS
aaacaaguuauauuagguuggugcaaaaguaauugugguuuuugccuguaaaaguaauggcaaaaaccacaguuucuuuugcaccagacuaauaaag

SEQ ID NO: 233 - RNA, 110 nt, HOMO SAPIENS
cuggauacagaguggaccggcuggccccaucuggaagacuagugauuuuguuguugucuuacugcgcucaacaacaaaucccagucuaccuaauggugccagccaucgca

SEQ ID NO: 234 - RNA, 94 nt, HOMO SAPIENS
gugcuguguguagugcuucacuucaagaagugccaugcaugugucuagaaauauguuuugcaccuuuuggagugaaauaaugcacaacagauac

SEQ ID NO: 235 - RNA, 86 nt, HOMO SAPIENS
gucugucugcccgcaugccugccucucuguugcucugaaggaggcaggggcugggccugcagcugccugggcagagcggcuccugc

SEQ ID NO: 236 - RNA, 100 nt, HOMO SAPIENS
gcucgguugccgugguugcgggcccugcccgcccgccagcucgcugacagcacgacucagggcggagggaaguagguccguuggucggucgggaacgagg

SEQ ID NO: 237 - RNA, 98 nt, HOMO SAPIENS
gguacuucucagucuguggcacucagccuugagggcacuuucuggugccagaaugaaagugcugucauagcugagguccaaugacugaggcgagcacc

SEQ ID NO: 238 - RNA, 110 nt, HOMO SAPIENS
ucaccuggccaugugacuugugggcuucccuuugucauccuucgccuagggcucugagcagggcagggacagcaaaggggugcucaguugucacuucccacagcacggag

SEQ ID NO: 239 - RNA, 96 nt, HOMO SAPIENS
acauugcuacuuacaauuaguuuugcagguuugcauuucagcguauauauguauauguggcugugcaaauccaugcaaaacugauugugauaaugu

SEQ ID NO: 240 - RNA, 67 nt, HOMO SAPIENS
gugacccucuagauggaagcacugucuguugucuaagaaaagaucgugcaucccuuuagaguguuac

SEQ ID NO: 241 - RNA, 87 nt, HOMO SAPIENS
gacagugcagucacccauaaaguagaaagcacuacuaacagcacuggaggguguaguguuuccuacuuuauggaugaguguacugug

SEQ ID NO: 242 - RNA, 70 nt, HOMO SAPIENS
uugaagggagaucgaccguguuauauucgcuuuauugacuucgaauaauacaugguugaucuuuucucag

SEQ ID NO: 243 - RNA, 96 nt, HOMO SAPIENS
acggaagccugcacgcauuuaacaccagcacgcucaauguagucuuguaaggaacagguugaagugugccguggugugucuggaggaagcgccugu

SEQ ID NO: 244 - RNA, 97 nt, HOMO SAPIENS
uauuaugccaugacauugugucaauaugcgaugauguguugugauggcacagcgucaucacguggugacgcaacaucaugacguaagacgucacaac

SEQ ID NO: 245 - RNA, 87 nt, HOMO SAPIENS
ucccaugcugugacccucuagagggaagcacuuucuguugucugaaagaaaccaaagcgcuucccuuuggagcguuacgguuugaga

SEQ ID NO: 246 - RNA, 68 nt, HOMO SAPIENS
guauccuguacugagcugccccgagcugggcagcaugaagggccucggggcagcucaguacaggaugc

SEQ ID NO: 247 - RNA, 99 nt, HOMO SAPIENS
ccgauguguauccucagcuuugagaacugaauuccauggguugugucagugucagaccucugaaauucaguucuucagcugggauaucucugucaucgu

SEQ ID NO: 248 - RNA, 78 nt, HOMO SAPIENS
auacuugaggagaaauuauccuugguguguucgcuuuauuuaugaugaaucauacaaggacaauuucuuuuugaguau

SEQ ID NO: 249 - RNA, 83 nt, HOMO SAPIENS
ucucaugcagucauucuccaaaagaaagcacuuucuguugucugaaagcagagugccuucuuuuggagcguuacuguuugaga

SEQ ID NO: 250 - RNA, 80 nt, HOMO SAPIENS
cugggggcuccaaagugcuguucgugcagguagugugauuacccaaccuacugcugagcuagcacuucccgagcccccgg

SEQ ID NO: 251 - RNA, 96 nt, HOMO SAPIENS
gcuccaguaacaucuuaaaguaaauaugcaccaaaauuacuuuugguaaauacaguuuuggugcauauuuacuuuaggauguuacuggagcuccca

SEQ ID NO: 252 - RNA, 96 nt, HOMO SAPIENS
uguauccuugguuuuuaguaguuuuacuaugaugaggugugccauccaccccaucauaguaaacuacugaaaaucaaagauacaagugccugacca

SEQ ID NO: 253 - RNA, 69 nt, HOMO SAPIENS
aguaccaaagugcucauagugcagguaguuuuggcaugacucuacuguaguaugggcacuuccaguacu

SEQ ID NO: 254 - RNA, 77 nt, HOMO SAPIENS
agcacggccucuccgaagccugcccggcuccucgggaaccugccucccgcauggcagcugcugcccuucggaggccg

SEQ ID NO: 255 - RNA, 84 nt, HOMO SAPIENS
cuggcugagguaguaguuugugcuguuggucggguugugacauugcccgcuguggagauaacugcgcaagcuacugccuugcua

SEQ ID NO: 256 - RNA, 86 nt, HOMO SAPIENS
ugcucccccucucuaauccuugcuaucugggugcuagugcuggcucaaugcaaugcaccugggcaaggauucagagagggggagcu

SEQ ID NO: 257 - RNA, 98 nt, HOMO SAPIENS
agaaugggcaaaugaacaguaaauuuggaggccuggggcccucccugcugcuggagaaguguuugcacgggugggccuugucuuugaaaggaggugga

SEQ ID NO: 258 - RNA, 97 nt, HOMO SAPIENS
acucaggggcuucgccacugauuguccaaacgcaauucuuguacgagucugcggccaaccgagaauuguggcuggacaucuguggcugagcuccggg

SEQ ID NO: 259 - RNA, 97 nt, HOMO SAPIENS
aaacccacaccacugcauuuuggccaucgaggguuggggcuuggugucaugccccaagauaaccagcaccccaacuuuggacagcauggauuagucu

SEQ ID NO: 260 - RNA, 81 nt, HOMO SAPIENS
gauacucgaaggagagguuguccguguugucuucucuuuauuuaugaugaaacauacacgggaaaccucuuuuuuaguauc

SEQ ID NO: 261 - RNA, 90 nt, HOMO SAPIENS
uuuccugcccucgaggagcucacagucuaguaugucucauccccuacuagacugaagcuccuugaggacagggauggucauacucaccuc

SEQ ID NO: 262 - RNA, 87 nt, HOMO SAPIENS
caauagacacccaucgugucuuuugcucugcagucaguaaauauuuuuuugugaauguguagcaaaagacagaauggugguccauug

SEQ ID NO: 263 - RNA, 114 nt, HOMO SAPIENS
uccugcuuguccugcgaggugucuugcaggccgucaugcaggccacacugacgguaacguugcaggucgucuugcagggcuucucgcaagacgacauccucaucaccaacgacg

SEQ ID NO: 264 - RNA, 67 nt, HOMO SAPIENS
guggcacucaaacugugggggcacuuucugcucucuggugaaagugccgccaucuuuugaguguuac

SEQ ID NO: 265 - RNA, 68 nt, HOMO SAPIENS
ccuuugcuuuaacauggggguaccugcugugugaaacaaaaguaagugcuuccauguuucaguggagg

SEQ ID NO: 266 - RNA, 97 nt, HOMO SAPIENS
auaaaauuuccaauuggaaccuaaugauucaucagacucagauauuuaaguuaacaguauuugagaaugaugaaucauuagguuccggucagaaauu

SEQ ID NO: 267 - RNA, 110 nt, HOMO SAPIENS
cggaaaauuugccaaggguuugggggaacauucaaccugucggugaguuugggcagcucaggcaaaccaucgaccguugaguggacccugaggccuggaauugccauccu

SEQ ID NO: 268 - RNA, 83 nt, HOMO SAPIENS
cgccggccgaugggcgucuuaccagacaugguuagaccuggcccucugucuaauacugucugguaaaaccguccauccgcugc

SEQ ID NO: 269 - RNA, 75 nt, HOMO SAPIENS
uacuuaaagcgagguugcccuuuguauauucgguuuauugacauggaauauacaagggcaagcucucugugagua

SEQ ID NO: 270 - RNA, 97 nt, HOMO SAPIENS
aucugaguugggagggucccucuccaaaugugucuuggggugggggaucaagacacauuuggagagggaaccucccaacucggccucugccaucauu

SEQ ID NO: 271 - RNA, 97 nt, HOMO SAPIENS
ccaguggcgcaauggauaacgcgucugacuacggaucagaagauucuagguucgacuccuggcuggcucgcgaugucuguuuugccacacuugaccc

SEQ ID NO: 272 - RNA, 110 nt, HOMO SAPIENS
gaucugucugucuucuguauauacccuguagauccgaauuuguguaaggaauuuuguggucacaaauucguaucuaggggaauauguaguugacauaaacacuccgcucu

SEQ ID NO: 273 - RNA, 95 nt, HOMO SAPIENS
cucaucugucuguugggcuggaggcagggccuuugugaaggcggguggugcucagaucgccucugggcccuuccuccagccccgaggcggauuca

SEQ ID NO: 274 - RNA, 101 nt, HOMO SAPIENS
gcgagaagaucucaugcugugacucucuggagggaagcacuuucuguugucugaaagaaaacaaagcgcuucucuuuagaguguuacgguuugagaaaagc

SEQ ID NO: 275 - RNA, 87 nt, HOMO SAPIENS; identical to SEQ ID NO: 262

SEQ ID NO: 276 - RNA, 98 nt, HOMO SAPIENS
acccaaacccuaggucugcugacuccuaguccagggcucgugauggcuggugggcccugaacgaggggucuggaggccuggguuugaauaucgacagc

SEQ ID NO: 277 - RNA, 84 nt, HOMO SAPIENS
gugcucgguuuguaggcagugucauuagcugauuguacuguggugguuacaaucacuaacuccacugccaucaaaacaaggcac

SEQ ID NO: 278 - RNA, 68 nt, HOMO SAPIENS
cuccggugccuacugagcugauaucaguucucauuuuacacacuggcucaguucagcaggaacaggag

SEQ ID NO: 279 - RNA, 97 nt, HOMO SAPIENS
aaugcuguuucaagguaguaccaguaccuuguguucaguggaaccaagguaaacacaagguauugguauuaccuugagauagcauuacaccuaagug

SEQ ID NO: 280 - RNA, 96 nt, HOMO SAPIENS
agaugugcucuccuggcccaugaaaucaagcgugggugagaccuggugcagaacgggaaggcgacccauacuugguuucagaggcugugagaauaa

SEQ ID NO: 281 - RNA, 82 nt, HOMO SAPIENS
ugagcccucggaggacuccauuuguuuugaugauggauucuuaugcuccaucaucgucucaaaugagucuucagaggguucu

SEQ ID NO: 282 - RNA, 110 nt, HOMO SAPIENS
uuggauguuggccuaguucuguguggaagacuagugauuuuguuguuuuuagauaacuaaaucgacaacaaaucacagucugccauauggcacaggccaugccucuacag

SEQ ID NO: 283 - RNA, 83 nt, HOMO SAPIENS
gcugcuguugggagacccuggucugcacucuaucuguauucuuacugaagggagugcagggcaggguuucccauacagagggc

SEQ ID NO: 284 - RNA, 128 nt, HOMO SAPIENS
uggaggccuugcugguuuggaaaguucauuguucgacaccauggaucuccaggugggucaaguuuagagaugcaccaaccuggaggacuccaugcuguugagcuguucacaagcagcggacacuucca

SEQ ID NO: 285 - RNA, 80 nt, HOMO SAPIENS
ugggaugagguaguagguuguauaguuuuagggucacacccaccacugggagauaacuauacaaucuacugucuuuccua

SEQ ID NO: 286 - RNA, 110 nt, HOMO SAPIENS
gcugcuggaagguguagguacccucaauggcucaguagccaguguagauccugucuuucguaaucagcagcuacaucuggcuacugggucucugauggcaucuucuagcu

SEQ ID NO: 287 - RNA, 83 nt, HOMO SAPIENS
cggggugagguaguagguugugugguuucagggcagugauguugccccucggaagauaacuauacaaccuacugccuucccug

SEQ ID NO: 288 - RNA, 97 nt, HOMO SAPIENS
caucauaaggagccuagacuucccauuugaagguggccauuuccuaccaccuucaaaugguaaguccaggcuccuucugauucaauaaaugaggagc

SEQ ID NO: 289 - RNA, 72 nt, HOMO SAPIENS
ugucggguagcuuaucagacugauguugacuguugaaucucauggcaacaccagucgaugggcugucugaca

SEQ ID NO: 290 - RNA, 95 nt, HOMO SAPIENS
auagcuguugugucacuuccucaugcugacauauuuacuagaggguaaaauuaauaaccuucuaguaagaguggcagucgaagggaagggcucau

SEQ ID NO: 291 - RNA, 99 nt, HOMO SAPIENS
ugggugaaaggaaggaaagacauaggauagagucaccucuguccucuguccucuaccuauagaggugacuguccuaugucuuuccuuccucuuaccccu

SEQ ID NO: 292 - RNA, 69 nt, HOMO SAPIENS
uugagcagagguugcccuuggugaauucgcuuuauuuauguugaaucacacaaaggcaacuuuuguuug

SEQ ID NO: 293 - RNA, 98 nt, HOMO SAPIENS
aacauguugucugugguacccuacucuggagagugacaaucauguauaauuaaauuugauugacacuucugugaguagaguaacgcaugacacguacg

SEQ ID NO: 294 - RNA, 84 nt, HOMO SAPIENS
ccagucacguccccuuaucacuuuuccagcccagcuuugugacuguaaguguuggacggagaacugauaaggguaggugauuga

SEQ ID NO: 295 - RNA, 73 nt, HOMO SAPIENS
ggccggcugggguuccuggggaugggauuugcuuccugucacaaaucacauugccagggauuuccaaccgacc

SEQ ID NO: 296 - RNA, 110 nt, HOMO SAPIENS
ccagagguuguaacguugucuauauauacccuguagaaccgaauuugugugguauccguauagucacagauucgauucuaggggaauauauggucgaugcaaaaacuuca

SEQ ID NO: 297 - RNA, 97 nt, HOMO SAPIENS
aacuaugcaaggauauuugaggagagguuauccguguuauguucgcuucauucaucaugaauaauacaugguuaaccucuuuuugaauaucagacuc

SEQ ID NO: 298 - RNA, 83 nt, HOMO SAPIENS
agcuuagguaccaauuuggccacaauggguuagaacacuauuccauuguguucuuacccaccauggccaaaauugggccuaag

SEQ ID NO: 299 - RNA, 97 nt, HOMO SAPIENS
cucaggugcucuggcugcuuggguuccuggcaugcugauuugugacuuaagauuaaaaucacauugccagggauuaccacgcaaccacgaccuuggc

SEQ ID NO: 300 - RNA, 94 nt, HOMO SAPIENS
gaucaggagucugccaguggagucagcacaccugcuuuucaccugugaucccaggagaggaagcagcugccucugaggccucaggcucaguggc

SEQ ID NO: 301 - RNA, 94 nt, HOMO SAPIENS
cgggcagcgggugccaggcacggugucagcaggcaacauggccgagaggccggggccuccgggcggcgccguguccgcgaccgcguacccugac

SEQ ID NO: 302 - RNA, 96 nt, HOMO SAPIENS
gcgggcggccccgcggugcauugcuguugcauugcacgugugugaggcgggugcagugccucggcagugcagcccggagccggccccuggcaccac

SEQ ID NO: 303 - RNA, 90 nt, HOMO SAPIENS
ucucaggcugugaccuucucgaggaaagaagcacuuucuguugucugaaagaaaagaaagugcuuccuuucagaggguuacgguuugaga

SEQ ID NO: 304 - RNA, 97 nt, HOMO SAPIENS
uagggugaccagccauuaugguuugccugggacugaggaauuugcugggauaugucaguuccaggccaaccaggcugguuggucucccugaagcaac

SEQ ID NO: 305 - RNA, 75 nt, HOMO SAPIENS
ugcccuggcucaguuaucacagugcugaugcugucuauucuaaagguacaguacugugauaacugaaggauggca

SEQ ID NO: 306 - RNA, 87 nt, HOMO SAPIENS
agcgguggccagugucauuuuugugauguugcagcuaguaauaugagcccaguugcauagucacaaaagugaucauuggaaacugug

SEQ ID NO: 307 - RNA, 68 nt, HOMO SAPIENS
ccauuacuguugcuaauaugcaacucuguugaauauaaauuggaauugcacuuuagcaauggugaugg

SEQ ID NO: 308 - RNA, 72 nt, HOMO SAPIENS
agauacuguaaacauccuacacucucagcuguggaaaguaagaaagcugggagaaggcuguuuacucuuucu

SEQ ID NO: 309 - RNA, 83 nt, HOMO SAPIENS
gcccuagcuugguucuaaaucccauggugccuucuccuugggaaaaacagagaaggcacuaugagauuuagaaucaaguuagg

SEQ ID NO: 310 - RNA, 87 nt, HOMO SAPIENS
accgcagggaaaaugagggacuuuugggggcagauguguuuccauuccacuaucauaaugccccuaaaaauccuuauugcucuugca

SEQ ID NO: 311 - RNA, 110 nt, HOMO SAPIENS
agauuagaguggcuguggucuagugcuguguggaagacuagugauuuuguuguucugauguacuacgacaacaagucacagccggccucauagcgcagacucccuucgac

SEQ ID NO: 312 - RNA, 96 nt, HOMO SAPIENS
gguauuguuagauuaauuuugugggacauuaacaacagcaucagaagcaacaucagcuuuaguuaaugaauccuggaaaguuaagugacuuuauuu

SEQ ID NO: 313 - RNA, 110 nt, HOMO SAPIENS
ggcuacagucuuucuucaugugacucguggacuucccuuugucauccuaugccugagaauauaugaaggaggcugggaaggcaaagggacguucaauugucaucacuggc

SEQ ID NO: 314 - RNA, 84 nt, HOMO SAPIENS
gugguacuugaagauagguuauccguguugccuucgcuuuauuugugacgaaucauacacgguugaccuauuuuucaguaccaa

SEQ ID NO: 315 - RNA, 98 nt, HOMO SAPIENS
cagagaggagcugccacuugggcacugaaacaauguccauuaggcuuuguuauggaaacuucuccugaucauuguuuuguguccauugagcuuccaau

SEQ ID NO: 316 - RNA, 95 nt, HOMO SAPIENS
gucgaggccguggcccggaaguggucggggccgcugcgggcggaagggcgccugugcuucguccgcucggcgguggcccagccaggcccgcggga

SEQ ID NO: 317 - RNA, 102 nt, HOMO SAPIENS
gggagccaaaugcuuugcuagagcugguaaaauggaaccaaaucgacuguccaauggauuugguccccuucaaccagcuguagcugugcauugauggcgccg

SEQ ID NO: 318 - RNA, 82 nt, HOMO SAPIENS
ccugccggggcuaaagugcugacagugcagauagugguccucuccgugcuaccgcacuguggguacuugcugcuccagcagg

SEQ ID NO: 319 - RNA, 94 nt, HOMO SAPIENS
gugugugugugugugugugguuauuuugguauaguagcucuagacucuauuauaguuuccugagcugcuguaccaaaauaccacaaacgggcug

SEQ ID NO: 320 - RNA, 115 nt, HOMO SAPIENS
ccaccuucagcugaguguagugcccuacuccagagggcgucacucauguaaacuaaaacaugauuguagccuuuuggaguagaguaauacacaucacguaacgcauauuuggugg

SEQ ID NO: 321 - RNA, 98 nt, HOMO SAPIENS
guacacaguagaagcaucccuugcaggggcuguuggguugcauccuaagcugugcuggagcuucccgauguacucuguagaugucuuugcaccuucug

SEQ ID NO: 322 - RNA, 87 nt, HOMO SAPIENS
ucucaagcugugagucuacaaagggaagcccuuucuguugucuaaaagaaaagaaagugcuucucuuugguggguuacgguuugaga

SEQ ID NO: 323 - RNA, 84 nt, HOMO SAPIENS
ugugcagugggaaggggggccgauacacuguacgagagugaguagcaggucucacagugaaccggucucuuucccuacuguguc

SEQ ID NO: 324 - RNA, 87 nt, HOMO SAPIENS
ucagagugagguaguagauuguauaguugugggguagugauuuuacccuguucaggagauaacuauacaaucuauugccuucccuga

SEQ ID NO: 325 - RNA, 97 nt, HOMO SAPIENS
ugcagggagguauuaaguuggugcaaaaguaauugugauuuuugccauuaaaaguaacgacaaaacuggcaauuacuuuugcaccaaaccugguauu

SEQ ID NO: 326 - RNA, 61 nt, HOMO SAPIENS
cccucuacagggaagcgcuuucuguugucugaaagaaaagaaagugcuuccuuuuagaggg

SEQ ID NO: 327 - RNA, 91 nt, HOMO SAPIENS
auuuucaucaccuagggaucuuguuaaaaagcagauucugauucagggaccaagauucugcauuuuuagcaaguucucaagugaugcuaau

SEQ ID NO: 328 - RNA, 84 nt, HOMO SAPIENS
cuccccauggcccugucucccaacccuuguaccagugcugggcucagacccugguacaggccugggggacagggaccuggggac

SEQ ID NO: 329 - RNA, 93 nt, HOMO SAPIENS
guagucaguaguuggggggugggaacggcuucauacaggaguugaugcacaguuauccagcuccuauaugaugccuuucuucauccccuucaa

SEQ ID NO: 330 - RNA, 87 nt, HOMO SAPIENS
ucuccugcugugacccucaagauggaagcaguuucuguugucugaaaggaaagaaagugcuuccuuuuugaggguuacuguuugaga

SEQ ID NO: 331 - RNA, 98 nt, HOMO SAPIENS
aaccucucuuagccucuguuucuuuauugcgguagauacuauuaaccuaaaaugagaaggcuaauaguaucuaccacaauaaaauuguugugaggaua

SEQ ID NO: 332 - RNA, 96 nt, HOMO SAPIENS
ucuauuugucuuaggugagcuaaaugugugcugggacacauuugagccaaaugucccagcacacauuuagcucacauaagaaaaauggacucuagu

SEQ ID NO: 333 - RNA, 84 nt, HOMO SAPIENS
uugguacuuggagagugguuaucccuguccuguucguuuugcucaugucgaaucguacagggucauccacuuuuucaguaucaa

SEQ ID NO: 334 - RNA, 110 nt, HOMO SAPIENS
aucauucagaaaugguauacaggaaaaugaccuaugaauugacagacaauauagcugaguuugucugucauuucuuuaggccaauauucuguaugacugugcuacuucaa

SEQ ID NO: 335 - RNA, 97 nt, HOMO SAPIENS
gagagggaagauuuagguuggugcaaaaguaauugugguuuuugccauugaaaguaauggcaaaaaccacaguuucuuuugcaccaaccuaauaaaa

SEQ ID NO: 336 - RNA, 96 nt, HOMO SAPIENS
cagugcuggggucucaggaggcagcgcucucaggacgucaccaccauggccugggcucugcuccuccucacccuccucacucagggcacaggugau

SEQ ID NO: 337 - RNA, 97 nt, HOMO SAPIENS
uuagguaauuccuccacucaaaacccuucagugacuuccaugacaugaaauaggaagucauuggaggguuugagcagaggaaugaccuguuuuaaaa

SEQ ID NO: 338 - RNA, 110 nt, HOMO SAPIENS
cggggcaccccgcccggacagcgcgccggcaccuuggcucuagacugcuuacugcccgggccgcccucaguaacagucuccagucacggccaccgacgccuggccccgcc

SEQ ID NO: 339 - RNA, 85 nt, HOMO SAPIENS
gcuaagcacuuacaacuguuugcagaggaaacugagacuuuguaacuaugucucagucucaucugcaaagaaguaagugcuuugc

SEQ ID NO: 340 - RNA, 111 nt, HOMO SAPIENS
gccgggagguugaacauccugcauagugcugccaggaaaucccuauuucauauaagagggggcuggcugguugcauauguaggaugucccaucucccagcccacuucguca

SEQ ID NO: 341 - RNA, 83 nt, HOMO SAPIENS
ccuuggaguaaaguagcagcacauaaugguuuguggauuuugaaaaggugcaggccauauugugcugccucaaaaauacaagg

SEQ ID NO: 342 - RNA, 74 nt, HOMO SAPIENS
gugguguccuacucaggagaguggcaaucacauguaauuaggugugauugaaaccucuaagaguggaguaacac

SEQ ID NO: 343 - RNA, 98 nt, HOMO SAPIENS
uggccgacggggcgcgcgcggccuggaggggcggggcggacgcagagccgcguuuagucuaucgcugcgguugcgagcgcuguagggagccugugcug

SEQ ID NO: 344 - RNA, 97 nt, HOMO SAPIENS
uacuuauuacugguagugagucucuaagaaaagaggaggugguuguuuuccuccucuuuucuuugagacucacuaccaauaauaagaaauacuacua

SEQ ID NO: 345 - RNA, 95 nt, HOMO SAPIENS
auauauaucuauaucuagcuccguauauauauauauauauauauagauaucuccauauauauggagauagauauagaaauaaaacaagcaaagaa

SEQ ID NO: 346 - RNA, 77 nt, HOMO SAPIENS
guggccucguucaaguaauccaggauaggcugugcaggucccaaugggccuauucuugguuacuugcacggggacgc

SEQ ID NO: 347 - RNA, 68 nt, HOMO SAPIENS
uaaaagguagauucuccuucuaugaguacauuauuuaugauuaaucauagaggaaaauccacguuuuc

SEQ ID NO: 348 - RNA, 97 nt, HOMO SAPIENS
uaccgacccucgauuugguucaggaccuucccugaaccaaggaagagucacagucucuuccuugguucagggaggguccccaacaauguccucaugg

SEQ ID NO: 349 - RNA, 85 nt, HOMO SAPIENS
cucaggcugugacccuccagagggaaguacuuucuguugucugagagaaaagaaagugcuucccuuuggacuguuucgguuugag

SEQ ID NO: 350 - RNA, 74 nt, HOMO SAPIENS
gggugagguaguagguuguauaguuuggggcucugcccugcuaugggauaacuauacaaucuacugucuuuccu

SEQ ID NO: 351 - RNA, 84 nt, HOMO SAPIENS
cguugcugcagcugguguugugaaucaggccgacgagcagcgcauccucuuacccggcuauuucacgacaccaggguugcauca

SEQ ID NO: 352 - RNA, 90 nt, HOMO SAPIENS
ucuaagaaacgcaguggucucugaagccugcaggggcaggccagcccugcacugaacgccuguucuugccagguggcagaagguugcugc

SEQ ID NO: 353 - RNA, 112 nt, HOMO SAPIENS
ccaccccgguccugcucccgccccagcagcacacugugguuuguacggcacuguggccacguccaaaccacacugugguguuagagcgagggugggggaggcaccgccgagg

SEQ ID NO: 354 - RNA, 124 nt, HOMO SAPIENS
gccaccaccaucagccauacuauguguagugccuuauucaggaagguguuacuuaauagauuaauauuuguaaggcacccuucugaguagaguaaugugcaacauggacaacauuugugguggc

SEQ ID NO: 355 - RNA, 80 nt, HOMO SAPIENS
gguacuugaagagugguuaucccugcuguguucgcuuaauuuaugacgaaucauacagggacauccaguuuuucaguauc

SEQ ID NO: 356 - RNA, 94 nt, HOMO SAPIENS
auaaaggaaguuaggcugaggggcagagagcgagacuuuucuauuuuccaaaagcucggucugaggccccucagucuugcuuccuaacccgcgc

SEQ ID NO: 357 - RNA, 95 nt, HOMO SAPIENS
ugcucggcuguuccuaggguguuucucucaucucuggucuauaauggguuaaauaguagagaugagggcaacacccuaggaacagcagaggaacc

SEQ ID NO: 358 - RNA, 89 nt, HOMO SAPIENS
cgggguugguuguuaucuuugguuaucuagcuguaugagugguguggagucuucauaaagcuagauaaccgaaaguaaaaauaacccca

SEQ ID NO: 359 - RNA, 106 nt, HOMO SAPIENS
gcgcagcgcccugucucccagccugaggugcagugcugcaucucuggucaguugggagucugagaugaagcacuguagcucaggaagagagaaguuguucugcagc

SEQ ID NO: 360 - RNA, 86 nt, HOMO SAPIENS
uugguacuuggagagaggugguccguggcgcguucgcuuuauuuauggcgcacauuacacggucgaccucuuugcaguaucuaauc

SEQ ID NO: 361 - RNA, 87 nt, HOMO SAPIENS
ugucccccccggcccagguucugugauacacuccgacucgggcucuggagcagucagugcaugacagaacuugggcccggaaggacc

SEQ ID NO: 362 - RNA, 100 nt, HOMO SAPIENS
agauaaauucacucuagugcuuuauggcuuuuuauuccuaugugauaguaauaaagucucauguagggauggaagccaugaaauacauugugaaaaauca

SEQ ID NO: 363 - RNA, 84 nt, HOMO SAPIENS
gugguaccugaagagagguuuucuggguuucuguuucuuuauugaggacgaaacacaccugguuaaccucuuuuccaguaucaa

SEQ ID NO: 364 - RNA, 83 nt, HOMO SAPIENS; identical to SEQ ID NO: 249

SEQ ID NO: 365 - RNA, 72 nt, HOMO SAPIENS
uacaucggccauuauaauacaaccugauaaguguuauagcacuuaucagauuguauuguaauugucugugua

SEQ ID NO: 366 - RNA, 95 nt, HOMO SAPIENS
ucuuaucaaugagguagaccauggguucucauuguaauaguguagaauguugguuaacuguggacucccuggcucugucucaaaucuacugauuc

SEQ ID NO: 367 - RNA, 85 nt, HOMO SAPIENS
ucucaagcugugacugcaaagggaagcccuuucuguugucuaaaagaaaagaaagugcuucccuuuggugaauuacgguuugaga

SEQ ID NO: 368 - RNA, 89 nt, HOMO SAPIENS
gucagcagugccuuagcagcacguaaauauuggcguuaagauucuaaaauuaucuccaguauuaacugugcugcugaaguaagguugac

SEQ ID NO: 369 - RNA, 94 nt, HOMO SAPIENS
cuuuggcgaucacugccucucugggccugugucuuaggcucugcaagaucaaccgagcaaagcacacggccugcagagaggcagcgcucugccc

SEQ ID NO: 370 - RNA, 79 nt, HOMO SAPIENS
cccgggcugagguaggagguuguauaguugaggaggacacccaaggagaucacuauacggccuccuagcuuuccccagg

SEQ ID NO: 371 - RNA, 102 nt, HOMO SAPIENS
gguccucugacucucuucggugacggguauucuuggguggauaauacggauuacguuguuauugcuuaagaauacgcguagucgaggagaguaccagcggca

SEQ ID NO: 372 - RNA, 98 nt, HOMO SAPIENS
cauauuagguuaaugcaaaaguaaucgcgguuugugccagaugacgauuugaauuaauaaauucauuugguauaaaccgcgauuauuuuugcaucaac

SEQ ID NO: 373 - RNA, 110 nt, HOMO SAPIENS
ccuggccuccugcagugccacgcuccguguauuugacaagcugaguuggacacuccaugugguagagugucaguuugucaaauaccccaagugcggcacaugcuuaccag

SEQ ID NO: 374 - RNA, 110 nt, HOMO SAPIENS
ggccagcugugaguguuucuuuggcagugucuuagcugguuguugugagcaauaguaaggaagcaaucagcaaguauacugcccuagaagugcugcacguuguggggccc

SEQ ID NO: 375 - RNA, 97 nt, HOMO SAPIENS
ggccuagccaaauacuguauuuuugaucgacauuugguugaaaaauaucuauguauuaguaaaccuguguuguucaagaguccacuguguuuugcug

SEQ ID NO: 376 - RNA, 73 nt, HOMO SAPIENS
cucugccucccgugccuacugagcugaaacacaguugguuuguguacacuggcucaguucagcaggaacaggg

SEQ ID NO: 377 - RNA, 81 nt, HOMO SAPIENS
ugugcaucguggucaaaugcucagacuccugugguggcugcucaugcaccacggauguuugagcaugugcuacggugucua

SEQ ID NO: 378 - RNA, 71 nt, HOMO SAPIENS
gccaacccaguguucagacuaccuguucaggaggcucucaauguguacaguagucugcacauugguuaggc

SEQ ID NO: 379 - RNA, 85 nt, HOMO SAPIENS
cucaggcugugacacucuagagggaagcgcuuucuguugucugaaagaaaggaaagugcauccuuuuagaguguuacuguuugag

SEQ ID NO: 380 - RNA, 88 nt, HOMO SAPIENS
cgaggaugggagcugagggcugggucuuugcgggcgagaugagggugucggaucaacuggccuacaaagucccaguucucggcccccg

SEQ ID NO: 381 - RNA, 84 nt, HOMO SAPIENS
gcauccggguugagguaguagguuguaugguuuagaguuacacccugggaguuaacuguacaaccuucuagcuuuccuuggagc

SEQ ID NO: 382 - RNA, 87 nt, HOMO SAPIENS
ugagggccccucugcguguucacagcggaccuugauuuaaugucuauacaauuaaggcacgcggugaaugccaagagaggcgccucc

SEQ ID NO: 383 - RNA, 95 nt, HOMO SAPIENS
gauauacacuauauuauguauaaauguauacacacuuccuauauguauccacauauauauaguguauauauuauacauguauagguguguauaug

SEQ ID NO: 384 - RNA, 84 nt, HOMO SAPIENS
gucagaauaaugucaaagugcuuacagugcagguagugauaugugcaucuacugcagugaaggcacuuguagcauuauggugac

SEQ ID NO: 385 - RNA, 119 nt, HOMO SAPIENS
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 386 - RNA, 96 nt, HOMO SAPIENS
cuccuaugcacccucuuuccauaggugaugagucacagggcucagggaaugugucugcaccugugacucaucaccaguggaaagcccaucccauau

SEQ ID NO: 387 - RNA, 61 nt, HOMO SAPIENS
aagaugguugaccauagaacaugcgcuaucucugugucguauguaauaugguccacaucuu

SEQ ID NO: 388 - RNA, 88 nt, HOMO SAPIENS
ucucaggcugugacccucuagagggaagcgcuuucuguuggcuaaaagaaaagaaagcgcuucccuucagaguguuaacgcuuugaga

SEQ ID NO: 389 - RNA, 78 nt, HOMO SAPIENS
cuuucuacacagguugggaucgguugcaaugcuguguuucuguaugguauugcacuugucccggccuguugaguuugg

SEQ ID NO: 390 - RNA, 98 nt, HOMO SAPIENS
uucucacccccgccugacacgggcgacagcugcggcccgcuguguucacucgggccgagugcgucuccugucaggcaagggagagcagagccccccug

SEQ ID NO: 391 - RNA, 87 nt, HOMO SAPIENS
ucucaugcugugacccuacaaagggaagcacuuucucuuguccaaaggaaaagaaggcgcuucccuuuggaguguuacgguuugaga

SEQ ID NO: 392 - RNA, 94 nt, HOMO SAPIENS
caguuccuaacaggccucagaccaguaccggucuguggccuggggguugaggaccccugcucuaggcugguacugcugaugcuuaaaaagagag

SEQ ID NO: 393 - RNA, 95 nt, HOMO SAPIENS
agugaaauugcuaggucauauggucagucuacuuuuagaguaauugugaaacuguuuuucaaaguagcuguaccauuugcacucccuguggcaau

SEQ ID NO: 394 - RNA, 110 nt, HOMO SAPIENS
ugcucgcucagcugaucuguggcuuagguaguuucauguuguugggauugaguuuugaacucggcaacaagaaacugccugaguuacaucagucgguuuucgucgagggc

SEQ ID NO: 395 - RNA, 93 nt, HOMO SAPIENS
ccuuccggcgucccaggcggggcgccgcgggaccgcccucgugucuguggcggugggaucccgcggccguguuuuccugguggcccggccaug

SEQ ID NO: 396 - RNA, 84 nt, HOMO SAPIENS
gcucuuccucucuaauccuuugucccugggugagagugcuuucugaaugcaaugcacccgggcaaggauucugagagggugagc

SEQ ID NO: 397 - RNA, 73 nt, HOMO SAPIENS
ccuggcacugagaacugaauuccauaggcugugagcucuagcaaugcccuguggacucaguucuggugcccgg

SEQ ID NO: 398 - RNA, 95 nt, HOMO SAPIENS
gaagaucucaggcagugacccucuagauggaagcacugucuguugucuaagaaaagaucgugcauccuuuuagaguguuacuguuugagaaaauc

SEQ ID NO: 399 - RNA, 98 nt, HOMO SAPIENS
cucuuguucacagccaaacucuacuuguccuucugaguguaauuacguacaugcaguagcucaggagacaagcagguuuacccuguggaugagucuga

SEQ ID NO: 400 - RNA, 94 nt, HOMO SAPIENS
aauucagcccugccacuggcuuaugucaugaccuugggcuacucaggcugucugcacaaugagccaguuggacaggagcagugccacucaacuc

SEQ ID NO: 401 - RNA, 96 nt, HOMO SAPIENS
uugccuaaagucacacagguuauagaucuggauuggaacccagggagccagacugccuggguucaaauccagaucuauaacuugugugacuuuggg

SEQ ID NO: 402 - RNA, 80 nt, HOMO SAPIENS
aggacccuuccagagggcccccccucaauccuguugugccuaauucagaggguuggguggaggcucuccugaagggcucu

SEQ ID NO: 403 - RNA, 71 nt, HOMO SAPIENS
ugggaaacauacuucuuuauaugcccauauggaccugcuaagcuauggaauguaaagaaguauguaucuca

SEQ ID NO: 404 - RNA, 86 nt, HOMO SAPIENS
ugcuuguaacuuuccaaagaauucuccuuuugggcuuucugguuuuauuuuaagcccaaaggugaauuuuuugggaaguuugagcu

SEQ ID NO: 405 - RNA, 75 nt, HOMO SAPIENS
agacagagaagccaggucacgucucugcaguuacacagcucacgagugccugcugggguggaaccuggucugucu

SEQ ID NO: 406 - RNA, 86 nt, HOMO SAPIENS
ugccagucucuaggucccugagacccuuuaaccugugaggacauccagggucacaggugagguucuugggagccuggcgucuggcc

SEQ ID NO: 407 - RNA, 83 nt, HOMO SAPIENS
gagaaucaucucucccagauaauggcacucucaaacaaguuuccaaauuguuugaaaggcuauuucuuggucagaugacucuc

SEQ ID NO: 408 - RNA, 96 nt, HOMO SAPIENS
accugaguaaccuuugcuaguccugacucagccaguacuggucuuagacuggugaugggucaggguucauauuuuggcaucucucucugggcaucu

SEQ ID NO: 409 - RNA, 94 nt, HOMO SAPIENS
caugcugugugugguacccuacugcagacaguggcaaucauguauaauuaaaaaugauugguacgucuguggguagaguacugcaugacacaug

SEQ ID NO: 410 - RNA, 86 nt, HOMO SAPIENS
gguccuugcccucaaggagcucacagucuauugaguuaccuuucugacuuucccacuagauugugagcuccuggagggcaggcacu

SEQ ID NO: 411 - RNA, 110 nt, HOMO SAPIENS
ccgcagagugugacuccuguucuguguauggcacugguagaauucacugugaacagucucagucagugaauuaccgaagggccauaaacagagcagagacagauccacga

SEQ ID NO: 412 - RNA, 73 nt, HOMO SAPIENS
acuuggagagaggcuggccgugaugaauucgauucaucaaagcgagucauacacggcucuccucucuuuuagu

SEQ ID NO: 413 - RNA, 85 nt, HOMO SAPIENS
augguguuaucaaguguaacagcaacuccauguggacuguguaccaauuuccaguggagaugcuguuacuuuugaugguuaccaa

SEQ ID NO: 414 - RNA, 111 nt, HOMO SAPIENS
agaguguucaaggacagcaagaaaaaugagggacuuucaggggcagcuguguuuucugacucagucauaaugccccuaaaaauccuuauuguucuugcagugugcaucggg

SEQ ID NO: 415 - RNA, 70 nt, HOMO SAPIENS
gugaauuagguaguuucauguuguugggccuggguuucugaacacaacaacauuaaaccacccgauucac

SEQ ID NO: 416 - RNA, 95 nt, HOMO SAPIENS
aaagacaugcuguccacaguguguuugauaagcugacaugggacagggauucuuuucacuguugugucaguuuaucaaacccauacuuggaugac

SEQ ID NO: 417 - RNA, 87 nt, HOMO SAPIENS
ucucaggcugugacccucuaaagggaagcgcuuucuguggucagaaagaaaagcaagugcuuccuuuuagaggguuaccguuuggga

SEQ ID NO: 418 - RNA, 84 nt, HOMO SAPIENS
acuggucggugauuuagguaguuuccuguuguugggauccaccuuucucucgacagcacgacacugccuucauuacuucaguug

SEQ ID NO: 419 - RNA, 67 nt, HOMO SAPIENS
aaaauggugagagcguugaggggaguuccagacggagaugcgaggaccccucggggucugacccaca

SEQ ID NO: 420 - RNA, 81 nt, HOMO SAPIENS
cucucugcuuucagcuucuuuacaguguugccuuguggcauggaguucaagcagcauuguacagggcuaucaaagcacaga

SEQ ID NO: 421 - RNA, 87 nt, HOMO SAPIENS
agcuucccuggcucuagcagcacagaaauauuggcacagggaagcgagucugccaauauuggcugugcugcuccaggcaggguggug

SEQ ID NO: 422 - RNA, 110 nt, HOMO SAPIENS
gccgagaccgagugcacagggcucugaccuaugaauugacagccagugcucucgucuccccucuggcugccaauuccauaggucacagguauguucgccucaaugccagc

SEQ ID NO: 423 - RNA, 85 nt, HOMO SAPIENS
ccuuagcagagcuguggagugugacaaugguguuugugucuaaacuaucaaacgccauuaucacacuaaauagcuacugcuaggc

SEQ ID NO: 424 - RNA, 95 nt, HOMO SAPIENS
gauaguaauaagaaagaugagcucauuguaauaugagcuucauuuauacauuucauauuaccauuagcucaucuuuuuuauuacuaccuucaaca

SEQ ID NO: 425 - RNA, 96 nt, HOMO SAPIENS
ggggacugccgggugacccuggaaauccagaguggguggggccagucugaccguuucuaggcgacccacucuugguuuccaggguugcccuggaaa

SEQ ID NO: 426 - RNA, 89 nt, HOMO SAPIENS
accaugcuguaguguguguaaacauccuacacucucagcugugagcucaagguggcugggagaggguuguuuacuccuucugccaugga

SEQ ID NO: 427 - RNA, 97 nt, HOMO SAPIENS
aacuuaacaucaugcuaccucuuuguaucauauuuuguuauucuggucacagaaugaccuaguauucuguaccagggaagguaguucuuaacuauau

SEQ ID NO: 428 - RNA, 88 nt, HOMO SAPIENS
ucccaugcugugacccuccaaagggaagcgcuuucuguuuguuuucucuuaaacaaagugccucccuuuagaguguuaccguuuggga

SEQ ID NO: 429 - RNA, 129 nt, HOMO SAPIENS
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 430 - RNA, 71 nt, HOMO SAPIENS
ugacgggcgagcuuuuggcccggguuauaccugaugcucacguauaagacgagcaaaaagcuuguugguca

SEQ ID NO: 431 - RNA, 110 nt, HOMO SAPIENS
gaccagucgcugcggggcuuuccuuugugcuugaucuaaccaugugguggaacgauggaaacggaacaugguucugucaagcaccgcggaaagcaccgugcucuccugca

SEQ ID NO: 432 - RNA, 79 nt, HOMO SAPIENS
ugguacucggggagagguuacccgagcaacuuugcaucuggacgacgaauguugcucggugaaccccuuuucgguauca

SEQ ID NO: 433 - RNA, 80 nt, HOMO SAPIENS
gguaccugagaagagguugucugugaugaguucgcuuuuauuaaugacgaauauaacacagauggccuguuuucaguacc

SEQ ID NO: 434 - RNA, 80 nt, HOMO SAPIENS
gcaggaaugcugcgagcagugccaccucaugguacucggagggagguuguccguggugaguucgcauuauuuaaugaugc

SEQ ID NO: 435 - RNA, 69 nt, HOMO SAPIENS
cuguggugcauuguaguugcauugcauguucuggugguacccaugcaauguuuccacagugcaucacag

SEQ ID NO: 436 - RNA, 82 nt, HOMO SAPIENS
agggggcgagggauuggagagaaaggcaguuccugaugguccccuccccaggggcuggcuuuccucugguccuucccuccca

SEQ ID NO: 437 - RNA, 66 nt, HOMO SAPIENS
agggcuccugacuccagguccuguguguuaccuagaaauagcacuggacuuggagucagaaggccu

SEQ ID NO: 438 - RNA, 97 nt, HOMO SAPIENS
cagaucucagacaucucggggaucaucaugucacgagauaccagugugcacuugugacagauugauaacugaaaggucugggagccacucaucuuca

SEQ ID NO: 439 - RNA, 87 nt, HOMO SAPIENS
ucucaagcugugggucugcaaagggaagcccuuucuguugucuaaaagaagagaaagcgcuucccuuugcuggauuacgguuugaga

SEQ ID NO: 440 - RNA, 87 nt, HOMO SAPIENS
cacuguucuaugguuaguuuugcagguuugcauccagcugugugauauucugcugugcaaauccaugcaaaacugacugugguagug

SEQ ID NO: 441 - RNA, 82 nt, HOMO SAPIENS
ggccugcccgacacucuuucccuguugcacuacuauaggccgcugggaagcagugcaaugaugaaagggcaucggucagguc

SEQ ID NO: 442 - RNA, 73 nt, HOMO SAPIENS
gcucccuucaacuuuaacauggaagugcuuucugugacuuuaaaaguaagugcuuccauguuuuaguaggagu

SEQ ID NO: 443 - RNA, 90 nt, HOMO SAPIENS
ucucagguugugaccuucucgaggaaagaagcacuuucuguugucugaaagaaaagaaagugcuuccuuucagaggguuacgguuugaga

SEQ ID NO: 444 - RNA, 91 nt, HOMO SAPIENS
cugugugugaugagcuggcaguguauuguuagcugguugaauaugugaauggcaucggcuaacaugcaacugcugucuuauugcauauaca

SEQ ID NO: 445 - RNA, 96 nt, HOMO SAPIENS
ccucaguaagaccaagcucagugugccauuuccuugucuguagccaugucuaugggcucuugaguuggccaucugagugagggccugcuuauucua

SEQ ID NO: 446 - RNA, 87 nt, HOMO SAPIENS
ucucaggcugugacccuccaaagggaagaacuuucuguugucuaaaagaaaagaacgcacuucccuuuagaguguuaccgugugaga

SEQ ID NO: 447 - RNA, 75 nt, HOMO SAPIENS
aacucacacauuaaccaaagaggaaggucccauuacugcagggaucuuagcaguacugggaccuaccucuuuggu

SEQ ID NO: 448 - RNA, 72 nt, HOMO SAPIENS
aaucuaaagacaacauuucugcacacacaccagacuauggaagccaguguguggaaaugcuucugcuagauu

SEQ ID NO: 449 - RNA, 79 nt, HOMO SAPIENS
agcaaagaaguguguugcccucuaggaaauguguguugcucugauguaauuagguugacauacguuucccugguagcca

SEQ ID NO: 450 - RNA, 99 nt, HOMO SAPIENS
uggcggccugggcgggagcgcgcgggcggggccggccccgcugccuggaauuaaccccgcugugcuugcucgucccgcccgcagcccuaggcggcgucg

SEQ ID NO: 451 - RNA, 97 nt, HOMO SAPIENS
cacucugcuguggccuauggcuuuucauuccuaugugauugcugucccaaacucauguagggcuaaaagccaugggcuacagugaggggcgagcucc

SEQ ID NO: 452 - RNA, 81 nt, HOMO SAPIENS
ccuuggccauguaaaagugcuuacagugcagguagcuuuuugagaucuacugcaauguaagcacuucuuacauuaccaugg

SEQ ID NO: 453 - RNA, 91 nt, HOMO SAPIENS
aaacgauacuaaacuguuuuugcgauguguuccuaauaugcacuauaaauauauugggaacauuuugcauguauaguuuuguaucaauaua

SEQ ID NO: 454 - RNA, 88 nt, HOMO SAPIENS
acaaugcuuugcuagagcugguaaaauggaaccaaaucgccucuucaauggauuugguccccuucaaccagcuguagcuaugcauuga

SEQ ID NO: 455 - RNA, 68 nt, HOMO SAPIENS
gaggcaaaguucugagacacuccgacucugaguaugauagaagucagugcacuacagaacuuugucuc

SEQ ID NO: 456 - RNA, 95 nt, HOMO SAPIENS
uuguaccuggugugauuauaaagcaaugagacugauugucauaugucguuugugggauccgucucaguuacuuuauagccauaccugguaucuua

SEQ ID NO: 457 - RNA, 78 nt, HOMO SAPIENS
cugaaauagguugccugugagguguucacuuucuauaugaugaauauuauacagucaaccucuuuccgauaucgaauc

SEQ ID NO: 458 - RNA, 96 nt, HOMO SAPIENS
cucgggaggggcgggagggggguccccggugcucggaucucgagggugcuuauuguucgguccgagccugggucucccucuuccccccaacccccc

SEQ ID NO: 459 - RNA, 81 nt, HOMO SAPIENS
ggguaaguggaaagauggugggccgcagaacaugugcugaguucgugccauaugucugcugaccaucaccuuuagaagccc

SEQ ID NO: 460 - RNA, 81 nt, HOMO SAPIENS
cuucuggaagcugguuucacaugguggcuuagauuuuuccaucuuuguaucuagcaccauuugaaaucaguguuuuaggag

SEQ ID NO: 461 - RNA, 68 nt, HOMO SAPIENS
cccucgucuuacccagcaguguuugggugcgguugggagucucuaauacugccggguaaugauggagg

SEQ ID NO: 462 - RNA, 94 nt, HOMO SAPIENS
uggggugucugugcuauggcagcccuagcacacagauacgcccagagaaagccugaacguugggcguaucuguaugcuagggcugcuguaacaa

SEQ ID NO: 463 - RNA, 95 nt, HOMO SAPIENS
gcuguugaggcugcgcagccaggcccugacggugggguggcugcgggccuucugaaggucucccacguuguggcccagcagcgcagucacguugc

SEQ ID NO: 464 - RNA, 79 nt, HOMO SAPIENS
ugcaugaguucgucuuggucuaggauuguuggaggagucagaaaaacuaccccagggauccugaaguccuuugggugga

SEQ ID NO: 465 - RNA, 87 nt, HOMO SAPIENS
ucucaugcugugacccucuagagggaagcacuuucucuugucuaaaagaaaagaaagcgcuucucuuuagaggauuacucuuugaga

SEQ ID NO: 466 - RNA, 94 nt, HOMO SAPIENS
cgccuccuaccgcagugcuugacgggaggcggagcggggaacgaggccgucggccauuuugugucugcuuccugugggacguggugguagccgu

SEQ ID NO: 467 - RNA, 106 nt, HOMO SAPIENS
cccagccuggcacauuaguaggccucaguaaauguuuauuagaugaauaaaugaaugacucaucagcaaacauuuauugugugccugcuaaagugagcuccacagg

SEQ ID NO: 468 - RNA, 99 nt, HOMO SAPIENS
caagcacgauuagcauuugaggugaaguucuguuauacacucaggcuguggcucucugaaagucagugcaucacagaacuuugucucgaaagcuuucua

SEQ ID NO: 469 - RNA, 85 nt, HOMO SAPIENS
accuacucagaguacauacuucuuuauguacccauaugaacauacaaugcuauggaauguaaagaaguauguauuuuugguaggc

SEQ ID NO: 470 - RNA, 88 nt, HOMO SAPIENS
guugucugugguacccuacucuggagagugacaaucauguauaacuaaauuugauugacacuucugugaguagaguaacgcaugacac

SEQ ID NO: 471 - RNA, 71 nt, HOMO SAPIENS
gcgacuguaaacauccucgacuggaagcugugaagccacagaugggcuuucagucggauguuugcagcugc

SEQ ID NO: 472 - RNA, 83 nt, HOMO SAPIENS
cugacuaugccuccccgcauccccuagggcauugguguaaagcuggagacccacugccccaggugcugcuggggguuguaguc

SEQ ID NO: 473 - RNA, 81 nt, HOMO SAPIENS
guuccacucuagcagcacguaaauauuggcguagugaaauauauauuaaacaccaauauuacugugcugcuuuagugugac

SEQ ID NO: 474 - RNA, 75 nt, HOMO SAPIENS
guggggagccugguuagaccuggcccagaccucagcuacacaagcugauggacugagucaggggccacacucucc

SEQ ID NO: 475 - RNA, 97 nt, HOMO SAPIENS
gcuugaugaugcugcugaugcuggcggugaucccgauggugugagcuggaaauggggugcuacgucaucguugucaucgucaucaucaucauccgag

SEQ ID NO: 476 - RNA, 80 nt, HOMO SAPIENS
ccuguugccacaaacccguagauccgaacuugugguauuaguccgcacaagcuuguaucuauagguaugugucuguuagg

SEQ ID NO: 477 - RNA, 64 nt, HOMO SAPIENS
ccccgcgacgagccccucgcacaaaccggaccugagcguuuuguucguucggcucgcgugaggc

SEQ ID NO: 478 - RNA, 98 nt, HOMO SAPIENS
aucugugcucuuugauuacaguuguucaaccaguuacuaaucuaacuaauuguaacugguugaacaacugaacccaaagggugcaaaguagaaacauu

SEQ ID NO: 479 - RNA, 90 nt, HOMO SAPIENS
ucccaugcugugacccucuagaggaagcacuuucuguuuguugucugagaaaaaacaaagugcuucccuuuagaguguuaccguuuggga

SEQ ID NO: 480 - RNA, 95 nt, HOMO SAPIENS
ggugagugcguuuccaagugugaagggacccuuccuguagugucuuauauacaauacaguaggaauguuccuucuuugccacucauacaccuuua

SEQ ID NO: 481 - RNA, 63 nt, HOMO SAPIENS
aagaaaugguuuaccgucccacauacauuuugaauauguaugugggaugguaaaccgcuucuu

SEQ ID NO: 482 - RNA, 67 nt, HOMO SAPIENS
ucuccaacaauauccuggugcugagugaugacucaggcgacuccagcaucagugauuuuguugaaga

SEQ ID NO: 483 - RNA, 72 nt, HOMO SAPIENS
ggagcuuaucagaaucuccagggguacuuuauaauuucaaaaagucccccaggugugauucugauuugcuuc

SEQ ID NO: 484 - RNA, 98 nt, HOMO SAPIENS
uugaggccuuaaaguacuguagcagcacaucaugguuuacaugcuacagucaagaugcgaaucauuauuugcugcucuagaaauuuaaggaaauucau

SEQ ID NO: 485 - RNA, 99 nt, HOMO SAPIENS
cccuggcauggugugguggggcagcugguguugugaaucaggccguugccaaucagagaacggcuacuucacaacaccagggccacaccacacuacagg

SEQ ID NO: 486 - RNA, 83 nt, HOMO SAPIENS
ugugggaugagguaguagauuguauaguuuuagggucauaccccaucuuggagauaacuauacagucuacugucuuucccacg

SEQ ID NO: 487 - RNA, 82 nt, HOMO SAPIENS
ugagcuguuggauucggggccguagcacugucugagagguuuacauuucucacagugaaccggucucuuuuucagcugcuuc

SEQ ID NO: 488 - RNA, 92 nt, HOMO SAPIENS
gggcagucuuugcuacuguaaacauccuugacuggaagcuguaagguguucagaggagcuuucagucggauguuuacagcggcaggcugcca

SEQ ID NO: 489 - RNA, 100 nt, HOMO SAPIENS
ccaaagaaagaugcuaaacuauuuuugcgauguguuccuaauauguaauauaaauguauuggggacauuuugcauucauaguuuuguaucaauaauaugg

SEQ ID NO: 490 - RNA, 94 nt, HOMO SAPIENS
cggggcggccgcucucccuguccuccaggagcucacgugugccugccugugagcgccucgacgacagagccggcgccugccccagugucugcgc

SEQ ID NO: 491 - RNA, 80 nt, HOMO SAPIENS
gguauuuaaaagguagauuuuccuucuaugguuacguguuugaugguuaaucauagaggaaaauccacguuuucaguauc

SEQ ID NO: 492 - RNA, 84 nt, HOMO SAPIENS
ucucaugcagucauucuccaaaagggagcacuuucuguuugaaagaaaacaaagugccuccuuuuagaguguuacuguuugaga

SEQ ID NO: 493 - RNA, 88 nt, HOMO SAPIENS
ugcgcuccucucagucccugagacccuaacuugugauguuuaccguuuaaauccacggguuaggcucuugggagcugcgagucgugcu

SEQ ID NO: 494 - RNA, 99 nt, HOMO SAPIENS
cgcccaccucagccucccaaaaugcugggauuacaggcaugagccacugcggucgaccaugaccuggacauguuugugcccaguacugucaguuugcag

SEQ ID NO: 495 - RNA, 84 nt, HOMO SAPIENS
gcucccccucucuaauccuugcuaccugggugagagugcugucugaaugcaaugcaccugggcaaggauucugagagcgagagc

SEQ ID NO: 496 - RNA, 65 nt, HOMO SAPIENS
gugacccucuagagggaagcacuuucuguugaaagaaaagaacaugcauccuuucagaggguuac

SEQ ID NO: 497 - RNA, 90 nt, HOMO SAPIENS
ugcccuucgcgaaucuuuuugcggucugggcuugcuguacauaacucaauagccggaagcccuuaccccaaaaagcauuugcggagggcg

SEQ ID NO: 498 - RNA, 70 nt, HOMO SAPIENS
ggagauauugcacauuacuaaguugcauguugucacggccucaaugcaauuuagugugugugauauuuuc

SEQ ID NO: 499 - RNA, 110 nt, HOMO SAPIENS
gacaguguggcauuguagggcuccacaccguaucugacacuuugggcgagggcaccaugcugaagguguucaugaugcggucugggaacuccucacggaucuuacugaug

SEQ ID NO: 500 - RNA, 97 nt, HOMO SAPIENS
ugaugcuuugcuggcuggugcagugccugagggaguaagagcccuguuguugucagauagugucuuacucccucaggcacaucuccagcgagucucu

SEQ ID NO: 501 - RNA, 98 nt, HOMO SAPIENS
cgaggggauacagcagcaauucauguuuugaaguguucuaaaugguucaaaacgugaggcgcugcuauacccccucguggggaagguagaaggugggg

SEQ ID NO: 502 - RNA, 73 nt, HOMO SAPIENS
cagggugugugacugguugaccagaggggcaugcacuguguucacccugugggccaccuagucaccaacccuc

SEQ ID NO: 503 - RNA, 90 nt, HOMO SAPIENS
ccgggccccugugagcaucuuaccggacagugcuggauuucccagcuugacucuaacacugucugguaacgauguucaaaggugacccgc

SEQ ID NO: 504 - RNA, 100 nt, HOMO SAPIENS
gggccaaggugggccaggggugguguugggacagcuccguuuaaaaaggcaucuccaagagcuuccaucaaaggcugccucuuggugcagcacagguaga

SEQ ID NO: 505 - RNA, 89 nt, HOMO SAPIENS
cuaauggauaaggcauuggccuccuaagccagggauuguggguucgagucccaucugggguggccugugacuuuuguccuuuuuucccc

SEQ ID NO: 506 - RNA, 97 nt, HOMO SAPIENS
ccuagaauguuauuaggucggugcaaaaguaauugcgaguuuuaccauuacuuucaauggcaaaacuggcaauuacuuuugcaccaacguaauacuu

SEQ ID NO: 507 - RNA, 79 nt, HOMO SAPIENS
acuguccuuuuucgguuaucaugguaccgaugcuguauaucugaaagguacaguacugugauaacugaagaaugguggu

SEQ ID NO: 508 - RNA, 71 nt, HOMO SAPIENS
uguguuaaggugcaucuagugcaguuagugaagcagcuuagaaucuacugcccuaaaugccccuucuggca

SEQ ID NO: 509 - RNA, 81 nt, HOMO SAPIENS
caugcugugacccucuagagggaagcgcuuucuguugucugaaagaaaagaaagugcauccuuuuagagguuuacuguuug

SEQ ID NO: 510 - RNA, 85 nt, HOMO SAPIENS
ucucaugaugugaccaucuggagguaagaagcacuuuguguuuugugaaagaaagugcuuccuuucagaggguuacucuuugaga

SEQ ID NO: 511 - RNA, 75 nt, HOMO SAPIENS
uggagugggggggcaggaggggcucagggagaaagugcauacagccccuggcccucucugcccuuccguccccug

SEQ ID NO: 512 - RNA, 80 nt, HOMO SAPIENS
gguaccugaagagagguuuucuggguuucuguuucuuuaaugaggacgaaacacaccugguuaaccucuuuuccaguauc

SEQ ID NO: 513 - RNA, 96 nt, HOMO SAPIENS
gugacccugggcaaguuccugaagaucagacacaucagaucccuuaucuguaaaaugggcaugauccaggaaccugccucuacgguugccuugggg

SEQ ID NO: 514 - RNA, 94 nt, HOMO SAPIENS
acugauauauuugucuuauuugagagcugaggaguauuuuuaugcaaucugaaugaucucagcugucugaaaaugucuucaauuuuaaaggcuu

SEQ ID NO: 515 - RNA, 94 nt, HOMO SAPIENS
aucacagacaccuccaagugugcagggcacuggugggggccggggcaggcccagcgaaagugcaggaccuggcacuuagucggaagugagggug

SEQ ID NO: 516 - RNA, 91 nt, HOMO SAPIENS
cgacuugcuuucucuccuccaugccuugaguguaggaccguuggcaucuuaauuacccucccacacccaaggcuugcaaaaaagcgagccu

SEQ ID NO: 517 - RNA, 81 nt, HOMO SAPIENS
aacacagugggcacucaauaaaugucuguugaauugaaaugcguuacauucaacggguauuuauugagcacccacucugug

SEQ ID NO: 518 - RNA, 110 nt, HOMO SAPIENS
acccggcagugccuccaggcgcagggcagccccugcccaccgcacacugcgcugccccagacccacugugcgugugacagcggcugaucugugccugggcagcgcgaccc

SEQ ID NO: 519 - RNA, 110 nt, HOMO SAPIENS
ggccuggcuggacagaguugucaugugucugccugucuacacuugcugugcagaacauccgcucaccuguacagcaggcacagacaggcagucacaugacaacccagccu

SEQ ID NO: 520 - RNA, 110 nt, HOMO SAPIENS
aggaagcuucuggagauccugcuccgucgccccaguguucagacuaccuguucaggacaaugccguuguacaguagucugcacauugguuagacugggcaagggagagca

SEQ ID NO: 521 - RNA, 86 nt, HOMO SAPIENS
uggggcccuggcugggauaucaucauauacuguaaguuugcgaugagacacuacaguauagaugauguacuaguccgggcaccccc

SEQ ID NO: 522 - RNA, 110 nt, HOMO SAPIENS
aaagauccucagacaauccaugugcuucucuuguccuucauuccaccggagucugucucauacccaaccagauuucaguggagugaaguucaggaggcauggagcugaca

SEQ ID NO: 523 - RNA, 94 nt, HOMO SAPIENS
uuuuuuuuuaguauuuuuccaucaguguucauaaggaauguugcucuguaguuuucuuauaguguggcuuucuuagagcaaagaugguucccua

SEQ ID NO: 524 - RNA, 96 nt, HOMO SAPIENS
agacaugcaacucaagaauauauugagagcucauccauaguugucacugucucaaaucagugacaacuauggaugagcucuuaauauaucccaggc

SEQ ID NO: 525 - RNA, 94 nt, HOMO SAPIENS
agagcaucgugcuugaccuuccacgcucucguguccacuagcaggcagguuuucugacacaggcugcggaauucaggacagugcaucauggaga

SEQ ID NO: 526 - RNA, 81 nt, HOMO SAPIENS
cuucaggaagcugguuucauauggugguuuagauuuaaauagugauugucuagcaccauuugaaaucaguguucuuggggg

SEQ ID NO: 527 - RNA, 99 nt, HOMO SAPIENS
uuuagcgguuucucccugaagugauguguaacugaucaggaucuacucaugucgucuuugguaaaguuaugucgcuugucagggugaggagaguuuuug

SEQ ID NO: 528 - RNA, 79 nt, HOMO SAPIENS
agccucgucaggcucaguccccucccgauaaaccccuaaauagggacuuucccggggggugacccuggcuuuuuuggcg

SEQ ID NO: 529 - RNA, 85 nt, HOMO SAPIENS
ugguucccgcccccuguaacagcaacuccauguggaagugcccacugguuccaguggggcugcuguuaucuggggcgagggccag

SEQ ID NO: 530 - RNA, 66 nt, HOMO SAPIENS
aaaagguggauauuccuucuauguuuauguuauuuaugguuaaacauagaggaaauuccacguuuu

SEQ ID NO: 531 - RNA, 96 nt, HOMO SAPIENS
ggagugaacucagauguggagcacuaccuuugugagcagugugacccaaggccuguggacaggguaagcugaaccucugauaaaacucugaucuau

SEQ ID NO: 532 - RNA, 97 nt, HOMO SAPIENS
gauugaugcuguugguuuggugcaaaaguaauugcagugcuucccauuuaaaaguaauggcacacacugcaauuacuuuugcuccaacuuaauacuu

SEQ ID NO: 533 - RNA, 85 nt, HOMO SAPIENS
ucucaagcugugacugcaaagggaagcccuuucuguugucugaaagaagagaaagcgcuucccuuugcuggauuacgguuugaga

SEQ ID NO: 534 - RNA, 81 nt, HOMO SAPIENS
cccauuggcauaaacccguagauccgaucuuguggugaaguggaccgcacaagcucgcuucuaugggucugugucagugug

SEQ ID NO: 535 - RNA, 89 nt, HOMO SAPIENS
cuggccuccagggcuuuguacaugguaggcuuucauucauucguuugcacauucggugaaggucuacugugugccaggcccugugccag

SEQ ID NO: 536 - RNA, 89 nt, HOMO SAPIENS
ugcugcuggccagagcucuuuucacauugugcuacugucugcaccugucacuagcagugcaauguuaaaagggcauuggccguguagug

SEQ ID NO: 537 - RNA, 78 nt, HOMO SAPIENS
uugugcuuucagcuucuuuacagugcugccuuguagcauucaggucaagcagcauuguacagggcuaugaaagaacca

SEQ ID NO: 538 - RNA, 78 nt, HOMO SAPIENS
cugaggagcagggcuuagcugcuugugagcaggguccacaccaagucguguucacaguggcuaaguuccgccccccag

SEQ ID NO: 539 - RNA, 90 nt, HOMO SAPIENS
gagagaagcacuggacuuagggucagaaggccugagucucucugcugcagaugggcucucugucccugagccaagcuuuguccucccugg

SEQ ID NO: 540 - RNA, 89 nt, HOMO SAPIENS
ggagaggcugugcuguggggcaggcgcaggccugagcccugguuucgggcugccugggucucuggccugcgcgugacuuugggguggcu

SEQ ID NO: 541 - RNA, 83 nt, HOMO SAPIENS
ucaggcugugacccucuugagggaagcacuuucuguugucugaaagaagagaaagugcuuccuuuuagaggcuuacugucuga

SEQ ID NO: 542 - RNA, 97 nt, HOMO SAPIENS
accucucuaacaaggugcagagcuuagcugauuggugaacagugauugguuuccgcuuuguucacaguggcuaaguucugcaccugaagagaaggug

SEQ ID NO: 543 - RNA, 98 nt, HOMO SAPIENS
ggauucuuauaggacaguauguucuuccaggacagaacauucuuugcuauuuuguacuggaagaacaugcaaaacuaaaaaaaaaaaaaguuauugcu

SEQ ID NO: 544 - RNA, 89 nt, HOMO SAPIENS
cugauggcugcacucaacauucauugcugucgguggguuugagucugaaucaacucacugaucaaugaaugcaaacugcggaccaaaca

SEQ ID NO: 545 - RNA, 65 nt, HOMO SAPIENS
cuugaauccuuggaaccuaggugugagugcuauuucagugcaacacaccuauucaaggauucaaa

SEQ ID NO: 546 - RNA, 67 nt, HOMO SAPIENS
gugggccucaaauguggagcacuauucugauguccaaguggaaagugcugcgacauuugagcgucac

SEQ ID NO: 547 - RNA, 110 nt, HOMO SAPIENS
gagcugcuugccuccccccguuuuuggcaaugguagaacucacacuggugagguaacaggauccggugguucuagacuugccaacuauggggcgaggacucagccggcac

SEQ ID NO: 548 - RNA, 62 nt, HOMO SAPIENS
ucauugguccagaggggagauagguuccugugauuuuuccuucuucucuauagaauaaauga

SEQ ID NO: 549 - RNA, 110 nt, HOMO SAPIENS
cgccucagagccgcccgccguuccuuuuuccuaugcauauacuucuuugaggaucuggccuaaagagguauagggcaugggaaaacggggcggucggguccuccccagcg

SEQ ID NO: 550 - RNA, 90 nt, HOMO SAPIENS
ggaggcccguuucucucuuugguuaucuagcuguaugagugccacagagccgucauaaagcuagauaaccgaaaguagaaaugauucuca

SEQ ID NO: 551 - RNA, 110 nt, HOMO SAPIENS
gugauaauguagcgagauuuucuguugugcuugaucuaaccaugugguugcgagguaugaguaaaacaugguuccgucaagcaccauggaacgucacgcagcuuucuaca

SEQ ID NO: 552 - RNA, 73 nt, HOMO SAPIENS
cuccucagaucagaaggugauuguggcuuuggguggauauuaaucagccacagcacugccuggucagaaagag

SEQ ID NO: 553 - RNA, 81 nt, HOMO SAPIENS
ugugcaucguggucaaaugcucagacuccugugguggcugcuuaugcaccacggauguuugagcaugugcuauggugucua

SEQ ID NO: 554 - RNA, 119 nt, HOMO SAPIENS
aggauucugcucaugccagggugagguaguaaguuguauuguugugggguagggauauuaggccccaauuagaagauaacuauacaacuuacuacuuucccugguguguggcauauuca

SEQ ID NO: 555 - RNA, 96 nt, HOMO SAPIENS
agauaaaucuauagacaaaauacaaucccggacaacaagaagcuccuauagcuccuguagcuucuugugcucuaggauuguauuuuguuuauauau

SEQ ID NO: 556 - RNA, 97 nt, HOMO SAPIENS
augggguaaaaccauuaugcauuguauuuuuaggucccaauacaugugggcccuaaaaauacaaugcauaaugguuuuucacucuuuaucuucuuau

SEQ ID NO: 557 - RNA, 96 nt, HOMO SAPIENS
cgggccccgggcgggcgggagggacgggacgcggugcaguguuguuuuuucccccgccaauauugcacucgucccggccuccggcccccccggccc

SEQ ID NO: 558 - RNA, 110 nt, HOMO SAPIENS
ccgccccgggccgcggcuccugauuguccaaacgcaauucucgagucuauggcuccggccgagaguugagucuggacgucccgagccgccgcccccaaaccucgagcggg

SEQ ID NO: 559 - RNA, 85 nt, HOMO SAPIENS
aggguagagggaugagggggaaaguucuauaguccuguaauuagaucucaggacuauagaacuuucccccucaucccucugcccu

SEQ ID NO: 560 - RNA, 84 nt, HOMO SAPIENS
gaugcacccagugggggagccaggaaguauugauguuucugccaguuuagcgucaacacuugcugguuuccucucuggagcauc

SEQ ID NO: 561 - RNA, 96 nt, HOMO SAPIENS
ugggggagugaagaguagauaaaauauugguaccugaugaaucugaggccagguuucaauacuuuaucugcucuucauuuccccauaucuacuuac

SEQ ID NO: 562 - RNA, 87 nt, HOMO SAPIENS
ggaagcgaguuguuaucuuugguuaucuagcuguaugaguguauuggucuucauaaagcuagauaaccgaaaguaaaaacuccuuca

SEQ ID NO: 563 - RNA, 76 nt, HOMO SAPIENS
gagggggaagacgggaggaaagaagggagugguuccaucacgccuccucacuccucuccucccgucuucuccucuc

SEQ ID NO: 564 - RNA, 88 nt, HOMO SAPIENS; identical to SEQ ID NO: 470

SEQ ID NO: 565 - RNA, 94 nt, HOMO SAPIENS
ugacuccuccaggucuuggaguaggucauuggguggauccucuauuuccuuacgugggccacuggauggcuccuccaugucuuggaguagauca

SEQ ID NO: 566 - RNA, 96 nt, HOMO SAPIENS
uucauuccuucaguguugaaacaaucucuacugaaccagcuucaaacaaguucacuggaguuuguuucaauauugcaagaaugauaagauggaagc

SEQ ID NO: 567 - RNA, 99 nt, HOMO SAPIENS
uggcuaagguguuggcucgggcuccccacugcaguuacccuccccucggcguuacugagcacugggggcuuucgggcucugcgucugcacagauacuuc

SEQ ID NO: 568 - RNA, 127 nt, HOMO SAPIENS
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 569 - RNA, 98 nt, HOMO SAPIENS
guguaguagagcuaggaggagaggguccuggagaagcguggaccgguccggguggguuccggcagguucucacccucucuaggccccauucuccucug

SEQ ID NO: 570 - RNA, 94 nt, HOMO SAPIENS
uguuuugagcgggggucaagagcaauaacgaaaaauguuugucauaaaccguuuuucauuauugcuccugaccuccucucauuugcuauauuca

SEQ ID NO: 571 - RNA, 96 nt, HOMO SAPIENS
ugguacuuggagagauaguagaccguauagcguacgcuuuaucugugacguauguaacacgguccacuaacccucaguaucaaauccauccccgag

SEQ ID NO: 572 - RNA, 102 nt, HOMO SAPIENS
cccaagucagguacucgaauggagguuguccaugguguguucauuuuauuuaugaugaguauuacauggccaaucuccuuucgguacucaauucuucuuggg

SEQ ID NO: 573 - RNA, 88 nt, HOMO SAPIENS
aucucuuacacaggcugaccgauuucuccugguguucagagucuguuuuugucuagcaccauuugaaaucgguuaugauguaggggga

SEQ ID NO: 574 - RNA, 97 nt, HOMO SAPIENS
cagacuauauauuuagguuggcgcaaaaguaauugugguuuuggccuuuauuuucaauggcaagaaccucaguugcuuuugugccaaccuaauacuu

SEQ ID NO: 575 - RNA, 88 nt, HOMO SAPIENS
uguuaaaucaggaauuuuaaacaauuccuagacaauauguauaauguucauaagucauuccuagaaauuguucauaaugccuguaaca

SEQ ID NO: 576 - RNA, 86 nt, HOMO SAPIENS
acugcuaacgaaugcucugacuuuauugcacuacuguacuuuacagcuagcagugcaauaguauugucaaagcaucugaaagcagg

SEQ ID NO: 577 - RNA, 109 nt, HOMO SAPIENS
ugaguuuugagguugcuucagugaacauucaacgcugucggugaguuuggaauuaaaaucaaaaccaucgaccguugauugucccuauggcuaaccaucaucuacucca

SEQ ID NO: 578 - RNA, 70 nt, HOMO SAPIENS
guuguuguaaacauccccgacuggaagcuguaagacacagcuaagcuuucagucagauguuugcugcuac

SEQ ID NO: 579 - RNA, 109 nt, HOMO SAPIENS
ggucgggcucaccaugacacagugugagaccucgggcuacaacacaggacccgggcgcugcucugaccccucgugucuuguguugcagccggagggacgcagguccgca

SEQ ID NO: 580 - RNA, 110 nt, HOMO SAPIENS
ccagaggacaccuccacuccgucuacccaguguuuagacuaucuguucaggacucccaaauuguacaguagucugcacauugguuaggcugggcuggguuagacccucgg

SEQ ID NO: 581 - RNA, 85 nt, HOMO SAPIENS
cgcuggcgacgggacauuauuacuuuugguacgcgcugugacacuucaaacucguaccgugaguaauaaugcgccguccacggca

SEQ ID NO: 582 - RNA, 92 nt, HOMO SAPIENS
cggcuggacagcgggcaacggaaucccaaaagcagcuguugucuccagagcauuccagcugcgcuuggauuucguccccugcucuccugccu

SEQ ID NO: 583 - RNA, 2073 nt, HOMO SAPIENS
  misc_feature (888)..(888): n is a, c, g, or u
  misc_feature (923)..(923): n is a, c, g, or u
  misc_feature (992)..(992): n is a, c, g, or u
  misc_feature (1706)..(1706): n is a, c, g, or u
  misc_feature (1773)..(1773): n is a, c, g, or u
  misc_feature (1780)..(1780): n is a, c, g, or u
  misc_feature (1794)..(1794): n is a, c, g, or u
  misc_feature (1810)..(1810): n is a, c, g, or u
  misc_feature (1821)..(1821): n is a, c, g, or u
cgaacaggagggaaagacagggaaagaccgggcggaggagagcggacgagcagagaagaaagccaacggaaccgaagccagccacgcagcaaggaccaggaacaaaaccaaaacaaaacagaccaaacacaggccacagacaggaaggaaaggcgggaggccaggcaaacaaggaagggcagaccagacagaaggagagggcagcacgcaggagccacggcagacggccaccgcaaccccgcccgcacaagcaccgccagccccagagagcggggaacaagcaggccaacagcccggcaagagcgggagacaggcgccaggccaggggccaaggaccacccgcccagccccacaaaggcgggaacagcggagccaccgacccagccacaccgcacacagcacaaggacacaagagaaaagacaggagcccagccaacccggcgcacacccggccaacccaagaaagcagagcagcgcacgaggcagccccaggcaaaagagaaagcaggggcgagacagggagaggagacgaaaaaagcagaagcaagacaccagagaaaaacacaaaagaacccgcgacacaagacgaggggagaaagaccagacagaagggagaaaggcaaaaggagcgagccccacggagaaaaagagcaaacaccagcaagaacacaccaaccacaacgaccagcgagagagggccaacggagacacagagagggcgcgccaaagcgaagaaggaggaagggaaaagaaggaaagaaaagccaaaaaaaccagaacagaaaagcaagacaccaggacaggaaaacgaggaggggaacaagacaaaagaggggggaacggacacagacaaagacggaaggaaaagcgggggcagagaaagaacanccaaaaggaggagaaagaaagcgccggagccgccncggagaaagaggaaaaccccagagggccaggcgaccaggcagagcgaagggagaagagaaaaagagccnccacaacgaaagaagaaaaagcagaaggagaaaaaagggaaaggccagaaggagggagggacagggggcacccagaggggcaacagagaagcagggaggcggcccagcccaccaaacaacccaggggggaccacagagacaaaaaaaccccgaaaaaagaaccggaagagagacacggaggcggggcacggggcggggcacgggggggggggggagggaggaggagggggcaagagagaacaacgaaggagacacccaccgcaaagggagcggagggccagccgcaacagacaaagaagcgagagagggaaggaagagagcagaagaggcaaagcaaagagagaggaggaaaacaaaaagcaaaaagggggaacagccgaggagaagggagacaggaaagagagaagagagcagggaaaaaggaaaacgcagggaagggaacaaagggggagaggagcagaaacaaacagaagcgaccagaaaagaagcagcacccccagagccaacccgagggaccaaaaaggaaagccacgagagaaagcgaagggcgaaaagccgaaaaaagaagaaaaaggaggacagaagccggagcaggagaaaaggggaaagaacaccgagggcaaacacagacgggccgaccaggcggcaaggaaagagagaaggaacaagcagacacagacaggaccaacagaggcaaagcaagacacgaagganaaagcaagaacacagacgcaaaaaaggacagaccagggaacgaaaggcagaaaagaaacaagagaanccaaacncaaggaaaaaggangacagagccaaggcanagcagggaggncgaaacaccaaaccggaaaacagacaaacacaaaaaaacacaaaaaaaaaaccacggaagaggaaacgagcagaaaccagcaggaacaacaaccagaacaaaaaaaaagagaaacagccacaaacagccaaaaacacgggaaaagaagacaaaaccgaaaacagagaaaaaaaaaacaaagagcaggagaccaagaaaaggggaacgccaaaccacccggaggaaggaaaggaaccgggaagaaacgaaccc

SEQ ID NO: 584 - RNA, 752 nt, HOMO SAPIENS
aggagacacccggggggagccgagacgggcaggcggggggcaggggccgccccaggggaaagggacagagggcaggagggcgcagcccccagggagggacagggcccggggaggcaggcagcaagaagcagggaccagcaaggcgggagcaggcgggcccgggcggacagagaaaggggacgaagggcacagcggggccccagccgcacagcaccacccacgcacacccaagggcaggacaggagggccggggggcaaggccgccagagcacgagggcaggagagcgagacagagggcgcaggcgcagggacaccgcaaaaggcaccaccgggagggagggcgccggggcgggcccagcacacccggccaagccccacgcggcacgcggcccaggggcgggcgggaagggcccacagggcgggacacgagccaaccgcccagcccggggcggcagaagacaggcagccaggggagcaagggcacgagcgaggccaggcagggagcgggcgggcggaggaggagcccgggggagggggcgagagcaggcagggcaggagcagagcggcgggcacagcgggcacggccgcgcgccagggccaaggcgggccggcagggacagagcaagaccggccgccgggagacggagacgagggaggcccagcgggggacacaacagccagccaccaggaaggccaggccccacgccggcagaggcgagaaggacagacaggccggggacggcgaggacaga

SEQ ID NO: 585 - RNA, 752 nt, Elephantidae sp.; identical to SEQ ID NO: 584

SEQ ID NO: 586 - RNA, 1426 nt, Felis catus
aggagacacccggggggagccgagacaggggggcggggagcgggggcccccaggagaaagagacaggaggggggcaggagacgaggcagggcgccagcgaggaagggcgaggcgaccacccaggagggacaggggccgggggaccagcaggcgggggcaccaggcccgggcggacaggaggaggggggaccaggacaaacagggccccagccgccaccacaccacccaccgaaacaagagcaggacaggaagcggcccggccaggcccccaaggccaggcggagccgagggcggagaacaggaacgaaggagcaggcgcggggcggcccgcagcaagggcacccggagggagcggcggccacacggacgggccggagagacgcagcgaggaagcgccggggcccggccgccggggccgcccacggcacgcggcccaagggccggcgcggaaaaggccacaagcccggaccgagccccaccgccagccccgggcggcacgacaggacagcccaggggagcaaggcacaggcgagaccagaaggcgaggaggggggcggaggaggagaccggcgggggggcggggcgcaggcaggggcgaagagcaggagccggcggcgagccaggggaggcacggccccagagcccgggccaaggcgggcccgcgggcgcgacggagcagggacgggcccgggccggacggagagaggccaggcccagcggggggagcccgccagccaccgaaggccaggcgcgaggcgcgccagagggacaggacaggccggggaggcgaggacagacggagccgccggccccgaacacaggcggcaccgcggggagcagaacagccacggcccgccgcgccgaaggagcaacacacgcgcccggaccccaaaccccaaacgaaagaaagggcacccagccagccgggccggaaaccggacccaagcccagccgacgcaagaagagcgcacggaaccacacacacgacccaagaacggcggaagggacgcaaaagaacaagagcaacgaccacaacaccgcaggagcaagcccccgggcagccgccacccacccccggcaagaccccgggccacccgccaggcaggcgaccaccagccgccacgccccacgcccccgcccccccccccagcccaccccccccgccgggagacaaagcccccagaccgccccgcccccgaaaagcacaagcggcacggggagcggacggccgagcccgagcggggcaagcaagcgcgaggcaccggccccgggcccacagcgcccggcacccaccccgccaccggggggggcccggggcggcgagagcggaggccccacccccccgccagcggaagcccaacaccgagcagaccgaagcaccagagcagaccgccggccccccccaccagggcccaacagaggccgg

SEQ ID NO: 587 - RNA, 764 nt, LYNX LYNX
agggacacccggggggagccgagaccggcgggcggggggggccgcggcccccaggagaaagagcacgggagagcaggcaggagagaggcagggcgccggcagagagggacaggagcgggggaccagcacggcgggggggggggggggcaccaggcccgggcggacaggagaggggggaccaggacgcagcaggggccccagccgccaccacaccacccacgaaacaagagcaggacaggagcggccggccaggccagacgcgagggcggggagcgggaaagaaggggcaggcgcgggggcccgaacaggacgcaccggaggagcggcggccgagaccggcccggagagacgcagcgaggagcgcgcgggcccagccgcggacgcgccgcccacgggcacgcggcccagggcggcgcggagaggcccacagcgacgggacacgagcccaccgccagcccccggcggcagacagacaggacagcaccagggagcaagggcacggcgagacccgacaggcgaggcgggggggggaggaagagaaccggggggggccggggcgcaggcaggggcgaagggcgggacggcgcgagccgccggggcgcggccccagagcccgggccgagggggcccgcgggcgacgacggagcagggacgggcccggaccgggacggagacgaggccaggcccagacggggacggcgcgccccgaaggccgggcgcgagcggcgccagagaggcagacaggccgggcacgggaggacaga

SEQ ID NO: 588 - RNA, 1354 nt, PONGO PYGMAEUS
aggagacacccggggggagccgagacgcgcaaggcggggggcggggcccacccaggcagaaagagcaagagggcagggagggagcacaggggggccagcgagggccagcacgggggggaccccaggccgggcagacagggacaaggcaggggacacaggacagaggggccccagcgccacccacccaccgcaacaagagcaggcacaggggcagcccggcacggcccaggccagccggagcccgagggcggagagcgggaagacaggcaggccggggagcgcagcggacgcacaggaggggaagcgcccacgggaggggggccccgggacagcgcagcaaggaggcgcaggggccggccgcggcgccggcccacgaggcacgcggcccagggcgggcggagaggcccacagggacgggacgcgagccccaccgccaccccggggcggcgcagacagacagcaccaggggagcaagggcaggggcgagaccagacagcgagcggcggggcggaggaagagccaggaggaggcgggcaggcaggggaggagccaacgacggcgaacgggaggcacggccccagagccgggccaagggggcccgcgggcggcggcggaccgggacgggcccagcgcgggcggagacgaggccaggcccagcggggggaggcccaccagcgccgaaggccaggacgccaggccggggacggccaaggggacagacaggccggggagggaggacagaggaggcgcgcggccccggaacaccaggcggggggcgcggcgagaagcgggcgcacccccacggagcggcacacacggcgcccgggccccagaacccacaacagaaagaaagggcacccagccaagccgggccgaaccggacacaaaaccccagcggaaggaaagcgacacaaccacgcacaccgaccaagaacggccggaaggggaggggaacagcagcggccgcagaccacccccacggcaaccaccaggccaccccgcggcggacgggccaccagccaccacgcacccagagagagccccagcgggagaccccgccccaccacccccccccacccgccgcgagcccgccccgagagacaaagccccacgaccgcccccgccccgaaaagcacaagcagcacggggaggggagacggccgagccggggagcggcggagggagccaccggcaaggcccgcccagagaccggccgcccccacgggccggggacgggcgccggcgggcccgaggcccacccgccccgccagcggaagcccgaccggcacacggagcagcccaagcaccaacgccccaccgccggccccccccaccagggcgcagcagaggccgg

SEQ ID NO: 589 - RNA, 761 nt, THOMOMYS MONTICOLA
aggagacacccggggggagccgagacagcagaaggcgggggggcggggcgccccagacagaaagagcacccagagggcggggggggagcgcagggcgccagcggggacccggagggaccagccaggcgggagcacccaggcggggccggacagggcggggcggggcacaggacagagagggccccagccgccaccacacacccaccgcaacacaggaggaggcccgggcgccccggcgggcccccacgggccagccagagcgcgaggacaggagaacgggaaagaagggcaggcacagcaggacgcaccagagggggaaggggccgcgggggcgggcccgagggacgcacaaggagcgcacgggcccggccgcggcacagcgcccccgcggcacgcagcccagggcggcgcggaaaggccacagggacgcacgcgagccaaccgccagccccgggcggcagcagacaggacagcaccacgggacaagggcacggggcaaaccagacaagcaagcagcgggccgaagaggcgaaaagggagagaggggggggcagcagggcgaggccaaggaacaggggcgagccgaggaccgaccccagagcggcgaccaaggcgggccagcgggcggcgacacaggagcgacggcgcgccgacgcgggagcggagacgaggccaggccagcgggggggacgggccagcagcagaaaggccaggacgcaggcaagaacacaagacagacaggccggggaggcgaggacaga

SEQ ID NO: 590 - DNA, 687 bp, HOMO SAPIENS
atgacgcgctgccccgctggccaagcggaagtggagatggcggagctgtacgtgaagccgggcaacaaggaacgcggctggaacgacccgccgcagttctcatacgggctgcagacccaggccggcggacccaggcgctcgctgcttaccaagagggtcgccgcaccccaggatggatcccccagagtccccgcatcagagacttctcctgggcctcccccaatggggcctccacctccttcaagtaaggctcccaggtccccacctgtggggagtggtcctgcctctggcgtggagcccacaagtttcccagtcgagtctgaggctgtgatggaggatgtgctgagacctttggaacaggcattggaagactgccgtggccacacaaggaagcaggtatgtgatgacatcagccgacgcctggcactgctgcaggaacagtgggctggaggaaagttgtcaatacctgtaaagaagagaatggctctactggtgcaagagctttcaagccaccggtgggacgcagcagatgacatccaccgctccctcatggttgaccaagactgtcccactgggcctgacccacctatgagggaagaagtcccacctgggccagagggagttcatgtgttactcataacatgcatttcaataaaaacatctctgcggaagggatctgctgggtaa

SEQ ID NO: 591 - DNA, 918 bp, HOMO SAPIENS
tcctttggtgccttgtgaccagggccctgatggttcattagatggagccttcgagtcttagggagttgccgcagggtccccacagcggctcccgacggttgtgaaccagcatccatcctccacggattccggcaacccgcctggccctggacgtgtctcaactggcccgcgtgaggggccgccccggaaatgacgcgctgccccgctggccaagcggaagtggagatggcggagctgtacgtgaagccgggcaacaaggaacgcggctggaacgacccgccgcagttctcatacgggctgcagacccaggccggcggacccaggcgctcgctgcttaccaagagggtagccgcaccccaggatggatcccccagagtccccgcatcagagacttctcctgggcctcccccaatggggcctccacctccttcaagtaaggctcccaggtccccacctgtggggagtggtcctgcctctggcgtggagcccacaagtttcccagtcgagtctgaggctgtgatggaggatgtgctgagacctttggaacaggcattggaagactgccgtggccacacaaggaagcaggtatgtgatgacatcagccgacgcctggcactgctgcaggaacagtgggctggaggaaagttgtcaatacctgtaaagaagagaatggctctactggtgcaagagctttcaagccaccggtgggacgcagcagatgacatccaccgctccctcatggttgaccatgtgactgaggtcagtcagtggatggtaggagttaaaagattaattgcagaaaagaggagtctgttttcagaggaggcagccaatgaagagaaatctgcagccacagctgagaagaaccataccataccaggcttccagcaggcttcataatcctcggttccccagact

SEQ ID NO: 592 - DNA, 918 bp, HOMO SAPIENS
tcctttggtgccttgtgaccagggccctgatggttcattagatggagccttcgagtcttagggagttgccgcagggtccccacagcggctcccgacggttgtgaaccagcatccatcctccacggattccggcaacccgcctggccctggacgtgtctcaactggcccgcgtgaggggccgccccggaaatgacgcgctgccccgctggccaagcggaagtggagatggcggagctgtacgtgaagccgggcaacaaggaacgcggctggaacgacccgccgcagttctcatacgggctgcagacccaggccggcggacccaggcgctcgctgcttatcaagagggtcgccgcaccccaggatggatcccccagagtccccgcatcagagacttctcctgggcctcccccaatggggcctccacctccttcaagtaaggctcccaggtccccacctgtggggagtggtcctgcctctggcgtggagcccacaagtttcccagtcgagtctgaggctgtgatggaggatgtgctgagacctttggaacaggcattggaagactgccgtggccacacaaggaagcaggtatgtgatgacatcagccgacgcctggcactgctgcaggaacagtgggctggaggaaagttgtcaatacctgtaaagaagagaatggctctactggtgcaagagctttcaagccaccggtgggacgcagcagatgacatccaccgctccctcatggttgaccatgtgactgaggtcagtcagtggatggtaggagttaaaagattaattgcagaaaagaggagtctgttttcagaggaggcagccaatgaagagaaatctgcagccacagctgagaagaaccataccataccaggcttccagcaggcttcataatcctcggttccccagact

SEQ ID NO: 593 - DNA, 921 bp, HOMO SAPIENS
tcctttggtgccttgtgaccagggccctgatggttcattagatggagccttcgagtcttagggagttgccgcagggtccccacagcggctcccgacggttgtgaaccagcatccatcctccacggattccggcaacccgcctggccctggacgtgtctcaactggcccgcgtgaggggccgccccggaaatgacgcgctgccccgctggccaagcggaagtggagatggcggagctgtacgtgaagccgggcaacaaggaacgcggctggaacgacccgccgcagttctcatacgggctgcagacccaggccggcggacccaggcgctcgctgcttaccaagagggtagccgcaccccaggatggatcccccagagtccccgcatcagagacttctcctgggcctcccccaatggggcctccacctccttcaagtaaggctcccaggtccccacctgtggggagtggtcctgcctctggcgtggagcccacaagtttcccagtcgagtctgaggctcgactgatggaggatgtgctgagacctttggaacaggcattggaagactgccgtggccacacaaggaagcaggtatgtgatgacatcagccgacgcctggcactgctgcaggaacagtgggctggaggaaagttgtcaatacctgtaaagaagagaatggctctactggtgcaagagctttcaagccaccggtgggacgcagcagatgacatccaccgctccctcatggttgaccatgtgactgaggtcagtcagtggatggtaggagttaaaagattaattgcagaaaagaggagtctgttttcagaggaggcagccaatgaagagaaatctgcagccacagctgagaagaaccataccataccaggcttccagcaggcttcataatcctcggttccccagact

SEQ ID NO: 594 - skipped

SEQ ID NO: 595 - DNA, 2554 bp, HOMO SAPIENS
ttttgcaaagccgcggaggtgaagtgaacttagaggttgttgattctaccagaaaggaatgaagaacagaaccttcaggaattgagtcacaatgcagacaaatatcaaatgggagattgttgcaaggaagagattgatgatagtattttctactagccattgggaagataaaaggagacagaagattgaagcctttgccagccattctttccctttttgcttccaaactcctcaactgggaaccttcatatgtgcagtatttatattggatcatactggtgattataaaagttcctaggaggctagaagagccaaccaacagagaagggaaagcagtctgttctgaacatagggacataagttcattcatgccaagtatctttccagcatgtttctcccatttagaatatctagcatgtaaggcctttcaatattaatataagcccaatatcagctctttctctttgtatttcatctctttctactctcctatttgtattttgtgttcctatcaaagtgtcgtatctgggagatgacctgccttatcctgttctataacagttttgtttgctgctgtgtctttagaacagtgcctggcacacagtaagcactcaataaatctttgatgaatgaatgaatggttatcttttctctctggcaatccaaattcaccactccttaaaaattctacataagatttgctggctgggcgcagtgactcacgcctgtaatcccagcactttgggagactgagacaggtggatcacctgaggtcaggagtttgagaccagtctcaacatagcgaaaccccatctctactaaaagtacaaaaattagccgggcgtggtggcaggcgcctgtaatcccagctacttgggagactgaggcaggagaatcacttgagcccaggggggcagaggttgcagtgagctgagatcacgccacttcactccagcctgggcaaaagagcaaaactccgtctccaaaaaaaaaaaaaaaagatttgcctattccagcagacttctacccaatatcaatctagatttaaatcacttgattttccacatccctttagcaattatgtatagtttgcacttcattaacctacccttatttatgttacttctcacatacacccatatctcttctgtattttcaatcaaaagataatgcctcaaatgttatatatattgtatgtgccactcaacagtgcctcatatgctgcaaaaaaaaaagacagtaaacaatcggggtacctttttgtcttttaagccatattttctgggtcagtgtggtctttcaagggagtttagacaggctctattttttgttacttcactcctatagtatgtgactgtaaattgttccttcagtgttcaatttaagtacaaactaaagagaaacagtttcctaaagaaaaacattttaaagttacagagacacctatgtgttcgtaacatttctccagaaatatttgaagtaacgtaaaacttctggactggtcagtttaaatattttcttggaatttctatagaaatttctgtggaatggtgttatattggaaatattaattaactataagaagactcctacttcttcacagagaacaagcagggatatcctggattttcacagtatttaggccactccttagttgtctcatgaaggaattaaggtagtggaacacattcaagaattgcttaggtgtcaaaataagacaaagtgtaacagtttttttctgtagtagatttttaaatagttaaaagactctgccctctttatctatactgtattctggcaataataaagttgggcagcctatacataaaagcaatttctaacaatgccaaattaacagcagtttcttggccctttcctttccctccatgaaagttacaatattaggttatttcacaggtttcctccatgccccccatacccaacctaaaactgtgttaggctgtcaaagaaaacaaatcaggtaagattagtgtttgcctttacgcagtcattctaagtacacataaaatcatacataatatgaaaatacagcaggaagagaaacatcaaaccatagaactgagttttagttttactgacatttatatggcatacttcctgtttttgatttcagaagtgcaaccatgcagtgcttctatgaaaaactgaatagttgagtacagtttgaggattataacaacttatccaaaggaaacaaacagacctaacttactgctgttttcatttgtggaactgctatctttgttgtaaaaagttgagtatttttcaaacatcatttaataatagggtagcccctaaaacaagtaaggagctataagtctgagttgtgaatttgactagtaatggaataagaaaaccaagccaaactccatctaaatatgtatgtaaaggtaatgcttgtaaaaatttgctttttcctcattttgagatttcagttgccagttgactattggtatgcaaaattaacaattctgcctaaaggaggtaaacgatagaggttaattcttaaataaaagtccctttgtttcactat

SEQ ID NO: 596 - skipped

SEQ ID NO: 597 - DNA, 700 bp, MUS MUSCULUS
cccgtcaccgggggtgggaggagggggcagcggtggcagcaatggcagcagcagcagcggctgaacaaaacagatcacctgcagatattttattcgtggggtttgtaaggaaggcgataactgtcaatactctcatgacctctctgacagtccatacggtgtagtgtgcaagtattttcaaagaggatactgtgtttatggagaccgctgcagacacgaacacagcaagccactgaaacatgcagaagtgactgctacagatctgagtgcaaaaccatccctcactgcttcctcaagtctctcttctggtgttggatcacttgctgaaatgaattcaggcgaagctgagtcacgaaacccaaactttccaactgtaggagcaggttcagaggactgggtgaatgccattgagtttgtccctgggcatccctactgtggccttactgccccttcctgcactgaagtacccctgcagggttcagtgaccaaggaagagacagagaaggagccaactacggtggagacaaagaagcagctttgcccctatgctgcagtgggagagtgttgctacggggaaaactgtgtatctccacggagactcatgtgacatgtgtggcctgcaggtcctacactcagtggatgctgcccagaggtcacagcacataaaatcttgcattgaggcccatgagaaggacatgga

SEQ ID NO: 598 - DNA, 920 bp, HOMO SAPIENS
tgtctgtcagagctgtcagcctgcttaagcagagtaaaatggtacaggcagtgcagcctggtagcgagaaaaaaggctgcctgtgaaatcccactgtgggaccataagtggggacctcagggccccttcatggcatctccatggccatgtcatgctggagaaggaggcgtttcaagaatgtgagctgatcgctggaaactgctcatctgactccagtctcaaaagaggctatgtgcaagaatcgggtgaagttgtgagaccccatccacccctcacaagattgtatccccaccctgtctgaccttactgctgctcaaactatctgtccaaggatgaaaacccaggacaaaggaggagtaaccctcatgatgtgaagcacgtgttcacctgtgaatataacctgaggatcatgagactatctgtggatttcacagagaagacagacgagaagacaccgctgacacttctccacggaggtctccttttccaccaagatgcagatgcttcttgcaaggactatcctgtgaatcccacagagaagacaggtgtggttccaatgccggtgcacctccagggaattctccttctctaccaagctccaggccttctgccatgatcatgagactatttgtggatttcacagagaagataggtgaaggtacagcatggcatccacccctcaccagaggggtatccccacccctatctgaccttattaccttattgctgttcaaagtctctatcccagactgaaatcccaagacaatggagaagttccccctgatgatgtgaagcaccaactcctctgggaatcaaattcgaggtaaatttaataggcccggtagagatgaatgatagtgtctctccttggattggctgaaagacaattaaacactggtatatttctgttaaaaaaaaaa

SEQ ID NO: 599 - DNA, 399 bp, HOMO SAPIENS
aatcaccacagatagccactgagaccctctctcaacttcatctgcatgtgtgagaggccagtttgaggtgtgagagtacttctccaatttggacttgcctttgtcatgtttcctgcctatgtgagagagaccctgtgaaacccaggtagaagggaggcagtaaggttaatggcccagccatcttttgctgacccccatctctggggtctcaggtatgattctatcacccaaagacacctaaacaaatcaccagactacattccaatccccatgggacctgattattgcacacagcttctttcagcaatgagtcagaagagcagtttttcagaaccaactcacagtctgaaaaagcaacctcctttttcatatatacacttaacatcaaaaaaaaaaagc

SEQ ID NO: 600 - DNA, 575 bp, HOMO SAPIENS
cactccgttgcactcccgtttgactctgggtatgaaagccatcaaatcaggagttagccaggatggacccaatttgcacaccaaatgtttttggcgtgttggagtactcccacctcaacatcgggccattgtatggactgcttgtgcaattaaaggaatgtgcgaatggtgttggaagcaccttctgtgtcatctgtctttctcagtgttgcaggtgaagttgtaggacccaatccatctctcaccagattgtatcctcaccccatctgactgtattgcttcttacactgtatgtcccaggatgaaatcctaagatgatggaggagggtcccctcatgatgtaaagtacctgctcagataggaatctaatttgaggtaaattccaggggtccagtggacaggattgctagtgtctctctctgagatggctgcaggacaataaaatacaggaagatgtctgttttttggtggggtgtgttcctcttctttctagaagagtggcttttcttttgcagggggagttgatttggatgccggcaggtcttggcatgcctcccaattcacagtggattcgc

SEQ ID NO: 601 - DNA, 3177 bp, HOMO SAPIENS
  misc_feature (1334)..(1334): n is a, c, g, or t
aggcttgccatcaccacagatggcctctgagacactgtttgaaccacatctgcacctgtgagaggccagtttgaggtatgagaacactgtttcaatttggacttgcctttgtcttggttcctgcttttcccagatggcacctacccaacccaggatgaatgagtgcagagaggtcaagtgccaggccatcttttgctgacacccttttctggtatttcaggtataagtccatcatccaaagactgctcaacatctcaccagaatatatttcaatcctcatggggcatgattctttcacaaaacccctttcaggaatggagtcagaagagtagtttccagagacaacctcacagtcttggaacggctctgcctcccatgtgatctgaccatggagatggcatataagggccctaagtttgagacttttagggtactgcaatgcgttatcacaggcagcctttatcctgataccaagccagctctgcctgtaccattttcctctgcttaggcaggctgacagccctgacaccctggtgctccagtttgagtcactatatgtggatgtgctagtcttggggcaatggacctgagctgtgagctgtagctagtgtcacaatgaatgccagctttgctagtaacaattccctttggcttggtagagaaggggacctctgtggaggtacaatggtggtgcactgtcacctgtcttctctgtgggatccatgggacagttccatgatcctaggagagggtagatgtgagccagcctgaagaaatgtcaagcagagccccaggaatgaagcacaaaatcactacagatccaaaaggatctgcagaatttgtcaggcctgcctagacattgtaggggttagtcttattgaaatgtgtcccactgtaatttccaacttcagccttcctgtgttcccagcagtttctctctcccaggtggggctttctgcagaatgacacagcctcagaagctactgggctgtgtgttactgtgggagtgttgcgagtgttggatgtcagcatgtgtgtgtggctttgtgtgtgtgtgtaggtgtctgtgtgtgtgtatgtaaatgaattctgtggatcaggaatcagcaatgactagttaagctgtctgtgaccagccgggttccccatcgtctgcccctgccaaaaaaacaggtactcttctacaaagaagaggagagcaccacacccaagaacagacatctcccagtgttgcattataaagcagccaacccacagacactagcactctggtctgcatagcccctttaatttacctagaattcagttcccagccaagtaggtgcttcatgtcntgagggtgcaatcctccatcatcttgagatttcatgctggtacagagagtgtgacagcaataaggtcagataggggtgagtatacaacctggtgaagggtggatggggtcccgtaccttcaccagcaaaaagggtgaaaatagatgacacagaatgtgcttccaactccatccccacattcccataattgcaaaatcagtcaacaacatggcctggtgtttaggtgggagtactccaacctgcaggaagaatttggagtgcaaattgtggccaatctggaaaactcctggtttgagggttttaatacctgtagtcaaatggaagtggaatagattgatgctgggtgggttgtggcctccacatttgtgtcctcttttactgacttccattgtcctcattggtgtagggcttcctggatctggctcaacatcttccacactaaactcttccctgttcacagaagaccatcataaaaatgcattgtagaggccctgcaaggaccaggatgaagggagacagtgaggtcaagagcccagccatctttcactgacacccacttctgggttctcaggctggctgacaggtctgacagcccatcacgaaagcctgcatactcttagacacaaggactgagctatgggctccagctagcatcacaatgaaggccaccattgcctagggataagtccctgtgactttgtggataagaactccgtggagccaatccaaggagagacactatcattcatctctaccgggcctattaaatttacctcgaatttgattcccagaggagttggtgcttcacatcatcagggggaacttctccattgtcttgggatttcagtctgggatagagactttgaacagcaataagtttccttggtctggctcaacgtcttctaaactcaacattccccagttcatggaaaatgatcctcatgggattctattgtaaaagctgcatgattcctggagaatgttttattgtcctggagtcaacccaaaaatcaacactaccagtcatgttgcagagctccttgaattaaccttgaattcagtttccagctgagcagctgcttcacgttgtgagggggcaatcctccatcatcctgggatttcattctgggacacagagtttgagcagcaataaggctgggtccacattgcccctcaacagcattagtggacatgattgtcagacttgcaatttccgcagacaccttctgtgaacatttttcaacatcatctacatgagtgagagacccgttcgacatgtaagaatactgcttgactttggacctgcctttgtcgtggttcctgcctttctcatagatcccctgccaggcccaggatgataggaggcaatgaagtcaagggccgagccccattcattgaaagctgactctggggtctcgggtataattccatcacataaaatcccctcaacaactcaccagactatattccaatctccatggaacctgattcttgcacacagcctctttcaggaatggagtcagaagagcagtcttcagagaccacctcagtttggaaaagcctcctccttcagtggttcccagccatggagtcatcgtgaaggggctccatggtcaataattttacggtactgcacttggttatcacagacagactttttcatgatagcatgccatctctgtctatatcattttcctctgcttaggcaggctgacaactctgacagccaggggcccgaatctacctggcaaatgtgcatgctctactctcagtgcaaaaggcctgtttgggagttctgactagtgtcacaataaatgccgccattgcctagtgaaaaaaaaaa

SEQ ID NO: 602 - DNA, 3923 bp, HOMO SAPIENS
acagaagaaatagcaagtgccgagaagctggcatcagaaaaacagaggggagatttgtgtggctgcagccgagggagaccaggaagatctgcatggtgggaaggacctgatgatacagaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 603 - DNA, 5435 bp, HOMO SAPIENS
  misc_feature (9)..(9): n is a, c, g, or t
  misc_feature (1239)..(1239): n is a, c, g, or t
cccgggagntggagcttgcagtgagccaagatggtgccactgmmctccagcctgggtgacaaagcgagactccatctcaaaaaaagaaaaaaaagaaagcattgggaaagcctaaatggtctacaggccacttggaagagaagtgacacaactctcagaaagggaaagaatgaatgaatgaaagaatgtgctttctggggaggaacagtcagcatttttgataatttctgagaggcaggtcagtggagtagttaagaccatgaacgctagggccagattctgcttcttcatatttttacagccttgggaaagttattctctctgtgccacactttgtttatctgaaacatgaagacamctacagcatcttctttacagagtctttctaaaaattaaatgagttaatttttgtaaaatccttcaaagagtcccagccatatgttaagttccatgtaaatactttgctaataaaaataaaattttggagttataagcgcagagtttcttggataatactgttctatgtcctaaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagttacatggtagaacgaagaatagaccagaagccagaagtgctggcctttaaccatatacctgctattcgctagctgtgtaccctctgcaaatgtcctgggtcttagattggtgtctatgaattagacaatctttaagatctcttctagcctcccccatgttacgtgtcattttccttaggtttcttttaaagtgcccaaaacagtccacatagtaggtattgtggtgattaaacagaaaggcactgaaccacaaagcagacacttgattaacatcagttgtatctgactatgcctttttttgtttgtttgtttttttggaaacagagttccactctgtcgcccaggttcgagtgcaatggcmmaattacagctcactgcaacctccgcctcccatgttccagcaattctcctgcctcagcctcccaagtagctgggattacaggcatgcaccagcacgcccagctaatttttgtatttttagtagagatggggtttcaccatattggccaggctggtcttgaactcctgacctcagntgacccacctgcctcagcctcccaaagtgctgggattacaggtgtgagccactgctccccaagaagtcatatgacctgtgggrtctctagaggggacctcaagagaatggtgtgtgtcaggcactgcactagtgactttatgtagattgcttcatttgttcctgccaacagcccactgaaataagtattaaaattctgtgcccagtgaagaaacagagagggtgtttgtgatgctcggaaaccaataatattgtgtgtagctctcaactcaagttcagggwwwgctgacaccttgccatttgtctcctcaaatggtcgtatgtatttccaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagaggtaagtgctttataaagcactcaatttctactcagaaatttttgatggccttaagttcctctactcgtttctatccttcctactcactgtcctcccggaatccactaccgattttctatttcttgcctcgtattgtctgactggctcacttggatttatctcacggagtctggattttctacccgggctcacctccgtccctccatatttgtcctccactttcacagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 604 - DNA, 3638 bp, HOMO SAPIENS
gtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 605 - DNA, 1782 bp, HOMO SAPIENS
gtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactca

SEQ ID NO: 606 - DNA, 720 bp, HOMO SAPIENS
gtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttca

SEQ ID NO: 607 - DNA, 1603 bp, HOMO SAPIENS
aaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaaaaaaaaaaaaaaa

SEQ ID NO: 608 - DNA, 3414 bp, HOMO SAPIENS
cgcatcccaggaagaaggagttggagctccgtgatgccattcaaaaacagatgagatggggacagatttaacccaaaggacatagttcaactcctggtcttgacagaggagaagccctcaaagatggagtctcactctgttgccaggctagaatacaatggcacgaacttggctcactgcaacctccgcctcctgggttcaagagattctcctgcctcagagtcctgagttgctgggactacgggcacacaccatcatgcccagctaatttttgtatttttagtagagacagggtttcaccatgttgaccaggatggtctcgatctcttgacctcgtgatctgcccacgtcggcctcccaaagtcctgggattacaggcatgagccagtgcgcctagcaaacgacagctttttgagcctctgtttaacaatctcttaaaccttataggtaggcagttctgagacagctcctaatcatctcccctcctggtattcaagccctgatggaatactctcttcttcaatgtgagctggacttagtggttttcttctaatccattgaatacacacaagtgatgagctgtcatttccaaaattagttcacacaaagctgtaacttttgttttgttcacagcctctctcaattgccttctcagcttgtatgctttgaagaagcaagctgcagtgttggagaggcccatgtggcaaggaaatgagggtggcctccagtcaatggctggtgagaaactgagaccttccttccaacaattgtcaagtaactgaatactcccaacaatcatatgggttaggttagaagtgaacctttctctagttgaggtttcagataagaccacagatatttatctaacaccttgattgcaggcttatgagaaaccttgaaatgagaagactaagtaaaaccatatctggattcctgacttacagaaactgtgtgatgataaatgtgtgttatcttaagccacaaagtttttggggtgatttgttctacagcaattgataatgaatacttcaacctatagtgacaactggatgtctatttactcattttccccaccaaagtgtaaagttcctacaaggcagaggttacaacatattcatcctgctacctctcatagcccataatatacaacctggcccataaaacatgattaatcaatgtttgttaaattaacatgtattttgagtttcattgaaatcagttagttgaagtgataataacatttaaatgtgcatgagcagtaggacaaggcagatagctggttaggatagaaagtttttgaaaattgtgaggggaaaaattatttctatttgtaccctgttgtacccagcttaggtttcaaaaactttcagggggtttttattcatttaccaaagacttcaaagttggtgcctccccaccactctaaagaggtctttgcccttattgacccaaccatactgatttgcctggtgcacagtgggagctattataaggagaggggagaagaaggaaatagaaacaactgtgaaaaaggttgaaaaaaatagcgaaaaggatagaaaaagagcagaggaaagagaagaagtagtcaacatccaacccagatgcagagaaatgattcaaaagaaaaagtctgattttcctgtcggaaatatttttaaaaggaggtaggcgtttatcaaaaaggaaaaaaaaaaaaaaagaataacagagtttgtcttccaaaggacccaatactcttggaattcaatgacctaattctgtgtatggttgcagtgtggagattgcctcttgtctgatctctacggcattgagacctctgatttctcaacagcacatgccacagcactatcagaagtcaggaataaagaacagagggtgtggggggcagtctagatattatgctcagcaaacatatttttaaatcagtgtaaattctatcttgctggttgcttatcttagcagttaaatcaacttttcgttacttttgtagacattatataatatcttctccccgcagctccagccaccagctgttagtcagcagaatgggagagggactgggtcagcttaagctgaataacttccatggtgacatttgagggaaccaattcccagccactgaacaaacctaaatgttctgctatggccatagaaccaaatagatcttaacaaagaaacctgagagcattcaaaggtcaaaacaattggagctagttccttcaagcttaaatatgctcatctgtaaagtggggacaataactatatatcttatagggttattttgaaggctaaatatgtgtaaaagcctcagtctactgaaaggttgctcaactgtaacactcttttcattttgtgagggatcattctttgttggaggttagggacactgtctcgtgcattacaggatgtttagcaacatcctcagcttctatccatcagatgttagcaccccccccacggaccaccatcttctccatcatcaccatcaaatatgtctccagacatttccaagtgtcccctgggaggctaaaactgcccctggttgagaaccactgttctagtacttggcattgttccataaacatcagatgatatttttatcattataattattactaaaggcaacatcaccctgcaatggagacaggtcttggtctcacggaatcctgcaatagttgctgcatttctgcatctttctgagagtaagggttgaaaagagataaggtcaattcatctgacaacaatcaatgggaaacctactgtctccgtagaaattcttttcatggaaagaaaaggggctaccattttgatctgctgcaggaactaggtctattcctcagcctcacagtttgcaaaacgctacagacttttccatgggcataaattctccagctttagggattgctggtatagttgactcttgaagctgctcttggatttccaagggtagcaccattttttacctgagaaaagaaggaaaaagatattcaagaaggagaacttttcaacagaagttactccatttgctgtttttgtggttttgtgcttgtttaggtttgcaagtgtgttttctgcccaaattgttcttgcatattctaacagtgtttgctttacgtgttttgatgccacgctgttttgaaggtacaaatgtttttcatggcatgtcttgtgttaagtgtacaaactgagacgtgaatgtcagtgactgaatagagtacagtattcctcaaagtttgtaactaaataaaccctaagagaaaagtgaagcgagatttctggaggtctggaagtggccagctgacaatatacatctctgtatctcctatttttatctttatgctaaatgcaaattttactttctgtgctaaaataatgtttcattagtttcaaggtaaaaatgtgtaaatagac

SEQ ID NO: 609 - DNA, 1421 bp, HOMO SAPIENS
agcggagccccgagccgcccgcagagcaagcgcggggaaccaaggagacgctcctggcactgcagataacttgtctgcatttcaagaacaacctaccagagaccttacctgtcaccttggctctcccacccaatggagatggctctaatggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaagataaaaagatttaatcaaaaatttcatatttgaaatgaagtcccaaatctaggttcaagttcaatagcttagccacataatacggttgtgcgagcagagaatctacctttccacttctaagcctgtttcttcctccataaaatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgattt

SEQ ID NO: 610 - DNA, 1266 bp, MUS MUSCULUS
aattctagaactttcctcatgaaaccagctcatctgagaaaacagcaaagtttaaaaaagaaatactatcagtgctgcaaaccaggaaggggaagtgtgtggtttaagttgcatatcccttatcctctggctgctggaggcttgctgaaggctgtatgctgttaatgctaattgtgataggggttttggcctctgactgactcctacctgttagcattaacaggacacaaggcctgttactagcactcacatggaacaaatggccaccgtgggaggatgacaagtccaagagtcaccctgctggatgaacgtagatgtcagactctatcatttaatgtgctagtcataacctggttactaggatagtccactgtaagtgttacgataaatgtcatttaaaagatagatcagcagtatcctaaacaacatctcaacttcaagcccacatgtttattttttatcttgaatggaaagtgaaacttgtatcatttttatttcaaaattatgttcataaccatcttcaatgattcaaccagaatacaaaatgaatgcactaaaaaggacatttctatatttctgtagttaaaattcaggacggccattctcgactgacatctaggattgtctggaatacctcttgtaagacttggaattggcattttttccacattacaatgtattagtcaactttgatttaaaatttgtaactcttgtgttttagtgtaaggaaaaatttagggttagtgttagagtttagggctaggtaaggaaaaactgagtcacactgaatgatttttttaaaatctatgagccagctgttggtagtttactcctttaatcccagcactcgggaggcagagacaggcagatggctgagtcgaaggccaacctggtctacagagtgagctccaggacagccttagctacacagaaaaatgccttatcaaaaaattaagaaaataagatgaagtattaaaaagtgacatgacaaatcattcctgagggctacctatatattcctcacacggtataaatatttaatttaattaatatttaatttcaaatattcacatttgaaatgaaacccaaatctgggttcaagcttactgctttagctgcacagtaaagctgtgtagtaaggagacccacgtttcctacgcatttcttcatgaatgcggatgaaactttacaaggttggtgtgcagctcactggagatgaacaactctttgtaaggtaataaaatcccacagtgatgtcttaaaaaa

SEQ ID NO: 611 - DNA, 2251 bp, HOMO SAPIENS
agcagggtgagggagggggtgggatgggtggggggtaacgggggaaactggggaagtggggaaccgaggggcaaccaggggaagatggggtgctggaggagagcttgtgggagccaaggagcaccttggacatctggagtctggcaggagtgatgacgggtggaggggctagctcgaggcagggctggtggggcctgaggccagtgaggagtgtggagtaggcgcccaggcatcgtgcagacagggcgacatcagctggggacgatgggcctgagctagggctggaaagaagggggagccaggcattcatcccggtcacttttggttacaggacgtggcagctggttggacgaggggagctggtgggcagggtttgatcccagggcctgggcaacggaggtgtagctggcagcagcgggcaggtgaggaccccatctgccgggcaggtgagtcccttccctccccaggcctcgcttccccagccttctgaaagaaggaggtttaggggatcgagggctggcggggagaagcagacaccctcccagcagaggggcaggatgggggcaggagagttagcaaaggtgacatcttctcggggggagccgagactgcgcaaggctggggggttatgggcccgttccaggcagaaagagcaagagggcagggagggagcacaggggtggccagcgtagggtccagcacgtggggtggtaccccaggcctgggtcagacagggacatggcaggggacacaggacagaggggtccccagctgccacctcacccaccgcaattcatttagtagcaggcacaggggcagctccggcacggctttctcaggcctatgccggagcctcgagggctggagagcgggaagacaggcagtgctcggggagttgcagcaggacgtcaccaggagggcgaacggccacgggaggggggccccgggacattgcgcacaaggaggctgcaggggctcggcctgcgggcgccggtcccacgaggcactgcggcccagggtctggtgcggagagggcccacagtggacttggtgacgctgtatgccctcaccgctcagcccctggggctggcttggcagacagtacagcatccaggggagtcaagggcatggggcgagaccagactaggcgaggcgggcggggcggagtgaatgagctctcaggagggaggatggtgcaggcaggggtgaggagcgcagcgggcggcgagcgggaggcactggcctccagagcccgtggccaaggcgggcctcgcgggcggcgacggagccgggatcggtgcctcagcgttcgggctggagacgaggggtggacgtgacaagcaggacatgacatggtccggtgtgacggcgaggacagaggaggcgcgtccggccttcctgaacaccttaggctggtggggctgcggcaagaagcgggtctgtttctttacttcctccacggagtcggcacactatggctgccctctgggctcccagaacccacaacatgaaagaaatggtgctacccagctcaagcctgggcctttgaatccggacacaaaaccctctagcttggaaatgaatatgctgcactttacaaccactgcactacctgactcaggaatcggctctggaaggtgaagctagaggaaccagacctcatcagcccaacatcaaagacaccatcggaacagcagcgcccgcagcacccaccccgcaccggcgactccatcttcatggccaccccctgcggtggacggttgaccaccagccaccacatcatcccagagctgagctcctccagcgggatgacgccgtccccaccacctccctcttcttctttttcatccttctgtctctttgtttctgagctttcctgtctttccttttttctgagagattcaaagcctccacgactctgtttcccccgtcccttctgaatttaatttgcactaagtcatttgcactggttggagttgtggagacggccttgagtctcagtacgagtgtgcgtgagtgtgagccaccttggcaagtgcctgtgcagggcccggccgccctccatctgggccgggtgactgggcgccggctgtgtgcccgaggcctcaccctgccctcgcctagtctggaagctccgaccgacatcacggagcagccttcaagcattccattacgccccatctcgctctgtgcccctccccaccagggcttcagcaggagccctggactcatcatcaataaacactgttac

SEQ ID NO: 612 - DNA, 2313 bp, HOMO SAPIENS
agcagggtgagggagggggtgggatgggtggggggtaacgggggaaactggggaagtggggaaccgaggggcaaccaggggaagatggggtgctggaggagagcttgtgggagccaaggagcaccttggacatctggagtctggcaggagtgatgacgggtggaggggctagctcgaggcagggctggtggggcctgaggccagtgaggagtgtggagtaggcgcccaggcatcgtgcagacagggcgacatcagctggggacgatgggcctgagctagggctggaaagaagggggagccaggcattcatcccggtcacttttggttacaggacgtggcagctggttggacgaggggagctggtgggcagggtttgatcccagggcctgggcaacggaggtgtagctggcagcagcgggcaggtgaggaccccatctgccgggcaggtgagtcccttccctccccaggcctcgcttccccagccttctgaaagaaggaggtttaggggatcgagggctggcggggagaagcagacaccctcccagcagaggggcaggatgggggcaggagagttagcaaaggtgacatcttctcggggggagccgagactgcgcaaggctggggggttatgggcccgttccaggcagaaagagcaagagggcagggagggagcacaggggtggccagcgtagggtccagcacgtggggtggtaccccaggcctgggtcagacagggacatggcaggggacacaggacagaggggtccccagctgccacctcacccaccgcaattcatttagtagcaggcacaggggcagctccggcacggctttctcaggcctatgccggagcctcgagggctggagagcgggaagacaggcagtgctcggggagttgcagcaggacgtcaccaggagggcgaagcggccacgggaggggggccccgggacattgcgcagcaaggaggctgcaggggctcggcctgcgggcgccggtcccacgaggcactgcggcccagggtctggtgcggagagggcccacagtggacttggtgacgctgtatgccctcaccgctcagcccctggggctggcttggcagacagtacagcatccaggggagtcaagggcatggggcgagaccagactaggcgaggcgggcggggcggagtgaatgagctctcaggagggaggatggtgcaggcaggggtgaggagcgcagcgggcggcgagcgggaggcactggcctccagagcccgtggccaaggcgggcctcgcgggcggcgacggagccgggatcggtgcctcagcgttcgggctggagacgaggccaggtctccagctggggtggacgtgcccaccagctgccgaaggccaagacgccaggtccggtggacgtgacaagcaggacatgacatggtccggtgtgacggcgaggacagaggaggcgcgtccggccttcctgaacaccttaggctggtggggctgcggcaagaagcgggtctgtttctttacttcctccacggagtcggcacactatggctgccctctgggctcccagaacccacaacatgaaagaaatggtgctacccagctcaagcctgggcctttgaatccggacacaaaaccctctagcttggaaatgaatatgctgcactttacaaccactgcactacctgactcaggaatcggctctggaaggtgaagctagaggaaccagacctcatcagcccaacatcaaagacaccatcggaacagcagcgcccgcagcacccaccccgcaccggcgactccatcttcatggccaccccctgcggcggacggttgaccaccagccaccacatcatcccagagctgagctcctccagcgggatgacgccgtccccaccacctccctcttcttctttttcatccttctgtctctttgtttctgagctttcctgtctttccttttttctgagagattcaaagcctccacgactctgtttcccccgtcccttctgaatttaatttgcactaagtcatttgcactggttggagttgtggagacggccttgagtctcagtacgagtgtgcgtgagtgtgagccaccttggcaagtgcctgtgcagggcccggccgccctccatctgggccgggtgactgggcgccggctgtgtgcccgaggcctcaccctgccctcgcctagtctggaagctccgaccgacatcacggagcagccttcaagcattccattacgccccatctcgctctgtgcccctccccaccagggcttcagcaggagccctggactcatcatcaataaacactgttac

SEQ ID NO: 613 - DNA, 2276 bp, HOMO SAPIENS
aagcagggtgagggaggggggtggtgggagggatgggggggttgggggaaactggggaagtgggggagccgaggggtaaccagggaagcgggggtaccgggggcaaccaggggaagatggggaagcggggtgcaggggcgtttgcgcgggccaaggaccaccttggaaatctggagcctggcaggagcggcgcagggttgaggggctggcttgggcagggctggctggcacctgggagcctggcggggttgaggtccgggctcccaggtgccctataggcagggcaacatcggcatggggggtgacaggcccgagctggggtgcggagggaagaggggggagccaggcattcatcccggtcaattttggtttcaggtcgtggcggctggtggtcagggggagttggagagaggttcgccccggggcctggggcagcggaggtgtagctggcagctgtgggcaggtgaggacagccgtctgccgggccaggtgagtccccttccctccccaggccttgtttctctggcctcctgcatccggaggttctggggagcgagggccggcgaggcgaagcggctgaccccccggcagagtggcggcggacgacaggcaaggcgggcagaacaggtgacacgtctcagggggagctgggaccgggcggggctggggggccggggccgtcccaggtggaaagagcatctcaagcgagtctggtgggagacgaggcagggctgccagcagggaggagacgcaacaggcggggggcattccaggcccgggtcggacaggacccgtcgggggtgtcaggacagtggggtccccagccgccacttcacccactgcaattcatttagtagcaggtacaggagcggctctggccgggcctcttgaggcctgagctggagcctcgagggccggagaatgggaaagaaggtgcagtgtgccagacagacgtcacctggagggagcacggccgtggggacgggccccagagagatttcggcagcagggaggctgcgcgggcccagcctgcggacgtgcgttcccacgcagcactgcggcccaggggctggcgcggcagggcccccggtgtccttggtggcactgtgcgccctcgccgctcgcccctgggactggcacggcagacaggacagcacccaggggagtcaagggcactgacgagaccagactaggcgaggcgggtggggtggaatggatgtgacctctggggggagggaggtggggacgcaggcaggggcgaggcgccggagcctggcggcgagcgaggccaaggcgggcctctgcgggtgacaactgagcacacatgggtacctttgcgctcgcaccggagacagcctcgtctccagtgggggtggacacactgccagcaccacaggccggacgccaggatgtgcttggagggacatgacacagtccggtgtgacggagagggacagacgtgacgccgtccggccttcctgaacactctcggctggccccgcgggggagctgccacacccagcgtctgttcctttgccttcctgaaggagcacgtgcatgactgctgctctctggaccccagaaccctcaaacgacaagagatggtgctacccagctcatgcctgggccttggacccggacttcttcaagtcctcctagctctgactcaagaatatgctgcattctggagccactacactacttgactcaggaatcagctctggaaggttgagccaaaggaacagacgtcccacaccaccggaccaacggcacccgcggggttccccaccccccgcccggccactccacctcggcggccaccccctgctgcgccctggagacaccaccagcctccctctctccccttcctcctttttttcctctgtcttttctcttctcttctttcctctcctttgctcagaagactcggggcatccaggactctgtgtccccgtccttcctgaattaatttgcactaagtcgtttgcactggtttggagtcctggaaccagccccgggtctcggagcgggtgtgtgagctgccgagtggcctggcctcctcggcccgcgccccctcagcacctgccattgtccatctctgtctgggggtgactgggtgggggcctgagtgtgtggggccccgccctcccctctcctagtctggaagctccgaccaccgagcagacctcaaacgctgcactgagtgtccatctcgtcatgtgcccctcctcgccagggccaccccagagccctggactcatcaataaactcagtta

SEQ ID NO: 614 - DNA, 1899 bp, MUS MUSCULUS
agggggcctggtgagaagaagcggctggcctcgcagcagaatggcacatagaaaggcaggatagttagcaaaggagacatcgtctcggggggagccgagacagaaggaggctgggggaccattggcgaccccaggtggaaagagctcttagagagaagaaagaagaggtgcagggttgccagtaaagactgaggccgctgcctccagggaggtgataggagtccttggagacagtggcagagaccatgggatccagcaagaacagaagcattctaggctggggtcaaacagggcaagatggggtcacaagacacagatgggtccccagccgccacaacatcccacccaccgtaattcacttagaagaaggttcaagagtggctctggcaaagtcccaagtttgccagagcctcaataactggagaatggaaaagaagggcagtgcagggtgtcaccagaaggggagtgggggctgcaggtatcggactccagagggattttacagcaaggaggctgcagtgggtccagcctgcagacacaccattcccatgaggcactgcggcccagggactggtgcggaaagggcccacagtggacttggtacactgtatgccctaaccgctcagtccctgggtctggcatgacagacagaacatttccaggggagtcaagggcacaggatgaagccagacgaggcgaggcaggcggggcagaatgaatgagtttctagggagggaggttgggtgcaggtagagcgagtagctggggtggtgagccaggggaggcactggcctccagagtccgtggccaaggagggccttgcgggcggcgacggagcagtgatcggtgtctcgaagagctcggactggagactaggccaggtctccagcagaggtggatgtgcctgccagtcactgaaggcgaggatgacaggtgtggtcaatgtgacagaaagacatgacatggtccggtgtgatggagaggacagaagggcagtcatccagccttcttgaacaccatgggctggcgccttgtcgtagaagccgtctgttctttcacttttcccaaagagctaacacttctctgctgctctctggatcctcctccccctaccttgaaccctcaagatgaaagaaatggtgctacccagctcatgtctgggcctttgaatccggggacttctttaagtccgtctcgttctgaatcaagaagatgctgcaatcagaaccactacactacctgcctcaggaatctgctccaaggtgaagctgaaagaacagatggtgtcaacattttgaaagagcagactcatagcacccacccacccctgagaatccatcttcatggccaactctgcctgacccgggagaccaccacccacatcatcctggagccaagcctctaccccgggatgacttcatcatctccctcctgtctttttcttcttcctcctttcctgtaattctgtttctttccttttgttccttccttgcttgagagactcaaagcacccgtgactctgtttccccatttacccccttttgaatttgcactaagtcgattgcactggtttggagtcccggagatagctttgagtctctccgtatgaatgtatacagcgagtgtgtaaacctctttggcaatgctgccccagtacccacctgtcgtccatctccgtctgagggcaactgggtgtggccgtgtgcttgaggcctcgccttcccctcgcctagtctggaagcagttccatcataaagtgttcaacatgccctacttcatcctttgcccctcctcaccagggccacaccagaggtcctgggtccatcaataaatacagttacagtcaaaaaaaaaaaaaaaaaaaaaaaaaaaacaaaacaaaacaaaacccttgaccgt

SEQ ID NO: 615 - DNA, 395 bp, Ovis Aries
aaatggtgctacccagctcatgcctgggacttggaacttggacttcttcaagtctttctagctctgactcgagaatatgctgcatcttggaaccactacacaccttaactcaggaatcagctctggaaggttgacctaaaggaacggaagacccacaccatcggaagagcggcgcccgcggcgacacccaccccggcccgacgacaccatctctgcggccaccccctgccccagccccgagaccaccaacagcctccccccctcctttccctctttttctgtctctatcttctgatcttctctctgctgtcctttgctcaggagactcagagcgtccagagccccgttccccccgtcccccttgaattaatttgcactaagtcaattgcactggt

SEQ ID NO: 616 - DNA, 397 bp, Ovis Aries
agaaatggtgctacccagctcatgcctgggacttggaacttggacttcttcaagtctttctagctctgactcgagaatatgctgcattttggaaccactacacaccttaactcaggaatcagctctggaaggttgacctaaaggaacggaagacccacaccatcggaagagcggcgcccgcggcgatacccaccccggcccgacgacaccatctctgcggccaccccctgccccagccccgagaccaccaacagcctccctccctcctttccctctttttctgtctctatcttctgatcttctctctgctgtcctttgctcaggagactcagagcgtccagagccccgttccccccgtcccccttgaattaatttgcactaagtcgtttgcactggt

SEQ ID NO: 617 - DNA, 2748 bp, Ovis Aries
gaattccaggacgggtgaggtacgtgcgctggaccgtcccgggtgggggcgggcctcgcgggggccccaacacggtccccgtgccgattggctggccgggcaggggcgggaatgctgggcggggctgcctccagttagaaaaagcccgggctagggcccgaggagcagggtgtgggaggggggtggtgggagggatgggggggcacgggggaaactggggaaactggggaaactggggaagcggggtagcggggtagcggggtagtggggtagcggaggggtaaccgggggaagtggggtgcagggaagcttgcgtggggtggcccaaggatcaccttggacatctggaccctggcaggagcggacccacagaggggctagcttggggagggccggggggcgcccgggacccttttggggatcattttttgctcccgggcgccccgcagacagggcaacatctgcgtgggagggtgaaagacccaagctagcagcacggaggagaagaggggggagccaggcattcatcccggtcaattttggtttcaggtcgtggcggctggtggacaggggggcttctggagagaggctcgccccggggcaggggcaacggaggcgtagctggcagccacgggcaggtgaagacagcgctctgccggccaggtgagtccccttctcctcgctgggccttgctctcctggcctcctgcagagcagaaagacagaggctggtcaggcagcacaggtgataccgtctcagggggagctgagacagggcgggctggggggccgcgggcgcccgggtggcaagagcatttccagagagtcaggcgggaagagcaggcagggctgccagcacgtgcagggggcactcaggcccccgagcgagcaggacagggcggggactcaggacagaggggttcccagccgccacttcacccactgcaattcacttagtagcaggtcacaggagcggctccggccaggcgtcctcaggcctgagccggagcctcgagggccggagaatgggagagaaggcagcgcagtgcgcgggcaaccgcggcgggatgtcatctggggggcggtggccgcgggacaggcccggagagatacccgcagcagggaggctgcacgggcccagcccgtggacgccagccccacgcggcactgcggcccaggggccacgcgggagggccccagtgcgcttggggacactggggacacttgacccgaaccgctggcccctgggatgggcatgatagacaggacagcacccaggggagccaaggacacgggcgagaccaggctaggcggggcgggtggggtggcatgagtgtgcactctcgggggaagggaggtcagggcacaggcaggggcgaggggccggagctgggcaccgagccagtgggtggggcactggacttcgagcccgtggccaaggcgggcctctgctgggcgatgacggagcgggatcggtgcctttgagcttggaccggagacgaagccacgtctccagtggggtggtggtgcctgccagccacggaggcccagcggccaggacgcgcacagagggatatgatatggtccggtgtgatggagagagcaggcgggaccgtccggcctctcagagcgctggccccgcgggagccgggatatcgccgttcctttagcctcctgacggagcagacgctactgctgctcttcagacacccagaaccctcaagatgacaagaaatggtgctacccagctcatgcctgggactggaacttggacttcttcaagtctttctagctctgactcgagaatatgctgcattttggaaccactacacacctaactcaggaatcagctctggaagttgacctaaaggaacggaagacccacaccatcggaagagcggcgcccgcggcgacacccaccccggcccgacgacaccatctctgcggccaccccctgccccagccccgagaccaccaacagcctccctccctcctttccctctttttctgtctctatcttctgatcttctctctgctgtcctttgctcaggagactcagagcgtccagagccccgttccccccgtcccccttgaattaatttgcactaaaagtcgtttgcactggttggggttctggagccagccctggtctctgagcgtgtgtaagcgcgtgtgtgtgccaactgctgaatgtgtagcctcctcggcccgtgccccccgcccagcacccttcccaccctcgtgggtgcctgggagggggcccgagtgggggggcccggtgtgagggcccgccctcccacacctagtctggaggctccaaacagcgggatcactcacaccaagcattcccactgtgtgcccatcttgccctgtgtccctccctgccagggcctcctcagatgccctggactcatcaataaactcagttacaggaatttgtctcaggactcttgcatttgagatgaacgggtgtcgggggtttttgggggaaatggggcgggggaacatgggaaggcacgcgggccacaaggggttgcacacaagggtgggggcaacccatgggcggggacacatggaggggagggggcggcccggagctggaacacagagtggggggctgcggacacgagggggccgggggggggggctcctgtaggaggggcagccaagggggctgggacataaggaaccggggcaagcaccacgtgggggctggggtgaggggcagggggacccagggttcctgcag

SEQ ID NO: 618 - DNA, 1842 bp, ORYCTOLAGUS CUNICULUS
ggtcactttttggtttcaggacgtggcagctggtcggacagggggagtcgtcgggcagggtgcacgccggaggcctggggcagcggaggtgtagccggcagcgtgggcaggtgagtcccttccctccccggccctggcgcccgggcctccgcagccaggaggtttcgcggcgtgagggccggcgaggagaagcggctggcctcccggcggagcaggacagagcgcaggagagcgagcgcaggtgacatcttctcggggggagccgagacggggcgaggccggggggggccctgtgggcctcccgggaggaagggacattgcaggagggcagggcagagagcggggtgggctgccagcacggcgcagggagcgcagggcggatcccagcaggtgtggggacaccccaggcccggcgggacaggacgaggggggagggcacaggacactggacagacgggtcgccagctaccaccacatctcacccaccgcaacccacttagaagcaggtacaggagacgccccggctgggcattcccaggcccgcgccggggcgtcgagggctggagagcggagaagaggggcagtgcagcggcgtggcagagagaaggtggccgcgtggcactgcagcccaggggctgagcggggagggcccacagtatatgccctccccgctcagccacgggcgctggcacgacagacagtccaggggagtcaagggcacaggcgaggccaggctaggcgagacgggcgaggtggcgtgtgtgagcactttcaggggggaggtggggcgcaggcaggggcgaggagcggggcgctggcctccagagcccgcggccaaggcgggccttgcgggcggcgacggagcagtgatcggcgcctctgcgttcccggtggagaccacggcgagggggagctgtgcccgccgccacgggaggcccggacgccaggtgcaggcgagccgagggacatgacatggtccggtgtgacggtgaggacagaggggctggcgtgcggccttccagaacatcctcggggggcaccgcgggggagctgccatcaggagcgagtctctgtcccttccgtcgcccaaggagctgacacacacacacatacccactactgctgctctctggacccttggaactctcagagaaagaaatggtgctgcccagctcaagtctgggccctcgaacccggggactcgtgagtccgtcctgttgtgaatcaagaagactttgcctttggaacctcgacacagccttgctcaggagtcagctcccaggtgacgcccaaggaaggagacctcgcccacactgccatcagcagcagcagcaacgcccccacccctcgccccctgccactccacctgcgcacgcaccctgcggcatccgggtgctcccggagctgggccctccgcggggtggcccccaccgcctccctcctttctttcttcttctctctctctttctctctctgcctgagtgtttgccctcccattgcctgagagactcggagcacccctgactccgccccccatcccctccacgtttattttgcactaagtcgtcggcactggtctggagtcggcggcggccctgagtctctcgcgcgtgtgtgcgagcccttggccaagcccggccccggcacctgcctatccgcccatctctgtccggggtcctgggcgctggcgtgtgtctgaggcctcgccccacctcgcctggcctgggtgctccggcccagcggagctgccttagaagacccacaaggcccctctcgccctgtgcccctccccaccagggccccagcaggagccctggcctcatcaataaacacagttccggc

SEQ ID NO: 619 - DNA, 2088 bp, PEROMYSCUS MANICULATUS
atggggaccagacattcatcccggtcacttttggttacggacgtggcggctggtcggatacaggggctgctgggaagggttcgaccccagacctggcagtggacgtgtagctggcagcagtggcacgtgaggatcgccgtctgctgggcaggtgagtctccttcttctctctgggcctcgcttgctctgaccttttaaaagaaggtttagagagcgaggccggtgagaagaagtggcttggcttcccagcagaatggcacatgagaggcaggacagttagcaaaggagacattgtctcggggggagccgagacagaacaaggccgggggaccattggccaccccggagtggacagagcatttagagggaaggagaagagggcgcagggctgccagtcaagactgaggctgctgactccagggaggtgataggtgaccttggagacagtggcagaggccatgggatccagcaagaatagaagcattccaggctggggtcaaacagggcaggacgggggtcacaagacacagatgggtccccagccgccaccacatcccacccaccgtaattcatttagaagaaggttcaagagtggctctggcagggccgtccaaggcctttgccagagattcgagggccggagagcgggaaagaaaggcagtgcgcaggtctacatagcagggtgtcaccaggagaggggtgggggctgcaggtgttggactccaggggatttacagcaaggaggctgcagtgggtccagcctgcagacaccactcccacgaggcactgcggcccaaggactggcgcggaaagggcccacagtggacgcttgtgcactgtatgccctaaccgctcagcccctgggtctggcatggcagacagaatatttccaggggagtcaagggcacaggatgaagccagacaaggcgaggcaggtggggcagaatgagtgagttttctaggaagggaggttgaggcgcaggtagagagaggcaaagcaaatggggtgatgagccagcgaggcactggcctccagagtccgtggccaaggagggccttgcgggcggcgacggagcagtgatcggtgcctctgagagctcggacggagattaggccaggtctccagcagaagtgggtgtgccagccaggcactaaaggccaggtggcaggtgttggtcaatacagacagaaagacatgacatggtccggtgtgaggagaggacagaaggacggccatccagccttcctgaacatcatgggctggcgccgtgggacactgccaccatacaagccgtctgttcttttagttttcccgaagagctaacacatctctgctgctctctggatcctcctccccctactttgaaccctcaagatgaaagaaatggtgctacccagctcatgtctgggcctttgaatccggggactcctggaagtccgtctagttctgaatcaagaatatgttgtgcaatcgaaccactacactacctgcctcaggaatcggctcgaaggtgaagctgaaggaacagacggtagcggcacttaaacggcagacccacagcacccacccaacccacccctgaaattccatcttcatggccaactctgcctcgcctgggagaccaccacccacagcatcccagagccaagcctctgctctgggatggatccatcactatcatctccctcctgtctttttcttcttcgtcctttcctgcaaccttatgtttctttccttttgttttttctttgctttgagagactcaaaagcgtccgtgactctgtttcccccgcctatcccctttgaattcaatttgcactaagtcgtttgcactggttggagttgcggagacagccttgtgtctctccgtgtgaatgtaaacagcgaatgtgcgggcccctttggccatgccgccccagtgcctgcctgtcatccacctctgtctgggggcgactgggtgtggccgcgtgcttgaggcctcgccttcccctcgcctagtctggaacagttccatcataagtgttcaacatgccctacttcatcctttgtcccttccctgccagggcctcaccagaggtcctgggtcc

SEQ ID NO: 620 - DNA, 2276 bp, SUS SCROFA; identical to SEQ ID NO: 613

SEQ ID NO: 621 - DNA, 259 bp, HOMO SAPIENS
  misc_feature (59)..(59): n is a, c, g, or t
  misc_feature (104)..(104): n is a, c, g, or t
  misc_feature (116)..(116): n is a, c, g, or t
  misc_feature (179)..(179): n is a, c, g, or t
ctgggcagtgcgctcccatctgcaccttatggacagcgtggccagggtcgaggtccganttcctggccgcgtcccaaggatcggattccgggtctatagttctnatggtggttcanagtgggctgaattgggattggagtctggaatccgcatcgtggttctaagtccgcgctattggnatggaagttgggaatccatgttgtggttttaagtcaaagcccgaattgggaccggagtcggaaatccacttagtggttct

SEQ ID NO: 622 - DNA, 462 bp, HOMO SAPIENS
tccatgtagcccagtaattttcaaagtgtagtctggggattccagagagccccccagaccctttcaggaaaggtcaaaaacattttcataataatactcgacattatttgccttttctttcattctcacaaagataaagagttttccagaggcagcacaccatgaccccagtggaatatgtgcttgtatagttctgctttaaacatttcttagttttaacttctaagatggtaactatcaacagatagaatctctatcaagaaaagccctttgggggtttcaataacttttaagaatacaacaagtccgtaacaccaaaatgtttgagaacctttatggagaccatcaacccctatgagctacactaagttttatcacttcctacctgatcctcacatccccatttctttttcttgtattagggtatggccaggatgacatccttgggcctactccttatct

SEQ ID NO: 623 - DNA, 310 bp, HOMO SAPIENS
gccatgatggatttatttaacagaaatccttaatccataatcattaatgcattcaagttcatacattttagtcttgaggtttgtgctttctggatcctgtttaagaaccccacggccaccctggccctggagattattcattttttctcctattaaacaaatggcaactaccttttgctttcaaatatttagtccatttggagtctacttttcctaggggctcagttaggaatccatattcctattcctccattttagggctggctttccaacatcatcttctgaacagtccccactgatatgtggtgcc

SEQ ID NO: 624 - DNA, 289 bp, HOMO SAPIENS
  misc_feature (7)..(7): n is a, c, g, or t
  misc_feature (31)..(31): n is a, c, g, or t
  misc_feature (92)..(92): n is a, c, g, or t
  misc_feature (94)..(94): n is a, c, g, or t
  misc_feature (115)..(115): n is a, c, g, or t
  misc_feature (159)..(159): n is a, c, g, or t
  misc_feature (167)..(167): n is a, c, g, or t
  misc_feature (225)..(225): n is a, c, g, or t
gatcctntccaggcagcttcttccacacctnattagctctgaccatcagaccccccgtgccaaaagggccccttccctggaatctgtaattntnacaggctgtcccatacatttnaaatccaggaaatgtgctcacccatgagacagggatggcaagangatatgantgaaaagcaaccagtccgtaaaattggaagccatttcactccttaccaggtacaaagntaacatgctgcatggatcagggacacaaacttacctaccttatggatgtagtgaaagattttta

SEQ ID NO: 625 - DNA, 422 bp, HOMO SAPIENS
ttcagacagaaaatggtggcacctggagaggtgtcaggtctgtgtggaagaaaggacaggagtccttctgccacccagactgtgcattcagacatctcccttgtgctgtgcagccttagttctgcctcctgagagaggtgaccttctcccacttcttacaggcaaggctcttgctagcctgaggaccaggagccttcagattttcttgatcccaggatatatcgcaccaactcaggaggtgtggggatgggctagcattaggaatgggtattagcatctgcattaaaaggctcccactgacagctcatgcattgtgatggaactagaggtgagatacagagcaggagtccatggcacctccagaacctgtcagcctgagtatgggcaatggcgttttagtttgcaaaaccagacacatagag

SEQ ID NO: 626 - DNA, 462 bp, HOMO SAPIENS
acatttaaatccttgctttatcgggattttgacagagtaattaaatccaatttggtttcccatatatttgtccagttgtccaacactatttatttgagtacatcatcatctctctactaatttcaactgctacctttatcatatttcaaactctcatttaatttgtgtccatttctagactctattctggactgctgatgggcctgttgataggccagagctcagttattttaattatgtaactttatgatgcacttatctgtgtagcaggactccccctcatctccattacttaagagccaataatgggaagctcattttcacaaatgcagtcaaccaggggaattcatggcatggctaagatctaaacactcttaagatttaaacacaggattagctcttgagttagacaggaagctggggtgtttgggactgaggcttcatcgtggcagctaataatgt

SEQ ID NO: 627 - DNA, 328 bp, HOMO SAPIENS
  misc_feature (147)..(147): n is a, c, g, or t
  misc_feature (204)..(204): n is a, c, g, or t
  misc_feature (218)..(218): n is a, c, g, or t
  misc_feature (259)..(259): n is a, c, g, or t
  misc_feature (261)..(261): n is a, c, g, or t
  misc_feature (267)..(267): n is a, c, g, or t
  misc_feature (279)..(279): n is a, c, g, or t
  misc_feature (290)..(290): n is a, c, g, or t
  misc_feature (300)..(300): n is a, c, g, or t
  misc_feature (305)..(305): n is a, c, g, or t
aagtcatatttttttaatgagtttaaaacacgtgtgtgcattagaaaacatttaaacacagatattgggcaaattaattgaaaaatatattgtcattttgcgttaaaataaaagatgatctgaaaatggaaaaaagttacataaaancgtaaatatttcatgagacatagcagtcacaacatgccatacacatttcttccagantcactccactgganattctgtaataggacatacgtaacagtttcacttcctgggngntcccanttgctcctcagntagggtggagngcaccccttnttagngggtggttgggctctctaggggc

SEQ ID NO: 628 - DNA, 334 bp, HOMO SAPIENS
  misc_feature (206)..(206): n is a, c, g, or t
  misc_feature (223)..(223): n is a, c, g, or t
  misc_feature (321)..(321): n is a, c, g, or t
tatatatgcaaccaacaacactggagtacccaggtatattaaacaaatattattagtgctaaagagatagaccccaatacaataatagttagggatggcaacatttcatactcagcattggacaattcatttggatggaaaaatatcaacaaggaaatattagatttgaactgctgcttcgttagcacacagcacattctccaggntataccatatgttaggncacaaaacgggtctcaataaatttttaaaagtcaaaatcttatcaagtatcttctcagaccacaatggaataaaactggaaatcaataacaagagggnacttcttgaaatt

SEQ ID NO: 629 - DNA, 423 bp, HOMO SAPIENS
gtccttccttctgttaatgtgatgtatcacatttattgatttgtatatgttgaaccatccttgaatccctgggataaatcccacttgattatggtgtattatctttttgatgtgcgattggattcagattgctagtttgtgtgtctgtgtgtgtgttttgtttctgaggattctgcatctatgttcatcagggagattggcctgtagttttcttttttgctgttattgtgtccttatctggttttggtatcaagatactgcttgcctcacagaatgagttagggagaattcctttctcttcagtcttttggaattgtctgagaagaattcatgttagttctttaaaggtttggtagaattcagtagaaaacccgtgcaatcctgaggttttctttactgggagactttattactgatacaatc

SEQ ID NO: 630 - DNA, 358 bp, HOMO SAPIENS
ttttttgaccgatacataatacttatggggtacatgtgctattgttacatgcatcgaatgtgtaatgatcaagtcagggtatccatcaccctcaatttatcatctctatagttaggaacatttcaagtcctcttttttattttgaaatatacattatattgttaaatatagtcaaccaactcttatcaaacattataaattatttattctaactagctgtgtatttttacccactaaccaacctctcttcaccctccagacatacctttcacagcctttggtatctatccttctactctctacctccatgagatcaacttttatagctcccacatatgataataacataagataatta

SEQ ID NO: 631 - DNA, 4773 bp, MUS MUSCULUS
tggagtgcctggtggcaaagatggggggggggctgtgcgccccaacaggcccactaaatcctgggtgtctatccctaaaaatcgaggtcccatgtcatgtttccggggtgccctgtgcttgtgcaacatgggcgcgtggtgcgaaccaacgtgcgtggcaccctaggcctcagtggtagtggcaggacctgtgctcagttagagggttacaaggtaggcgcatcccggccgactgagttaaggcgcaggtagctgggtagagggttctcacagggacctacttgctaagcaggggacagtcccctggatcggctgggggggggggggagcgcgcagcctccacctgcctgagcaggcagggcgggctgtgaggcacctcgaatagcagccagatagatcttactaagcgcccctaacctctgctttgcctgggatctcctagcagcctttggggggccaccaggtgcaccatgaagggaaggctggcttggccacgccaaatctgggccccgcgtggagccccccgcacaatgtctgcgcccttcagcagaaaaagaagcagggcgggaaacgtagctcaaaccagacctgtaaaccagagctcactgaagtgcttctaagcggcgcgcctttgggaccccacccagtatgagcagataacagttccctgtcctcagcactttgaatttgcttagttgcaggttcggccccaggggcaatgcgataattccgggctattaagaaactctccccgggtggtttcagatataagaagccattgtgatttttaccaagtcaaaatcacttctaaaccggtgcaccttgtttccctctcccgacctccgcacctctaggcctctaaagggccactactggcggaaaccagtgcacccggggctaacctcccggcccgaccatcaatcaccgcgcccgcgctctacactcgcttccccaagccctcgacctgtagggttggaggctccaagccgcccgagcgctctgcccttcccgcttcctggccgcccaggcgctttccctaccaccgcgttgtttgcagagtgcacacaaagtggcgcgcgtttcggagcgcccagcccaaaggagcgctgccgccgagggagaagccagcagcccgctcccgcgctactccgcagcagccctgaggacaaactgcgaggtagccggtgccccgcagtggaagagctggggcacgagggagcgcgccacggggagctcaccacaaggccgaaggtcgggtccgctggcgcaggaggatcggaggagaagggcgggagagcggatgaagtgggagctgctgaagtggacgctgctgaagaggtctctggagaagccgctgactcacaccccggagatgaactcctggactttgccagggtccccagcatgctcttcctcatgcctgtgctggatgcctgccacagaggacacactcaactgggctgcggtcttggacaccacatagggactccacaccaggaagacccaagcaatgctctatgccaatctgcaagcctgggaagaagtcactacctgaaccaccaacatgaggattgcaccaaactgatcaacacaacccacaccaaaaattctttcctcatctggaaccctgcctttccctctggagctgtctgacctccccccttccccagcatttgctacctttgcccctggactcctggaagcattacagagactgctgcctcgtgtctgcttggcttacaccccttgttcccaagaagtgacacctggagcgcacctgaatcccagatactgtgactgactagacttcctagccttggctggttccttggctgagtcttgcatttgcaggagctctgatgctcctgtgctagagatacctctgttctgctgtatacctgaaggacactcacctctgaggaaagactggggctggtgggaggtttctgaggtttcattcagttctgggagcgtggggatctcctgggccacaggcacagcacttgggcacggggctggagtctcgggatgctctagcctgtttcttctcaggttccggctcaggcttcagctcgggctcaggctcaggctcaggcttcagctcaggcctcggtggcagaggcctcggctcgggccttggtggcagaggcctcgactcgggccttggtggcagaggcctcagctcgggccttggtggcagaggcctcagctcgggcctcggtggcacgggcctcggctcaggcctcagtagcagaggtctcagctcaggccttggtggcacgggcctcggctcagacctcagtagcagaggtctcagctcaggcctcggtggcagaggcctcggctcaggcctaggtggtacaggcctcggctcaggcctaggtggtacaggccttggctcaggcctaggtggcacaggcctcggctcaggctttggtggctcaggcctcggctcaggctttggtggctcaggcctcggctcaggctttggtggctcaggcctcggctcaggcttaggtggctcaggcctgggctcaggcttaggtggctcaggcctcggctcaggccttggtggcacaggcctggacttaggcttctgcagctctggctggacgagaagtttagccttaggagactcagaaccctgggttaggggcctgcagtcgtcgtagtcgttctcctcttcctcctcctcctcttgcacagtgagctctgttttaattactcgtcccctgaggaggagaagcaaaaatccttgagattatctcaaaaacaaatcttctccagctgagtctggccccctccccccaccttgctgctggatggggaagcccaagggtcaccttgtcatggttggagcactcccccctaccctgtcccttcagctgcctactctccccacccccttctcagtagaagggaggaagcagttggtgagaagtcactttgccaagtttgacaactgttcagggagaggcaggagaccctcctttgggaaggacaatctggctcctgcctggctgggggcgggggtggtggtggtgtaaaactgctgctgcacacaaaaggctttctttgaggggtcacagctaaaaatgtgaatgtctcccattcctctcctcacacttctaccttggatgggaacaaaaaacaaagggcacatggatctgccttaggaaggcaacttctggaaggggtggcagggcttctccccataaacattataccggtggattaaggagtctgggatcccagttgcagggaaaaaaagctttgtgcacaaatgaatgtgcaactgggagtgctgtggctgctgccccaaattcctgcctgtgcacagggaacacggagggggggggggagggggagttaaagacagacacagccaggagaaaagcagtttttgtacttaaaccctgctagggaatagcaaggcacatcatcctatctatctaccctccagtatcctggtggtagttaggaggtacaggatataaaaacagggaaactttgcttttaaaattttatatatatatatatatatatatacatacatctacataatatatatgtataataataatacacacccccacactgctctagaatctctggcccctctgcacagtttagccgggagtggggtgggggtgggggggaggagcggggggagcatacaccagggggaaaatgttcacacagaaaaatttcacatttgcaaaaacagctgtgtactcctgctgggaagagaaagagcgctgcctctcccctttgcatggtagattagagaacgaaaccatactttccttgtagctttgtgcagccctttaagtcccagtcccagttgtccttttgtcccctcagaagttgaagtcactcttaagaccgtccggtttagcggcaaccttccccctcttcagagactaactgaaggaccctaactctcccaagtcccagccacctacccacaccagaatatttaaactgcaccttcactcatacacaaaaggcatgaacaaggggaaatcaattagttacctgtactctagtcgcttcgtagttcctgaagttcccggacggtggataaagaggaccggggagtcactggtgcccaagattctcttccgaaattgcccggacgatgatccgctggctgggtcaatattagcagccccctccaaagcagggggtgctctggttgtggtgcccgggccctccagccggggcgacgatgttgcggcggggctggaaggggctgagtctgagtctgaagagacggtgagagggagttttagagcgtgcatgataacagagcccctagaagtggcttctgctctccaccactgagtaaggcacctgctcgaatgacaccacgcttttatccctcacctcgtcccctccacgcccacttccagccccctccaaatttgggaaatgatatgccagtaaagtactgcaaagacttatggggcagctggctatggcgctttcccacactagggtatgggggcattagagggtgggctttggcccctgcctgacagacaggaaggcgtctatgcaggtttgaattaagctagaagcacttcctgttagctagggggtagggactatgtgcaggggtggagagtgggtggggcttcactggattgacacctggcagtcacctgacaccctggaatgtctgtgggcgtggcacaggtaaaaaggggcgtggcttatgcaaattagggtccggatttaaaataaatattaaaattactttctaaggtc

SEQ ID NO: 632 - DNA, 2075 bp, HOMO SAPIENS
ccaaatcatagatcaagatattttatttatgtatgtcttttaaatgaagagtggttaggatagtcctgattattgcacataaatattgcctttcactagattattgttttgggccaaattttagactattattttaaaaaagagtaccttagtttaatccaacctttccagatatttagaaattattgaaatgtatattttgtgaagatggtcttatgaaagttgttgagctagaaacatgtccttcaccagtggaattcttagaagctttccttgggcttcactgggtgctaatagaccatgatcgtattgctctgtatataatggtacaaattttgttttataacacttagtcttaatagaatgggaatattttatcttactaacgtagtcattagctggccactctctgtgtccattggaccttatagggtaaagggagggcagtgcgtatttgaagcctgtgcacgtgacacacaaagggcttagcaacctccgtgttttcccagcacaggatacccattcttcctgaatgtttcattgttaaattagattgtgtgtgtgtgtgtgtgtgtgtgtgtgtattttatattaacatacagatgtatgtattttagttgcattttttttcttaaaatgttattttttctctttttcttttatgtcaggtgatgatagagcccctagggaagcaaaactgtccatgcccttgccttaactttcagatacgataaaaacaacatttataaaatacctgtcttagaaatattagctaaaattatgttggggaaagtatgtttgaaaatagaattgtaagctaccacattgtgataaatattactaaatttgtttattgacttgtcaggaagcaagagaatgaattaaagaaaactccagagaatgagctgacaacctactccctggtcagtggctctccatgcctacctgtggtctcttggacacccttgcagaagatgacttcctgggaactcttctgggagtgaatgttatcagcaaattcccaaaacaacaccccttagacaaaaagcctcctcacctatgcattgcctagaccacccactaaaggtgatggccacaaagaggtggatttgttgagttggtgccattccttctactgccagcttcaccaaaacaatgacacagtcttccttccgcatggagactcttctctttcctgtgttgcagtggatgcctcctgcagataagaaatgcttaactgagaaatgctcatgttgggatatcatcgatgagactttaatttgggcatggtgactgtttcccttgctgccacttgactcaatcccaggagcctcagtagaggatataatattacacaagatgctcaaaatggtggaattgctcagtgctcccataaatatacatttataattttcatttgccacacggtaaactgaaaaatacagactatattaaaaagaaactgcatgattttttttcaaaaattcaaagcatatttaaataatatataaattaaatgccctcctctcttccaattctgtccctatagttttcctctattaagtgaactacatgcattcttttagtggatagatgcacacaaacacacaagccattatggggaaggatccacgtgtgtggccatattgtaacacatttttctgcaaatcacctctttcatttaacagcccttattcaatggcctttttctttttcagtagtacatacacatctgtgtcatttgttgaatgacgacatgaatgttttgtagataaaatataattaaatgttttaagcatttctcactcatagatatttatgttagcaccaaatttctgtgcaatgaacctccctatatatcagtaattttgcatttgtctataatatcataataaattacttatcagttgaattactgggattaagtatgtgcacactttatatttatattttgaaaattattgcaagattgccttcaaaaatgtggttaacaactttatcttcaaatggcaatgaatgagattgccagctttcctacagcctcgacagacaatggattttataaatatttaact

SEQ ID NO: 633 - DNA, 1194 bp, HOMO SAPIENS
ctcatcccattcatattttaaaaatactaatattttgtgtttgagcattttaataggctcagaaacttaaaaatgatgtttcttttctaacaacatactcttttcaatgggatattatggttgttattatttaacatgacatttagggactcaacatacattaaggtgatggcactaagataaatgtagaaataaccagtaccatgtaattttcataagtgcttaaattgttggtaaacaattttatgagttggaggtgttgaagcaaatattattaatatttgaacataaaagctgatcaaaggggcctggtccacagaagatgtttatttgatgtaacaaaacaatacagttagtgttagatccaaccacaaagagcaaaaggaatgaaaaatttgtacaatgtacatagaaaaaacaagatatttactgtgacaactatatattcctaaaataatgcttctaaaattactcaattattgaaatctacatgaaaaaaaggatgttaatagcgacaaagtgcataaaatcaaacattgtattttgagcaaattaacatactaggcaattttgctaagaatgcatgattttttttttcttgtttacagtctgctcaaaatatctttataccaacagggtaggcagaacatttaggtttaatatcagttacacaatattagcataaacttccacaactacatagggtattgttttcttttgagctggcaaagtgactatagaaacatcagatgatttctctgaattgagaattttatccaaataaatgccacataccttctagatatatgcatatctttctatattatgtaaatggctttacccatttaaataataaaccatacagcatttaagaatcattattatatgattaacaatgtcatgttccaggtttaacaatttcataggccaaaaaaaatttcttcttaaaaactagttttataaacgcagaatatattccatgagtaactgctggtattttttaagaaaatatattgtgcaattgtggctaccacgtactgctggcaaagcattattatttatgtaaaatgtgaaaaaaaaggtgtaaaaatttttcaactgcctatgatcatgatgaaaggttactgccttcttacaaaaattatattggcatcttcttaaaaataattcgaaaaagggataaactccctagcc

SEQ ID NO: 634 - DNA, 152 bp, RATTUS NORVEGICUS
ggggttggggatttagctcagtggtagagcgcttgcctagcaagcgcaaggccctgggttcggtcctcagctccgaaaaaaaaaaaaaaaaaaaaaagacaaaataacaaaaagaccaaaaaaaaacaaggtaactggcacacacaaccttt

SEQ ID NO: 635 - DNA, 152 bp, MUS MUSCULUS
ggggttggggatttagctcagtggtagagcgcttgcctagcaagcgcaaggccctgggttcggtcctcagctctggaaaaaaaaaaaaaaaaaaaaagacaaaataacaaaaagaccaaaaaaaaacaaggtaactggcacacacaaccttt

SEQ ID NO: 636 - DNA, 142 bp, MESOCRICETUS AURATUS
ggggttggggatttagctcagtggtagagcgcttgcctagcaagcgcaaggccctgggttcggtcctcagctctggaaaaaaaaaaagacaaaataacaaaaagaccaaaaaaaaacaaggtaactagcacacacaaccttt

SEQ ID NO: 637 - DNA, 165 bp, CAVIA PORCELLUS
ggggttggggatttggctcagtggtagaacgcttgcctagcaagctggaaaccctgggttcggtcctcagctccaaacctgaaaaacaaaaaatccctataaaaaaacacaaaatataaaaaagaccaaaaacaaacaaggtaactgcgcacacaaacaaccttt

SEQ ID NO: 638 - DNA, 391 bp, PEROMYSCUS MANICULATUS
atgaagagagagaactaacttcaacttctgctttttggagggtgaatgcctataggattggagcggttggcagagtggctcctcctgctggcaagccgttcggcaccacctgatacttgactgtgtatgaaatacttcagttttgctttaaatactggcagcaagagctagcattcggggttggggatttagctcagtggtagagcgcttgcctagcaagcgcgaggccctgggttcggtcctcagctctgttaaaaaaaaaagaccaaaaaaaaaagaaaaaaaaaaagacaaaataacaaaaaagaccaaaaaaaaacaaggtaactggcacacacaacctttttcattttcaaaggcacccaaggacattttcctggctgggagcaat

SEQ ID NO: 639 - DNA, 359 bp, PEROMYSCUS CALIFORNICUS
agaactaacttcaatttctgctttttggagggtgaatgcctataggattggagcgcttggacagagtggctcctcctgctggcaagacgttcggcaccacctgatacttgactgtgtatgaaatacttcagttttgctttaaatactggcagcaagagctagcattcggggttggggatttagctcagtggtagagcgcttgcctagcaagcgcaaggccctgggttcggtcctcagctctggaaaaaaaaaaaaaaaaaaaaaaaaagaagacaaaacaacaaaaaaagaccaaaaaaaaacaaggtaactggcacacacaacctttttcattttcaaaggcgcccaaggacattttc

SEQ ID NO: 640 - DNA, 350 bp, MERIONES UNGUICULATUS
agaactaacttcaatccccgcttttaggagagtgggtgcctttaggattggagcgcttgaacagagcgactcctcctgccggcaagccctccagtaccgtcagatacttgactgtgtatgaaatatttcagttttgctttaaatactggcagcaagagcaagtgctcggggttggggatttagctcagtggtagagcgcttgcctagcaagcgcaaggccctgggttcggtcctcagctctggaaaaaaaaaaaaaaaaaaaaagacaaaataacaaaaagaccaaaaaaaaacaaggtaactagcacacacaacctttttcattttcaaagatccccgagggcattttc

SEQ ID NO: 641 - DNA, 197 bp, AOTUS TRIVIRGATUS
ggccgggcgctgtggctcacgcctgtaatcccagctcttagggaggcagaggcgggaggatagcttgagcccaggagttcgagacctgcctgggcaatatagtgagaccccgttctccacaaaaaggaaaaaaacaaaaagacaaaaaaaaaaataagcgtaacttccctcaaagcaacaacccccacccccccttt

SEQ ID NO: 642 - DNA, 205 bp, CERCOPITHECUS AETHIOPS
ggccgggcgcggtggctcacgcctgtaatcccagctctcagggaggcagaggcgggaggatagcttgagcccaggagttcgagacctgcctgggtaatatagcgagaccccgttctccacaaaaaggaaaaaaaaaaaaaaaaaaaaagacaaaaaaaaaataagcgcaactccccttaaagcaacaacccctcccccccccttt

SEQ ID NO: 643 - DNA, 204 bp, GORILLA GORILLA
ggccgggcgcggtggctcacgcctgtaatcccagctctcagggaggctaagaggcgggaggatagcttgagccctggagttcgagacctacccgggcaatatagtgagaccccgttctccagaaaaatgaaaaagaaaaaaaacaaaagacaaaaaaaaaataagcgtaacttccctcaaagcaacaacccccccccccccttt

SEQ ID NO: 644 - DNA, 200 bp, HOMO SAPIENS
ggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaaccccccccccccttt

SEQ ID NO: 645 - DNA, 203 bp, HYLOBATES LAR
ggccgggcgcggtggctcacgcctgtaatcccagctctcagggaggcaagaggcgggaggatagcttgagcccaggagttcgagacctgcctgggcaatatagcgagaccccgttctcccgaaaaagaaaaaaaaaaaaaaaaaaaaaagacaaaaaaaaaataagcgtaactcccctcaaagcaacaacccccccccccttt

SEQ ID NO: 646 - DNA, 200 bp, MACACA FASCICULARIS
ggccgggcgcggtggctcacgcctgtaatcccagctctcagggaggcagaggcgggaggatagcttgagcccaggagttcgagacctgcctgggtaatatagcgagaccccgttctccacaaaaaggaaaaaaaaaaaaaaaaaaagacaaaaaaaaaataagcgcaactccccttaaagcaacaacccccccccccttt

SEQ ID NO: 647 - DNA, 200 bp, MACACA MULATTA
ggccgggcgcggtggctcacgcctgtaatcccagctctcagggaggcagaggcgggaggatagcttgagcccaggagttcgagacctgcctgggtaatatagcgagaccccgttctccacaaaaaggaaaaaaaaaaaaaaaaaagacaaaaaaaaaataagcgcaactccccttaaagcaacaaccccccccccccttt

SEQ ID NO: 648 - DNA, 205 bp, PAN PANISCUS
ggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaaaaaaaaaaaagacaaaaaaaaaataagcgtaacttccctcaaagcaacaaccccccccccccttt

SEQ ID NO: 649 - DNA, 197 bp, PAPIO HAMADRYAS
ggccgggcgcggtggctcacgcctgtaatcccagctctcagggaggcagaggcgggaggatagcttgagcccaggagttcgagacctgcctgggtaatatagcgagaccccgttctccacaaaaaggaaaaaaaaaaaaaaaaagacaaaaaaaaaataagcgcaactccccttaaagcaacaaccccccccccttt

SEQ ID NO: 650 - DNA, 198 bp, PONGO PYGMAEUS
ggccgggcgcggtggctcacgcctgtaatcccagctctcagggaggcaagaggcgggaggatagcttgagcccaggagttcgagacctgcctgggcaatatagcgagaccccgttctccagaaaaaggaaaaaaaaaaaaaaagacaaaaaaaaaataagcgtaacttccctcaaagcaacaaccccccccccccttt

SEQ ID NO: 651 - DNA, 195 bp, SAGUINUS IMPERATOR
ggccgggcgctgtggctcacgcctgtaatcccagctcttagggaggcagaggcgggaggatagcttgagcccaggagttcgagacctgcctgggcaatatagcgagaccccgttctccacaaaaaggaaaaaaacaaaaagacaaaaaaaaataagcgtaacttcccccaaagcaacaacccccacccccccttt

SEQ ID NO: 652 - DNA, 196 bp, SAGUINUS OEDIPUS
ggccgggcgctgtggctcacgcctgtaatcccagctcttagggaggcagaggcgggaggatagcttgagcccaggagttcgagacctgcctgggcaatatagcgagaccccgttctccacaaaaaggaaaaaaacaaaaagacaaaaaaaaataagcgtaacttccctcaaagcaacaacccccaccccccccttt

SEQ ID NO: 653 - DNA, 15002 bp, HOMO SAPIENS
  misc_feature (12055)..(12055): n is a, c, g, or t
ttgtggaaaggatggatgggtgggtggatggatggatggatggatggctggatggatggatagaaatgcaacttcaagatctggttctacagtattacatatgaagcaactgagactcagataaaattcagtgagttgctgaagactattcactcattgagcctcacattctgatgctatatatatcaggtgctatgcagatgattagggaagggcaggactgagattaaactgacaccccctacatcctaatccaggctttttccactgtatggcgatgcctcccaaaagaattatcacagcgacttaataaaggagggagaaagttagttgcaaatttcaagcttagaaattcacaggcaaaaatggctgaagggaaataatcacactaatagtgatgcaaggatgtaactggaaaggaaaaaatggagagaaattaaaaacacaaacagatttagtcagggtcctgataaagtgataatgtaatcaataaggcaaatcaaagataaatgattagagatttaaaacttcagcctatcagaaaaacctgagggaaaagattttgaattttttttatttcttccaaaagatcctatttgacatatttttttaaatctaaatgaccaaagatttcgaatttggaaaggcagacacatatctatttcccatataaacatctacatttcttgccacatagcaccgcaacactcatctggttccaaagcagacggcttggccctctgttatcaagagcactatcaccgttctggcccggagagctaagttttcttatacataggtcgtagccaaagctcagaggaaagaagtgacatttggtgagtgtacaggatatgttctccagccactttttaggtgctaagttacatcttgaaataatgtcagaaattggaagatgccgtatttaaacttcactagcccaagccctaattcacctcgacagtttaatatcttaatgtttcttaaaatttgaggactcttagaatgacccctttaaaaatctgctgaaaaactggatctttttgtgggaaaaactgtccatacctacatatctaaaatgtgtatgtggtttcagtctctcctgtgcacagtccctcctgctttgaagggatccccaacatgtttttttcctagaggtagttggtcacaaccatcggataacatgcttatcacctgggtgataaaatattctggacaccaaaccccgatgacatgcaatttacctatataaagaacctgcacatgtattcctgaacctaaaataaaaagttgtaaaaaataataatacaagtaaaagagctttctgcatgtgttataaatctgttcaggaggactttccactatgctttcaagagaagtgtcttgaagaatttagcatcactctgaattcccaaaaagctctgtgcttttcaaggtgcttttgaggaagaaggatgatgcttcaactgtcttctttgctgtgacttctaggaagttcattatcacatcatattctcaaccaaataattaacacttgacattggcattttcttctgtctttgtttttaaattttattgttgtttgctaattttattgtttttatctttgttgtatgtattgtcccggctctccatttcctccctctccctgccaataaaagagacccatagattttattcttttgttctactttttcatcaatgtttcttctacatatatctgacatatatggaaaaaagaattactgcctatatatgcagaacttgtaactcacaggatttctaaactaaaagacttttcttaataggtgtgaagtgaaattctgcaagtagctgcatctgggaacttcagagttaaaccgatatctcagtataatctgtcactgctatagtttcttttctttgacctccaaaccttatgttgaaatttgacccccaatgttgaaggtagagcctaatgggaggtgtctgggtctgggggcagatccctcaagaatagcttggtcatcaatgagtgagttctcactctaatagtttctgggagagctggttgttaaaaagagtgtgtcatgtcccttctctctctttttgcttcctctctcaccagtgatctgcctaggccagctccccttcatcttctgccatgagtggaagcagcctgaggccctcaccagaagcagatgttggcactatgcttcttagacagcctgcaaaactgtgagccaaagaattcccttttctttataaatgacccagcctcaggtattcctctatagcaacgcagatggactaagacactcatgcttcctttcattagctatttcaagctcaaaaattgtgagactgtgccaagaattagggcaggatcttgtcattgagaacttacaacttttctatctaaagcaaaaggtcctaatcccaaaccataattggaaaggtgcctctactccaacctcattgcccttcaactgccccttctctgcctcccattcccctcttactcaccgaaagacaaatgagtttaggctgggcacagttagccatcgttctcagaaaaacaaaatcttgctgcttggtattctcacttagaatcccttcacttagcttatggatcaccctacacagattcctgacagctcactgggccatggaaccagccagatttttgagctacatctgcagtatcttttctactcaggcagctggcataaggtgggaaaattcattaacatgctaatgatgtcctatttaaaattctagtcctgattattctgtgattaaatctctccccttgacgtcggtttcctttatagctgagttacagtgcgaaatcaacaatattaataaagtacaatacagattaccaatttatacacaagggatggtgaaaaactgctgcgtaaatctgagggctgtccttttaacttgaaaagcagttgtaggccgggcgcggtggctcacgcttgtaattccagcactttgggaggcctaagcaggtggatcacctgaggtcaggagtttgagaccagcctggccaacatggtgaaaccccgtcactactaaaaaaatataaaaattagctggcctataatcccagctacctgggaggctgaggcaggagaatcgcttgaacctgggagacggaggttgcagtgagcagagttcgcgccattacacttcaggctggttgcaagaagcaaaacttttgtctcaaaaagagaatgtaaaagcagatgtagcaaacttgtaaaagttgctgatttttttcattccgcccttctttaaataaacacacacattggaatccacaataattaaatgtttatagttaatttccataaatattcaaggcttggaagatagatttagcttttcattctatttcactgcacttaacattgctccgaattcaaatgtgttatcctgaagtctcaagaacagctgacaggagttaaagcacacttgtttatcatctctttatagagattgtgtcttggcaacaaaagccaatggattcggtgatgaggtctgtctccctttgcccttgagtaccttggcatatttcagttccttttctatcaccgtattgttctattactgacactccaaactgaatatccaactcagacaactcaagcaccttccaagaaagacaaacttatcactaccctcattaatttggaaaatgaataaagaagcagaagattcaaggtccagatctgcaatctaaaatgcaggaagagaggactcattcatgtgtcattttaacatctggccccagtaaatgaacaaatattgatggtatagtatatttactattttaaaagacatcatgctggacaccaaagcagtacagtgaaacctaagaacaatgttctgacctcggtggactcacaatctagccagagacacaagatacaatgatattttaagtgatgcatgcagcaaattcatctcaggtgcatcattagagatacacttcacatcattaaaaggtctctgtacgagagacactgctaattgttccctaagactgcaaatctccccttttgctatggaaacagaattcatcactttagtggggcacattgctgcccagggtaaggacagctcccagcccctcttgcaagtagctgttaccatataacaaggttaaggtcacagggatgtggccgtagcagagatgtgtgcagatttctaaaagcgtacttaaaggggatagatgagctctcttccttctttgttttttttctgactggacttggacatgcatgtggctgggtcagccaggttggactgaatggcacatgttgtggggcagagaagaacaagatagaagagtctgggttcctgacaagcagggtggcttgatcatccatgagttgcctttttcccctgaacgctgcattccacaactattgttggaaatcaaaatctttttttaagaatactacatattgctcatttcttaaatggtttcattctctaaggctaaaaagagtcatggtacatgcatgcatatgtgtatgtgcataagtacactctcctttgtactttctctacatttaaacttgaacacagagctccctctgaatttttaaccctcaaaacacaaaaaaagctgaatatggactttgccccagttttatcacattttaataaataaaagcttcaatgtattcaggaattcattatttatttgtttcccacactctcaaccctagattattaatttggttcacctaaagccaactcacctcaaattggttaaaaaaaattaagcacgtgtaaatcaatgtatagggaaagggtggctgatccagcaaaatccaagtgtaaaaacataaagagaggcttcacaacagtaatgagccatctgcaatttagaatcatccatggtaacaatctctcattccatttgtttatgttttattcatcattgtgaaaatacttagtgaagtgacaatgatgagattcaaaaatactgcatggttctttttttaaaaaggatattaatggctttcagggactcaactattcaaatgaaaatattgctggactaaatttgctcaaatttgttcacttcctcttcattcttggatataatatattcttcattttatgacaaaaaactatgtggtgaaaacatgagaaattccgtagtttccatcgtttatacatttaaaaaatcaatagcagaaacctatttcaaatgtacaatcctcattcctatcatgacagagtaaaccaggctgcggtgtcttgggtgagcgtgttgtcattccctagtactacagttatttagcctcaaacgtactgtctctagttacatatatggcaaaaacaaggcttgacctttaagaatgaaaagaaaaaaagataagtactgagctgttaagccaacttaagggtattcattacaaagaagacagaataactactgaacatactctttgattctaatatctctatgaagaaacctctctctacacaaattgatagacattttaaaagaaaatttattcatcccaaattgatgcacttaacttcagctagctgattttatatttctcaatctagcttataaatactactcttcatttacttcaatttacggagtatttacaaaagccctatttcaaatctggttcctatcagccccgttttttcttatttcttcttaaataagcatactttgtaaaatatagccatttattcatatatatatttctttcatttgttttagtaaatgacatttatttaggaaccattttttaagaaataagtttttaccatctcaatttgcactgaaccagcgtcttctgagaccatccctctgggtgcaaatctcagagtaagctcagttactcctaacgactggaaaaggaaagaacacaggaacacttgaactcctctgacaagcagacactttggctaaggcggcattcacatcttctacatcatccttcctggggaaagaagctctcagaaaagtccacacatagaagagccactttatggtcttccctcaattttctcagatcagcctgatttagtctatgattgtgcasacaaggagacggagccaagggacaaaaggaagataattgtcacacaaagaaaatgctcaagaccttgccaagggagagactgctctaaataataacaaggctattagcaaacacaggttgagacagggaacagaaaaagaaaagatgtcwcatgtgaatatgaaaacaaaagctgacaagtwattccattcagtttgccaaggcacctgccaaaaatgtccccaagggaaaagggccatgtgctccatgacgctcttcacagtgaacgttcacatwaaggcacagaacattctgcctggccacaaaacacaacaaatatcatcaatatcttgccggggtaacagttgtgcactacctaccataggcagtctcctggtgctccacttctcttctcagtctgttgtaatcttctttaacacttcccagctttttgacattcctggaactcaacaccaacagcttgctgaggagtccctccagcccttctctctctgatgttaatgatctaatctcctcggtgaggtctttagccgtccagaaatggtttcctacagcacgttgtgggagagaagaagggaaaacaagctttacagcatccacacacatggaagatctagcagcagctaatgtacattcacagcatgggcaaagaaactgcactttgagattatgtwcaatttccttagcagtcctctggggaaagaacctggaaaaatattgctccccttaaactggaaaaatcaccgtcttttttatcaccttattcttgttatgtccattttaggagttccattggtgaagccatctcccgtaaagtatgatgacccaaaaatgtcccagtggaatccagagaaaccacaatgtgtcactccattccaataatatacaaatgtcaaagagatgacatattatagcagtgtggaaggggactgggccatataatcagagttgacctaaattaaattcctcccctgttactcaaattgtgtgacctggagcaagaaaattagcacatctggggcaaatttcttataaaatataattgcaataacacctgactcataggtagttacaagaataaacaagaaattctgtaaagcactgagaaaaatatatgataaattaaatgtagcaagattgcaggatacaaggtcaatgtacaaacatcaatcttatgtctaaataacaaaaaactagaagatgattttttaaaaaatacaatttacaaaatattttaaatctcataatactgagggatactttctttaaatatgtgccagagctctacagtgaaaactatttttccaaaactgctgaaacaaattaaagacaaataatttaaaagatacactgtatacatacactggaagttcaatattggtaagatttcaattttccccacattgattcattgatttaatataattccaatcaaaatgttatgaggttcttttgtagaaatttatgtgcttattctaaaacgtctaaaagtgagctgggcatgggtggctcacacctgtaatcccagcattttgggaggccgaggtggtgggtcccttgaggtcaggtgttcaagaccagcctggccaacatggcgaaaccctatctttactaaaaatataaaaattaatctggcacagtggtaagcacctgtaatcccagctactcaagaggctgaggcaggagaatcacttaaacccaggaggtgtaggttgcagcgagctgagatcctgccactgcactccagcttgagcaacagagtgagacttcatatctaaacaaataaaatctagaagaatgagcccatgagtttgaggctgtagtgcactatgattgcacttttgaacaggcactacactcttgtctgggcaacatagcaagtccccacctcaaagaaggagagggagaaagaagaagaaggaggaggaggaggtgaaagagggagggagggaaggaaggagaagacgaagaagaatgaagaagaataggaagaaagaagaagaaggaagaggaggaggaggatgacccaaamcaatccttaaaaagtgagtcttggtttcaagactaatgctaaagccactataaatcaaggtaatatggcattgaatatacatagacaaacagatcgatggaatagaataaacagtccaggagtggattaacacatatacagccaaccaaagtgccaaagcaagtcaatgaggaaaagaaagtattttcaataaatggtgctggaacaactagctatctacatggaaaaaatattaaccttgattcatacctcaaaccacacaaaaatttaattaaaggtggatcacaggcctacatgtaaaagttaaaactataaagcttctacaaaaaaaaaatgaatatttgattgactttggagtaggcaagaacttcagataagatacagaaaacactattttaaaaaggataaatgggctttatcaaaattaaaaacttctcatcaaaagacacaactaaaaaatgagacgacaagtcacagacttggagaaaatcattgcattatgtttaactgacaaggaattgaatactacaacttgacaattaaaacacaaatggtgccattgaaaaatggtcaaatgacttaacaggcattttacaaagtagacgcacaaatggctaataagcatatgaaaaaacatttgacatcattaatcattaagaaaagaaaaattaaaaccataaatattccactacaagcccactcaaatgtttcaaattaactcaactaaaaatgtcaacattcacaagggtatggagcaactggaactctcacgcatgtaaaatggtcccttcaagaaaagttctgccagtttctcataaagttcaatgtatacctaccctttaatccaacaattctacttccaggcatttacacaaaagaaatgaaaacatgtcatcaaaaatatttgcactcaaataaatggtcacagtgtcttatgcctgagagccagccccatactggaagcaacctaaatatccatcagcaggaagatgcaaaaataatactatagctatgcaatgaaagactactcagcaataaaaagaaacaaactcgtcatacatgcagcaacatgtatgtgtctttttctttctttctttttttttttttttgagacagagttttgttcttgttgcccaggctggagtgcagtggtacgatctcagctcactgcaacctccaccttccagtttgaaactattctcctgcctcagcctcccaagtagctaggattacaagtgcccaccaccacgcccagttaatttttgtattttttagtagagccggggtttcaccatgttggccagactgttctcaaactcctgacctygtgatccacccacctcagccttccaaagtgctgggattacaggcgtgagccaccgcgcccagccaacatgtacatgtctttagaaaagatgcaaaagagtagtgccacatgattccattaagttctagaacagccaaaattaatccatggtagaagatatcagaacagaggagggacgatgatgactgggacaagatagggtgttgtttcttgagtgatgagactgttctatctcttggtagggctgttcgtaacacaggtgtcaaaattcaagaactgtaaacacttaagacctgtgcattccactgaatataaaccatacctcaattaaaaagaggaaaaattaagaaatttttaagcatgaacattgaacaataaataaatatataacattaatgagtgtcccataaattagttagtgttaattagaaaaccccactgtgttttctcagaatttatagtaaccataatcacaataatagctacaaactttctcacctttaaatgtcatcacaactttaggaaacaggttatatcttatattattattattattattattattattattattattattttacattttaacatctctgaaacagggatgtgtcctacaatcaccatcagccaggaagtggtcaggatgcagtggttataactggattattttccctggcctggctgaccatctgcaaccccctggtgtttcaatgtacaaactattttaggaccatctgcgtactggctgttgcctgaaaatcttgtgttgatgtggatttgtaagactgggaaagcaccagcattcaaaacttgaagaaaggtgttagcagtttggcagacaatcctacagaaaactctttattcttgggggagaaaaagttgcctcccatcaacttcaatggttcacaggatgatgacgtgtggggaaacacaaacctcgacaacattgagtccaaatgtgtttcggaagactcagaatgaagaagaagatgccaaggaaaaccttaaccaatttattttcctttttaatgagtgatatgtgatttttcaaaagcctaaatacatctaaaagagctatatcagttaagtataagataaaaattctaaaccatagaaagcactgttactgcttaattgacagcaatttttctttctttgcaatgtataaaataatgggtgctttttatgttcactgaaattcatattgttgtctccttttacagctgggaaagcggaaatctggagaactaaaatatcttgctgaagatcacaatgcagtaaagcatagggcaagtttccaactcgaacatcaccccagaacacttccttttaaacataaattatgttgctaacttttttattgttacttagcaggcagatttcaaactgatacaaatgttgaaaatgatacaaaatgttttcccaccttaaaggcattctgaaacctagcacataacatctgtcactgaagtccatagtttccaatgccatgattggataaagaccctattgagactcatgtgtttgtctctcactgcttaaaacactcatgtgttttaagcaacatgacaagcacaaaagagaacaagtactgctcctgctctccaggaagttaccataaaataaaggggataaggtgccttccaaagaaaaaccaaatactaggagaggtcagaggagaagagagattattccagaaggagagactgaggcagatttcaggagggaagaatataagagtcccaaaagaagaaacaggaacccaggttagagagcaatgggcaaggggtaggaggtgagactaaactcacaaggaagttgcttcaaaacaatagtgccccaaatgccaagctgatgggttggacccatttaggaaaaacccacaaagtccacaggagagcagactgcacttccatgagcttcaagctgtaaacagtaacctcaaataaaacttttcccggatcggtaatttccatcctgaacaatggccaagtcaaggaagctgaggatcgagccccctaccgcatgccacaaagaacctcaagggaccccatgctctggggagctcgcaggaggaccagcacagaggtgtgactgcgttggggcagggtggccacatgtacaatgttgtaccttccaatgttcctccttaggctgtgcttatgaagtcccaacatgtcttctgggctgtgttttcaaccaagagagttaataaaaagtgggatgggcaccaactatgcctccagagacatcaggcagaatatgccacagcgtgccatgtcctcctgcaaacagtcagaaagaaaagatctctaaaagcaaattggaagagactatatatacagagcgagtgccggtacagaagttttgctgcaaatacaccctctaatcccttgcaacccactgcaagctcgagagaccagtgcccttggtaaaacacaggagagatcattaattaaagcctaggccctttctttgatcctgagggataaggcttgggaaaatgcacagtgataaaagtgaacattttaaaagagcagagtgagtgtgaattcccaaagagaccattcatatcaagacccaagccatgagaaaatgctacaatgcccttgattaatactttangaggcattttgacctcagatctaatcccatgaggcttagaccaagaccttgcttgatgcagacctctctgactgctgaaattggtcaggagctcagtgatgaaaatttggaaagtcaatgctgatacctgaagaaagtcccatcagagatctgtgacctgaatcttcataactttcacaggccctttctaaatgccaaggaggatgtgtgtggagaagaatatgccacctatggcatcccaatatgcctgtgcccgcctgctcttcaacaagggaatgaaacctgggtattctcataggttttttaaagcacttgtccacatattttattccgcaaataacacaatttaaaattcagctagaatttgccattttctgtcagggttgtgcatttcttccagtcaccatgacttcctgagatagccaaaacacaaaatttaaattgtgtttaaattaaataaacacaatttatgctgaagctgactttacccactctgttccaacattacttatcctcagcatctctgaattcaaatatcagccaggtgtggtggctcatacctgtaagcccagcactttggcaagccaagatgggaaggttgcttgaggagttggagaccagcctgggcaacatagcaagacccccatctctacaaaaaaattaaaaaattagccaggcatggtggtatgcacctttagtcctagctacttgggaggctgaggtgagaagatcacttgagccgaggagcttgaggtggcagtgagtcatgatcataccactgcactgtagcctgagcaacagagcaagaccccatcccttaaaaaaaatacctgccctctgtacttcatagcacctggggcattttgaacagttcttccttcacctgaggtcattatttgagaatccaattttagagactacaatgctctcattacttggattgtatcatttaatcttcccaacaatccaggaaggggagtctcttaatgattgtccttgatttccagttaaaaaatctagacccagaggtaaccttgtgaaggtcacagagtttcagagttctaagatcccatgcggagattgttgaacttgaacttgtttcttctgatctcatgtcaataagtgacttagtcaacacctgggtagaaatcatcactaaagtgagaaagcacatggtgagggggatgaatcctgtgcccacatggtccctttcacctgcccaatcctcaggcctctcacctggggtacccctcacacacatggcactttcctgaactcatggacatttcagtttgtaacgatcctcctaagattatctactttcacctatccacatttgaactatacttctttagtggaaaaaaagaaataaagccagtgctgagaatgatcaaactccagaaatagtagactgctgtataatttgtcttgcgtgacaccagatctttcaaggaactgcaagctcaagtgcacagttaagcaagatcaagatctacctttgtctttcagagccttcccttggcgaggtcaaggatatctgctgtgagcctgggctggctgaaccaaagggcaatggaggcggccaagaagccccagccaactgctcccctccccaacccccttctccctggaggtcccttcagctagaatttgccattttctgtcagggttgtgcatttcttccagtcaccatgacttcctgagatagccaaaagcatttgctgtgagcagagccccatggctggctttgcacatcaaacatttcagacagccatatatttcatagaatacaaggggaagataacactggacccacagaactttccaacagcttgagaaaatgtggtgcctggtgactgactaaatatacaatcggacaacagccaggccatagcaaaagatggaactctgatccacaaggtgcagcaactttcccagaaaacaatacccttacccacaaaaagcaaccccggaagctggcctgctctaaatcagacctgctggaagccaaattgctctctagtaactatccaggaagctaaataataacttctgtaacaattgactcaaaatgttcaggactggattaataactgatggcttccctaatttttgtccccgattccaacttaggacccaccagagaaagccaaatatggtcccctgaccaagcacacaggatgttcgcttctagtgagccgcctccagcttccccgcattgataacctccaatcagggcacacctgaagccttcgcctttttccactataaagctctcccactcgtctgcccacctttgagtctctgccaaattgcaaatgatggtgtctgactcccttgctgtagcaagctctgcataatagcttgtgcttattctctttgggtaggtcttcatttcttttcacaccagataggcatcatctttgaaaacatctttactgggtctcttgtctccctcctgcagccactgatctaaatcacagacttttttagaaagagggaaaatagtaaagaaagaaagcgattgagttgatgctcccagccactgtctgtaccctgaattttgtttttctgccatatgtcaggtggcctctcctggaccatcgtcactgtttctcctgcttccctgcagctgcagacacaggagcactgtgtaggtctggccttgagcacccacccacccagtacagagccagcccctgctgttctgaatcagtgcagtgaccacttagaacacagtgcccatgcctctgtgatctgaactgctctttccagttccatacaatgttaagaacccacttaagaatgttttaatcattcagtagtttttcgtggtatataataaaaaagtgatttaaatacaatctattttattttagattatatcattaggggttattttacaaagttacctttaaatactattgtaaataaaattaataacaaa

SEQ ID NO: 654 - DNA, 265 bp, HOMO SAPIENS
gttcgtgctgaaggcctgtatcctaggctacacactgaggactctgttcctctcctttccgcctaggggaaagtccccggacctcgggcagagagtgccacgtgcatacgcacgtagacattccccgcttcccactccaaagtccgccaagaagcgtatcccgctgagcggcgtggcgcgggggcgtcatccgtcagctccctctagttacgcaggcagtgcgtgtccgcgcaccaaccacacggggctcattctcagcgcggct

SEQ ID NO: 655 - DNA, 266 bp, HOMO SAPIENS
gttcgtgctgaaggcctgtatcctaggctacacactgaggactctgttcctcccctttccgcctagggggaagtccccggacctcgggcagagagtgccacgtgcatacgcacgtagacattccccgcttcccactccaaagtccgccaagaagcgtatcccgctgagcggcgtggcgcgggggcgtcatccgtcagctccctctagttacgcaggcagtgcgtgtccgcgcaccaaccacacggggctcattctcagcgcggctg

SEQ ID NO: 656 - DNA, 265 bp, HOMO SAPIENS
gttcgtgctgaaggcctgtatcctaggctacacactgaggactctgttcctcccctttccgcctaggggaaagtccccggacctcgggcagagagtgccacgtgcatacgcacgtagacattccccgcttcccactccaaagtccgccaagaagcgtatcccgctgagcggcgtggcgcgggggcgtcatccgtcagctccctctagttacgcaggcagtgcgtgtccgcgcaccaaccacacggggctcattctcagcgcggct

SEQ ID NO: 657 - DNA, 279 bp, BOS TAURUS
gttcgtgctgaaggcctgtttcctaggctacatacggaggactagttccttgtttgcgcctaggggaaagtccccggaccgtgggcagagagtgccacgtgcccgtgcacgtagacttcccctgcttctcacgactaaacccgccaagaagcgatcctaccctgggggtggggaagccgagcggcgtgttgcggggtgtcatccgtcagcttctaatagttacgcaggcagtgcctccatgcgcaccaacctcacggggctcattctcagcacgtctca

SEQ ID NO: 658 - DNA, 1116 bp, HOMO SAPIENS
tggcatccagtccactttaccatcagtgtttaaggaaaccatctctgataaggatgactgaggaagagtactctttggcttgttgacaccagcacagctgacacacccagatatctgtttggtctcctgtgaactttcaaccaggatttaaggatgccactctggttaaaagctgaaacaacttcaaggaaacttcagggaaaagagaaggcctggaatctgatcctccacttcagagaacagggagaaaaaagtacatgtgaaatactactacttatccatctcctggctctgagcagtacatgtcaggaacaagagggaattgtagctggaagacagtagaagagccataggacaagtctctaagagaaggaagaaaaggcgcaatgaaagaaaacaagtttaagatagtagaaaagcattcaaagacggcaacctgaggtggtggagtctatggcccttctccttgaatctgagtgaaattagtgagtctttcaatccaaagactacagtcatacctcaactatctacagaggactggttccaggactcccatggatcccaaaatccaaggatgcttcgaatcccttatataaaatggcatggtatttgcatataacctacatgcatcttcaaatatgttaaaccatctctagattacttataataccaaatacaatgtaatgctatgtaaacagttgttacaatgtattgtcaaagacaagctctccttgccaatccctgaccaaatgagagattcatgagcaaaataaatagttgctgatatttaaggcactaagtttggggcaatgtattacacagcaataaataactggaacagcatagtaaattctagttaaaaatactaaactactaagttttagttaacatacatgctcaagttaagggtgtggtatactaacaactgcaacttactttgaaaagcataaaaaaagacactagaagaagacaggatggatagatggacaagatagaagacaataacaaatagaacaaaatgttgtgaaattatattctacaattattttaaagtctgtgtggtttgaaaaattgtacgataaaatgaagagtgaaataaagacatttccatacaaa

SEQ ID NO: 659 - DNA, 3053 bp, MUS MUSCULUS
taattcttcttcttctctaagcaccatgacaggagcacagcctttccagagggtacctacctaagttgaaggtggccttaagttatctctataaccaggtgctccagaagagggggatgagcatcattaagcagtgagcagcatcaggaagactcctgtcacttgtcagcagacttgactttcagtaaacagttggtgtctatgaaaatctaactgtcttgttaaagagagtgatttctgcatttggcatttgaaagactatggaggaggcttgagtggcattcccaccatgtttcctgagcacagaggtcagctttgcaaagacactgcttggaagtggactgcgcaggcttcttagatatgtagccatcttctgggacctgccagtcttggagagagagaagatccagaattctacaagccgcaaacacactgtattagagcaggctgagatcagatggctggggagaaatacagatggatggagttaagtatgggcgaaatcaacgtttccagatgcacagggtggactaagggtgctgttctttttgatgaagagcaaggacaagaagcatctcttgtgaatcactaggcaggtgtttgctctctggggaagtgacaggaaactcatccctgacccgaggttactgggaggaaagagcttcttttggttttgtttctctgcttggtgtacaagtcagtgccctgtctagggaagaagaacgcagatgatccttggatgtccccaagggaagaatgaccactctattacagaattaaggtcatctagagatcctgtttcctattgcacatgaatatggaatttggtaacagaataaacatgacatttggaatgttcctagagcatacttagggcaactggcaaggctgacatagcttttactgatctgtctgtttagtgagtggacaggatgccagtctggcagtttgcattggcaataaatcactactagacattttatttaaaatctggcactggctttggagaggtgagggattgtcaatttgtttttattttgaggccaggcacaatcataacacaaacaggccacctggaaactttagcacatatgtaaaagtgtggccctggtctgtggccattgcatgcttggcaggagcagggagaggtaaatgagagtaagcactgttgaccaggaagactctggcagacctgtgcagcccgaggcagccctgctggaaaccatgctggatccgaaagtgtgatataggaaggctatcatcagcataatagatgagataaaatttgttttcctgttttatcatgagattctcacagaatacattaggaacacacaagcaatgcattccatccgagagagagagagagagagagagagagagagagagagaggtcactccaacaataaagttaagtattgagctggtttgttcctgcctttgccatcctccacctcatgtcttttcttcttctcttcctctggttttctgcctttatttttgtcttggattgccccttttttaaaggataaattgtgatcaatgtctcgcagttttgagtgaaaaaatatttcacggtaggcactggaaatgttcttcggtgtagagttactgcctagcaagcatgaaatttagcttcaatcatagtgctcacacacatgcatacacatgtgcgagaatgcatacacacttatgtacacatgcatgcatacgcaaggagaagtttagactcttacagctactgcagtggcagaagttagggctccttttccactctcagcaattgtgtgattttctgaatattgtgtaaggctgaatagtatcactgtgtagcttttttagttttttcattgtgaacatatgagctactccccctgccccctaggaaaaccatatttcaagtacaccgacttactaagacaccagtcttcttgtcttcatttgcatgttatatttacaaaccttaattagtctccaccttgttactgtgcaaactcttaggatcacttagatatatctttgtgaatggcttcatttgttcatttattagaaatatataatttactcattaagctacaaatatttaaatgaccccgacttggctttcaaagagcatttcacctcttttccttgattttatatgtatagttttcataatagtatatagctaaaagtaggaaaaatatagactggtatcttcttggtaaaatcatttgaagtgatgtaatggtcaaatgcaaaggaaggcaccattttaatgctgtactgaagacactcctggtattcaggacatttgtacctaaagatacctggtattaaatactcagaaaaactgtcgaaattagatttatgacctcaaggacttttctttgtttcgataaatgtagtattagactgtttacaaaacgttctcaaaagttttcttatgaatgtaatagggagtctgttgctacaatgtcccatgactaagccatgacttagttggttgaagacattcataaaaggatacgaagaaaagcagaaaatgagagccagcctcagtgaagccacttaaaaacaccacagttctggtagtttgaacatatccaatggctgctttgagtgtcaactcagcaaatctgtgccccgaaagatcaagttggggtgtgtaggtaatgacacagatccctatatttaggatcccgggagctctgagagatacagtcacttattgggcatttgcatttctatctgactgcccacaggtaacagtggcaaacagacttccatcctggtacctgtttgcagcatttttttttttttgattcaatatacacatgaatttggattgcagttttctgatctgggtgtcttctatctattttaagggcatacatacatctccatgtgtatgtgtttacagggctctcgggtttagaatgttaagctttatgtatttaattaattcattaattaatttctcctatgctatggttgttacagagcacaaattatttgtgtgtgttaattagagtgtaattttaaattaattaaatttgtggattcaaaatatt

SEQ ID NO: 660 - DNA, 3003 bp, MUS MUSCULUS
taattcttcttcttctctaagcaccatgacaggagcacagcctttccagagggtacctacctaagttgaaggtggccttaagttatctctataaccaggtgctccagaagagggggatgagcatcattaagcagtgagcagcatcaggaagactcctgtcacttgtcagcagacttgactttcagtaaacagttggtgtctatgaaaatctaactgtcttgttaaagagagtgatttctgcatttggcatttgaaagactatggaggaggcttgagtggcattcccaccatgtttcctgagcacagagatatgtagccatcttctgggacctgccagtcttggagagagagaagatccagaattctacaagccgcaaacacactgtattagagcaggctgagatcagatggctggggagaaatacagatggatggagttaagtatgggcgaaatcaacgtttccagatgcacagggtggactaagggtgctgttctttttgatgaagagcaaggacaagaagcatctcttgtgaatcactaggcaggtgtttgctctctggggaagtgacaggaaactcatccctgacccgaggttactgggaggaaagagcttcttttggttttgtttctctgcttggtgtacaagtcagtgccctgtctagggaagaagaacgcagatgatccttggatgtccccaagggaagaatgaccactctattacagaattaaggtcatctagagatcctgtttcctattgcacatgaatatggaatttggtaacagaataaacatgacatttggaatgttcctagagcatacttagggcaactggcaaggctgacatagcttttactgatctgtctgtttagtgagtggacaggatgccagtctggcagtttgcattggcaataaatcactactagacattttatttaaaatctggcactggctttggagaggtgagggattgtcaatttgtttttattttgaggccaggcacaatcataacacaaacaggccacctggaaactttagcacatatgtaaaagtgtggccctggtctgtggccattgcatgcttggcaggagcagggagaggtaaatgagagtaagcactgttgaccaggaagactctggcagacctgtgcagcccgaggcagccctgctggaaaccatgctggatccgaaagtgtgatataggaaggctatcatcagcataatagatgagataaaatttgttttcctgttttatcatgagattctcacagaatacattaggaacacacaagcaatgcattccatccgagagagagagagagagagagagagagagagagagagaggtcactccaacaataaagttaagtattgagctggtttgttcctgcctttgccatcctccacctcatgtcttttcttcttctcttcctctggttttctgcctttatttttgtcttggattgccccttttttaaaggataaattgtgatcaatgtctcgcagttttgagtgaaaaaatatttcacggtaggcactggaaatgttcttcggtgtagagttactgcctagcaagcatgaaatttagcttcaatcatagtgctcacacacatgcatacacatgtgcgagaatgcatacacacttatgtacacatgcatgcatacgcaaggagaagtttagactcttacagctactgcagtggcagaagttagggctccttttccactctcagcaattgtgtgattttctgaatattgtgtaaggctgaatagtatcactgtgtagcttttttagttttttcattgtgaacatatgagctactccccctgccccctaggaaaaccatatttcaagtacaccgacttactaagacaccagtcttcttgtcttcatttgcatgttatatttacaaaccttaattagtctccaccttgttactgtgcaaactcttaggatcacttagatatatctttgtgaatggcttcatttgttcatttattagaaatatataatttactcattaagctacaaatatttaaatgaccccgacttggctttcaaagagcatttcacctcttttccttgattttatatgtatagttttcataatagtatatagctaaaagtaggaaaaatatagactggtatcttcttggtaaaatcatttgaagtgatgtaatggtcaaatgcaaaggaaggcaccattttaatgctgtactgaagacactcctggtattcaggacatttgtacctaaagatacctggtattaaatactcagaaaaactgtcgaaattagatttatgacctcaaggacttttctttgtttcgataaatgtagtattagactgtttacaaaacgttctcaaaagttttcttatgaatgtaatagggagtctgttgctacaatgtcccatgactaagccatgacttagttggttgaagacattcataaaaggatacgaagaaaagcagaaaatgagagccagcctcagtgaagccacttaaaaacaccacagttctggtagtttgaacatatccaatggctgctttgagtgtcaactcagcaaatctgtgccccgaaagatcaagttggggtgtgtaggtaatgacacagatccctatatttaggatcccgggagctctgagagatacagtcacttattgggcatttgcatttctatctgactgcccacaggtaacagtggcaaacagacttccatcctggtacctgtttgcagcatttttttttttttgattcaatatacacatgaatttggattgcagttttctgatctgggtgtcttctatctattttaagggcatacatacatctccatgtgtatgtgtttacagggctctcgggtttagaatgttaagctttatgtatttaattaattcattaattaatttctcctatgctatggttgttacagagcacaaattatttgtgtgtgttaattagagtgtaattttaaattaattaaatttgtggattcaaaatatt

SEQ ID NO: 661 - DNA, 3055 bp, MUS MUSCULUS
tgtaattcttcttcttctctaagcaccatgacaggagcacagcctttccagagggtacctacctaagttgaaggtggccttcagttatctctataaccaggtgctccagaagagggggatgagcatcattaagcagtgagcagcatcaggaagactcctgtcacttgtcagcagacttgactttcagtaaacagttggtgtctatgaaaatctaactgtcttgttaaagagagtgatttctgcatttggcatttgaaagactatggaggaggcttgagtggcattcccaccatgtttcctgagcacagaggacagctttgcaaagacactgcttggaagtggactgcgcaggcttcttagatatgtagccatcttctgggacctgccagtcttggagagagagaagatccagaattctacaagccgcaaacacactgtattagagcaggctgagatcagatggctggggagaaatacagatggatggagttaagtatgggcgaaatcaacgtttccagatgcacagggtggactaagggtgctgttctttttgatgaagagcaaggacaagaagcatctcttgtgaatcactaggcaggtgtttgctctctggggaagtgacaggaaactcatccctgacccgaggttactgggaggaaagagcttcttttggttttgtttctctgcttggtgtacaagtcagtgccctgtctagggaagaagaacgcagatgatccttggatgtccccaagggaagaatgaccactctattacagaattaaggtcatctagagatcctgtttcctattgcacatgaatatggaatttggtaacagaataaacatgacatttggaatgttcctagagcatacttagggcaactggcaaggctgacatagcttttactgatctgtctgtttagtgagtggacaggatgccagtctggcagtttgcattggcaataaatcactactagacattttatttaaaatctggcactggctttggagaggtgagggattgtcaatttgtttttattttgaggccaggcacaatcataacacaaacaggccacctggaaactttagcacatatgtaaaagtgtggccctggtctgtggccattgcatgcttggcaggagcagggagaggtaaatgagagtaagcactgttgaccaggaagactctggcagacctgtgcagcccgaggcagccctgctggaaaccatgctggatccgaaagtgtgatataggaaggctatcatcagcataatagatgagataaaatttgttttcctgttttatcatgagattctcacagaatacattaggaacacacaagcaatgcattccatccgagagagagagagagagagagagagagagagagagagaggtcactccaacaataaagttaagtattgagctggtttgttcctgcctttgccatcctccacctcatgtcttttcttcttctcttcctctggttttctgcctttatttttgtcttggattgccccttttttaaaggataaattgtgatcaatgtctcgcagttttgagtgaaaaaatatttcacggtaggcactggaaatgttcttcggtgtagagttactgcctagcaagcatgaaatttagcttcaatcatagtgctcacacacatgcatacacatgtgcgagaatgcatacacacttatgtacacatgcatgcatacgcaaggagaagtttagactcttacagctactgcagtggcagaagttagggctccttttccactctcagcaattgtgtgattttctgaatattgtgtaaggctgaatagtatcactgtgtagcttttttagttttttcattgtgaacatatgagctactccccctgccccctaggaaaaccatatttcaagtacaccgacttactaagacaccagtcttcttgtcttcatttgcatgttatatttacaaaccttaattagtctccaccttgttactgtgcaaactcttaggatcacttagatatatctttgtgaatggcttcatttgttcatttattagaaatatataatttactcattaagctacaaatatttaaatgaccccgacttggctttcaaagagcatttcacctcttttccttgattttatatgtatagttttcataatagtatatagctaaaagtaggaaaaatatagactggtatcttcttggtaaaatcatttgaagtgatgtaatggtcaaatgcaaaggaaggcaccattttaatgctgtactgaagacactcctggtattcaggacatttgtacctaaagatacctggtattaaatactcagaaaaactgtcgaaattagatttatgacctcaaggacttttctttgtttcgataaatgtagtattagactgtttacaaaacgttctcaaaagttttcttatgaatgtaatagggagtctgttgctacaatgtcccatgactaagccatgacttagttggttgaagacattcataaaaggatacgaagaaaagcagaaaatgagagccagcctcagtgaagccacttaaaaacaccacagttctggtagtttgaacatatccaatggctgctttgagtgtcaactcagcaaatctgtgccccgaaagatcaagttggggtgtgtaggtaatgacacagatccctatatttaggatcccgggagctctgagagatacagtcacttattgggcatttgcatttctatctgactgcccacaggtaacagtggcaaacagacttccatcctggtacctgtttgcagcatttttttttttttgattcaatatacacatgaatttggattgcagttttctgatctgggtgtcttctatctattttaagggcatacatacatctccatgtgtatgtgtttacagggctctcgggtttagaatgttaagctttatgtatttaattaattcattaattaatttctcctatgctatggttgttacagagcacaaattatttgtgtgtgttaattagagtgtaattttaaattaattaaatttgtggattcaaaatatt

SEQ ID NO: 662 - DNA, 567 bp, MUS MUSCULUS
accaaccaaaagtatagtcatccgggaagagaagagcctgagagaaaaataacacttggactggcatcaggaggctagtgtctggatgttgttgatgtactgcaaggagaagcagactaaactagatagtgccagtggagttcagagcagccaccagtctggcaccattctttgacaccaagtcccctgaaaatcaccatgcacatccattggagccttcttcaggattccagcttattcagaagacagggtgaaactcccagcctcctgagactgagaaactactagattcttggactttccattcacagatggccattattgggctagttggactgcaacctacgctgcctcccaatgtttgacgacatcccgaaaacaactgggacacatgtgttcacactttatacatctaaactattaccgcttttgcagatttgaccaaatagaagaaaatgctaattaaacagagtacccgtgtgctggccgggcacactattctacacaatgagatcatgtttcttccacttgctgacgtccagccatcagccgcggacatgctccagc

SEQ ID NO: 663 - DNA, 431 bp, HOMO SAPIENS
gtattctccactggttgaatgcaaatggaagccagaggaaaagggaacccttgacacagttttcatatagattggcctccagggcagacagcagttgctctggctatatcaaataaaagtgtcaagagtgagcatccttgccttgtgctgaatcacaaaggaatacctttcagtttttctccattgattatgatagcagtgggcttttcacagtgggctttactgtgttgaggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccag

SEQ ID NO: 664 - DNA, 1454 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccaggttggcactttgcatgtgggaaacgatcagagaatggtgggggatgcttaagaggcatcactggaatcctacggtaccacagaccagagccttcccttcccttcctctcccacagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 665 - DNA, 1339 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 666 - DNA, 2436 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagattcagctggctaccccacttggctacagagtactaaagaagagactgaacttgatcctgctgcccttgcatacacaacactcccattgacgctagtgaagacttaagcgcatgaaaggattccaggatcaaaaggttagctctatggggagctgtgtctgggcaatccatttttttctattgacagtgaaaacgttttatagttctttttcaaagttttcccaaacttttacaataacaggcaacttgtgaaaaagtgagaaggcatctaattgaactatatccagaggagatcatggcagagaccaacaggaagaaaaacatttccaaaccacagcgatattggtcaaatcaattgtatttgaagaataaagcatttgaggcgtcggctgagaaaaattttcattatgaaacttggttggactatgaagcagaatgtgagataaaataaaacacagagctcatgagtttttatctttgtctgctaatgacttatgctagcaggattatactttttttttgcattttatgagatgtatgaactaggaagaaaatacatgctatatctaaatggattagacgtgcagattcttaactaacatttaattcattgctagtaaaacaaatgccttaaaacatttaaaaaagtgaaattatcttaaggagataattaacaagcagagtagaatagcatttttgaagaaagtgtaatatggttctacactggcattttctgcatttattgaagatacttagtctatccaacaattcactcattcattctttcatgaaaccctgtctctactaaaaatacaaaaaaaaattagctgggcacgggggtaggcccttgtagtcccagctactcgggaggctgaggcaggagaatggcgtgaacccgggaggcagagcttgtagtgaaccaagatcgcgtcactgcactccagcctaggtgacagagcgagactccgtctcaaaacaacaacaacaacaacaacaacaacaacaacaacaacaaatttctttgtgcacttactatatgcccaatgctaatctaatagttttacaagacctcaattgaacaagtgtctgaggaaatggattgcatctaataaagcaagacacaccaactgtggatgggtggtgtgtctagagtattttgcattcctaaatcactcttctggagaaaatttgtatcaataattttgtctagcttcacctggagttacacattttgattggtaagtggttgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 667 - DNA, 1450 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 668 - DNA, 1157 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactgagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatgatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 669 - DNA, 1140 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 670 - DNA, 1048 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtgctgctgtgacgaccagctttgtgtggcctccagtggacttcatatggacatagccgaccatgccttgccttgtcagtcccacatccctcaccttcggacctgggcttccctgttgatacagaggcactggatatcttgagacttgctcaccatgacttgctctggctatatcaaataaaagtgtcaagagtgagcatccttgccttgtgctgaatcacaaaggaatacctttcagtttttctccattgattatgatagcagtgggcttttcacagtgggctttactgtgttgagggtatgctattaatgtgccctgtgactgggtccatgccccagtcctattttcctggctttatggaagatgccacaacatcagctgagctgatataagaatttcctaactcatgaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccag

SEQ ID NO: 671 - DNA, 982 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcagttgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccag

SEQ ID NO: 672 - DNA, 917 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtgctgcgttgccatctttaaagtcatcacactggaccacatcagtattgcaagctgcttgggtatcattaatgaagacaaagtgggattattattttctatgatttgggcttttagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatg

SEQ ID NO: 673 - DNA, 483 bp, HOMO SAPIENS
atattgttaacttcttagtcctttgaagaaaaatcaaattttttcttaagaagagacttttgaaactcacaatatgaattcaagaagcaaaaaagaaaatcgaatttcacaaatgggataccaatagcttggaaaaatccattgcttttgcagggtttgttattgagggaaccctgcctttcccagcatccttaacatttcttcctcaaataaaagaggcacattgatttgaatagttaaagatttgtctcatttccttgccaaggaggattatgtttccagctttttgttaattctgggcatttcaaccactgcttctgataacatcctgctttactgtgctaggcattccttggacccaggactggggagtggggtttaatctggactgaattggaggaaaggaactggcaccagagtggaggctggaaggatggtggaactgacactgggacagaacacagaaaacagaatctaatgcta

SEQ ID NO: 674 - DNA, 566 bp, HOMO SAPIENS
cactgtggaagctttgttctttcgctctttgcaataaatcttgctactgctcactctttgggtccacactgcttttacgagctgtaacttaccgtgaaggtctgcagcttcactcctgaagccagcgagaccacaagcccaccgggaggaaagaacaactctagatgcgcggccttaagagctgtaatactcacctggaaggtctgcagcttcactcctgagccagcgagaccacgaaccccaccagaaggaagaaactccgaacacatccgaacatcagaaggaacaaactccagacgcgccatcttaagagctgtaacactcacggcgagggtccacggcttcattcttgaagtcagtgagaccaagaacccaccaattccggacacaatgtgactagttcttgccaatgcaatgtgagcaaagcagtgtgtcatttctaggccaaggcagagtggggtttaatctggactgaattggaggaaaggaactggcaccagagtggaggctggaaggatggtggaactgacactgggacagaacacagaaaacagaatctaatgcta

SEQ ID NO: 675 - DNA, 701 bp, HOMO SAPIENS
atgagaaaactgaagataagaaaaattctgcagcttgcctagaatatggctgctagcaaatgttgtaaaggaactcagatctgcccattcccagagcctgaggtcttaatctattctgctgacttaaattctgtatcatgagtttcacaacctgaagcttgggaactcctgtggagccctggagtgatgtttaaggaatacttcagtgagctgtacacactacaactcccatattgcattttagtttttaaaaatagtttctttaaaataaatgcaagaggggtctctgaacaatatgctcaggtctccaaactggctgaatttaccacggactatttttctagcatttggattttaacagaagcagagcaaagttgggatgaccacacccttcctgggcttcaaggactttaaaaatgtcctggctgccccagaagttggagagctctacatttcagcgttggtggtattctccactggttgaatgcaaatggaagccagaggaaaagggaacccttgacacagttttcatatagattggcctccagggcagacagcagattccaagaaccaggaggttgtttgctgtactctcccttgtacagttagctgtctctagtgcctgaatgcactaattgtcctttgggtatggatgaggtaatgaaggaaagggatgctgggaagagaggccacatgagaag

SEQ ID NO: 676 - DNA, 780 bp, HOMO SAPIENS
cagcatcatgtaagatgtgcctgctcccccttccgccatgattgtaagtttccctaggcctcctcagccatgtggaactatgagaaaactgaagataagaaaaattctgcagcttgcctagaatatggctgctagcaaatgttgtaaaggaactcagatctgcccattcccagagcctgaggtcttaatctattctgctgacttaaattctgtatcatgagtttcacaacctgaagcttgggaactcctgtggagccctggagtgatgtttaaggaatacttcagtgagctgtacacactacaactcccatattgcattttagtttttaaaaatagtttctttaaaataaatgcaagaggggtctctgaacaatatgctcaggtctccaaactggctgaatttaccacggactatttttctagcatttggattttaacagaagcagagcaaagttgggatgaccacacccttcctgggcttcaaggactttaaaaatgtcctggctgccccagaagttggagagctctacatttcagcgttggtggtattctccactggttgaatgcaaatggaagccagaggaaaagggaacccttgacacagttttcatatagattggcctccagggcagacagcagattccaagaaccaggaggttgtttgctgtactctcccttgtacagttagctgtctctagtgcctgaatgcactaattgtcctttgggtatggatgaggtaatgaaggaaagggatgctgggaagagaggccacatgagaag

SEQ ID NO: 677 - DNA, 319 bp, HOMO SAPIENS
gtcgcttagactcctaaatgcagttcttctctctcgagaccaccttggattggagaacacccaaatgagcaggattgcttttgctttcaaaagagtgatacctggagcacaggtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgcta

SEQ ID NO: 678 - DNA, 274 bp, HOMO SAPIENS
gtggaagggtggggaaatagaggaaatggaagagaagaaagagagtgattttcttcatcctgtcctggatctggatcccttttaataggatctctcctggacagttctgagcacttcgaagtgcatccaattcaggtcacagtaggtgggcaaaggacttcagaccctggagacacaaaggtattctccactggttgaatgcaaatggaagccagaggaaaagggaacccttgacacagttttcatatagattggcctccagggcagacagcag

SEQ ID NO: 679 - DNA, 310 bp, HOMO SAPIENS
actcactatagggctcgagcggcctccagggcaggtgtggaagggtggggaaatagaggaaatggaagagaagaaagagagtgattttcttcatcctgtcctggatctggatcccttttaataggatctctcctggacagttctgagcacttcgaagtgcatccaattcaggtcacagtaggtgggcaaaggacttcagaccctggagacacaaaggtattctccactggttgaatgcaaatggaagccagaggaaaagggaacccttgacacagttttcatatagattggcctccagggcagacagcag

SEQ ID NO: 680 - DNA, 332 bp, HOMO SAPIENS
ccaaagaaaaattatccttatcagtgatgtctgcctggtggctggtgaatggtaaaaactggcaggagagaccaatccgatgggaaatcaagttcaggtattctccactggttgaatgcaaatggaagccagaggaaaagggaacccttgacacagttttcatatagattggcctccagggcagacagcagattccaagaaccaggaggttgtttgctgtactctcccttgtacagttagctgtctctagtgcctgaatgcactaattgtcctttgggtatggatgaggtaatgaaggaaagggatgctgggaagagaggccacatgagaag

SEQ ID NO: 681 - DNA, 584 bp, HOMO SAPIENS
cttccaaaccaacatgtgtgaaacataatggagtctcactctgttacccagactggagtgcagtggcatgatctcggctctacaacctccgcctcccaggttcagattatttggaaatttccagctttgtgcactagacagaagacctttgacctctgggatgacagcataaaggcgagggtagatttctaaagaaaaggatctgtgaacatttaggaaagcaagcagtggtcaccaggaatttgtgtggattgctgataaagaagtctttctgcactaacttcatctcttctttggcattatggctcatctgataaataaatgcaatagacatatctttggatttcaggtattctccactggttgaatgcaaatggaagccagaggaaaagggaacccttgacacagttttcatatagattggcctccagggcagacagcagattccaagaaccaggaggttgtttgctgtactctcccttgtacagttagctgtctctagtgcctgaatgcactaattgtcctttgggtatggatgaggtaatgaaggaaagggatgctgggaagagaggccacatgagaag

SEQ ID NO: 682 - DNA, 262 bp, HOMO SAPIENS
cttccaaaccaacatgtgtgaaacatagtattctccactggttgaatgcaaatggaagccagaggaaaagggaacccttgacacagttttcatatagattggcctccagggcagacagcagattccaagaaccaggaggttgtttgctgtactctcccttgtacagttagctgtctctagtgcctgaatgcactaattgtcctttgggtatggatgaggtaatgaaggaaagggatgctgggaagagaggccacatgagaag

SEQ ID NO: 683 - DNA, 961 bp, HOMO SAPIENS
cgaggcaagaattcggcacgagggtgaagacaggcagagactggggtaatgcagctacaagggaatgtcagagattgctagcaaacaccagaagctggaagaggcaattaaggattgctccctagagcttttagagggaggacggctctgcacctgccttgttcttggactcctagcctccagaattaggtcacctgttgggcaatatcaccatagactgaatacagaagcagatattagcctccagcctcctgctcatctggacactagagagagtggatattctaaaagcagaaatcttgacacttaagccaaaaagccctcccattcagaaggatgtatggatttttcatacttgcatctgcgggacgcagatggtccgagtcacaagtaaagaatgtctggtggtcttgggcagagagattcatgtcataaggaatcactaacaccttgactccagatcatggcttgaaacacactggaatgggattttgcataaaagaagaaagctcaggaatgataaatttacacaccatggatctagaagggagtctataaccccagaatcaccattattttgggtgatccatatggctatcaattaaagtgctacctgagtgctgttcaacttcttgagatggatgaaaaaggaatgaaaggagacatagcaatcttcaactccctcctttacttttgaataagaggtgatggaaggcaaggtcagagagatctgggttcaaatctcatgatttaactgctatataaccttggccaaattatttaaactttgtgtctgtttttacttagcaatataaagacactggtcatacccaccactcagaatttcaatcagaactaaatgaaataatatatgcaaagtgcttaatatagtacacctggcatggagtggtaactcaataaatagatcttttgttataaaaaaaaaaaaaaaaaaaaaaaa

SEQ ID NO: 684 - DNA, 1052 bp, HOMO SAPIENS
tggtattcagagacgtagagtggcacagctcctcagactgcacgttttccctgagctctctactggaaaacatttctataaaacataagatggcaaagaggtggaagaaaaattaatgccagcagttcagctttgactaaatataagcttaccaaagtcagcaacaatatgctacacagctgctttgctcaaagcaagagctgtctctgacaaatcgccagtacctactgcaacatcttttctccctacacagcgactccagcttgggagggcagggccagggttgtcacagcttcccctgtggtgtctgcctgccaagcacagctctggagttagccctgggtgtgagattctctcctgaggctgcatctcggcgggggctgagcacagcagagatgaatgcagtagaggccattcatagagctgtggaattcaatcctcatgtgccaaaatatctacatgctgtattacgtaattatcatcttctctgagggaggaaaaaaagtagctgagagccactttcagtcccgctattaagtacattttggcctctaagaaacttcgtgtggttcacctcaggctaggtggaaatagaggctgcctagaaacatgcattaacaggtgcttcttgggccaccctagcctgaagaaggacaaccagagagtataacagtggcacctcaatgccttggccttgaggaccctcagtgtcccttcaggcctccagatttgaggggaggctctagcatgttctggctgcagctattgtcttcagggattctgcagcttgtagctcgttggggtacttctaccaattggagctgggctgttggagtcatctccactgcctaccaccaggacaagcagagaagttacctactagaaatgaaaagcttaatcctacccccagaacatatcctgaagagaggagacagtgaagcaatagcatatgcattctttcatcttgcacactggaagagagtggaaggggctttgaatcttttgcattgtacgtgggaaggcacttttcgaaaaaaaaaaaaaaaaaaaaa

SEQ ID NO: 685 - DNA, 2970 bp, HOMO SAPIENS
gggacctttagagagttagcaataaggctggagaaggaaaagccggcgcgcccgcggcccgcgcgatttcccgccgagcccgcgtccccggcggcgggcgggctccgggggcgcccgggccgcggcggcggcggcggcgcgagctgcgcctccgggccggtgaatcatcccggcagacaccaagagccgcggcagcagagaggagcgctgaaacatggctgaagcggccacgggctttctggagcagctcaagtcctgcatagtttggtcttggacgtatctgtggaccgtgtggttcttcatcgtgctattcctggtctacatcctgcgggtgcctttgaaaatcaacgacaacttgagcacagtgagcatgtttttgaacacattaacaccgaagttctacgtggccctaacaggcacttcctcactaatatcagggcttattttgatatttgaatggtggtattttcgcaaatacggaacttcattcattgaacaagtctcagtaagccacttgcgcccccttctgggaggggttgacaacaactcttccaacaattctaattccagtaacggggactcagattccaataggcaaagtgtctcagaatgcaaagtatggcgaaatccactaaatttatttaggggtgctgaatacaatcggtatacttgggtgacaggacgagagcctcttacttactatgacatgaatctctctgcccaagaccaccagacattctttacttgtgactcggaccatctgcgtcccgcagatgcaataatgcagaaagcctggagagagagaaacccccaagctaggatttctgcagctcatgaagccttggagataaatgaaattaggtccagagttgaagttcccctaattgcttcctctaccatctgggagataaaattgttaccaaagtgtgcaactgcttatattctcttggctgaagaggaagcaacaaccattgctgaagcagaaaaattatttaagcaggccctgaaggctggagatggctgttaccgacgctctcagcagctacaacatcatggatcccagtatgaagcccaacatagacgagacaccaatgtcttggtgtacatcaaaagaaggctagcaatgtgtgccagaagactcgggaggaccagggaagcagtgaaaatgatgagagatttaatgaaggagttcccccttctgagtatgttcaatatccatgaaaaccttttagaagcccttctggaactacaagcatatgctgatgttcaggcagtcttagcaaagtatgatgatataagcttaccaaagtcagcaacaatatgctacacagctgctttgctcaaagcaagagctgtctctgacaaattctctcctgaggctgcatctcggcgggggctgagcacagcagagatgaatgcagtagaggccattcatagagctgtggaattcaatcctcatgtgccaaaatacctactagaaatgaaaagcttaatcctacccccagaacatatcctgaagagaggagacagtgaagcaatagcatatgcattctttcatcttgcacactggaagagagtggaaggggctttgaatcttttgcattgtacgtgggaaggcacttttcggatgatcccttatcccttggaaaaggggcacctattttatccttacccaatctgtacagaaacagcagaccgagagctgcttccatctttccatgaagtctcagtttacccaaagaaggagcttcccttctttattctctttactgctggattatgttccttcacagccatgctggccctcctgacacatcagttcccggaacttatgggggtcttcgcaaaagctatgattgacattttctgctcggcagagttcagggactggaattgcaagagtattttcatgcgtgttgaagatgaactggaaatccctccggcacctcaatctcaacatttccaaaactgaactcatcacccctcttcccccaccaccaaaactgctcctcctcctgtattcctgacctccgccatccaccccgttgctcaagccggaaactcggcagcccttccaaactcttccctctctcactccccacatcccatcgtctcggccttcacaatctgtcagttctaacctcctaagcaactaggccttcagtaaatgtgattcacctcttctttccctccttttcccaaagcatccctcttagtctaggtccttgttgtttcttgcctgaactcctgccatagtcttaactggtctccttgcttcctgtcttatcctccaccgtctttcttttatactgacacaagaatggtctttctgaattgcacatgtgagcatgtcacttccctgcttaaatttctccagtggattcccagggacttcaggatcaagtcctagttgttcagcatggcatccaagactctttatgatctggcccttgcttacctctcagccttagctctcccaactcttgcacagtcactgctcttcagccatagtggatcactcaccattcccagatgtaccaggctctcgcacacctctgcacctttgcacgtgctgtttgctgtgcgtggaatgcccttcactgtcaccaccctgctcatccactctactaatgcctcttcattcttttatactcagctttctttaaagttcttctaagctgagttaggtgtctgtcctttatgatcccgcagtattccatgaatacgtatattctcacatttattgtactgtattataattgttgaaaacttgtctgtcccatttagaatgtgagctccttgagagcagaacggtgtcttcattatctctgtatccccaaggctttgcacagtgccttgctcatagtaggttttcaataaatgattattaaataaataaaaaaaaaaaaaaaaaaaaaaaaaaaaaaaaaaaaaaaaaaaaaaaaaaaaaaaaaaaaaaaaaaaaaaaaa

SEQ ID NO: 686 - DNA, 735 bp, HOMO SAPIENS
atggcgggcgcccctcccccagcctcgctgccgccttgcagtttgatctcggactgctgtgctagcaatcagcgagactccgtgggcgtagcaccctccgagccaggtgcgggatataatctggtggtgcaccgttttttaagctggtccgaaaagcgcagtattcgggtgggagtgacccgattttccaggtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccag

SEQ ID NO: 687 - DNA, 534 bp, HOMO SAPIENS
atggcgggcgcccctcccccagcctcgctgccgccttgcagtttgatctcggactgctgtgctagcaatcagcgagactccgtgggcgtagcaccctccgagccaggtgcgggatataatctggtggtgcaccgttttttaagctggtccgaaaagcgcagtattcgggtgggagtgacccgattttccaggtattctccactggttgaatgcaaatggaagccagaggaaaagggaacccttgacacagttttcatatagattggcctccagggcagacagcagttgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatg

SEQ ID NO: 688 - DNA, 400 bp, HOMO SAPIENS
atggcgggcgcccctcccccagcctcgctgccgccttgcagtttgatctcggactgctgtgctagcaatcagcgagactccgtgggcgtagcaccctccgagccaggtgcgggatataatctggtggtgcaccgttttttaagctggtccgaaaagcgcagtattcgggtgggagtgacccgattttccaggtgctgcgttgccatctttaaagtcatcacactggaccacatcagtattgcaagctgcttgggtatcattaatgaagacaaagtgggattattattttctatgatttgggcttttgtattctccactggttgaatgcaaatggaagccagaggaaaagggaacccttgacacagttttcatatagattggcctccagggcagacagcag

SEQ ID NO: 689 - DNA, 285 bp, HOMO SAPIENS
atggcgggcgcccctcccccagcctcgctgccgccttgcagtttgatctcggactgctgtgctagcaatcagcgagactccgtgggcgtagcaccctccgagccaggtgcgggatataatctggtggtgcaccgttttttaagctggtccgaaaagcgcagtattcgggtgggagtgacccgattttccaggtattctccactggttgaatgcaaatggaagccagaggaaaagggaacccttgacacagttttcatatagattggcctccagggcagacagcag

SEQ ID NO: 690 - DNA, 534 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcag

SEQ ID NO: 691 - DNA, 481 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccaggtattctccactggttgaatgcaaatggaagccagaggaaaagggaacccttgacacagttttcatatagattggcctccagggcagacagcag

SEQ ID NO: 692 - DNA, 1118 bp, HOMO SAPIENS
agtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccaggaaccttcagaaagggcagaaaggaagtttacttctgcctctacaatggatttgcctactttagacatgttttcaagatttatccacgtcatggcatataccagaacttcatttcttttatgattgaataatattacatggttttaatatactatattttgtttatctgttcatcaattaataaatctttgggttgttttcacttttaggctataataattaacgctgttaggaatatttacttgtaagtttttgttatatgtttttaattctcctgggtagatacttagggataaaattgctgaattagattgtaactttatgtttagaaactgcccaactgttttccaaagtaactatcattttaatatttcaccagcattgtatgacagttctggtctttccttatttaaactaatacttaattattgccttttattgtagctatcctagtagatatgaagtgttatctcattatggttttgatttgcattctctaatgactaataatatcaagcatcttttcatgtgtatacaagatatttgtatatcttctttagagaaatgtctactcaaatcctgtgcctatgttttaattggataatttatgtttttattgttgagttgtaatagtttttaaatatattctggatcctagacctttaaca

SEQ ID NO: 693 - DNA, 1030 bp, HOMO SAPIENS
agtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggaaccttcagaaagggcagaaaggaagtttacttctgcctctacaatggatttgcctactttagacatgttttcaagatttatccacgtcatggcatataccagaacttcatttcttttatgattgaataatattacatggttttaatatactatattttgtttatctgttcatcaattaataaatctttgggttgttttcacttttaggctataataattaacgctgttaggaatatttacttgtaagtttttgttatatgtttttaattctcctgggtagatacttagggataaaattgctgaattagattgtaactttatgtttagaaactgcccaactgttttccaaagtaactatcattttaatatttcaccagcattgtatgacagttctggtctttccttatttaaactaatacttaattattgccttttattgtagctatcctagtagatatgaagtgttatctcattatggttttgatttgcattctctaatgactaataatatcaagcatcttttcatgtgtatacaagatatttgtatatcttctttagagaaatgtctactcaaatcctgtgcctatgttttaattggataatttatgtttttattgttgagttgtaatagtttttaaatatattctggatcctagacctttaaca

SEQ ID NO: 694 - DNA, 667 bp, HOMO SAPIENS
agtggggtttaatctggactgaattggaggaaaggaactggcaccagagtggaggctggaaggatggtggaactgacactgggacagaacacagaaaacagaatctaatgctagaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 695 - DNA, 719 bp, HOMO SAPIENS
agtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatgaacttatgatctggtggaaggacatgataactaatcaaataatcacaaatatgtatttcagagaaatgaggggcaaatgcactagctgccctatgtaagctctaagtctcatcatttcggggatcctctcctgtgatgcagctttgctagaaagaactaatatttgtatttttttgcaccatctgttctgctgtatcgtggtagaagtcatgtaggttcatatgagatctttcttccaagcaccttaatgttttgaagagataaaagcaagaagcaagctttcttacaattcatgaaaaagcccaatcaatatgcctcaagaaacaaagctgatgtataataaaaattattatccca

SEQ ID NO: 696 - DNA, 638 bp, HOMO SAPIENS
agtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggctggagtgcaatggcgtgatctcggctcactgcaacctctgccttctggtttcaagcaattctcctgcctcagcctcctgagtagctgggattacaggcacccaccaccacgcctggctaatttttgtatttttagtagagacagggtttcaccgtgttgttcaggctggtctcgagctcctgacctcgtgatccgaccacctcagcctcctaaaatgctgggattacaggcgtgagccaccgcgccggccaaggcaatgatttttaatggtttttgggaagcagattttataatatattaaaggaggtgtgtgtgtgtgtgtgtgtgggtacgtaatacatacaaatatatccattcatcttttaataaactaggttcagcatcc

SEQ ID NO: 697 - DNA, 1595 bp, HOMO SAPIENS
  misc_feature (1534)..(1534): n is a, c, g, or t
attccaagaaccaggaggttgtttgctgtactctcccttgtacagttagctgtctctagtgcctgaatgcactaattgtcctttgggtatggatgaggtaatgaaggaaagggatgctgggaagagaggccacatgagaaggctgcagcaaactaagtgttgcttgctaaagaaaaatgaaagctggaatttcagcagggaagaaagtatatgccccaccgtgatgagtacaaacacacagcatcagggaagccacacagaatgaagctctgagaagactgttagcattttccctccacgccccaggggaaaaggttggttctctaacttgatgtatacaaattttctgagaagattatttaaaaagtagtttccacccaatccaaggattccgattcagcagggctgaggtactccgaagcagggtatggattctgctttcatcttccaatgcaagcctgtttccacagggttgagatctggcatggacaatttcagcctgaaacgattacttttggaaatttgcagtgagtggaaacagtcatgtagagctgtggatcttgctacctggatcaataacttcttctagttaagcttcttgccaaggaaaagcaaggaatatcaaatttcatagacctaatgaaccactggaaactgcttgcacccatgtgtgcaaggatcgctttttatgatttatacaaaccaaagcactcattggaataagaataatagctaacgtttatttatcatttattatcctggccactgtgccaagtatttatatacagtgagttatttaatccctgcaacaacccatgggagtggggtttaatctggactgaattggaggaaaggaactggcaccagagtggaggctggaaggatggtggaactgacactgggacagaacacagaaaacagaatctaatgctagaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgtacatagtgcctgtgggtttaatttaagcctacccaaatgtgcttttntcccacctattttgaaccttgcatgtttccgggattagaattaaaataagctataaatatt

SEQ ID NO: 698 - DNA, 2015 bp, HOMO SAPIENS
attccaagaaccaggaggttgtttgctgtactctcccttgtacagttagctgtctctagtgcctgaatgcactaattgtcctttgggtatggatgaggtaatgaaggaaagggatgctgggaagagaggccacatgagaaggctgcagcaaactaagtgttgcttgctaaagaaaaatgaaagctggaatttcagcagggaagaaagtatatgccccaccgtgatgagtacaaacacacagcatcagggaagccacacagaatgaagctctgagaagactgttagcattttccctccacgccccaggggaaaaggttggttctctaacttgatgtatacaaattttctgagaagattatttaaaaagtagtttccacccaatccaaggattccgattcagcagggctgaggtactccgaagcagggtatggattctgctttcatcttccaatgcaagcctgtttccacagggttgagatctggcatggacaatttcagcctgaaacgattacttttggaaatttgcagatattgttaacttcttagtcctttgaagaaaaatcaaattttttcttaagaagagacttttgaaactcacaatatgaattcaagaagcaaaaaagaaaatcgaatttcacaaatgggataccaatagcttggaaaaatccattgcttttgcagggtttgttattgagggaaccctgcctttcccagcatccttaacatttcttcctcaaataaaagaggcacattgatttgaatagttaaagatttgtctcatttccttgccaaggaggattatgtttccagctttttgttaattctgggcatttcaaccactgcttctgataacatcctgctttactgtgctaggcattccttggacccaggactggggagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggtgtagttattattcaatttataatttggcatcctaccatatatataatttaacatctactcttttaaccagcttaggacttgtcaacccactgcttgttctgtgttccacactggtattcaaagatgtgaaagcaagaagaaggctaggatgagcaacacaacagaataactcaagcaacagagcaattctgaaacacaaggaaaaaggacttactcatacttccctcatggattatctgagaattgaatgaaaacaatcagtaatgtattcaattcataacaaatgtcaggagtgaaaagaacagagaactgacaaaagctaaaactacccaaagtgagatttgctcatgccttatgtaaacaggaaaaaataaaatcctgggaatttgtatgggtaactgcactggctattgagagatcaaatgatatgcaccttcaggaactcttccagcaagagctactcagtgaggagacattcatcattagcacattcagaaattatagctctgatatgttttctgggtggccggatgcaaaggatcatccatagttatgaaaactcaagaacagggaagtcaattagtatatttataaatctgtattcctctcctgtcaaggaatctccctgtatatgaatgatagctgtgtccttgatttgagtaactctttttcctaatcaaaagaaactcaaatgaacaggcccatatgtcttatcagaacaaactatcctgtaataaaatggaaagaggtttt

SEQ ID NO: 699 - RNA, 22 nt, HOMO SAPIENS
uagcagcacauaaugguuugug

SEQ ID NO: 700 - DNA, 1305 bp, HOMO SAPIENS
attccaagaaccaggaggttgtttgctgtactctcccttgtacagttagctgtctctagtgcctgaatgcactaattgtcctttgggtatggatgaggtaatgaaggaaagggatgctgggaagagaggccacatgagaagtggttctctaacttgatgtatacaaattttctgagaagattatttaaaaagtagtttccacccaatccaaggattccgattcagcagggctgaggtactccgaagcagggtatggattctgctttcatcttccaatgcaagcctgtttccacagggttgagatctggcatggacaatttcagcctgaaacgattacttttggaaatttgcagtgagtggaaacagtcatgtagagctgtggatcttgctacctggatcaataacttcttctagttaagcttcttgccaaggaaaagcaaggaatatcaaatttcatagacctaatgaaccactggaaactgcttgcacccatgtgtgcaaggatcgctttttatgatttatacaaaccaaagcactcattggaataagaataatagctaacgtttatttatcatttattatcctggccactgtgccaagtatttatatacagtgagttatttaatccctgcaacaacccatgggtaggtactattatttttccttctttatagataagaacattgaggcacaaaaatgctaaattacagagtgggatcaatgtgttgctcgcttagaaaatgagtcaagttccatatgtgtgagtaaatgtgaacattcccacttccttgtcacttggttttctttctcaagtttggtgtgtttgctctgtgttcacaggatattgttaacttcttagtcctttgaagaaaaatcaaattttttcttaagaagagacttttgaaactcacaatatgaattcaagaagcaaaaaagaaaatcgaatttcacaaatgggataccaatagcttggaaaaatccattgcttttgcagggtttgttattgagggaaccctgcctttcccagcatccttaacatttcttcctcaaataaaaggtaataaaatgcaaccaactgactcattttctaagtgaatgacatatttactctactctttaagtttaaactctaagcaattccccaatccttagttcccatgaaatgtaatgttctcttcctaaaatgtttggccaagttggggaaggtgggcatggcagtgtgagcctgtagccaggttccaaggttcgcttgaggttaggaggttgagactagcctgggcaatatggcaagactctgtatccacg

SEQ ID NO: 701 - DNA, 1050 bp, HOMO SAPIENS
gtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtttaggtgcagtcatcttggatttgagtcgtgcttgtgaattttcatgtgttacaacaaccagattaaatttcttcaagtggaaatattaagtcaaagaaattacctatttctaataattcttactgaagaagtgaaacctagcaggaagaattgtatttggttctggaggttggaagtctgagaccagggtgtgactgtggttgggttctggcgggagcgtctttggtttgttgacagttcaccttcttgctgtgtcctcatgtgaggggaagtagagggagaaaaagctctctggtgtctttttttataagggcactaatcacatcatgaaagccccaccctcaaaacctcgtctaagcctatttatctcccaaaggccccatctccaaattccatcacattggagcttagggcttccacctattaatctttctggggtgcagtatggttcagtctatagcagaaaaaaataataaaaaaaatatatatatgtacacacacacatatatatatataatttatattcatgtatacttgtatagttgaaagtctccaagataatgaacaaaatgctgaatggttatttctgggtcatagaaggagagcaaattctagaacttttttctttttgtttctctgtgttttccaagttttattcactgagcatgtactaccattgtgtttataaaatattaaattttt

SEQ ID NO: 702 - DNA, 1185 bp, HOMO SAPIENS
gtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctagtaccctaatgacaagttcaccccagtgttttctgaggagtctacctggaaatgagaaccatctgtccttcctatagtagttcttctcagaatgccgactctatgctaatggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccaggaaccttcagaaagggcagaaaggaagtttacttctgcctctacaatggatttgcctactttagacatgttttcaagatttatccacgtcatggcatataccagaacttcatttcttttatgattgaataatattacatggttttaatatactatattttgtttatctgttcatcaattaataaatctttgggttgttttcacttttaggctataataattaacgctgttaggaatatttacttgtaagtttttgttatatgtttttaattctcctgggtagatacttagggataaaattgctgaattagattgtaactttatgtttagaaactgcccaactgttttccaaagtaactatcattttaatatttcaccagcattgtatgacagttctggtctttccttatttaaactaatacttaattattgccttttattgtagctatcctagtagatatgaagtgttatctcattatggttttgatttgcattctctaatgactaataatatcaagcatcttttcatgtgtatacaagatatttgtatatcttctttagagaaatgtctactcaaatcctgtgcctatgttttaattggataatttatgtttttattgttgagttgtaatagtttttaaatatattctggatcctagacctttaaca

SEQ ID NO: 703 - DNA, 612 bp, HOMO SAPIENS
gtattctccactggttgaatgcaaatggaagccagaggaaaagggaacccttgacacagttttcatatagattggcctccagggcagacagcagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggctacccacagatccagggccataaataactgccaaagaagagaagccttctctcttctctgctgttgcttcattttatagtctcaggaaatctcatcatagaagtgcctttcaccttctgccatgattgtgaggcttcgctagctacgtggaact

SEQ ID NO: 704 - DNA, 837 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagagtggggtttaatctggactgaattggaggaaaggaactggcaccagagtggaggctggaaggatggtggaactgacactgggacagaacacagaaaacagaatctaatgctattgctctggctatatcaaataaaagtgtcaagagtgagcatccttgccttgtgctgaatcacaaaggaatacctttcagtttttctccattgattatgatagcagtgggcttttcacagtgggctttactgtgttgaggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccag

SEQ ID NO: 705 - DNA, 615 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactgtgggccgactctggaaccgagctgcctgggtgaatcccagctccactacttactagctgtgccttgttgggtaaattatctgttggaccttttataaaacaagtactattaatacctcattggactgttgtaaggatgtaaatgagctaacatattaaaggaatttcaacagtgg

SEQ ID NO: 706 - DNA, 1032 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactgtgattctaccagaaaggaatgaagaacagaaccttcaggaattgagtcacaatgcagacaaatatcaaatgggagattgttgcaaggaagagattgatgatagtattttctactagccattgggaagataaaaggagacagaagattgaagcctttgccagccattctttccctttttgcttccaaactcctcaactgggaaccttcatatgtgcagtatttatattggatcatactggggattataaaagttcctaggaggctagaagagccaaccaacagagaagggaaagcagtctgttctgaacatagggacataagttcattcatgccaagtatctttccagcatgtttctcccatttagaatatctagcatgtaaggcctttcaatattaatataagcccaatatcagctctttctctttgtatttcatctctttctactctcctatttgtattttgtgttcctatcaaagtgtcgtatctgggagatgacctgccttatcctgttctataacagttttgtttggtgctgtgtctttagaacagtgcctggcacacagtaagcactcaataaatctttgatgaatg

SEQ ID NO: 707 - DNA, 979 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagtgattctaccagaaaggaatgaagaacagaaccttcaggaattgagtcacaatgcagacaaatatcaaatgggagattgttgcaaggaagagattgatgatagtattttctactagccattgggaagataaaaggagacagaagattgaagcctttgccagccattctttccctttttgcttccaaactcctcaactgggaaccttcatatgtgcagtatttatattggatcatactggggattataaaagttcctaggaggctagaagagccaaccaacagagaagggaaagcagtctgttctgaacatagggacataagttcattcatgccaagtatctttccagcatgtttctcccatttagaatatctagcatgtaaggcctttcaatattaatataagcccaatatcagctctttctctttgtatttcatctctttctactctcctatttgtattttgtgttcctatcaaagtgtcgtatctgggagatgacctgccttatcctgttctataacagttttgtttggtgctgtgtctttagaacagtgcctggcacacagtaagcactcaataaatctttgatgaatg

SEQ ID NO: 708 - DNA, 543 bp, HOMO SAPIENS
gttctggaggttggaagtctgagaccagggtgtgactgtggttgggttctggcgggagcgtctttggtttgttgacagttcaccttcttgctgtgtcctcatgtgaggggaagtagagggagaaaaagctctctggtgtctttttttataagggcactaatcacatcatgaaagccccaccctcaaaacctcgtctaagcctatttatctcccaaaggccccatctccaaattccatcacattggagcttagggcttccacctattaatctttctggggtgcagtatggttcagtctatagcagaaaaaaataataaaaaaaatatatatatgtacacacacacatatatatatataatttatattcatgtatacttgtatagttgaaagtctccaagataatgaacaaaatgctgaatggttatttctgggtcatagaaggagagcaaattctagaacttttttctttttgtttctctgtgttttccaagttttattcactgagcatgtactaccattgtgtttataaaatattaaattttt

SEQ ID NO: 709 - DNA, 98 bp, HOMO SAPIENS
attaaatttcttcaagtggaaatattaagtcaaagaaattacctatttctaataattcttactgaagaagtgaaacctagcaggaagaattgtatttg

SEQ ID NO: 710 - DNA, 64 bp, HOMO SAPIENS
gtttaggtgcagtcatcttggatttgagtcgtgcttgtgaattttcatgtgttacaacaaccag

SEQ ID NO: 711 - DNA, 668 bp, HOMO SAPIENS
gaaccttcagaaagggcagaaaggaagtttacttctgcctctacaatggatttgcctactttagacatgttttcaagatttatccacgtcatggcatataccagaacttcatttcttttatgattgaataatattacatggttttaatatactatattttgtttatctgttcatcaattaataaatctttgggttgttttcacttttaggctataataattaacgctgttaggaatatttacttgtaagtttttgttatatgtttttaattctcctgggtagatacttagggataaaattgctgaattagattgtaactttatgtttagaaactgcccaactgttttccaaagtaactatcattttaatatttcaccagcattgtatgacagttctggtctttccttatttaaactaatacttaattattgccttttattgtagctatcctagtagatatgaagtgttatctcattatggttttgatttgcattctctaatgactaataatatcaagcatcttttcatgtgtatacaagatatttgtatatcttctttagagaaatgtctactcaaatcctgtgcctatgttttaattggataatttatgtttttattgttgagttgtaatagtttttaaatatattctggatcctagacctttaaca

SEQ ID NO: 712 - DNA, 108 bp, HOMO SAPIENS
  misc_feature (47)..(47): n is a, c, g, or t
acatagtgcctgtgggtttaatttaagcctacccaaatgtgcttttntcccacctattttgaaccttgcatgtttccgggattagaattaaaataagctataaatatt

SEQ ID NO: 713 - DNA, 612 bp, HOMO SAPIENS
aaaccttgcaaagctctccatttcacccagagtaaaagctgaaatccttactgtgatcaataagccctgtatgatctttaagctccctcccttctctcccctgggcttattctgttctatccacccaagattcctttgctgttgcttgaacactccagacatactgctgcctcagggcctttgaaaatacatcacatgactaactttggcatctccttcagtcttcagtcacatatgcctttacagtgcgatggcactgtagaagtgtaggtgatatcacaacttcctcaattctttgacttgtcacaatgagcataaattacttttgtaattttataggacactcatggagggaaatctatacacagaaacacacacacacagggagaaagccatgtgaagacaggcagagactggggtaatgcagctacaagggaatgtcagagattgctagcaaacaccagaagctggaagaggcaattaaggattgctccctagagcttttagagggaggacggctctgcacctgccttgttcttggactcctagcctccagaattgtgagagaataaatttctgttgtttaaactaaaaaaaaaaaaaaaaaaaaaa

SEQ ID NO: 714 - DNA, 355 bp, HOMO SAPIENS
atggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgt

SEQ ID NO: 715 - DNA, 115 bp, HOMO SAPIENS
gttggcactttgcatgtgggaaacgatcagagaatggtgggggatgcttaagaggcatcactggaatcctacggtaccacagaccagagccttcccttcccttcctctcccacag

SEQ ID NO: 716 - DNA, 199 bp, HOMO SAPIENS
gaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccag

SEQ ID NO: 717 - DNA, 365 bp, HOMO SAPIENS
gtatgggtaactgcactggctattgagagatcaaatgatatgcaccttcaggaactcttccagcaagagctactcagtgaggagacattcatcattagcacattcagaaattatagctctgatatgttttctgggtggccggatgcaaaggatcatccatagttatgaaaactcaagaacagggaagtcaattagtatatttataaatctgtattcctctcctgtcaaggaatctccctgtatatgaatgatagctgtgtccttgatttgagtaactctttttcctaatcaaaagaaactcaaatgaacaggcccatatgtcttatcagaacaaactatcctgtaataaaatggaaagaggtttt

SEQ ID NO: 718 - DNA, 321 bp, HOMO SAPIENS
cagcttaggacttgtcaacccactgcttgttctgtgttccacactggtattcaaagatgtgaaagcaagaagaaggctaggatgagcaacacaacagaataactcaagcaacagagcaattctgaaacacaaggaaaaaggacttactcatacttccctcatggattatctgagaattgaatgaaaacaatcagtaatgtattcaattcataacaaatgtcaggagtgaaaagaacagagaactgacaaaagctaaaactacccaaagtgagatttgctcatgccttatgtaaacaggaaaaaataaaatcctgggaattt

SEQ ID NO: 719 - DNA, 54 bp, HOMO SAPIENS
aagtgcctttcaccttctgccatgattgtgaggcttcgctagctacgtggaact

SEQ ID NO: 720 - DNA, 102 bp, HOMO SAPIENS
gctacccacagatccagggccataaataactgccaaagaagagaagccttctctcttctctgctgttgcttcattttatagtctcaggaaatctcatcatag

SEQ ID NO: 721 - DNA, 69 bp, HOMO SAPIENS
gtgtagttattattcaatttataatttggcatcctaccatatatataatttaacatctactcttttaac

SEQ ID NO: 722 - DNA, 294 bp, HOMO SAPIENS
aaatgaggggcaaatgcactagctgccctatgtaagctctaagtctcatcatttcggggatcctctcctgtgatgcagctttgctagaaagaactaatatttgtatttttttgcaccatctgttctgctgtatcgtggtagaagtcatgtaggttcatatgagatctttcttccaagcaccttaatgttttgaagagataaaagcaagaagcaagctttcttacaattcatgaaaaagcccaatcaatatgcctcaagaaacaaagctgatgtataataaaaattattatccca

SEQ ID NO: 723 - DNA, 63 bp, HOMO SAPIENS
aacttatgatctggtggaaggacatgataactaatcaaataatcacaaatatgtatttcagag

SEQ ID NO: 724 - DNA, 111 bp, HOMO SAPIENS
gtaccctaatgacaagttcaccccagtgttttctgaggagtctacctggaaatgagaaccatctgtccttcctatagtagttcttctcagaatgccgactctatgctaatg

SEQ ID NO: 725 - DNA, 387 bp, HOMO SAPIENS
gctggagtgcaatggcgtgatctcggctcactgcaacctctgccttctggtttcaagcaattctcctgcctcagcctcctgagtagctgggattacaggcacccaccaccacgcctggctaatttttgtatttttagtagagacagggtttcaccgtgttgttcaggctggtctcgagctcctgacctcgtgatccgaccacctcagcctcctaaaatgctgggattacaggcgtgagccaccgcgccggccaaggcaatgatttttaatggtttttgggaagcagattttataatatattaaaggaggtgtgtgtgtgtgtgtgtgtgggtacgtaatacatacaaatatatccattcatcttttaataaactaggttcagcatcc

SEQ ID NO: 726 - DNA, 112 bp, HOMO SAPIENS
ggtatgctattaatgtgccctgtgactgggtccatgccccagtcctattttcctggctttatggaagatgccacaacatcagctgagctgatataagaatttcctaactcat

SEQ ID NO: 727 - DNA, 300 bp, HOMO SAPIENS
agtggaataaggacaatgatgcaaaaaagagccattctcggagggagtcagtcaggggcaagctcagcgtgtcaggagacaccagggaggcccaggtgggaacctctcatgcttgcactgcgaaggggagccaatgccaaatggagtcctggggccaggacagaagtgaaccctagacttgtgagagctgaatatacaaatggatgacggctggaccccacataaaaatagaactctgacccacagcctgcagcaacctgcctaaaaaaccaatcctcttatctacaatgaaaagcccag

SEQ ID NO: 728 - DNA, 138 bp, HOMO SAPIENS
ttgctctggctatatcaaataaaagtgtcaagagtgagcatccttgccttgtgctgaatcacaaaggaatacctttcagtttttctccattgattatgatagcagtgggcttttcacagtgggctttactgtgttgag

SEQ ID NO: 729 - DNA, 113 bp, HOMO SAPIENS
agtggggtttaatctggactgaattggaggaaaggaactggcaccagagtggaggctggaaggatggtggaactgacactgggacagaacacagaaaacagaatctaatgcta

SEQ ID NO: 730 - DNA, 154 bp, HOMO SAPIENS
aggcacattgatttgaatagttaaagatttgtctcatttccttgccaaggaggattatgtttccagctttttgttaattctgggcatttcaaccactgcttctgataacatcctgctttactgtgctaggcattccttggacccaggactgggg

SEQ ID NO: 731 - DNA, 248 bp, HOMO SAPIENS
gtaataaaatgcaaccaactgactcattttctaagtgaatgacatatttactctactctttaagtttaaactctaagcaattccccaatccttagttcccatgaaatgtaatgttctcttcctaaaatgtttggccaagttggggaaggtgggcatggcagtgtgagcctgtagccaggttccaaggttcgcttgaggttaggaggttgagactagcctgggcaatatggcaagactctgtatccacg

SEQ ID NO: 732 - DNA, 216 bp, HOMO SAPIENS
atattgttaacttcttagtcctttgaagaaaaatcaaattttttcttaagaagagacttttgaaactcacaatatgaattcaagaagcaaaaaagaaaatcgaatttcacaaatgggataccaatagcttggaaaaatccattgcttttgcagggtttgttattgagggaaccctgcctttcccagcatccttaacatttcttcctcaaataaaag

SEQ ID NO: 733 - DNA, 196 bp, HOMO SAPIENS
taggtactattatttttccttctttatagataagaacattgaggcacaaaaatgctaaattacagagtgggatcaatgtgttgctcgcttagaaaatgagtcaagttccatatgtgtgagtaaatgtgaacattcccacttccttgtcacttggttttctttctcaagtttggtgtgtttgctctgtgttcacagg

SEQ ID NO: 734 - DNA, 292 bp, HOMO SAPIENS
tgagtggaaacagtcatgtagagctgtggatcttgctacctggatcaataacttcttctagttaagcttcttgccaaggaaaagcaaggaatatcaaatttcatagacctaatgaaccactggaaactgcttgcacccatgtgtgcaaggatcgctttttatgatttatacaaaccaaagcactcattggaataagaataatagctaacgtttatttatcatttattatcctggccactgtgccaagtatttatatacagtgagttatttaatccctgcaacaacccatggg

SEQ ID NO: 735 - DNA, 115 bp, HOMO SAPIENS
ctccgaagcagggtatggattctgctttcatcttccaatgcaagcctgtttccacagggttgagatctggcatggacaatttcagcctgaaacgattacttttggaaatttgcag

SEQ ID NO: 736 - DNA, 97 bp, HOMO SAPIENS
tggttctctaacttgatgtatacaaattttctgagaagattatttaaaaagtagtttccacccaatccaaggattccgattcagcagggctgaggta

SEQ ID NO: 737 - DNA, 175 bp, HOMO SAPIENS
gctgcagcaaactaagtgttgcttgctaaagaaaaatgaaagctggaatttcagcagggaagaaagtatatgccccaccgtgatgagtacaaacacacagcatcagggaagccacacagaatgaagctctgagaagactgttagcattttccctccacgccccaggggaaaaggt

SEQ ID NO: 738 - DNA, 141 bp, HOMO SAPIENS
attccaagaaccaggaggttgtttgctgtactctcccttgtacagttagctgtctctagtgcctgaatgcactaattgtcctttgggtatggatgaggtaatgaaggaaagggatgctgggaagagaggccacatgagaag

SEQ ID NO: 739 - DNA, 453 bp, HOMO SAPIENS
cactgtggaagctttgttctttcgctctttgcaataaatcttgctactgctcactctttgggtccacactgcttttacgagctgtaacttaccgtgaaggtctgcagcttcactcctgaagccagcgagaccacaagcccaccgggaggaaagaacaactctagatgcgcggccttaagagctgtaatactcacctggaaggtctgcagcttcactcctgagccagcgagaccacgaaccccaccagaaggaagaaactccgaacacatccgaacatcagaaggaacaaactccagacgcgccatcttaagagctgtaacactcacggcgagggtccacggcttcattcttgaagtcagtgagaccaagaacccaccaattccggacacaatgtgactagttcttgccaatgcaatgtgagcaaagcagtgtgtcatttctaggccaaggcag

SEQ ID NO: 740 - DNA, 94 bp, HOMO SAPIENS
gtattctccactggttgaatgcaaatggaagccagaggaaaagggaacccttgacacagttttcatatagattggcctccagggcagacagcag

SEQ ID NO: 741 - DNA, 466 bp, HOMO SAPIENS
atgagaaaactgaagataagaaaaattctgcagcttgcctagaatatggctgctagcaaatgttgtaaaggaactcagatctgcccattcccagagcctgaggtcttaatctattctgctgacttaaattctgtatcatgagtttcacaacctgaagcttgggaactcctgtggagccctggagtgatgtttaaggaatacttcagtgagctgtacacactacaactcccatattgcattttagtttttaaaaatagtttctttaaaataaatgcaagaggggtctctgaacaatatgctcaggtctccaaactggctgaatttaccacggactatttttctagcatttggattttaacagaagcagagcaaagttgggatgaccacacccttcctgggcttcaaggactttaaaaatgtcctggctgccccagaagttggagagctctacatttcagcgttggtg

SEQ ID NO: 742 - DNA, 159 bp, HOMO SAPIENS
gtgctgctgtgacgaccagctttgtgtggcctccagtggacttcatatggacatagccgaccatgccttgccttgtcagtcccacatccctcaccttcggacctgggcttccctgttgatacagaggcactggatatcttgagacttgctcaccatgac

SEQ ID NO: 743 - DNA, 170 bp, HOMO SAPIENS
ggtagatttctaaagaaaaggatctgtgaacatttaggaaagcaagcagtggtcaccaggaatttgtgtggattgctgataaagaagtctttctgcactaacttcatctcttctttggcattatggctcatctgataaataaatgcaatagacatatctttggatttcag

SEQ ID NO: 744 - DNA, 781 bp, HOMO SAPIENS
ttcaagtatccctcgggatcgtgtcctgggaggcggataggattcagcactctagaattgtctcaccatttgaataggtgctgctcagcccttttgagtctgtcatcggaaagaccatggctttctcgcttttcatctttttggatggctgcttgtcgcaatattgtcttcttcgctttagacaaccctgtggtgcttactcttcctgtgatgaaggcaagtgtgttcctggagaggttactgaatctggagctgcttttagggagttgcagatggcgcaggcctcccttcctcctcaaggaagccaaagacaccatcacaatcaggcagccaggagccagggcttccataggcccagctctgtacctgtagggggatggaatctgctaaaagtgttctggagtcgttcttggatatctgcttggtatcttctcaatatcttgaccatctgtgacaattttaatcctcatttgatcatattttatggaccagctggcatgctgccttctgatcctttcagctgcagaaaagacaggcttaccttggagatattgcaagtttggttcccggccactgcagtaaatgtcttactatgttgaccgtgctggactcgaattcctgggctcaagcaatccttctgcctcaaccttctgattatttcgactacgggtgtgcgccaccatgcctggcttagaaaaggactttatgttgaacctcttttctcttacaatcaaataataaattcattgagtcatactaaaaaaaaaaaaaaaaaaaaaaa

SEQ ID NO: 745 - DNA, 912 bp, HOMO SAPIENS
ccgcctccgtggccggagttgcgtcttgccgggcgtggggctgtctctctggttgggtagtggggagaggggttcccgaaggcactcttgggagtgtatggatgacgtgtcaggtcaccaaatggattgtcgaagggaccggagggaagaggggagagctggaatttctgagggtttcggttgcagttttcagtgatctttgctcgtctcccttccctgtaaagctacatatgctcagcaagtgccgcgtgcctcccatccttggtcggtggtaaatgcaggaggtaattaacaggatcttcacaaacttggagccagcccactgagtcgttaaaaaaacacccgcaaagaaaacacacgagccttaacactccagagagttccgaaacacaaagtaaatttcgagctaagggttacagtcctttaaatctggaaatttcaagtatccctcgggatcgtgtccctgggaggcggataggattcagcactctagaattgtctcaccatttgaataggtgctgctcagcccttttgagtctgtcatcggaaagaccatggctttctcgcttttcatctttttggatggctgcttgtcgcaatattgtcttcttcgctttagacaagaccagctggcatgctgccttctgatcctttcagctgcagaaaagacaggcttaccttggagatattgcaagtttggttcctggccactgcaataaatgtcttactatgttgaccgtgctggactcgaattcctgggctcaagcaatccttctgcctcaaccttctgattatttcgactacgggtgtgcgccaccatgcctggcttagaaaaggactttatgttgaacctcttttctcttacaatcaaataataaattcattgagtcataaaaaaaaaaaaaaaaaaaa

SEQ ID NO: 746 - DNA, 429 bp, HOMO SAPIENS
cgaaacacaaagtaaatttcgagctaagggttacagtcctttaaatctggaaatttcaagtatccctcgggatcgtgtccctgggaggcggataggattcagcactctagaattgtctcaccatttgaataggtgctgctcagcccttttgagtctgtcatcggaaagaccatggctttctcgcttttcatctttttggatggctgcttgtcgcaatattgtcttcttcgctttagacaatgtcttactatgttgaccgtgctggactcgaattcctgggctcaagcaatccttctgcctcaaccttctgattatttcgactacgggtgtgcgccaccatgcctggcttagaaaaggactttatgttgaacctcttttctcttacaatcaaataataaattcattgagtcatactaaaaaaaaaaaaaa

SEQ ID NO: 747 - DNA, 74 bp, HOMO SAPIENS
attatttggaaatttccagctttgtgcactagacagaagacctttgacctctgggatgacagcataaaggcgag

SEQ ID NO: 748 - DNA, 79 bp, HOMO SAPIENS
cagcatcatgtaagatgtgcctgctcccccttccgccatgattgtaagtttccctaggcctcctcagccatgtggaact

SEQ ID NO: 749 - DNA, 36 bp, HOMO SAPIENS
actcactatagggctcgagcggcctccagggcaggt

SEQ ID NO: 750 - DNA, 180 bp, HOMO SAPIENS
gtggaagggtggggaaatagaggaaatggaagagaagaaagagagtgattttcttcatcctgtcctggatctggatcccttttaataggatctctcctggacagttctgagcacttcgaagtgcatccaattcaggtcacagtaggtgggcaaaggacttcagaccctggagacacaaag

SEQ ID NO: 751 - DNA, 112 bp, HOMO SAPIENS
gtcgcttagactcctaaatgcagttcttctctctcgagaccaccttggattggagaacacccaaatgagcaggattgcttttgctttcaaaagagtgatacctggagcacag

SEQ ID NO: 752 - DNA, 115 bp, HOMO SAPIENS
gtgctgcgttgccatctttaaagtcatcacactggaccacatcagtattgcaagctgcttgggtatcattaatgaagacaaagtgggattattattttctatgatttgggctttt

SEQ ID NO: 753 - DNA, 78 bp, HOMO SAPIENS
atggagtctcactctgttacccagactggagtgcagtggcatgatctcggctctacaacctccgcctcccaggttcag

SEQ ID NO: 754 - DNA, 97 bp, HOMO SAPIENS
ccaaagaaaaattatccttatcagtgatgtctgcctggtggctggtgaatggtaaaaactggcaggagagaccaatccgatgggaaatcaagttcag

SEQ ID NO: 755 - DNA, 27 bp, HOMO SAPIENS
cttccaaaccaacatgtgtgaaacata

SEQ ID NO: 756 - DNA, 191 bp, HOMO SAPIENS
atggcgggcgcccctcccccagcctcgctgccgccttgcagtttgatctcggactgctgtgctagcaatcagcgagactccgtgggcgtagcaccctccgagccaggtgcgggatataatctggtggtgcaccgttttttaagctggtccgaaaagcgcagtattcgggtgggagtgacccgattttccag

SEQ ID NO: 757 - DNA, 1246 bp, HOMO SAPIENS
attcagctggctaccccacttggctacagagtactaaagaagagactgaacttgatcctgctgcccttgcatacacaacactcccattgacgctagtgaagacttaagcgcatgaaaggattccaggatcaaaaggttagctctatggggagctgtgtctgggcaatccatttttttctattgacagtgaaaacgttttatagttctttttcaaagttttcccaaacttttacaataacaggcaacttgtgaaaaagtgagaaggcatctaattgaactatatccagaggagatcatggcagagaccaacaggaagaaaaacatttccaaaccacagcgatattggtcaaatcaattgtatttgaagaataaagcatttgaggcgtcggctgagaaaaattttcattatgaaacttggttggactatgaagcagaatgtgagataaaataaaacacagagctcatgagtttttatctttgtctgctaatgacttatgctagcaggattatactttttttttgcattttatgagatgtatgaactaggaagaaaatacatgctatatctaaatggattagacgtgcagattcttaactaacatttaattcattgctagtaaaacaaatgccttaaaacatttaaaaaagtgaaattatcttaaggagataattaacaagcagagtagaatagcatttttgaagaaagtgtaatatggttctacactggcattttctgcatttattgaagatacttagtctatccaacaattcactcattcattctttcatgaaaccctgtctctactaaaaatacaaaaaaaaattagctgggcacgggggtaggcccttgtagtcccagctactcgggaggctgaggcaggagaatggcgtgaacccgggaggcagagcttgtagtgaaccaagatcgcgtcactgcactccagcctaggtgacagagcgagactccgtctcaaaacaacaacaacaacaacaacaacaacaacaacaacaacaaatttctttgtgcacttactatatgcccaatgctaatctaatagttttacaagacctcaattgaacaagtgtctgaggaaatggattgcatctaataaagcaagacacaccaactgtggatgggtggtgtgtctagagtattttgcattcctaaatcactcttctggagaaaatttgtatcaataattttgtctagcttcacctggagttacacattttgattggtaagtgg

SEQ ID NO: 758 - DNA, 1299 bp, HOMO SAPIENS
gaagacaggcagagactggggtaatgcagctacaagggaatgtcagagattgctagcaaacaccagaagctggaagaggcaattaaggattgctccctagagcttttagagggaggacggctctgccacctgccttgttcttggactcctagcctccagaattaggtcacctgttgggcaatatcaccatagactgaatacagaagcagatattagcctccagcctcctgctcatctggacactagagagagtgaggtcagctaaaaacaagaaagaaatttaaagcatcctgggaagtcatcataccctttttctccaagaaaatctagttttgcttgtttttgtgtatcccacaacacctgtgtttgactatctttttcactttggatttggattttggtgtgatatacacagttccaaaaaatcaatggatagaaaactcaatggcataaataagtgcttaaaacaatacttcaagaaatgttcaacattccacgagaacaatatccacgtgagatgaaaagaaaagtctaagttagttcctgattcctggaagctgggaaagtctcattccagtaactacttaataccccctgtcagtctccttcaaaggctgaagccagcctctgactgaagcgcagccgcaatgtttcctgtctggtgccagcacagcttgcctggacttctgaaaattgcaagcgcttgctccttgctagtcaccacactccccctttcttagttgcagactccctcttgctgtggtttaccaggtgcccttattgcccctcatgcttgtgtgccactttggacttaatctggcacacttaactcccttcatctacgcctgaagctggacaagctgaagtgactgcgtcactttctcaattctggctcctgcccaccgaccttgctctgctaggaggaattagctctgcctggccagaacccaagaagtgacaaatcaaactcgctcaataggtggtaattaaacttctggctctaaagcccagaataggttgtcaatgggatttcacagtggctccctgacactaagcaaggatgtatttctacatctagttttaaatcttaaactctaataggattctcagaaaagacagtaaagactaaggccaaaaaaagttgtttaaaggggaaaatgacagtaagtacaaaagataatctggggaagacctggcaattctgaaatcaaaacatctgttactaagtggatcatgactgcttagtttctaggtgagctgaataaaaatactaactgaaaaaaaaaaaaaaaaaaaaaa

SEQ ID NO: 759 - DNA, 1161 bp, HOMO SAPIENS
tcgaggcaagaattcggcaaggttccttaactttacctgacagaactacaaagatttaggagtgcttaaaacaatacttcaagaaatgttcaacattccacgagaacaatatccacgtgagatgaaaagaaaagtctaagttagttcctgattcctggaagctgggaaagtctcattccagtaactacttaataccccctgtcagtctccttcaaaggctgaagccagcctctgactgaagcgcagccgcaatgtttcctgtctggtgccagcacagcttgcctggacttctgaaaattgcaagcgcttgctccttgctagtcaccacactccccctttcttagttgcagactccctcttgctgtggtttaccaggtgcccttattgcccctcatgcttgtgtgccactttggacttaatctggcacacttaactcccttcatctacgcctgaagctggacaagctgaagtgactgtgtcactttctcaattctggctcctgcccaccgaccttgctctgctaggaggaattagctctgcctggccagaacccaagaagtgacaaatcaaactcgctcaataggtggtaattaaacttctggctctaaagcccagaataggttgtcaatgggatttcacagtggctccctgacactaagcaaggatgtatttctacatctagttttaaatcttaaactctaataggattctcagaaaagacagtaaagactaaggccaaaaaaagttgtttaaaggggaaaatgacagtaagtacaaaagataatctggggaagacctggcaattctgaaatcaaaacatctgttactaagtggatcatgactgcttagtttctagtctagaatctttaagtaagccattcagaagatgtgatactatagaatgataccaactgaggaatagagtggagaggaagggctgaagatgacttcagaaaaaggcctgaggcgtgacaaatgttgatctcactcactgatctatgctgtatttcttttaagaatgtattaatataaatgtccttttcttcccacatccaatccctattactacaccaaatacttgttcctctaggaaatgtgaagttgaaaaaaaatttcccccattaaatgagaattattctgcttatgaaaaaaaaaaaaaaa

SEQ ID NO: 760 - DNA, 1393 bp, HOMO SAPIENS
gaggcaagaattcggcagagggttacaggaaacttgataatgaagatctccaccctggtcaagaacagaggaaagacctaaagtgaggtcagctaaaaacaagaaagaaatttaaagcatcctgggaagtcatcataccctttttctccaagaaaatctagttttgcttgtttttgtatatcccacaacacctgtgtttgactatctttttcactttggatttggattttggtgtgatatacacagttccaaaaaatcaatggatagaaaactcaatggcataaataagtgcttaaaacaatacttcaagaaatgttcaacattccacgagaacaatatccacgtgagatgaaaagaaaagtctaagttagttcctgattcctggaagctgggaaagtctcattccagtaactacttaataccccctgtcagtctccttcaaaggctgaagccagcctctgactgaagcgcagccgcaatgtttcctgtctggtgccagcacagcttgcctggacttctgaaaattgcaagcgcttgctccttgctagtcaccacactccccctttcttagttgcagactccctcttgctgtggtttaccaggtgcccttattgcccctcatgcttgtgtgccactttggacttaatctggcacacttaactcccttcatctacgcctgaagctggacaagctgaagtgactgcgtcactttctcaattctggctcctgcccaccgaccttgctctgctaggaggaattagctctgcctggccagaacccaagaagtgacaaatcaaactcgctcaataggtggtaattaaacttctggctctaaagcccagaataggttgtcaatgggatttcacagtggctccctgacactaagcaaggatgtatttctacatctagttttaaatcttaaactctaataggattctcagaaaagacagtaaagactaaggccaaaaaaagttgtttaaaggggaaaatgacagtaagtacaaaagataatctggggaagacctggcaattctgaaatcaaaacatctgttactaagtggatcatgactgcttagtttctagtctagaatctttaagtaagccattcagaagatgtgatactatagaatgataccaactgaggaatagagtggagaggaagggctgaagatgacttcagaaaaaggcctgaggcgtgacaaatgttgatctcactcactgatctatgctgtatttcttttaagaatgtattaatataaatgtccttttcttcccacatccaatccctattactacaccaaatacttgttcctctaggaaatgtgaagttgaaaaaaatttcccccattaaatgagaattattctgcttatgaaaaaaaaaaaaaaaaaaaaa

SEQ ID NO: 761 - DNA, 592 bp, HOMO SAPIENS
tgattctaccagaaaggaatgaagaacagaaccttcaggaattgagtcacaatgcagacaaatatcaaatgggagattgttgcaaggaagagattgatgatagtattttctactagccattgggaagataaaaggagacagaagattgaagcctttgccagccattctttccctttttgcttccaaactcctcaactgggaaccttcatatgtgcagtatttatattggatcatactggggattataaaagttcctaggaggctagaagagccaaccaacagagaagggaaagcagtctgttctgaacatagggacataagttcattcatgccaagtatctttccagcatgtttctcccatttagaatatctagcatgtaaggcctttcaatattaatataagcccaatatcagctctttctctttgtatttcatctctttctactctcctatttgtattttgtgttcctatcaaagtgtcgtatctgggagatgacctgccttatcctgttctataacagttttgtttggtgctgtgtctttagaacagtgcctggcacacagtaagcactcaataaatctttgatgaatg

SEQ ID NO: 762 - DNA, 175 bp, HOMO SAPIENS
tgggccgactctggaaccgagctgcctgggtgaatcccagctccactacttactagctgtgccttgttgggtaaattatctgttggaccttttataaaacaagtactattaatacctcattggactgttgtaaggatgtaaatgagctaacatattaaaggaatttcaacagtgg

SEQ ID NO: 763 - DNA, 53 bp, HOMO SAPIENS
atgaggacacctgaggttcagattaagaaatctgccccaaagtcttagaactg

SEQ ID NO: 764 - DNA, 386 bp, HOMO SAPIENS
gcacatgcgcagaatcatcgtggtgcagggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttacca

SEQ ID NO: 765 - DNA, 1908 bp, HOMO SAPIENS
tacactcccaagagtgccttcgggaacccctctccccactacccaaccagagagacagccccacgcccggcaagacgcaactccgggccacggagggcgccgcccggacagcccggccgggtcagggggcttcgccgccgagcaccctccggcagccgccctcgggcctccgcggccgggcgggagggcgcctccccgaccacacttcggccgcttggcacccacagccgggagggcgtgaggaaggactcggtctaactccggggaaccctcggagttccggcgcagacacccgagggggtgtcctgggaactccttcgcccgcgacacccgttcttctccgtctcccgaccaccagcgacgcagcagggcggctgccggcccggcccggcccgccttgcgggctgtgcgcggcgcagctcttcctccgcggggccgtgaaggccaccggccgcccccgcgcgcccagctgcccggccccgccgcctaagccgccgcgttgctggcgggtagccgcgggcctccggtgcgcgggccccggcgcgtccccgagcgcgcgcctggccgcggccccgcgcgagttgcactaactttccggggcgggggaggggtgggcgccgcgggcccggctcccaggccttacctgtgctcaagttgtcgttgattttcaaaggcacccgcaggatgtagaccaggaatagcacgatgaagaaccacacggtccacagatacgtccaagaccaaactatgcaggacttgagctgctccagaaagcccgtggccgcttcagccatgtttcagcgctcctctctgctgccgcggctcttggtgtctgccgggatgattcaccggcccggaggcgcagctcgcgccgccgccgccgccgcggcccgggcgcccccggagcccgcccgccgccggggacgcgggctcggcgggaaatcgcgcgggccgcgggcgcgccggcttttccttctccagccttattgctaactctctaaaggtccctacaagtggctttcgtggagcgatcacgtggcccctgccccctcgcgttctgggtccatttatatttattatgctaatatttagaaagagaaggccaagtggcgcgccgaaggaaacgcctcaggcagttacaacccgcataagttgagcgtttaccacacgccaggcgctgctccacgatccgtacaatctcatttaacccctgcaacaattccccactacgatcctctttctataggtagggaaactgaggctcacagcagctgcgtagtttaccttaaggtggaaccaaaattcaattgcagtggtgtattctggggtccgaggtcttaaaccctactctgctcccttatcaggattcaggctcagggaatgaataaacccgggacagctaggagaagggaggaggtgcgctcctgtcgccggctagtaggcctgaagataaatcactctattcagcaagatttgggtggcagatgctatttacctttgttctgtctctttgaagcagcgcagggattttccagagaaatcttgacagatgaatgtttatcctccaggttttttggtgatcagaggtcaaactattctcagcctgtttttaatataaacaattctctgggataattttgactgtgaaacggccacctctaacagaaagtccattttaaacattgttttgttgtttttgttttgttttgactgtgctgaataaaatgtttcttttattctaaaatgttttctcagctgaaattttcaagtctgtttctgtccgtaagtgatttgactttctatgtattagatggtaatgaggaaattgcaggttttccagtctacatctatttttaaaattctgagcataaagaatattttattataaaaaaaaaaaaaaaaaaaaaa

SEQ ID NO: 766 - DNA, 2045 bp, HOMO SAPIENS
gggacctttagagagttagcaataaggctggagaaggaaaagccggcgcgcccgcggcccgcgcgatttcccgccgagcccgcgtccccggcggcgggcgggctccgggggcgcccgggccgcggcggcggcggcggcgcgagctgcgcctccgggccggtgaatcatcccggcagacaccaagagccgcggcagcagagaggagcgctgaaacatggctgaagcggccacgggctttctggagcagctcaagtcctgcatagtttggtcttggacgtatctgtggaccgtgtggttcttcatcgtgctattcctggtctacatcctgcgggtgcctttgaaaatcaacgacaacttgagcacagtgagcatgtttttgaacacattaacaccgaagttctacgtggccctaacaggcacttcctcactaatatcagggcttattttgatatttgaatggtggtattttcgcaaatacggaacttcattcattgaacaagtctcagtaagccacttgcgcccccttctgggaggggttgacaacaactcttccaacaattctaattccagtaacggggactcagattccaataggcaaagtgtctcagaatgcaaagtatggcgaaatccactaaatttatttaggggtgctgaatacaatcggtatacttgggtgacaggacgagagcctcttacttactatgacatgaatctctctgcccaagaccaccagacattctttacttgtgactcggaccatctgcgtcccgcagatgcaataatgcagaaagcctggagagagagaaacccccaagctaggatttctgcagctcatgaagccttggagataaatgagtgtgcaactgcttatattctcttggctgaagaggaagcaacaaccattgctgaagcagaaaaattatttaagcaggccctgaaggctggagatggctgttaccgacgctctcagcagctacaacatcatggatcccagtatgaagcccaacatagacgagacaccaatgtcttggtgtacatcaaaagaaggctagcaatgtgtgccagaagactcgggaggaccagggaagcagtgaaaatgatgagagatttaatgaaggagttcccccttctgagtatgttcaatatccatgaaaaccttttagaagcccttctggaactacaagcatatgctgatgttcaggcagtcttagcaaagtatgatgatataagcttaccaaagtcagcaacaatatgctacacagctgctttgctcaaagcaagagctgtctctgacaaattctctcctgaggctgcatctcggcgggggctgagcacagcagagatgaatgcagtagaggccattcatagagctgtggaattcaatcctcatgtgccaaaatacctactagaaatgaaaagcttaatcctacccccagaacatatcctgaagagaggagacagtgaagcaatagcatatgcattctttcatcttgcacactggaagagagtggaaggggctttgaatcttttgcattgtacgtgggaaggcacttttcggatgatcccttatcccttggaaaaggggcacctattttatccttacccaatctgtacagaaacagcagaccgagagctgcttccatctttccatgaagtctcagtttacccaaagaaggagcttcccttctttattctctttactgctggattatgttccttcacagccatgctggccctcctgacacatcagttcccggaacttatgggggtcttcgcaaaagctttcctcagcactttgtttgcccccttaaactttgtcatggagaaagtggagagcatcctcccatccagtctgtggcaccagctaacacggatctgagagaagccctgtcctccactcacctcacccgccgctgccaccatctcctctgtgccaactccttgtggaccgcaagaaagcatgactttgaaaaagggaagccattccgagattttaaaatgttcatggactattccatattaaaagctgtttttgttgtacaaaa

SEQ ID NO: 767 - DNA, 1588 bp, HOMO SAPIENS
  misc_feature (1125)..(1125): n is a, c, g, or t
ggccccgcggaggaagagctgcgccgcgcacagcccgcaagcggccgggccgggccggcagccgccctgctgcgtcgctggtggtcgggagacggagaagaacgggtgtcgcgggcgaaggagttcccaggacaccccctcgggtgtctgcgccggaactccgagggttccccggagttagaccgagtccttcctcacgccctcccggctctacatatgctcagcaagtgccgcgtgcctcccatccttggtcggtggtaaatgcaggaggtaattaacaggatcttcacaaacttggagccggcccactgagtcgttaaaaaaacacccgcaaagaaaacacacgagccttaacactccagagagttccgaaacacaaagtaaatttcgagctaagggttacagtcctttaaatctggaaatttcaagtatccctcgggatcgtgtccctgggaggcggataggattcagcactctagaattgtctcaccatttgaataggtgctgctcagcccttttgagtctgtcatcggaaagaccatggctttctcgcttttcatctttttggatggctgcttgtcgcaatattgtcttcttcgctttagacaaccctgtggtgcttactcttcctgtgatgaaggcaagtgtgttcctggagaggttactgaatctggagctgcttttagggagttgcagatggcgcaggcctcccttcctcctcaaggaagccaaagacaccatcacaatcaggcagccaggagccagggcttccataggcccagctctgtacctgtagggggatggaatctgctaaaagtgttctggagtcgttcttggatatctgcttggtatcttctcaatatcttgaccatctgtgacaattttaatcctcatttgatcatattttatgcaatgttcacagcatcttcaccaggagtagattctatctcaaaacactttctttgctcatcaaaagaaacagctctttatccgttcaagttttacaaggaattacaacaattcagtcacatcttaaagcttcagttctaattctagttattttgctgtttatggcatgcctgcagttactttatcttatatgaattatggtatatgtgtgtgatntatgtgggtcagtgtttgtgtatatatgcatctttttttgtatatgtatgttttattacttcttctagattctagttcttcatcttccagtgttaattccttgtacaaagtaggtccttatcattcactggatgaatgaatatccaagttacggtgtatttagtgagctgggattggaacaatacaattgataaaatgcattactgagtatgtgccccatctaagtttgcagctttagattgtggtcagaggctgttacaagatcttatatattctatgatgggccatatgcatctaaagcatggtggttcagtttatgaatatataaaaggacaattttttgattaatgtcagctttgagtttagaatgtatcccagacatcttagttgcccttaattatttttggagatatttttctcagccattaataaagcaatccgaatgacgaaaaaaaaaaaaaaa

SEQ ID NO: 768 - DNA, 8352 bp, HOMO SAPIENS
gatcagagtgggccactgccagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagagaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcgtgttacggtgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgtgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggtgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttggaataaaaaaatcttgattgggaaaaaagatgggtgttctgtgggcttgttctgttaaatctgtggtctataaacacagcacccataattacagcataatcttcaagtagggtacggactttgggggattggtgcgagggtagtgggtgagtggcctactaaaaagcccagtaacccccacaggaaaatagggaacttctttttaagtagcctcctttccactatttagtaattggctgtgagctgggctgggggagaaatggggcggggtgtgtgtgtcattggaaagctctcttttttgtttttttgagacagtctcactttgtcccccaggctggagtgtagtggcatgatctctgcaaactgcaacctccacttgtggggtccaagtggttgtcctgcttcaccctccctgtagctgggactacaggtgcacaccaccacgcctggctaatttttgtatt

SEQ ID NO: 769 - DNA, 8110 bp, HOMO SAPIENS
gatcagagtgggccactgccagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagagaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcgtgttacggtgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaaacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggtgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttggaataaaaaaatcttgattgggaaaaaagatgggtgttctgtgggcttgttctgttaaatctgtggtctataaacacagcacccataattacagcataatcttcaagtagggtacggactttgggggattggtgcgagggtagtgggtgagtggcctactaaaaagcccagtaacccccacaggaaaatagggaacttctttttaagtagcctcctttccactatttagtaattggctgtgagctgggctgggggagaaatggggcggggtgtgtgtgtcattggaaagctctcttttttgtttttttgagacagtctcactttgtcccccaggctggagtgtagtggcatgatctctgcaaactgcaacctccacttgtggggtccaagtggttgtcctgcttcaccctccctgtagctgggactacaggtgcacaccaccacgcctggctaatttttgtatt

SEQ ID NO: 770 - DNA, 113 bp, HOMO SAPIENS
ggctggtccgaaggtagtgagttatctcaattgattgttcacagtcagttacagatcgaactccttgttctactctttccccccttctcactactgcacttgactagtctttt

SEQ ID NO: 771 - DNA, 101 bp, HOMO SAPIENS
ggctggtccgagtgcagtggtgtttacaactaattgatcacaaccagttacagatttctttgttccttctccactcccactgcttcacttgactagccttt

SEQ ID NO: 772 - DNA, 92 bp, HOMO SAPIENS
ggctggtccgatggtagtgggttatcagaacttattaacattagtgtcactaaagttggtatacaaccccccactgctaaatttgactggct

SEQ ID NO: 773 - DNA, 118 bp, HOMO SAPIENS
ggcgaattcgagctcggtaccccgggatcctctagagttggtccgagtgttgtgggttattgttaagttgatttaacattgtctccccccacaaccgcgcttgactagcttgctgttt

SEQ ID NO: 774 - DNA, 84 bp, HOMO SAPIENS
agttggtccgagtgttgtgggttattgttaagttgatttaacattgtctccccccacaaccgcgcttgactagcttgctgtttt

SEQ ID NO: 775 - DNA, 2418 bp, HOMO SAPIENS
  misc_feature (418)..(418): n is a, c, g, or t
  misc_feature (462)..(462): n is a, c, g, or t
  misc_feature (1048)..(1048): n is a, c, g, or t
  misc_feature (1163)..(1163): n is a, c, g, or t
  misc_feature (1203)..(1203): n is a, c, g, or t
  misc_feature (1465)..(1465): n is a, c, g, or t
  misc_feature (1468)..(1468): n is a, c, g, or t
  misc_feature (1529)..(1529): n is a, c, g, or t
  misc_feature (1533)..(1533): n is a, c, g, or t
ggcacaaggctatgttcatgcactacttcggtataatctcaaaataattgccagctccccagtggaacgcctgccttggtccatgtggaatgtgggcatatggtaggatttccttggaggaactagaagaggaacataaagaatgttaggcatataaattattgtttctttttcttcttgtttctttccactgagttatgtgtgagcccccacacccaccctaccttcctgtcctctaccttactgatgctattttcaggctgttgtagaattgggtgtgtaaaattaaattagcctttctcctcttaatatctgtaaaatgaacaaatttgacaaagaaactacctatctgatatataagaaggttattagcctgtaccacaagtgaaaaagtttcatcctgatatatcaaaatagnaccacttgaaggaaaacgttggggccatctgtttctctcattgnagttagagtacagatttttaaaatatgagcctcaggcataaaaatgcagtattagcaactcaacttgaagaagcagttactagtgtcctgggtgggccacagggaaaatagtaccaactgcctgttatatttaactgcaattaaaaatggcagtgctgggtatcttctgtctctgggtcatctcctaaagatcctgcagagggagccatagctagtgtagaggccagccataactagccatggcaaagaccacaggggaacgctcttcaacatgctctgcactgatcctctaccctggtgtggagaaatgtcagattcatcactaacaaggttaaaggtgtaaattgagggaaaaaaatgagaaaagttatctttcacataagacttcagaaagaaattgcgtcgtcaggtttggaactggtttggtaatttcatccagttattttcattttaattactgtcactgttttcttcatccttgacaaactatgagctctttgagattgtctttttcatatcctgatgtcagagcttcctgtattttctgatcatggtatgtgctaagtaaatgcaatttttagcattnggaatggtttatcaaatgattgaaggagtgggtaataatatagattacatatacagtcagccacatatgtgtatgtatgtgtatttgtgtgtgtgtgtgtatatgtatatactgncgaccctccgtatcaatgggtttcacatgtgtggattcanccaatcatggatcaaaaatattctttaaaaatatggttgcatctgtactgaacaggcacaaactttttttcttatcattattccctaaacaatgcagtatgacaactatttatcatagcattgacactgcattagatatgataagtaatctagagattaaagtatataggaggatgcatgtggatcatatgcaaataatacactacatcttatataagggacttgaacatccatggagtttggtatgtggtgggtgataccnccnccttgaggaggtgagtatcagagacgcaggtaaatatgatccagagggcacagaggcattnccancctcacactggctgtcaacaccatgctgagttccacgaaagcagcgaggagagggtggtgttcttgtttctggtacaatacctggcatagagaagatgtcacaccaatgttcgtctattgaataagtcaccaaaattgtgaatgactgaataaatgggcaaagggatttgttccctacttgctgtgggaagtttcttgtagttatgacctccatgaaagagagactcgtatctggagaaagttcatagctgaaaccatgagatttaacttttgaagttataatggatctatttttttaacatcataaatgaagattatgcaatactgtatgtgcttcaattgacacaaccccttcccattcagggctgatttatgttctgtactgaatgacaggcacagaagtaaatgtgataatcctgtcccgtcgttaccctttattgtggtcttgctgtgtcccagccacacacttcatactgtatctgtgtcatcattctcatatacatctatataggtacacttccatagacttaggcccatttaatggaggagcaactccaacatttagagatcatgaaaggtacctcatgacatatattgacctagtggcagagctgcttagttctacagcctctgagtggaatcctcacgacagtaaaaacacaagcttggtttctcacacactctacctgctttttctcatcccctttagtctctattccttgctgagatgtgttaggcattaaatggttacagtctacttctgtgtgtagtctccaaacactcagaaacctcaaaatttaaaaaaatacagaacaatatgttattaaataatcagggacaagttaaaatggtaaagagaataggccactttgtgaaatcatatcc

SEQ ID NO: 776 - DNA, 10156 bp, HOMO SAPIENS
agacgggttgataggtgcagtacaccaccatggcactttatacctgtataacaaaccagcacgttctgcacatgtatcccacaaaagtaaaaaataaaataaaatacatctacatgttgaataattttaaaaaacaaagacattaaaaaaagaaaattaaaaaaatattgttcacatatcaatctccattcctccacttctcctcactaattactgacaatatcccttcacctgtgtgggtagaagaaaaattctagcagttcaaatccttggcctaaaccatcattgctaaaatattcactttttaacttccaagaagttagttaaatcatattctcactcaaaatacaagtaatttaaatgagtttttatcagtgagtttcttctggaggctttgtttctttcattactctcagcagcatgtaagaagcaatagggtgatactaaaatggttttgtctaacctgaacgctgtgtgtctggatcttcattcactctgtggaatctgtattctatggaattttaagtccttttggcaggaaattgtaaagtgaacagaaacatgggtcccatgagacttcttgtgccaaaacactatgcattatcctggtacctttaagtccaaagagaattgtttaacttcttcattcttaatattatgttgataacaataacaaataaaggattctaagctactcacacatgacatataaattaaagagtgttttttgtcacccaaagtcaaaacacaaattaggatatttactctcttgctttccgagggtgatttctattgctgtagacaatttttagagaaaataaaagcttttaattttaagtgacctgatagtctgatgttagttatctctgttgagcattttctgttgaagataggctcactttatctttaggctatgaggtcttagtgtgatggatttgtctaaatgtaaaacaaaacaaaacaaaactatgctctctaagtaaccactaaattctgtttataaatagaaggctaaaaaatgaagggtgatatgctacttggaattcaattaaaaggaactaaaaaataaattataatattatgcagaagcatcctcagatctatttaagtatttcagctttatagtcctaaggaaattcaaaagcagtgacagtaatgggtgaccattcaatgtaaccttgattggacaactttgactatacaactaacttcagattaatttttagtgggagtttttaaattgctctttgtaaggtggacagtgatgtgagtggtccttttagaagttttttttttcctgatctcaaccactagtatattttattttcattagcatatccaggggatacagtagtgccttcttatctgcagattctctttccacaatttcagttacttgtggccaaccatggctaaaaatattaaatgaaaaattacaacaataaacaattcatcagttttacattgtgtgccttttgagtaacatgctgaaatcatgtgctatcccactctatgccacaaggaatgtaaaatgtccctttgtccagggtattcacactatataaactagctgcccattagccattaacattgtttgatcctgacatctaaccaacaacatcatcatggcttgaagatccaggatcacctaaagctatgtcgcaatgcctgcatcactcacctcacttcatcttatcatacaggcattttgtcatctcatatcatcacaagaaggttgaggactactgtaaaatggaaagcatatccattaacctcacagagatcagtaccctggcaaaggttgtttgcatactggaggacaaatattttatcgtaaagaacttagaaaactttcaaaaatgttttggaaacaatttaatgaaccagaattttttaagcatctaatgattttctggcatgcacaaaggtatgcacattaggaaagcaatacagaataatttttttaagagatggggtcttgctctgtcacccaaggatagattgcagtggcatattcatagcttattgcggcctcaaactcctgggctcaagtgatcctcctgcctcaacctccaaagtgttgggattacaggtgtgaggccatgcctggcctggaataatgttttgtaatggtttatagtatggaaatcagagttggaaataatgactttaattctttggtcctgcatattactggctctggatgtccaggcaaccacttagttccttaaagcttcagttttcatacctgcaaaatgggagattaaaatacccatctcatagggataaatgtgtgtagcacagtacctggtgcatagtaggtattcatgaaatgtggctcccgctataagcaatgaaaaccagcaccaccttaacattgacccttttaggatacaacgttccaaggaaaagtttaattttgcactggttaaaggaatttcaaatcattaccaggggaaaagtgtaataaactgccaagataactcaataatattgagttattctcaatagatacacgaaataatattctacttggaaattcaattaaataagctccacaaatgtttattgaatgcccactattcactaggtattgcattgggttcaggagatttaaacattagttaagacagggatcttgtttccatggggcttacaatctcatgagggagacagattcacaaacacaaaaatcccttaaaggagtttcaaagaaggggaggcaacagcttaacctgggaaggtcaggaaaagctttgaagaatggataatactgaaatataagctaggtttagttgtagagtgtaacaggttttctttatcatcaatctctgtcaactgcctcatcctctgccacaaccccaagatatttcctagagctactttcaaattttcatgtgcttttggggagcagaaaagacaatatgcttattgattatcatatttatagaagaaacaacaagatcattttctttactgttactcattgtctaccaatctattatggtaatgaatgagaagaaaaaataaggaagatgtttagaggttattttccttaaagggagattccacatcctgtgcctgccttggaagccttgaagaaaagactatgcttacccacaaacatctcttggcaccactgtcaggtttgagtggaccacagcaacaactcatgagatcatttatatttgtatattctttgtgtataaacttggaatttatctaaaaatagcacattagaaggtgcaaaaccatttaatttgccaagtacttgtcataaccttaatgaagcagttcacttggttgggaaccaataataatatagacaggtagaaaacacagtgatcaggtgaggacatgaagaatgtttgacagaatgtatttatttctgtttatcttcacatgcaaccttgtcaaacacggtcaaattttcaatgcaaaagattaagcatgctaatgcataattctgagatacctattttaaactgcaggttgcatgaaattaatctttttaaactactcatcttcaggagttctcacccttctatcaaagtagtatataagctcagattctcattcagattctaattatatcaaacaatcacaagaatgctactaaactgaagagattctctctctgttaagggagaagtgttgatgtggacttgaaactatacagtcacttttccttttccatatattttcagttatacaatgttaaattcatactttaggttctgatatgaaagagatacagagcacctttaaaaagacactgaaaagcaccatctgttcatgctcaaatcttgaagaaaaaatttttaacttgttaatcataaattgctctacaaatagtatactggaaacatccggatgtgaggttataattaaaaatgaaagcaaaacagcttccctgaaaagaaagaaaactcttaattcaaacctaccagatagctgatttatgaaagtgaaaatgctggcatgtatagatattgggaagtcttgggatctggattttatcactgaccatctggttatagtcagagtgaaattttaaaatagcaaaatattcattaaaagcctaaagaagctcataccaacttgacatgccacaattagttatatatcccatgtatccccttctgatatcaattatgtcagttccattggtgacatccccgttaatatttgatgacacataaacgtccagccactttaattaggatcaagagaatgggcatcaaatgagtatttttccgtcaagctgggaataataatactttatcaagaccaccttttgatccctacatgtgggaaaactgaggctcagacagttaaattacttgcccatagtcacatttctagagattgggagaggaggaaatcaaatctaggtctatctgcctccataccgtttcttcacaagaagagactatcgattccctgagctgcataatgggatttgtttgttcctttcagtttcctcaagcatgaccactacatctgctgtaactagtatccacaattacccagctgcatttaactaatttaaagctcctcataccaattcattacctcaaaaactatttgagagttgaagaagtttctcagaaattaatgttgtaataaacaacaacaacaccaatactgggccctctcattgattcatattttcttatttatcccttctccatagaacactgcaaattgataaccaaacagaaatttctctttgtgatagtttattttacatgtcaacttggctgggctaagagatgcccagatagctgataaaacattgtttctgaatgtgtctggagggtgtttcggagtaaattagcatttgaatgagtagaatgagtgaaaaagatcgactctcaccaatgtggacaggtattatccaatcgattcagggtctcaataaaacaaaaaggcaaaggaagggtgaatttttgctctcccttcttgagctgggctatctatcttttcctgccctcagacatcggacctgcttgttcttggtcctttggactctaggacttaaaacagcaccattcctacttttcagttcttaggcttttggagtgaaattgaatgataccattttccaacttgcagacagcaagtgatggaatttcttggcctccataatcatgtgagccaattctcataataaatctctttctctcctttttatatatctcctattggctatttctctggaaaaccctaatatactcttgttcatatattttgttactttttaactcatttgcatctcaggcaaaaaaaaatcttttcttattagcttctatcctccaatttctttcccttgtctccaagaaagtcactaccaccctttttcttccttcattctttccaagatgtcaacccatgtgggctggtctgctatcatctaaatcattctgctattactgtggtcagtgggaggcagaagtgataagcagcaaatatacatcatctagaacttcagttctgacccactggcaaagcgtcttcaccacaactgaaaaggttctgaggtagatctgggtttattatgaaagattgacacaattgaatattaatgtcttccactgaagaagaaaggtaatgatacctatgtcttatgttttctatccttggcataaggcctcaataatctcaattcaaatccaaaagttttcaggaggaaacctctggacacattaaaatctcttatattaaacttacagatatttatttagaccttttcatggagtctaccatggcttatcactgacaaacggacaaagaaatataaaggcacgatcctcatctctatagttacttaaaagggcaagacaaactctacagcattatggattgttaagcgctaggtaatagcaaatagaataaacagtataagaatgcattagatgataacactactgagggctgtaaaaatctctgaaggcttcaggtgagggggagatttaaactgggccttaactgatttgcaaaactaaacaagaagacagatatgggtgttccaagatagagtgggaagctttagcggtggtatttggggaaaattaattggtcagtattcctacggcatccattctacagacagcaacagtatagacaaggaactgtctgaagggacaaaagtgagatgttcagcagggtaaataggggatggtgtgataatgataatttctttgattatagctatctgctaagccctgtgctaatttaattttttaaattaaattaaaactgcataattcagagataattattatctctttttcttatcagaaaattataaacagttgaggtcttccaaggcaaagcccttgttgttacccatcatgctgagggttagttcaggtgcttgcgcaatggcatgtacatctatctcctttgttttgatacatccatccttgactcaggaatagacctagagacctacaaaagtctcgagtttgccgagacacatggcagatgagggaacacaccactgtggttccctcaggaaacagattctaatctggctttacattttccattgtaatcttgagatgcctcatagtcaaataatagttcctcagtgaaatatatggatagtcatatgatagtagaatgaataaagaccttatatagctacttggcatttcaggccaagttttattgacagactagtttgctacaaacttatgaacccatttttattttctagatagcaaaatgttaacaattagggacctgattgaagggtataaaggattgactgaattattgttgcagtttttatctaagcttattttttagtactttttggatattggaaatgtggctaagagactgtggacacttaagcgttcaataactgttagttcccttctcttgtgctgtagaaaaagtacaaaaggtccaagttacctctcagatatcaggcagatgattaattgtactgtgaagcggtcaagtcctccagaatactgcattttgggaacactgattagtcatattcttataagaattgggagctgccatttttaacagtttgaataagaaagttgcattgccaattctgtacctatctgatatgttctctccccggaggagaagaaacaggccttattcatctccccctccccaaccctaaacattcttcccaagtcctgaatatgcctgaaaagacatgtcattatggtttttcctgtggagtctagagttcccccacctgcaccctactgaagtcattcataggaaaaggcaatgacagccagatcttgacagatcacccctagcacagtttcactgtgatcccacaggggctttactgaatatcagtcccagagctgtctgtcttttccatttgggctgctgcagactgagctgagtaggtcagggatatgttttacaggcattacgcaggccagtcttcagaggccagtgtgcatggtgggtccagccatcctcagacatactctgcacatgtcatgctgggggacagagcagcgtgtgtgctgtctggatgattcctcttggaatgttggttgcctagcaacaagatcatgctttgctcttaagtccaattctcaggactttggcttagttacttctgatccttagactttttcttaccatccctcacccgggttcccagaaagtcaggggcttcaaattccaaataatgtagttattttagctacgtggtggcagtctatatgttagatttccctttggcatcctacctgtactttatacacaccaacatttctctaacagttttaaatatgctacattttaaaagactacacagttggctttccttttttaacacagacacatgtacagcatatgtatgtattagaaaaagaatggaaggataaccacaatataaaataacagcagctttccctttatggtaggaatattagatttttaaattatcttcaataaatgtgttaattttgtaataaggataaatattagcataatatatattgggattatatgctattaactgacatttaaaaatatatacaaagtaatggtaaaggaagaaatactgtcacacataagtacaccacaatttcaaccctatatatgtggaaaaatatgacaggaaattaatgaatgaagagggtttcatcaggataacagtaattttttcttaaattcttgaattttaatttaaaaataacaaaccttaaaaatatatagtattggcatatttacagccatatgttttaaaaggcagaggaaatcatctttatcatggcaacaagggtcatttgggcagattcccatagtaaaagattaaaatatattttggacccagagtttgttccaatcattccacttcttcattcactcaaaaagccatgtttaagcatctgctttgtgccagatatgtgaccagccctgtgaatacagacctaaagcagagcatctctgccctcaaagaatctatattaaaagtcagatgagggataaaaaaataggatgcaccatttattataatacttctcaaaccatgttttgtgggtgttttcaagtgaattttagatttctctacaagtcttaattttatatttatatggtaatctatttaattaagcaagtttttcagaatcatttaaatggcaccttataactgagtgctaacacaggacttcaaggtctacagttatacttgggaatataattgcatagtactgagtattatttcatagtcttggctaatgagaaagggacctaaatataaatgatatgttgtgacacatgaaaggcaactgatgtcataggatatttgccaataatttgaatagtgacaaatggccagataattgagtcatattgggatgtgcaggatggcatggtcatgccaaactcaaaagctgttaagactatattcatgctgtaatcagagatcttgtttcagcaaaatgtcattctttatattaaataaccattgttaatgttaatttcattccactagaatgcagctctatggcaaaaaaaaggctttatctgttttatctgctgctatacccctaggacctggctcataactgcctattaaacatttgctggatggattaataaataaattgagtatttttatttatttatatatgttttctaaatttatgcttatagtttaataaaagttatgcctgttttgtgttgacatgtgtttaaatatattttataataaaaataacactggtggacttaagagaatttttctcctttaaagaagaaatatttaatattaattaagggtgctgttggccctttaaaaaccatagggttaaacctaactcttcctttttctgtgatatccgttttctggctcttctcttctacttttgcttcccaccaattcacttttcaaaattcccacatgccctttctccctcttacccttttacttcctatacttgccaaccctagcaacttcctcactctatcatcatacactaaaacagaggaagagaagagaagaaaaaacaaaccagagaataaggcaggttttccacagccccccaagaccgtcaaagtgaaggtaagagactcagaagttaccagctaagggttcaaatgctaatttcaccatttatatactgcccatcagaaccgagtagacattaaccaattttgacggcgtactcttcttgtcaataaaaattttaagcgcttagcatattacttgcaatagctctgggctggagcatccgtttcctctgttcacatactttagaaggtctcactccgatctttctctgataagaactcctcctcatcaggtgaggagcttgtaggatcaaggggatgtgcccatctggtgcctaagcaccacctcatcaagagttcgaccctgcatcctgcccaaattgtcctgtttctagaccctagatatgtgcctaattcattttttaatggtggaccatctctgcgggtgtgtgtactcctaagtatcacaatgggtttcaaatggtgtggcttaaaagcatgtgctaaagtgtcaatagctctaaatgtgagtcatgagacagtgcagaagaaatcttcatggggagggaagaaaacgaggtggttcttagcccaggagctcaggtcccttcttcctatgctacctccttatggctaggaattc

SEQ ID NO: 777 - DNA, 451 bp, HOMO SAPIENS
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc

SEQ ID NO: 778 - DNA, 1649 bp, HOMO SAPIENS
  misc_feature (1127)..(1127): n is a, c, g, or t
  misc_feature (1130)..(1130): n is a, c, g, or t
  misc_feature (1253)..(1253): n is a, c, g, or t
  misc_feature (1313)..(1313): n is a, c, g, or t
  misc_feature (1488)..(1488): n is a, c, g, or t
  misc_feature (1621)..(1621): n is a, c, g, or t
  misc_feature (1623)..(1623): n is a, c, g, or t
ctcattttctcttgctgctaccatggaagaagcgactttcgcctcccgccgtgattctgaggcctttccagccatgtcaaactgcatccactgaaggtcttggaatgtatacctgatggataaggaggaactactattgcatgtgattcaacaattctacttctgacaaaatgtgactgaaaaacagaagcatttggaataccgtaacagaagtcattaaaaagagccatgtaatatgtgcctgcttccccttctgccatgattgtaagtttcctgatgccttctcagccatgcagaactacagagttttgctcttgttgcccaggctggagtgcaatggcacgatctcagctcactgcaaccaccgcctcccgagttcaagagattctcctgcctcagcctcccgagcagctgtgattacaggcacctgccaccatgccaggctaacttttttgtatttttagtaaagacaaggttttgccacgttggccaggctggtctcaaactcctgacctcagctaatccacctgtataggcctcccaaagttctgggattacaggcgtgagccaccatgcctggccaactatttgttttcttgatcttgtcctgtgtgtgtgtgtgtgtgtgtgcacgtgcacacgcgtgtgctatgaagcatagaattcatagatttagaaatctgtattctaggatctattttttcacacttcaaatatcccactatgagttatatcatgattatttgttcacatatttgccttctcctgaaactaaacttttggaagatgtgaatcacatgttatctacgttttgtctcccagaaacctatatgtctagttggtagttgaccgttgccgggtgaaagctctaaagcctgactgaactcctggaatgacaaataaatggataggatgataacctaagtagtctaatatcagcacttagggtggtctctaagtggaaaacttagagaactgctacaaagtcattgtaattgtatacacgtgctacactttggagattactgctaaataaagatcaagttagtgtttgtatttccccagagaaaatagagaattgaatctcaaagacaagttgttgctttggaggtagaagaggggttttntcngcaatgagaagaaaaagagtaagaaatgtaatcttctacccaatctctaagttaaatcattttacccttttagacatattcctcatagttttcacagagggaaaagggaaaaagtaattcccntcattgcttcctgttcccctggcagtctctatattaaagatgctggactgcagatcctcnctgctgagggaaaatattaacctcattctctccaaacacagtatgaagcctagaaaatgtccttcctcattttggaacttaaggttgtgtacagaacagtcttacaatgacagtgtttagaactgtaagaaatcatcaaaatgaggtaagtctttgtgaggtagctgtggagctnatttttgaaagtattgctgcctatgattttggaggtgcatcatattgtcaggaaaggtctaatgtgctttttaagttatgaaattttggtgtcacatttgtcttgtgggctgcagaaataaaaaataggttgntnatggaaaaaaaaaaaaaaaaaaaaaa

SEQ ID NO: 779 - DNA, 22 bp, HOMO SAPIENS
tagcagcacgtaaatattggcg

SEQ ID NO: 780 - DNA, 1407 bp, HOMO SAPIENS
gacacagagaagaccactcccacctccccgagtgcaaggtgtgaagggacagatcttttaaccatgcctgcccccttatacttgctgttcatagaattgcaactgaaagtgaccatgaggatccactggatggagttacttctttcttaagtgaggaggctaagatctgagttcttcacatctctctgtagataaaatttccggtctggtttcacattcctctgtcagaagaactttctttaatgtttcttaaagtacaggtctgctgcttatgtgagaagaaacgcagggagacagttcagtcactgcaatcttcatgcccatcagtttcttgtgagaagaaaacaagagctccaatctgttagatggtattttgaagcaggtctttgggtaaaggacacctagacccagtgaaggtcatggtgattattattggacaatgggacatcactctgctattaagtgagaaaatgagacaggttgcactgagggctgttagccagacagagtctcgaactggaagtccatctagatgttttgcataagagaatggaaacaatctgtctgtgatttagggacatactctggcagcaatatgggaatacagtttcaatcctcattaacaaaacaggtatgaaatacatatttatttagtaaggtgccagctgtatgaaaaatccatttcttatttcccataatgtttctgaaatgtcttagcagtgcatagagacagcatgtcatcattttctagggactgtgtgttattgcatttttcctagggaagatcttttctaggtcacctgctccttcgctaaagctctgaccaatctagcttgctaacctgtgactccattttcctaagtcctgagagagaaaaacgctttgcagcaaattatgccaggcatccttgtgtctaaatgaaaaaaggaaaaagcctccttccttccctctgttgagaagtgcacggtccacatatgcatgcacagcatatactgtgagggtatttgcagtcccttgggttgctttgataactggccaggttgctgttctatttttccacattctattaatcctcctacaggcagttattaggtattgagtgctcacacacccctggcatagtcaccacatgccattagctccagataaacttccagaaaaaagtccatcccccacttctctcagctgcctgccaacgctggacaccttctcaccaagccagcaggacagagaaaagcctgggctttaagatcaaacaaacacagcttcaaattaggactctgtcacttcctgtgtactgggcactttgctgagtatgtggtttctcatctgtaaaacagagaaagatgattatctcccaatctttctatgttatatgtttgaattaaataaggtactctccatgaa

SEQ ID NO: 781 - DNA, 1881 bp, HOMO SAPIENS
tttcatttgtgattcggatgatgcagctctaggtggattgactatgaaaggcgctgaatatcttcaggaaaatgggttccatgagaagtgacatcctcctcgaatcaaaagattctcaggatgatggagtgagtgatggaataggttgccaactgtagttactccactcagcacagaatggctgcggggaaatataatcagacatgcccaaagaagtctgcttagggtgaaacaaatacctttgaggtttaaatcaggaaggagagactgtttctacaggtgtcatacaaaaatcaatactgagccaaggattattggatcccatagaaccacggagacttggtagtgaaattcttccaaagcattccatccgtgcaggcagatggtgttcctaggtttggaaatataaatcccatgggaagtcagctgaagaccattttatgcatcactcatcctgttagagccctgtgctgttctgtctctctataagactggctgaaaccaccacggttgtttacaatttatgctctaactttggatcacctagttaatactctaactccgattattaccaaagacaatgttctataaaagatattgtctattagaaggatgcttctgcattattaataaaatcacagggaaaatgcaacattttggatgtcacatctgataagttggcagttaaagaacagaaacagcaataagactgcgtggctacattttcccagtgccaccaactgggatttaagctgactaacattttgaaagcccagcccatttcattcacagcaggatggcagtgggtgactgatcgtacccgccagctggctaatgtcataattaacaaagttgctactacatctgcaaagtttaatcgaaaaatttgccagtgatatgatggccttgtcacagagatcacaagggacacggagttgacttttgctcaaggtggaacgacagagctgtgtaggcaagatgagaaattgcgaacttgttcggccaaaggaggctaataatgaaggggccgggaaggaggccagtgctaccagggttatctcacaacctcagatgctggataagtcgacggaggcagaatcgctttgggatgacattcagaaagcctggagtgattacttgcaagagaaaagcctacctattctttccaaggtgcaggaatattcccctgacctgctcatccagtggtccagtaatgaattactggagacctgccttagacaaagaatgcactcggagccccaacacacctaaagccatgctttgaaggatctgccaagctcacttttaatcatgagaaagaaattacccacacggagtcagcagaaaatgagaaagtagagctcgtgctgtgaataattccagcaagtgccaaggggctagttgaggaatggcttcatgagaacatgctctaggggaactattaatagcccaccagtcgggtaggcagcattcaatccttctatgccttctttcgccacctgttgaggtctttcttctgaaacaaagaagaaatagacaaatcagacttgccctcttggaaatgtggtccagatttctctactcccaagctccaaaaaagatgttagtctaaaacagcaccttggatctaagcagcacctttgagaaagaaaagactgcttcaagaatgtctagttgcacctcctttccgtatgtggcctaaatgcctaggttggatcaatagtttaatttttttattgaactgtttaatattgactatggacttacattcacttttactgttttctgtatatttacttttgcttgaagtgttttaatattgactatttacctctgctcatttttattgattttctgtattttttcaatgaaaattataat

SEQ ID NO: 782 - DNA, 1472 bp, HOMO SAPIENS
atccttcacctgttgcctggctagagttgtctggctccactttgagctcttgcagaaccagccctttttcgtgtggtccaggaaagtccatgcctggcaccacctcctcctctagtgactccacgtagaagagagtcctggctggctgctgagtgccctgcccaggagccccttgctgcagcctcgtggcaactggaagcagggtgccattcagcggattgaaggaagaggaggaagaggacggggaggacgatgaagaggaagaggaggaaggcttcttccagaaagtgctcacaccgcttctctcttggcttttgagcaggcgactctggctgggtccccagtgctcaaagctgccactgccgtcctgttgcaggcagcctccccccgccgggccgccggtggaaggagacgggtggctgaagagtttccagcggagtcgcagaatgtgcttcacatcgaagtcttttcgcccagagcctgacatgctttacgcacagaaggcaaaaggctggcagctcacgcagggttctggaggctgggaagttcaagaccaatgcacgagaatttggtctaaagagaatcttcttgctctgaacacacatagtagaaggcagaagggcaagagagagaacaaagtctgtgtctccacatggcagaagagcagaggagacagaacctactcctctatggcaaccaccccatcaatgacaaaaatcctagaaggatgtatgtataggaagttgaagtgttgagaagagaatggctcagagtcaagcgggaacaagattcaaacttcagagagagagggaagaaaaacatttaaatatatctggcataatccaagactatttacgacaagtgttctgtgtttctaataataaaacagacttcacctcggagtacctgcagaactgggaccccaatgaccagggagaatgaagaacaacttgtttgaagattgccttttctgactcccagcttccacggagagattaactctgttggctgaagccctatcccaattccttggctagaccctgggtccttcatgttagaaaacctggctttactactactactactactactactactactactactgctactgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgcattttttaaaaatatattatcttattttactatttgatgttataattgttatatatttttccatacttcctcatactgcttatctcttacttaagaatttatgaataaagaattgatttttca

SEQ ID NO: 783 - DNA, 1730 bp, HOMO SAPIENS
gcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcacagccaagcttcttgaaaggcctgtctacacttgctgtcttccttcctcacctccaatttcctcttcaacccactgcttcctgactcgctctactccgtggaagcacgctcacaaaggttcccagtgcccccgacaagcccctgctggtgtctccatctcctgccaagcatcctccagtgcctcctcctgtgggcctggcctcagggctatggacagactcctgtcccatcccagagacccctcgtgatcgtgccctggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaa

SEQ ID NO: 784 - DNA, 275 bp, MUS MUSCULUS
agctcgctctgaaggcctgtttcctaggctacatacgagggacatgttccttatcctttcgcctaggggaaagtccccggaccacgggcagagagtgccgcgtgcacacgcgcgtagacttcccccgcaagtcactgttagcccgccaagaagcgacccctccggggcgagctgagcggcgtgcagcggggcgtcatccgtcagctcacatagtgacgcaggcagtgcgacctggctcgcaccaaccacacggggctcattctcagcgcggctac

SEQ ID NO: 785 - DNA, 341 bp, HOMO SAPIENS
gctgatggtggaaggagaatgagtctctgatgcctttggacttgatgctggaaagacttaagactttgggggactactggaaaggagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagtggatatacactgttccaagcagcatgtgttgaaaagatttgtcttttccccatttaatggtcttggtacctttctcaaaaattgaccatatatga

SEQ ID NO: 786 - DNA, 710 bp, HOMO SAPIENS
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagaaaacaaggactgaaatccacacaggaaggtggcagtgaactccacagacggacctggacgcctcaacactcctggccttacctcccttgctgaacgtctcaagtttctctgcgttcaggactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtgacgctgggcttctttaacaaattaatcccaagtgcaagacatttatttcttctgtacctaatgacctgagcaatccttctctgctgaacctggtagtgtcatctttagaagtgaagacacaattaacacatggtcatttcttcattatatcgttgttact

SEQ ID NO: 787 - DNA, 949 bp, HOMO SAPIENS
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 788 - DNA, 369 bp, HOMO SAPIENS
gctgatggtggaaggagaatgagtctctgatgcctttggacttgatgctggaaagacttaagactttgggggactactggaaaggagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagtttaggaaaacttcctacaccttctttgttgggatgttctctggactaatgactccaggcgagaccaccgttgatcatgaactcactttgaaacagaagctgggttggtaagactggagctact

SEQ ID NO: 789 - DNA, 799 bp, HOMO SAPIENS
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaaagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 790 - DNA, 834 bp, HOMO SAPIENS
gctgatggtggaaggagaatgagtctctgatgcctttggacttgatgctggaaagacttaagactttgggggactactggaaagcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 791 - DNA, 550 bp, HOMO SAPIENS
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtgccatgttttgaaccactagattagagggtcaagcaatttcttggaattttactctgaattctacgtagaccattttcatgtgtatacctcctctgagtcaccctcaggtagggacatttt

SEQ ID NO: 792 - DNA, 376 bp, HOMO SAPIENS
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagtggatatacactgttccaagcagcatgtgttgaaaagatttgtcttttccccatttaatggtcttggtacctttctcaaaaattgaccatatatga

SEQ ID NO: 793 - RNA, 80 nt, SALMONELLA ENTERICA TYPHIMURIUM
acaucagauuuccugguguaacgaauucaagugcuucuugcauaagcaaguuugaucccgacccguagggccgggauuuu

SEQ ID NO: 794 - RNA, 86 nt, ESCHERICHIA COLI
aacacaucagauuuccugguguaacgaauuuuuuaagugcuucuugcuuaagcaaguuucaucccgacccccucagggucgggauu

SEQ ID NO: 795 - RNA, 81 nt, SALMONELLA ENTERICA
cacaucagauuuccugguguaacgaauuuucaagugcuucuugcauaagcaaguuugaucccgacccguagggccgggauu

SEQ ID NO: 796 - RNA, 180 nt, PSEUDOMONAS AERUGINOSA
uccgcucccugguguguuggccagucggugaugucccugagccgauaacugcaacaacggagguugccaguuggaccggugugcauguccgcacgacggaaagccuuaaggucuacugcaaccgccaccuugaacuuucggguucaagggcuaacccgacagcggcacgaccggggagcu

SEQ ID NO: 797 - RNA, 106 nt, Escherichia coli
acgguuauaaaucaacauauugauuuauaagcauggaaauccccugagugaaacaacgaauugcuguguguagucuuugcccaucucccacgaugggcuuuuuuuu

SEQ ID NO: 798 - RNA, 174 nt, Escherichia coli
uaaaaucaauaacuuauucuuaaguauuugacagcacugaaugucaaaacaaaaccuucacucgcaacuagaauaacucccgcuaucaucauuaacuuuauuuauuaccgucauucauuucugaaugucuguuuaccccuauuucaaccggaugccucgcauucgguuuuuuuu

SEQ ID NO: 799 - RNA, 171 nt, Escherichia coli
caaaagucccugaacuucccaacgaauccgcaauuaaauauucugcccaugcggggaaggaugagaagcuucgaccaagguucgacucgagcgccagcgagagagcguugccgcaggcaacgacccgaagggcgaagcgcgcagcgcugaguaauccuucccccaccacca

SEQ ID NO: 800 - RNA, 90 nt, Escherichia coli
gcgaucaggaagacccucgcggagaaccugaaagcacgacauugcucacauugcuuccaguauuacuuagccagccgggugcuggcuuuu

SEQ ID NO: 801 - RNA, 360 nt, Escherichia coli
gagucagacaacgaagugaacaucaggaugaugacacuucugcaggacacaccaggaugguguuucagggaaaggcuucuggaugaagcgaagaggaugacgcaggacgcguuaaaggacaccuccaggauggagaaugagaaccggucaggaugauucggugggucaggaaggccagggacacuucaggaugaaguaucacaucgggguggugugagcaggaagcaauaguucaggaugaacgauuggccgcaaggccagaggaaaaguugucaaggaugagcagggagcaacaaaaguagcuggaaugcugcgaaacgaaccgggagcgcugugaauacagugcucccuuuuuuuauu

SEQ ID NO: 802 - RNA, 53 nt, Escherichia coli
uuucuggugacguuuggcgguaucaguuuuacuccgugacugcucugccgccc

SEQ ID NO: 803 - RNA, 110 nt, Escherichia coli
gaaacggagcggcaccucuuuuaacccuugaagucacugcccguuucgagaguuucucaacucgaauaacuaaagccaacgugaacuuuugcggaucuccaggauccgcu

SEQ ID NO: 804 - RNA, 96 nt, Escherichia coli
gcugaauaugauucaauaucgcacgcuacucauccauccaaggauaaugaguacauagguugaaguuucaacacccccacuacggggguguuuuuu

SEQ ID NO: 805-806 - skipped (2 entries)

SEQ ID NO: 807 - DNA, 2060 bp, SOLANUM TUBEROSUM
cttcccggaacatctttcatctccagatctactcatcgtcatttctctgcttctgaatcggtacgcagatccgtgtagatcgttcgccgatctacagtttgagtatggtttcgacttgtatcggacattgattccctgcgaaatggttatgaattcagatatgtatagatcgtttgccgatctatcatttctgagttttgtctgtatgaatattgctcggaccatggaccatgggcgatgccgttggctggatgaagagtggatagatctgatggatttgttacgagattgagagctataaacctcagatccgttgttggtttgttttcaaacttttcaaggagagttctcggtgacctgagatgattttttgaccttcaattggtggaggcttcgttgttcgcggatatagttttgtcggatatcatcgtttcaatcgggtgattttaccgtatctctgtagagctcggtgcaattggatatcactggtgttctatttatgctagatctcgagggatggtaatcggcaagcttgaggttgttagataaggttatcatagttaaatttgttgtctattctcagcttaattatgaaaatttgcattagttttattgatatgcatgcatatgtacgagtaaataatgagaccgtagggaaaagagaggaaatgtgaggttctattattgttatttaccggcctttgccatgacgggtgcgctcgcatggcaggtcacaaaacctaataaagttgcgatttttgtgtttggaggggagatggtacggggttttttgcccgactttcctctccgtgtctcgtctctggcctcccttctctgttttctagaattgtctgcaagatctactcagataatcgtctcatcaaatttggcgttaatgtccccgaacgatggtttggttatctgtcaagttatagcagcgctaccttttcaagggataggtatatcatgcactcttaatacaacagtacaacagcggacaggcgaattctaggattattcagctccacactcctaccaaaggtacacttatgaattcctttcactaactttgacacttaactttcatagcatatgttcgctccataagtaaataagccttgccaatagtagtccgtaaaatctttaattgtctctgactacaggtagtaatattgttaacgacatataagagttaattcagtctacttataattattgatttatttaacaaaagaaatagcaaaatgatttattttgaatgatggaacagtttaaattaagtcaatctacttcacaaaaatataagttaatttctctgttgcgaataataattaactaacatctgtagcttaattatatctggaatgacttgactagttttccaaatgaggttttgcaattttgacgatctgaaaattcgggtttgagatactgatcttccttttttgatcgtttaagcctgccactgatcaattggtctattaaatgagactagattataacttgatgaatcatccattttcaatatctttgtcaagtctgttaaatcaacagtagtttaatcaatatccatcactaatgcaaaaggaatctgtgtacatgattgcctgttatttatttatcatcattgcgaaaatttcaatacataattacttattatttaaaggcagtggaactatcttcgcatttttttttggttgatgtaataatttataatagtggatttcaatggagtctcttgttgagctcgttctgtaatcctagagggtgattgtatggcttgagatactttcgtacgatggttgttcctcagctggttggttctctctgcatgctggagagtttaagctttttgcctcgagtaatcattgattgacctggatgttgccgtgttatactgcaatggatctgctccatgctgaaatcaattattatgttgtactttttgactcttttcctagattacactcacctgtaatttcctgtttgcttagctatcatactactgctgaaatcaaatattatgtctttacctggaaaaaaaaaaaaaaaaaaaaaaa

SEQ ID NO: 808 - DNA, 2467 bp, SOLANUM TUBEROSUM
atctccagatctactcatcgtcatttctctgcttctgaatcggtacgcagatccgtgtagatcgttcgccgatctacagtttgagtatggtttcgacttgtatcggacattgattccctgcgaaatggttatgaattcagatatgtttagatcgtttgccgatctatcatttctgagttttgtctgtatgaatattgctcggaccatggaccatggaccatgggcgatgccgttggctggatgaagagtggatagatctgatggatttgttacgagattgagagctataaacctcagatccgttgttggtttgttttcaaacttttcaaggagagttctcggtgacctgagatgattttttgaccttcaattggtggaggcttcgttgttcgcggatatagttttgtcggttatcatcgtttcaatcgggtgattttaccgtatctctgtagagctcggtgcaattggatatcactggtgttctatttatgctagatctcgagggatggtaatcggcaagcttgaggttgttagataaggttatcatagttaaatttgttgtctattctcagcttaattatgaacatttgcattagtttttctcatatgcatgcatatgtatgaggaaataatgagattgtagagaaaagagaggatatgtgaggttctttattgttgtttaccggcctttgccatgccgggtgcgctcgcatggcaggtcacaaaacctaataaagatgcgatttttgtgtttggaggggagatggtacggggttttttgcccgactctcctctctgtgtctcgtctttggcctcccttctttgttttctagaattgtctgcaagatctactcagataaacgtctcatcaaatttggcatttaagtccccgtacccgaacgaagcttttgttatctacaaagttatagcagcgctctaccttttcaagggataggtatatcatgcactcttaatacaacaatggacaggcgaattctaggattattcagctccacactcctaccaaaggtacatgaattcctttcactaactttgacacttaactttcatagcatatgttctttccatgagtaaataagctttgccaatagtagtccgtaaaatcttttattgtgtctgagtacaggtagcaattttgttaacgacatgtaaagagttaattcagtctacttataaatattgatttatttatcaaaagaaatagcaaaatgatttattttgaacaatggaacagttaaatttaagtctaatgtacttcacaaaaatataagataatttctccgtttgtgaataataattaactaacatctgtagcttaattaaatctggaatgagtaaactagttttccgaatgaggttttgcaattttgacgatctgaaaaattcgggtttgagatactgatctcccttttatgatcgtttatgcgtgccactaatcaattgggaacactattaaatgagactagattataacttgatgaaccatccattttcaatatctttgttaagtctgttaaatcaacagtagtttaatcaatatctatcactaatgcaaaagaaatttgtgcacatgattgcctgttatttatttattatcattgcgaaaatttcaatacataattacttaattaaaggcagtggaactatctttgcattttttttcttgttgatgtaataatttataatagtggatttcaatggagtctcttgttgagctcattctgtaatcttagagggtgaatgtatattgtggtatgattcagtaagtttatggcttgagatactttcatacgatggttgttccttagcttgttggttctctctgcatgctggagagtttaagctatttgcctcaagtgaaatcattgattgacctggatgttgctgtgttgtgctgcaatggatctgctccatgctgaaatcaatttttatgttggtactttttgactctttcctagatcacactcacttggaattttctgtttgctatgctatcatactactgctgaaatcaaatactatgtagaatttctcaactcttttcctggaccatcttcacttggaatacacagtgaacaaacccaattgtcagctgttttgttcttttatgatgtctgttggattagctagcatcatgcaagtgtgttaggatgactatcggaaacttgcttgacagctctcagtgttgcttggctgaattgttgagactctgtgattttcagttcataagtgaatcttggtactgtcatccaaagctgtatacattttctatgctgtgtctcagagaaagtaagatcagtccttcctgcagaaagagggagtgtaccattttattcttgtccttgatctatttgtgcaagttatgtgtacatttttgaagcaggcccatctgtgatggagtatggttacgactaattattttgccttaaaaaaaaaaaaaaaaaaa

SEQ ID NO: 809 - DNA, 2395 bp, SOLANUM TUBEROSUM
cttctctcctcttcccggaatatctttcatcttcagatctgctcatcgtcatttctctgcttctaaatcggtatgcagatccgtgttgatcgttcgccgatctacagtttttgagtatggtttcgacttgtatcggacatcgattccctgcgaaatggttatgaactcagatttgttttagatcgtttgccgatctatcatttctgagttttgtcggtatgagtattgctcggatcatggactatgggtgatgccgttggctagatgaagagtggatagatctgatggatttgttacgagattgagagctataaacttcagatccgttgttcggattgttttcaaccttttcaaggagagttcccggtgatctgagatgattttttgaccttctattggtggaggcttcgttgttcgaggatatagttttgtcggttatcatcgtttcaaacgggtgattttaccggatctctgtagagctcggtgcgattggatattactggtgttctatttatgctagatctcgaaggatggtaatcggcaagcttgaggttgttagataaggttatcatagttaaatttgttgtctattctcagcttaattattaaaaatttgcattagttttactgatatgcatgcatatgtataaggaaataatgagatcgtagagaaaagagaggatatgtgaggttctttattgttgtttaccggcctttgtcatgccgggtgcgctcgcatggcaggtcacaaaacctaataaagatgcgatttttgtgtttggaggggagatggtacggggtttttagcccgactctcctctctgtgtcttgtctttggcctcccttctctgttttctagaattgtctgcaagatttactcagataaacgtctcatcaaatttggcgttaatgtccccgaacgatggtttggttatctatcaagttatagcagcgctctaccttttcaagggataggtatatcatgcactcttaatacaacaatggacaggcgaattctaggattattcagctccacactcctaacaaaggtacacttatgtgaattcctttcactaactttgacattaactttcatagcatatgttctctccattagtaaataagccttgccaatagtattccgtaaattctttaattgtctctgagtacaggtagtaatattgttaacctcataacagttaattcagtctacttctaactattgatttatttatcaaaagaaatagcaaaatgatttaatttgaataatggaacagtttaatttaagtcgatctactttgcaaaaatataagataatttctctgtttgcaaataattattaactaacatctgtagcttaattatatctggaatgagtaaactagttttccaagtgaggttttgcaattttgacgatctgaaaattcgggtttgagataatgatcacccttttatgatcgtttaagcgtgccactgatcaattggtctatcaaatgagactagattataacttgatgaaccatccattttcaatatctttgttaagtttgttaaatcaacagtagtttaatcaatatctatcactaatgcaaaagaaatctgtgtacatgattgcctgttatttatttatcatcattgcgaaaagttcaatacataattacttattatttgaaggcagtggaactatctttgcatttttttcttgttgatgtaataatttataatagtagatttcaatggagtctcttgttaaactcattctgtaatcctagagggtgaatgtataatggtatgattcagcaaatttatggcttgagatactttcgtacaatggttgttcctcagctggctggttctctctgcatgctggagagtttaagctttttgcctgaagtatagtcattgattgacctggatgttgctgtgctatactgcaatgggtctgctccatgctgaaatcgattattatgttgtacttttggactcttttcctagatcacactcacttggaatttcctgtttgctatgctatcatactactgatgaaatcaaatattgtgtagaacatgtcaactctttacctggaccatcttcacttggaatacacaggagcaaacccaattgtcagctgttttgttctgttaggatgtcggttggttagctagcatcttgcaagtgtgttaggatgactattggaaacttgcttgacagctcctagtgtaccttggcttagttgctgtggctgtctgtgattttcagttcataagtgaatcttggtactgtcatccaaagctgtatacattttctatgctgtttctcagagaaagtaagatcagtccttcctgcagaaggagggggtctcccattttattgttgaaaaaaaaaaaaaaaaaaa

SEQ ID NO: 810 - DNA, 758 bp, ARABIDOPSIS THALIANA
cggtccgatgaagatcgtcactgatctccttccgatctacagaggcggtcttatacgaagagatccagaggctccggtttgattcctcgtcagaagacggatcgtcgcggttgagctccgaagatcggttgaagatctgatctgatcttagatcatgaaccggcctcgaggttcgtcagatctgcgttactgagcttttgatgaggtgatgaatgatgaatccgtcaatcctctatggagctagagagatgagagggtgaagaagagagaagaagctgatatgtgatattaatgtgagttaagttgattgatggatgtatgagatgtttatagggacgatatagtcctccgtaaacatctttttcatccgacctttgccatgacaggtgcgcttgcatggcaggtcaccaaaatccttaataaaaaaaaaaaagtttcgtgttgtcgaggggagttcgcacgggggattttttttgcccgggttctcccctcttgcgtgtgtgtcttgcttctcgctcttcgtacacactacctttgatctcgattcattcacgtaaattctctcaataccacaggaatattctcgatccggatcttaagatcagcggatctggagacaagctggccaaatcttaatccaacatcctccaaccaaggtaccgtgaattttgagtttaaattctccgttattctctttatttgcatcatataacaaacctgacaccaaatatttattacaatctcgaatcggccttccg

SEQ ID NO: 811 - DNA, 1108 bp, CUCUMIS SATIVUS
atcggcggacttccttagggttccatatctctgtattttgcgtctatgctcggagctctcaacggtgatcttgttgatttctgttgaacgctgccagagaatggatccgtggacgtctgaacccgatgttcgttcctgtaattattctgctgtgcgagtttcggcggcggagtctggtgaatgatccagatctgataggtacgaacgaaatgaagtcggatcttggatctcttccattctgaactccttctggagccgtggccgcttgtcatcgtcgtcttgtgtgatgtctatataatcaagactttgttgatcgtgtttgaagaagcgatgaaagagtctgttgagacttatattcatgttcgattgaagcattaactctgtatattaatatagagcgcgtcggaaaagattagtttagtgttgctctggatgtgaaaggtatcgggagaacatcctccttgtgggggatgaagctttatgtttctttgtcatccgacctttgccatgacaggtgcgcttgcatggcaggtcatcggaaaaaaaataaaatagcatcgtttttgttttgaggaagaagccgcacggggtttgcccggggtttcttcctcgttgttctgtgttttcttctcgttctctcttactctcagttcgtctatcttcaaccaaccgccatcaactgcttctcggatttctctgatttcgcgccgtttaagccaagattggggaaatttcaacactgcttgctcgctagccaagattaatcgaggattttacaagctacttggcgttggagaatggttattgctcgtgggtgaatggaagataaaatctggaacaagtatcactcgtctctgccgagtcttttcattaatggaaggtcttgtattatttctgtttttcattcgcttctgaattcagcatttttttcaatagttgcattatgcagtgctaattctttatatctattgaggtctttttttagtatgattacgtgattggacaaagtttctatttagggtttagggtggatggatgtctaattaattatgttttctcactgatttgtcaccgaagtttggaatactatgttctattattactcgat

SEQ ID NO: 812 - DNA, 471 bp, GLYCINE MAX
gcaccaggatgtaggagttttcgggatgaatggcctcaaaacgctttgtcgtttgtggttattgagtcctgagtgatcttgtcatccgacctttgccatgacaggtgcgcttgcatggcaggtcaaaaaatgttaataaaaaaagttttattggtttggggagagatcgcacgggggttgcccggggtctcttcctcttgtgtggtgtggtgtttgctgtgctctccttttcgttatgccttttattattacctatctgtcgctccactccgtattacgactctggatcagaggatacatcagaggattcgctccaacccctatcgagtaaggtattatctgtcaatttacattcgaaagggttaggctattgaattttaaataattacagataaaataatcgatagctgtgattgtaaaaattaatcataatcatcggttaggttgcaacataatacttaattcgcttaa

SEQ ID NO: 813 - DNA, 679 bp, MEDICAGO TRUNCATULA
  misc_feature (526)..(526): n is a, c, g, or t
cctctttcgaaaacaacgattcttccttcaatctcagatctcgttcgaagcttcgttctctaacctcgatcgataccagatccgtatagatccgatggagatctacatgccgatggtggttttgttcagaaatttcatagatctgaattttcgtgcatcagatctacaatccagaggtgtgaacttgttcgtcttggtttctggatccgtatcgtagataattcattcgatttttcctaagtcaaggctctggaggtatcttttttgcaggtatggtggtggatccttgaggaagatcgtagatccggtgttgaatcatagcgattcaacctatgatgatgatgatcgatgtatcagcttccgatttgttctctgtggcgaattttggcgaatcaacgttgatgaatctcgttttcgtttaacagattcagatgttacacatctcggttattgtaaatatgtatatatgatgatgaagattggaagttttcgggttgggataaaatggatgtaggatatatcttanggatttaagtgcctttatgcgctgagcgtatgaaggattaaatcctgagtgatcttgtcatccgacctttgccatgtcaggtgcgcttgcttggcaggtcaaaaaataataattaaaaaagttttgttggtttttgagagagatcgcacgggg

SEQ ID NO: 814 - DNA, 316 bp, LOTUS JAPONICUS
gaattcggcacgagctcattccattgaatggatttgaggtattgaatcctgagtgatcttgtcatccgacctttgccatgtcaggtgcgcttgcatggcaggtcaacatgttaataaaaaaagttttcgttgggtttgggggagatcgcacgggaattcccgggatctctttcccctgtgtgtgtggttcgtttgcctgatcctttcccttttgcttttaactttttgccctgtcgctcggctctgaattactactactcaccactcggtagatccatcatcagataagctcctgcaagtcatcgacgaccctatc

SEQ ID NO: 815 - DNA, 448 bp, POPULUS X CANESCENS
  misc_feature (411)..(411): n is a, c, g, or t
gatttatttacgaggatgatgtataagatatatatcgggaccagaaagccaagaggccaaggtcttgtttattatctttgtcatccgacctttgccatgacaggtgcgcttgcatggcaggtcaaccactatcttaataaaattatcattttcgtgtttggaggggagatcgcacggggatttcccgggttctcccctctagtctctgtgtcccgctttctcttctctttgccttacaatccatggctaaccttgatagcgcggccgactccagctagcaatcatggcacgatattgcaatttttgacggtgacccaaaggtattctcctacttatatcctatatcctcaattcgaatttcctgcacttagaatcatgtagtcacataccctttcacgtagaatcagtcgntagcaagagacatgaatttgtaatctgggttctcatg

SEQ ID NO: 816 - DNA, 385 bp, CITRULLUS LANATUS
taggaatttccttttcctctctgtattttgtgatctccctcggagatgactgtgacttattctctgttgaaggaggcgtgagaatggatctgtggaattctgaagccgatgatcgtttcgctgattattgtacttgtccattgtcggtggtggattctggcggatgattcagatctggcaggtacgaacgatatgatgtccgaactcggatctcttcgattctgatctccttctggagccgtcaccgcttgtcatcgtcgggctcgtttgatatctatccaatcagatcttgttaatcgcgggcgatgaagcgattttaagttgatgtatgagtagtgtactcatcttcgatggaagttatgtctttgtaacataatatcgagac

SEQ ID NO: 817 - DNA, 432 bp, Unknown
  Synthetic contruct
tcgagtttttttttttaattttcagattcgtgtgcccttcggaatacctggaccggaggcggacaagaggctggccgatatgttttacaagttttgatggcaccgaacggtttgttcaccgttctgggatttgatgtgaaggctaagctcattgttcatctttaaggtgcaaagcctggatgtgtgataaataattttgagccgctaatttggcatggctaattgatcatgcgggtaggcttgtgccgcataattgggagtgaattgcgggattgtggagggttttataatggatgtaagagatgcatcaggacagagaagcctgaaattaagatggctgagacctgattattctttgtcatccggcctttgccatggcaggtgcgcttgcatggcaggtcaccaaaatcttaataaaaatatttattttgg

SEQ ID NO: 818 - DNA, 661 bp, LYCOPERSICON ESCULENTUM
ccggcctttgccatgccgggtgcgctcgcatggcaggtcacaaaacctaataaagatgcgatttttgtgttcggaggggagatggtacggggttttttgcccgactctcctctctgtgtctgtctttggcctcctttcttttctagaactgtctgcaagatctattcagataaacgtctcatcaaatttggcgttaatgtccccgaacgatggtttcgatatccgcaaagttatagcagcgctctaccttttcaatggataggtatgtcatgcactattaatacaacaatggacaggcgaattctaggaatattcagctccactctcctatcaaaggtacacttgtgaattcctttaactaactttgacacttaacttttttccatgagtaaattgccaatagtagtccgtaaaatctttaattgtttctgagtacaggtagtaattttgttaacatataagagttcattcagcctacttataactattgattaatttatcaaatgaaatagcaaaatgacttattttgagtactggaacagttcaattctaagtcaaatctgtttcacagagatataagataatctaaccatttgcgaataatgataaattaacatctgtggcttaatatatctggagtgaactaattttccaaatgagg

SEQ ID NO: 819 - DNA, 638 bp, GOSSYPIUM HIRSUTUM
cttcttcctcagccttacggtttctgtttgcagatgagatttatcctccgatgtctttccttacggggattctgtcctcggatctgctcgcatcggttcaacatctcctctttgctttctgatgttgtactatgaaaggagtagcgttgttggagctatggttttaaaggcctttgctgggaaaggaagcaaagattggcagggttcaagttcttgcggctttttaaggatggtctcatcaagggctgagatttatacgctcttcagatgtaagttgaaaataaacaagacatctgattgtgtgaaacggcgacgtgagattgaatcggctggtttgtttatgaaaggatgtgtaagatgtcagggcaaaaagtcctactgataatatgtttattgaaggggactgtgtcctgatctatctttgtcgtccgagacctttgccatgacaggtgcgcttgcatggcaggtcacccaaacttaataaaaattgaacttttttgtggtgttttttgaggggagatcgcacgggggtttgcccgggttctcccttctgctgtggtgtctcgtatcgtgtcttctcttcttttttcttctctttcccttgctctctttccattattttgcatcatacgggatgc

SEQ ID NO: 820 - DNA, 557 bp, ZEA MAYS
  misc_feature (523)..(523): n is a, c, g, or t
attagtattacaagactgaaaatcgagctgggtgaagcggacgtcaacggtattaacgtgcgtttcaggatcttcagttcgattggtgtcaggatttcttcgattcattatgcaagcacagaatataaaacgttgctcgtaatagaaggagcgcagaacgacttaaaaattgtttgaacataattaccgattgtaccttgggagtcgcgtcgtagctccatgtttttgttgtagatctccgtgctcgatgcaaaaatccggcgtgcgcagcggaccaacaggcgcggaaacaacggatacaggaacacggaacaagatgagggaggagaccccggggccaaaaacatcaacccccgtgcgatctcctcccccggcacacaataatcatctttttattaatacctggcctgccatgcgagcgcacccatcatggcaaaggcctcctctaggatgtcacatctttttttctcttttttgctgtcgacgcacgcagacttttacatgctgcagcttacctcgcctnttccaggcggcatatctcatagatctactaataa

SEQ ID NO: 821 - DNA, 711 bp, ORYZA SATIVA
  misc_feature (608)..(608): n is a, c, g, or t
  misc_feature (635)..(635): n is a, c, g, or t
aaaaaaaacgagagatgtgacatcgcaagattggcctttgccatgttgggtgcgctcgcatggcaggccaccgtaataaagatgatctttttgtgtcctggggggagttcgcacgggggttattttgtttagccccgggttctccccccaatcttgtgtcccgttgtcccctggttgtcctcagatcccactgcgactacatcgttcaacgatcgcgcgtgaatctaaacgtggagctacagcgcaacaaccaagaaattgtagcattctctcgtcgttttaaggtgacctgaagacaagttcaacctgaagtcacccgaagaaaggagtattcttgcgaatcaaacttggttgaggtggccgtctgggatcttgacatctgaagcttgacatctgaagcaagatgatgatgttttgctgtgatcacctgtgctcattacatggtgcttcaggaatgacttgtctaatgttggtaatgttggtgctcatcaattttcatggttattgttggaccgactttccggtatgctggattaagctgttgcagctttgaagtctggggtgccaaccttgtttggactaattgccagatgtatcattataaatgncttcggtgagagttgagcaccatgganggtctggataacagttccgtctctgctgcggaaggttgtttcttgaggtcaatccggaggtaaatggctgaatttg

SEQ ID NO: 822 - DNA, 747 bp, ARABIDOPSIS THALIANA
ccacgcgtccgcccacgcgtccgcaaagagagaagccataaaaaccctaacaacatccattgttgaaactctctttggcacaacaccaaacacacaaaatatggccatcccctaaagaaactgaagctcaagaaccctctgaatctatcagaagatctcaccaataaagggttttgagaggttcaagtttctctttatgggatggccccaaacacaagagtactcacaaaaggtaagaagaagaagagatctcttggagattatgattgtgaatgttcgtgtgtgggtgttgtgtaaatgatcatcatcatcatcatcatcattatccaatatcataattatcaatatggtgatgatgatgatgatgaggattgtgtgtgaatggagcgatgaagattgcatgaaggttgatgaagacaagcttgatgtgtcttttgttatccctcgtttgggcaacttcgatcctttggcaagcttcggttcccctcggaatcagcagattatgattactatagctcttatatgtttatgttccgtgtttactttgtttccggtttagtttggtttattgtctttaatttcgtcatgtttgtgttgaatgtgtgttgttgttatcccgtgtgatgcaccattcaggtgtgttcctatgtgtgtgtgaggaataaaagaatattcggttttgatatctaaaaaaaaaaaaaaaaaaaaaaaaaaaaaaaaaaaaaaaaaaaaaaaaaaaaaaaaaaa

SEQ ID NO: 823 - DNA, 504 bp, MEDICAGO TRUNCATULA
aatcctataacaaccaaaccaagctaccatttcaaaccctctttcattttcactccatctcaaacatgtaacattacttatacacctctatcctttatggaaccacatgggatagaggagaagtggatggtgtgtgtaatggtttccaattttcatgctccttttctttcaagattataagcaatatgttaatgattgctgggaatgaaccttctttgttgatgggattttctctttggaaagggcaacttcgatcctttggcatttttcaattccttcatcacaattcacactcttgtgatttgatggagctttcatatggcaacacccctattatgtaataagtattgtactctcttcttttcttatagtaccctttttttgtttgtgtgttgttgtgtgacttcaattttcaattgcaagctatgtttgtataatatgcctactatagactatagaaggtatatccaataaatgtacgtttctaaaaaaaaaaaaaaaaaa

SEQ ID NO: 824 - DNA, 542 bp, ARABIDOPSIS THALIANA
aaaacaccacaaaaacaaaagaaaaatggccatcccctagctaggtgaagaagaatgaaaacctctaatttatctagaggttattcatcttttaggggatggcctaaatacaaaatgaaaactctctaattaagtggttttgtgttcatgtaaggaaagcgttttaagatatggagcaatgaagactgcagaaggctgattcagactgcgagttttgtttatctccctctagaaattgggcaacttctatcctttggcaagcttcggttcccctcggaatcagcagattatgtatctttaattttgtaatactctctctcttctctatgctttgtttttcttcattatgtttgggttgtacccactcccgcgcgttgtgtgttctttgtgtgaggaataaaaaaatattcggatttgagaactaaaactagagtagttttattgatattcttgtttttcatttagtatctaataagtttggagaatagtcagaccagtgcatgtaaatttgcttccgattctctttatagtgaattcctctt

SEQ ID NO: 825 - DNA, 474 bp, LYCOPERSICON ESCULENTUM
ttcctttgcatggttttactagtgggcaaccacccttttgccaccaacttttagggcaaaagggtggtgatggaagaggtggctctcgtcgttgattcaccggagatggtggcttccttcatggaactattgtccattgataagttctcttggtggtgtttttttggttggaaagggcaacttctatcctttggcattttgatggaggaaatggttaatgaaaaagaagagcaatctcaaggataaggcagattattcatgttaatcctatagatctctacttaattactagtaataattagttgttgagtttaattataatgaatattattgttgtgtgtttgtgatctattttagtttatgatcatgtttgtgtactacctcttggcccaaataaaaaagagtatttgtgtttgtattataatgtttgagacttgtatgttatttcaaattattgatgaattttgattgtgg

SEQ ID NO: 826 - DNA, 393 bp, LOTUS JAPONICUS
gcaaacatgtaacaatgcttttactaaagcttttctcacccaccccactgccccatggtgacacaacttggcagtgggggtggtggagatttggatggtgtgtttagtggtatttgtgtttctatgctccttttctttcaaggctaagattgctgcagggaatgaacctccaatttcttgaaggtgatttctcttggaaagggcaacttcgatcctttggcatttttcaacttcttcacctccttcgtattggcaacatctctattatgcagtactgtactcttctctgattttcttctggtccttgttttctttgtttgtaatgtgattttaacttcaagctatctctatctgtatgatgttgtctactatgagtatgaactaaagtagctt

SEQ ID NO: 827 - DNA, 431 bp, GLYCINE MAX
ggtggatatatggtgtgtttggtgtggctataatggtatcttccttatctttccatgctccttttctttcaaggctaagcgaaaacgctccttcttgaggggcggtttctgttggaaagggcaacttcaatcctttggcatttttcaactcctccatctcacctcacacactgtgatttgggctcttgagggacatctcagaatggcaacacccctttttatgtgtaccgtactattttctagttctttttcttgtacaacgaaatttagatgcagtttcttagatgttggtgttgttttttaatcaaaattagagttttatcttgatagtaccggattatcaaggtaaaactctgattcttatcacaaaatggcaccaaaaatactttaagaactgtatgcaagttttttgttgtttttatatggtaatg

SEQ ID NO: 828 - DNA, 444 bp, ARABIDOPSIS THALIANA
  misc_feature (14)..(14): n is a, c, g, or t
  misc_feature (41)..(41): n is a, c, g, or t
  misc_feature (79)..(79): n is a, c, g, or t
  misc_feature (164)..(164): n is a, c, g, or t
  misc_feature (192)..(192): n is a, c, g, or t
  misc_feature (285)..(285): n is a, c, g, or t
  misc_feature (342)..(342): n is a, c, g, or t
  misc_feature (402)..(402): n is a, c, g, or t
  misc_feature (428)..(428): n is a, c, g, or t
  misc_feature (433)..(433): n is a, c, g, or t
  misc_feature (441)..(441): n is a, c, g, or t
accaaatatttatnacaatctcgaatcggcctttatttttnagcatggaaatagttgcctcattttcaaattgactgtnataatacattattcacaaagtacttctaccaaagtattcgtaggaccttaaattgtctaatcgaacggttccatggcagtttgtnatattctccactttatctatctactgtngtttgatgtttgtacttttcctgtaagaattctcattggaagaaattgtacccagttttatgattacgtaaagaaatatcagtagtgaatatnctgacctattctagaaaatagcgctgtagtatttagtctctcgtatggggtttacgntttgggtttatcacggctgcattcattagcaagggctatggctagagtagctagctcccnggggaagtaatacgggtatcgaaggnctctnattttggngga

SEQ ID NO: 829 - DNA, 506 bp, ARABIDOPSIS THALIANA
  misc_feature (415)..(415): n is a, c, g, or t
  misc_feature (480)..(480): n is a, c, g, or t
  misc_feature (490)..(490): n is a, c, g, or t
  misc_feature (498)..(498): n is a, c, g, or t
ctcccaaccctctatatataacaacgcgtcgacctaattaagttctcatacccgctttattcattgattcattcaatttttgttcatcgttttctctagatctctcttctatatagtcctctcggaggttttccgatgaagatcgtcactgatctccttccgatctacagatcggcgtcttatacgaagagatccagaggctccggttgattcctcgtcagaagacggatcgtcgcggttgagctccgaagatcggttgaagatctgatctgatcttagatcatggaccggcctcgaggttcgtcagatctgcgttactgagcttttgatgaggtgatgaatgatgaatccgtcaattctctatggagctagagagatgagagggtgaagaagagagaagaagctgatatgtganattgatgtgagtaagttgattgatggatgaatgagatgttatagggcgaatagtctgcggaacancttgcaccgncttgcatncagggggg

SEQ ID NO: 830 - DNA, 403 bp, ARABIDOPSIS THALIANA
  misc_feature (30)..(30): n is a, c, g, or t
  misc_feature (253)..(253): n is a, c, g, or t
  misc_feature (321)..(321): n is a, c, g, or t
  misc_feature (324)..(324): n is a, c, g, or t
  misc_feature (332)..(332): n is a, c, g, or t
  misc_feature (335)..(335): n is a, c, g, or t
  misc_feature (368)..(368): n is a, c, g, or t
  misc_feature (370)..(371): n is a, c, g, or t
  misc_feature (377)..(377): n is a, c, g, or t
  misc_feature (379)..(379): n is a, c, g, or t
  misc_feature (384)..(384): n is a, c, g, or t
  misc_feature (386)..(386): n is a, c, g, or t
  misc_feature (389)..(389): n is a, c, g, or t
  misc_feature (397)..(397): n is a, c, g, or t
tgacttattaaatttgaaagatcccatcanaatgcaaaagtctaaaaaaaactactgtacgaaatgatacaacaacagagtatatatcaatcgaaactgcaggctcgaatcgagtccaaacgccatcatatcaaaaatcagagaccttcgatactcgtattacttctccaggagctagctactctagccatagctcttgctaatgaatgcagccgtgataaatccaaatcgtaaaacccatacgagagactanatactacagcgctattttctagaataggtcagaatattcactactggatatttctttacgtaatcatnaanacctgggnacnatttccttccaaatgggaattccttagcaggtnanngtacantncatcnanacnaacagggngatagt

SEQ ID NO: 831 - DNA, 646 bp, ARABIDOPSIS THALIANA
tgcaacatttttttttttgttaaagactactaaaacaatctaggtactttcatttcaatatattcaacttgaaattatcaaatttgttcatgctacacaccaaactcaaacttgcactactacacaccaaactcaaaagtaaaggaaaaacagaaaaataaaatcacctatcagtttgttgttgatcagtcttcacaccataacacctatatatagctttgagaactacaaacctttcggaaagaaataatataaacatcaaaaaccttttttttttgataaaaaaaacattgaaatcctgtttcactcaggtgttcctttgttttgaactactaattaacatatatatatatatatatatatatatatacataaccattccaaggcataacaatctaaaatctatcccggataaatttttgttagatttttactatttatttaactatttaagggcattattaatcacattccctctttttgtcaatgactaggtaaatataaaccgaaatctatagaaatccttgattacttatacaaggcaaggtttgcaaaccacatagattgtttatagttatcaaagaggttacgagtgaaggttcatatatttgagaattcgttggaccaaaaaaaaaaaaaaaaaaaa

SEQ ID NO: 832 - DNA, 597 bp, ARABIDOPSIS THALIANA
atcatcgtcaccaactaataacagaggatttggaaaagtaattctagagatccaaactatcaccttcgcttctcactatcgctcaactcaccatcgtcgttgaaactcaccatcgtcgccaacgctaattggaggtcgagatcttgccgtctttttcatcacgagacatgattcatgagtcaccgccgttgcttcttaccgttgtcaaaatcgttgttgttgcaggagctcaatcaccattgtaggttcagtggtaagggtaaactcgtaaaatacttgcatagaaaaatacttaaatctataaggcatatgagatatgtctgttttcataaagatataaacttaattgtgtcaatcccataattaatccctaaattgtgagttgcagttttagagtttatgagtcttttttaaaaaaattattatgttccattttaatatattttagtaccaggtttacttccccttttttttgggttttaacttctttcttgtttgagttttgctttagagattttagtttttgtaaagcaaaaatttgctcctgatgaatcatgcctaatgctacctgttcttattgtcgaaaaaaaaaaaaaa

SEQ ID NO: 833 - DNA, 1967 bp, ARABIDOPSIS THALIANA
ctaaatattattcaagaagtcaatttacaaatcaatgcagatcataagtctcgtttctccaaaagtacacttgttggtttcccttcttctcttcttcaaaatcttctgatcatctcattcttagttcataatgtccaatataggtcaccgagtaattttcattctcgtctggcagccaatgaaacctgaaaattttctttacttgtccctcaaagcaccaagagatacttcaccgtgtgtgttttttcacttacgttgtactgaaaatcatagttcatccatcctataatactacagtttagacacaattaacaaaggaacaggttatgaaaaaaaaaattgcgatcaaaaaaaataccaattgtttagacatccacatgcaaacgtcaattttaaaaaagtcttacaatcacttcactcataaaacgtattgcatattgcaaaaaaatgttactattaccctcaaacgatgtgagcatacaatgcttgacgctcatctaagtttggttatgatatcacttggaaagatccgactacaattatttgtggacctttctgttcactcctagcccgtaggcttcatccacgtctgaccaccgatgtcaaacttttgaaacctctaagaaacttgttattggtcatatatttcaccacaaacgtaaaaatacgtgtttttacatctcatgatattaataatcatttataataggtcattttctttagatatctctaacttagacacacttaatgtgtaggtttttcaaaatgagtcacgaaaaagttaattacactttggtgatacatgaaaaatcaaattaataattttgatcatctcaacacgcacaactatttttcaaattcaagttagtacaaatactatacgatcctcatttactagacaaaggttatattcttaaaaccaaattaactccggaaaaactttgccccctccaaaaccaaattatagcttcagatcatgtattatgtatatgaatcacaaaatgtgcaggtcaactagttgttctccgttgccttaatttctcttcttctccacatgtcgttccctcaccatcagactcccaacacatgttaccatatagatattaagccataaataactcgtcatacaattgtacaaagtatttatagttacgttgtacaagaaaagtatttagaattggttaatttgcgataagacctttagtctacttgatatatttttgcatacacgtgagaaatttggtcaatagaaatgctgatctaccgataacggttttgatccttttcacgccaaattttatagaaaatgatcatatgccaaaaacatatacaacaaatgaaattgcatgttgtggtaaaatattgatacaacgttttaattaattgtaatcatttgatagaaaacaagctattttaacattaataagttatatataataaataagttactattaatattttacatttacttttataaagatttatttattttaaatagtttttaggtttcatggtatggtggcaatgaacatgctttcaagccattttctgcataatagtgtttaacattacattacattacatatattcattaaacctaaatgttaaaattatatatgctaaagttgcaatagacatgcaatttgaaattggaagaaaatgctaaattaaatctcatgccattttacagcataatttgatattttagacataggaaaaacttaaattttatcttgattgactaggaaaacttaaattaaccgttagtgcaaaaaaagaaaggattatcttcacagaaagtatatagactaatctagtgcagtttcctgtttgacatcattacacctaaccttaaaattttcggagttataaaataggaccgacctctgtttctaaatgtttcagcggaattaaaaaaaaaaaaaaaaaaaaaaaaaaaaaaaaaaaaaaaaaaaaaaaaaaaaaag

SEQ ID NO: 834 - DNA, 1359 bp, ARABIDOPSIS THALIANA
ggagagttttcaaaagtctgaagcagagagacatacttacatatgtgtgattcttccctaagaggaattctcaaaaatcaaagcatctctcctcttcttcctaaacgtcgacgagtggagtccttggcgcgatcaagattcaaaagttcacgaccctccttgccgtgactggagaggactcccctctatttttctctcgccgatctactttctagtcaggacctctgtgcaagatttcgaagaaaaaaaacatcatcaatcacaaatcctctgcttaagcattcctcgctggtggtggtggcgagaattctcttcgaagaggttcctttcccgacgctctaagcttcatcctctttgaggcggtacactacttgagggcggttgtcctagccctctctgaatcctacgtggatctcattctttggattcacagttgggtggtggttgttcttctaaaaagaactgcagtgtggtgaggtcgtatgactctagacggttagtaacggtggtggagctccttctgttgatcatgcggttatttccctgaaaagaagaagttgctccgaggacggcaacacggtctctcaaaggagcgttctgtcaaaattttgggtgaggcaagacaagatcccttcgtattggtcacgacagagacgccattttgatggtgtgagtttgttggttcctgttaagtttttcggcgtgggaaggttggagtctaatagtgtgattagcttctccgttagtgttatggcaaaggttttctcagggtggtgtgtttctacgttttcagcagtcactcggttctctctaagtagggagtcggcggatctaagcccaaatcccacaagtgcgcacgccagatataaccagtgtttgtaggcagcgttaggagtgacgatcgcgttaacgatgaccgtttcagcaggtagaggtgtctatcaacacaatacaggtaagagcagaggctttctctggcaaacgacaatacgttgtcgtttcggctaatggagcaaagaggaaaatgaaacagatgggcctcgcacaaaccaagcccaagaagcagaggcaacggtcatctgtttcaagtaaacagagtctagaatcttcctcaagtaatgacaaaagtggagctggcaataaggagacaacgacctcttgtacggcaaagcagttcaacgtaaagctcgagaatcgtttctcaacccttgattcagaagactcaacttgatgtatgacatctgtccaatgtctctagggttttagctaagtctccacctttgtatttaagtattctcaaactcaagttgtaagctttaagcaaactatcaatgaaaacctaaaaagattgattttgtc

SEQ ID NO: 835 - DNA, 1406 bp, ARABIDOPSIS THALIANA
cttcaaatggcttccttatcaaccacttccctaagcttcaaagctccgtccaccacaatttcacaggttttaaggaaagcctcaagttctcagtctgtgacctttggtcgtttcacgtcttcaacaaagagccttcgtcttcaaatcagttgtgccgcgaaagcagagacggtgcagaaagtgagtgacattgtaaaagaacaattggctttagctgctgatgttccgctcactgctgaatccaagttctctgctcttggtgccgattctctcgacaccgtggagatagtgatggcgttggaggaaaagtttaacataagtgtggaggaatctgatgctcaaaacattacgaccatccaagaggcggctgatttgatagaggaccttgttcaaaagaaacccgcggcggaaacttcctgaaccggctgtcttttctttcctgacctattcttgccttcagagcgttttattacggtatattagaccgtttgttatttttatttatttcggaagctaagattgggtgttgacaatgagacaactttggtacgttgagattgattctttgtggtgattggtcttatgacgaattcataattgtgtattgtttcaaaaaagctgagtatgtgtggtgatatagagtttgtgtggtgagagcaacaatggagtttgtgaagattgtgtagcttttgtactttacatcacggagaaacctctctagattttgattttgtgtagatttagtgcaaaacaacgacaaaagaggcggtgatgacgtgacacaactccataaatcgaaaaccagttctgattgatttcatcttcttcttgaatgaagcttttttgatttttttttgaatcactggtacagttctgatttacttatttttggccagttatatatgttagttatacaaaaaatgaattttgattgaattttttattggccaagttatatatgtgagcttgatcaaattcaatttgttatagtcatcgaagccaacaaaattcgaaagtgaaaacataataagaaagtttgtttcctttagttgccatttcaggcggtgagagagttagcacatcgtgttttgggttgatgtaggaagaacgaagagaaatctctggacttgaaaaatcatggaatctttgctttcacagccgatgctttgcgtttatactctaacgctttacaagtatccatccacaagtgaccatttcatgttgaacatatagttagagttatatggatcctttacatttttctattgacttaatcatatccaaaattgtttatggatgttgttggacgcacccgaccatttatagtcgtccatatggataaaccaatttgtaaaattacatatgaacacaagttatatgaagggaaactcaagttaacaccaaag

SEQ ID NO: 836 - DNA, 1596 bp, ARABIDOPSIS THALIANA
ttaaactaaggtgtatagctttctaagaaaaggaattgtgttaaaatatagatcggttgttagacatttcctaattagatgacaaaactctgtatctgaggttaaactaatgcttctattttaataacaaaaaggaattatgttaatatatagagagtcggttatagaagttgtgaaaatgtgaatgcatgttttagtccacttacaaaaagccacaaaaagtcacaaacagtggactttggattttcttgaaacgtgattacaaaggtcaaaatataagaatagtatgttctttaacatcttttgccttggaaataattgtaggcaaaaaaaaaatgacccattatctaaatttttaatttaacaaaaaaataaagtaaaataggcaagattggttttataccccaatggagaaatatttcaatatgtcacagtattataaattttaacggataaattttctttcaaaacatgtaaagatttacgaaaatacgtacgaatatatttaccctacttaattaaaaaaaaaatgattcaaaattataatgatactataaagagaatgcagctgtagtttcacttgtattgtttactttatcttactaaaaagataagaaaatccaaacacataataaataaactaagacgtaatatgctttttgttagattcttcgccattaagctaagctctcatcttcttcctcttgtgtcaagacctacacaccattttctcccctaagtcttaacttaataagttgtgtagaaacttaaaaaaaatcttttatgtatagagttttatctgatattttttttgtcaattacatgtacttaatatgcgagcttgaatctggaatcgtttctagcagtagaaggataaagtgcgtgccctctttttatcacattgaaaaaaaagtatatgaaaatgatgcattttattggtttgaactctgaacctactgagtatctataccaagaaatcttacacaaaattggtctaataactaatatatattttgaggccccaaatcaaaattttatgtgcttgtgccaaggaaggatcggtcatgatagattgatagtaacacaacgacaatactaacagaaaccaataggctataaatcctattcattttattttttttacaatatagttatattttacaagtttttcagatattattgaccctctagtattttaccaattatatagtttaaatggtgacaaccttaaacacccagtcctttatattaatcaaaaaaacaaaaaaaaacaaaaaaaacaataatcttacaatgacaatgtgaaattaatacactatctctgtagttaaaaagtagtgctacgaaagtgataaaagttaaaaagcagtatccaaacaaaaggtataaaaggataaaaaaacaaaagttaaaataatacagtaataatgttatctagagcactaatcgacaaacattccaattaatctcttagattgactctgattatgacattaatacttagcacttttttagtggacaatataaccccaatgtgagtcccacatgttattcctcattcctctctataaatagccactcttggttcattttgaa

SEQ ID NO: 837 - DNA, 624 bp, ARABIDOPSIS THALIANA
ctcacccctctctttctctctctagcgatcacacttccaaaaccttgcggatcggatctttcttccgtggaagcagctctctagtctcacaattttgactgattttgaaacgattagggtttaatttgttggacttgggaagtgtaatggtgttttgctcagaaatcgtttgttttagtgccgacggtggttgagtaattagttagttaaaatccggcggagttagttttctctctgcggtggtggatatggctgcttcacctgcgaagttcttcttcgggtttctcattgtttccatcgttttgtggatgatcttcatactctgttccaggttgtttgcctggatgcttagtagagtgttgggagcatcggttgtgtttcgtgttggtggttggaaatgcctcaaggatgttgtggtgaagtttaaaaagggtgccattgagtcggtgtccgctagtgaaattaaactaagcttacgccaatccttggtcaaacttggcgttggttttctttctagagacccaaaagtgcaagtcttgatatctgatcttgaagttgtgatgaggtcctcaacttcaacaacaaatttacagaaagccaaaaaaaaaaaaaaaaaaaaaaaaa

SEQ ID NO: 838 - DNA, 2090 bp, ARABIDOPSIS THALIANA
cgaaccacgccatgtctctagtccccggcggaatcgctgttccccttttttgttaagactaggaaagctcagagtcagaggcatgtccagagtttgatcgtcgtatccttgggcttatcaatatcagctgactctcagagtttgctctcatgctttgcccaccacaagggcttagtggcggatgccttgatcgtcctctcggagttgtccctgaacagaatccaaaggctagaagctcttttcagaagatttgaaacctgcttcttgctcataccaacatcatttgaggagcatgacaatcttctttccttggcatatgacaactgctcatctgatcctgccccaccctcatcctcgctacacgaagcccattcacttcctgattccgatggttcccggagattatccttttcacacgtttttgcccgttccacaaccctatatgcaaacatttgaaaaggaacctgtgaaactttggttagctctgtagattcccaagaaagcttctgcaatagctttgtctcgatctcgcagtaatccacagtaagcgcaatggacgggtcttgttggctactactgtaacattccgggttcaaagctagaaaccgtttgatataccggagaattctgcttatcgatgagaaagttggacgctgattgggatcagcatgccagcatcgttttgtcaggtggctatcttcaaatggtactttacctgtcagaagctcaaagctaaccatcccaaagctatacacgtcagatttatcactatacttaaggcttcctgcagttccactctgttcttgctcctctagaacttctggagaataccatatgaatggaaaattctcattctgactcgtcaaagaagcacttttactagagaatcccttgacagaattcaacccgaacccaaaaatcttcccaagcagatacccatctccggattggttacttcttggtttgacaagaatgtttgatggattcaactctccgtggtatattctcttcgagtgaagatattccatacccaaagctatctgaagcatcagatcaaccgcgaccgggagggagagagtgttcttcttcctaggaccacatacctctttgatgtgcattccaagggttttcctcatcagttccatgaccaagaaacactctttcttctcctcatccgtgaatccgcagaggtaatatacaatgtttgggtgtgaaagagatagcaatggagtaatctgaggaagcaaagcatcaatatccccaaagaaatgcctaagcgcaaagctctcgcctaaccagaagagaagaaggaaatagcttggggctctcatttccatctcccaaatgcttcaaaaggaaatcagccatcttccgttcatgtttgctcgaaccggactgctttttctcctgaagctccttgatcagaatccaacggtcctctgtccaagcactctcgaaccggttgcagaaatcctcggtcaccaaatactctctcccaaacttccaggtaaacatctgagagtcattccattgcttcatatacttgttagaatgcaccaacctcttcttgctcatctcttcttcatcccaccctgaaacctcactcgctgtctcaatagcctcgacgatgatcggtagacaagagagcaagttatgtatgtgaagctctgtgcaatttctattgctatgcaacacaatagccttagcccaaaacccaagtttaggctctaaagacattctaacataagcctctccttctctaaccacccatagaagctcccttaagggctgttctagtatcttccatttagtgttcttctctttgaagcgtagattggatcgcatcgacacagaaattgactcgtaggctgcggtgaagaggtcgagtaatagagagcattgacgctggttgatttgaatgctgtccttgaacaccattaacgctcttatgcttcctaatacctctcctatctctcgaaattgatccatttcacacctcttcaattgtacacaaggccttcggaatttgtattataaattattgtactcataagaaatcgtatcacaggaatggatttgtgaaagaaatt

SEQ ID NO: 839-840 - skipped (2 entries)

SEQ ID NO: 841 - DNA, 1029 bp, ZEA MAYS
cacaaaccgtgaatattgcaaggctgggtcattactaccattgaaactgaccatgaaccatctgaatcgttcaggctgtacagacgaagattgacatcagggtcaagagttagccttctcttgatgcctggccctacgtcggaggccacaagcgcctgatcatcagcgaagtcgctcgagtcaagcaccccgctatcagtaagcattcctaacctcgtacggttatactggttccgaccatcctggtagatgttctggtcagggtctggccagtatatgtctgagacttgaggcaagtcgctgaagcagaagatgtagttaccaggactacgcgattgggttgtggggactaatctggtcgcagcagtgatgagctgcgtcggcaggaaagtgttcgttggggaatcaaaactctgccatacagtgttacctcctgagtcctcgatgacgaggttcccagtgttcaggagccgagcacgctggacaccggtgaagttgttgccatcagctcgccacacggctgcgtcgtcgtagtcggtgagcaccatgttgtcatccttttgcagggttagagccgacctcctggcatggacagggcggtcaaggtttgcgctccacacgatggtcttgttggccgccgcctttgagtaccatactgagaatgtgaaggcatgggtgtagacttcatagaagccagaggagaatgtcccgtctgatgattgtaaggcgctggattcctaggactcgactttgagagaggaacccagtggtaggatgtctcaggatgatgcagctcttggaaatagagcgatgaaggagaggatggcagcagtggaaaggagagctgcaagaggacgagacatcattgctgagatggggttttggtcgatgaacgcgcagggccagctacttgtatccgtggtatgccgagtttctgtcttagtcctgatagcgaaacgccattacctggtcaaattcagagtgccatggagttcaagttgtttaagtttaaaaaaaaaaaaaaaaaaa

SEQ ID NO: 842 - DNA, 3364 bp, BRASSICA OLERACEA
tctagataattttatgaaaatatatagatttttattttaaagagatggaagacttctttagaacttatgttaaatatggaccaacacatgatttgtgaattgttatcccaataatataacccaaatcgttgccaaaaaaaaaaaaaaatatatatatatatatatatatataacccaaatcaagttttcttttcaacacctttccctaaacttgatttgcttattctgagtaacctcagcattttgatattgtctttccgtttgagtggcttagtgaagatattagctacttgataatttgtcttcacatactccaattgcacgttcatattggtaacccattttctaatataatgataacgaatatttatgtgcttactccaatgatgaaagactggatttcaccaatgctattgttgatttgttatctgttggaggatggaccttcagcctgttcgcgagttgaacatatttgaataaggcccagtacggggaagccgacccgtttggaatgataaccctcgctcttcacgcaaacaaggacaacgaggaacagtggcctagaccgtaggctgcactcttcgccgatcatagccaaacacgaaccgattctctgacacaaccaccgctagacggaaggagaaagggaaataggtctttatatacaggagagaattcaatgagaaagggggatccagaacgagagagagagagagagagctattgacaacgagttcactttcttttgtatctttctgttcggcaaacagcttaacacttcaataaacaagacctttggcccctaaactctatttattattgtataagatccttaaacttggttacaggttttaggattcaacaatattggcccccacagtggagccttaaggattcctacgcacaaagttcgcaagcgcccgcaaagcaggaaaaatgtctacatcatccaccggacaacccacgactaaagcaagtgaccgacaaaccctagcaccgcgacaaagtggggtggagtcaggaaagagcagccagattaaccccggaaatatgcgccacccatcctcgtccgccatcgacgccttgctctcaaacgccaggaccgcttctaccacgacaccatctacggcacagacgaaccaccctcatgaaatcatgaccagcccggcgcaacccctgatcacccgatggaactgccgaacccaacccacacgcggctaaaaaagatccccaaatactgacgttacggccgtactacacacactagtcgaaaaagtagatgaactcacttgccgaggaacgcaagaaccagcgacctaaacctagatgccgactccacataactgctggaggaggagcagacgacccgaaatgttgggcgcatacccatcgagttgaccgcttagattcagcgaaacctttttggagactcggaaacaccgacccaaaggaaaacggcacctccaactcacttttagacaattgtacgtcccatcaactttttatggatatccttgaactatgctcctaattgatttgcttggcttgcattaatttccaagagctggttgagaagaagatcagtactaatctcctttggttgagggtttgaagaacttccttttctttggtttggtgtattgtctttgtaaggacagaaactgggtggatagttgttgagtttgggttgatagcctccttgttggttgttgtaagatggcttttgttggtagtctccttggtactgaggttttcttaaaaagattccttatagagtgttctgctcatttggtttgaaaggtggatagttttgatctttttgtctacattgtgcttatgtttgagaggttatagttgtagtagttgttatagttgttgttgatgtagccttacctactgacatagttaacatcttcttgaccctcttgctcatgattctgtcttgctcatgattctgttgagattcattgtagccttgctattgatcaaattcacccattaaatttacatttttctgccttttcaaaaaggatgtcgatttggtcatgtactgctttcgctcccttcttgtgtgctcatcttgagctccacctctattgttggttctgtcataatcatcacaatagatagtcactttgagcaagattctctaccaactgcatccccttgtttgaaatcatcccaagagatgcccactgcttacagtgtcaagcatgttcttaaatttgggcaagactcctctgtagaagggtctcaacaaagtctcctttctgaattcatggtgagggccctgcataatctagcccttgatgaaccgctccatccttcaccaaagttctctattcttttatattataactcaatacttttactcctcaaagacatttcacctttcattgacaaatatagataaagaaagtaagttagtaaccaaatagaaaataagatttagtctcaagcaatgactgaattccaatgtcaaaatcaatcttaggatttgcaacggtatcaaaactcaataagaaatcagtttaattaatcttgtataactctagttacaagctctaatgttgttgtaatataagagttgtcaatcagaacctaatatgactctaagcaaagagaatgaaaataattaccaaatataattaagcaatataagttttgtgaataagtaattaaacataaactaaacaagattcaaacttaatgatctgaaactcaaacaaacttaagaacaataacaaccttttaatcaaaggttaaggtatggatccatgggcaagactttcatttaagataattaatattcaaccaaaggggctcaaactaacaatcattcaagttacatacgtctagacacaaatgtttcagcaaacttatttcggacatgaaatgtttggatctagatatagagagatcaagactaattcagtattaggatcactaaagaagcatagaacaagatatgaaggatctagcaatatacacccaagtcttcactaaatcatcctaatctacgtaaccatagatccaaatagtgtctctcactaatcatcatgaaagaccttaaaccatgtaacgtttaagaataatcatgttagaagacaagataaacacactaaacataagacttataagagatattgcattattgtttttttgtcagaaaaagaagagaaattgcttaaaagatgataaatgaataacttttcatcagaatcaattgtcttaatagataaaataagataattaaaaaatttggcctaaaaaacagatatataggcctccaaagtcggacttgatcgactcatcaaccctagattgacccaatagaaactgggtaaaattctggtgaaagctt

SEQ ID NO: 843 - skipped

SEQ ID NO: 844 - DNA, 710 bp, Saccharomyces cerevisiae
aacaaagtatctaaacaaaatacataagtgtactcaaactgagtagaatcgtcgattaaacttccttctccttttaaaaattaaaaacagcaaatagttagatgaatatattaaagactattcgtttcatttcccagagcagcatgacttcttggtttcttcagacttgttaccgcaggggcatttgtcgtcgctgttacaccccgttgggcagctacatgatttttggcattgttcattatttttgcagctaccacattggcattggcactcatgaccttcattttggaagttaattaattcgctgaacattttatgtgatgattgattgattgattgtacagtttgtttttcttaatatctatttcgatgacttctatatgatattgcactaacaagaagatattataatgcaattgatacaagacaaggagttatttgcttctcttttatatgattctgacaatccatattgcgttggtagtcttttttgctggaacggttcagcggaaaagacgcatcgctctttttgcttctagaagaaatgccagcaaaagaatctcttgacagtgactgacagcaaaaatgtctttttctaactagtaacaaggctaagatatcagcctgaaataaagggtggtgaagtaataattaaatcatccgtataaacctatacacatatatgaggaaaaataatacaaaagtgtttt

SEQ ID NO: 845 - DNA, 710 bp, Saccharomyces cerevisiae; identical to SEQ ID NO: 844

SEQ ID NO: 846 - DNA, 550 bp, Saccharomyces cerevisiae
attgcacaacttaagtttgtcgaggatcatttttttgaactgaatcatgctctttttaagtgctttgaaaccctcgatgaatgtgtcaatgtgcaaagataaaccattgttctctgttgatcagtgacttaatgtttgctttggagaatgatattttccctttcctatatttgacttttgttctaaaagttatttggagagaaaaggcatgattgaggttgcgactttttcgtttttgcttttgcatggataattcatccatgcacatctcactttattggaccttcaagattggtttcccatgtaatttaattttctctcctctacatttaatatgttctatattaattaataccaattgagttgtgcgtacttcattgcagatattttaccagacctgtctgagtttttcgttcaagtttggttgaaatcggcttgaggtatatgaacgtggttgggatatggagattgggagatcaaagaagcgaaaatacctgagacagtttttttaaaaaagaagctaaggaacatgactcaaagagacacatta

SEQ ID NO: 847 - DNA, 268 bp, Saccharomyces cerevisiae
atggctcttcaacacatttcaacatgttcaagtaatttgtgttagtggatgaccatttaggggctgctggcctggttaccgggagtttttcttggatccaagctagcttttccgtctgattatccttaagcttcacaaattacaatttttcccacgcattaagaaataagctcaagatgcctaaaataagttctatcccgccttttttcgctaacaatgactgagtattcccacagtctatagtttgatagtagatgggcggaaattt

SEQ ID NO: 848 - DNA, 306 bp, Saccharomyces cerevisiae
acccaaaaacatcaagaaaagcctttcaataaattgctcttctcttggcgaaagaaagcggggggcaaaaagaatcacgggacttatgtttcgggatctctttgtttcttctttttttcccggagaataattttttaggaccaattaccgtagttgcgactacaacaattgttgttcatacccccacgatttactttttgaaaactagtttttggaataataatgttgtaaaatttccctttttccaccccgatttgtattttatttttcgttacaaaattgggactaatattaagggcgacagtt

SEQ ID NO: 849 - DNA, 136 bp, Didymium iridis
aatcgggttgaacacttaattgggttaaaacggtgggggacgatcccgtaacatccgtcctaacggcgacagactgcacggccctgcctcttaggtgtgttcaatgaacagtcgttccgaaaggaagcatccggta

SEQ ID NO: 850 - DNA, 160 bp, Naegleria jamiesoni
gatggggttcataccttaatctgccaaaacgggacctctgttgaggtataaccaatattccgtactaaggatttcgatccagaacgtctagagactacacggtagaccaattttggtggtatgaatggatagtccctagtaaccatctaggcatcccata

SEQ ID NO: 851 - RNA, 183 nt, ESCHERICHIA COLI
auuucucugagauguucgcaagcgggccaguccccugagccgauauuucauaccacaagaauguggcgcuccgcgguuggugagcaugcucgguccguccgagaagccuuaaaacugcgacgacacauucaccuugaaccaaggguucaaggguuacagccugcggcggcaucucggagauuc

SEQ ID NO: 852 - RNA, 107 nt, SALMONELLA ENTERICA TYPHIMURIUM
cgguuauaaaucaacacauugauuuauaagcauggaaauccccugagugaaacaacgaauugcuguguguagucuuugcccgucuccuacgaugggcuuuuuuuuua

SEQ ID NO: 853 - RNA, 93 nt, SALMONELLA ENTERICA TYPHIMURIUM
gcuaucaucauuaacuuuauuuauuaccgucauucacuucugaaugucuguuuaccccuauuucaaccggaugcuucgcauucgguuuuuuuu

SEQ ID NO: 854 - RNA, 93 nt, Klebsiella pneumoniae
gcuaucaucauuaacuuuauuuauuaccgucauucaguucugaaugucuguuuaccccuauuucgaccggaugcuucgcauccgguuuuuuuu

SEQ ID NO: 855 - RNA, 199 nt, Serratia marcescens
aaaaucauguaguuauacaaaucuuuaagaaaaaaaagccaaccauacaauuguacuggacaauaagcacauugugccaaaacgccgccugcacgcagccgcuauaaucaccucgcuaucaucauuauuuucauuauuaccuucauuauccgaagauaauuucugcauaccuuuaaccggcuucuggccgguuuuuuau

SEQ ID NO: 856 - RNA, 357 nt, Salmonella enterica
accagucggcaaguccauucuccgcaaaaauacagaauaauccaacacgaauaugauacuaaaacuuuuaagauguuacaguuaucuauauagauguuucaaaauaugaauuuuacggaacuuuuuuaaagcaaaaaucaaguaaaaauaagcacaaauagacaaaauauauucacgaaacuuuuaaaaucaacggguuaaauugaugaaauucauagcacugaaugauaaaacagaaucuucauucgcaacuaaaauagugaccgcuaucaucauuaacuuuauuuauuaccgucauucacuucugaaugucuguuuaccccuauuucaaccggaugcuucgcauucgguuuuuuu

SEQ ID NO: 857 - RNA, 363 nt, SALMONELLA ENTERICA TYPHIMURIUM
gucgacagggagucguacaacgaagcgaacgucaggaugaugacgcuucagcaggacacgccaggaugguguuacaaggaaaggcuucaggaugaagcaaaguggaaagcgcaggaugcguuaaaggacaccuccaggacggagaacgagagccgaucaggauguucggcgggucuggaugaccagggacgcuucaggaagaagcuaucacaucgggcgaugugcgcaggaugcaaacguucaggaugaacaggccguaaggucacaggaaaaguugucacggaugagcagggagcacgaaaaguagcuggaaugcugcgaaacgaaccgggagcacuguuuauacagugcucccuuuuuuuu

SEQ ID NO: 858 - RNA, 356 nt, Salmonella enterica
gagucguacaacgaagcgaacgucaggaugaugacgcuucagcaggacacgccaggaugguguuacaaggaaaggcuucaggaugaagcaaaguggaaagcgcaggaugcguuaaaggacaccuccaggacggagaacgagagccgaucaggauguucggcggaucuggauaaccagggacgcuucaggaugaagcuaucacaucgggcgaugugcgcaggauguaaacguucaggaugaacaggccguaaggucacaggaaaaguugucacggaugagcagggagcacgaaaaguagcuggaaugcugcgaaacgaaccgggagcacuguuuauacagugcucccuuuuuuuguu

SEQ ID NO: 859 - RNA, 183 nt, Salmonella enterica
agcauagcaacgaacgauuaucccuaucaaccuuucugauuaauaauacaucacagaacggagcgguuucucguuuaacccuugaagacaccgcccguucagaggguaucucucgaacccgaaauaacuaaagccaacgugaacuuuugcggaccucugguccgcuuuuuuuugcguaaaaaa

SEQ ID NO: 860 - DNA, 18 bp, Unknown
  PCR primer
gattgtcttgggataccg

SEQ ID NO: 861 - DNA, 33 bp, Unknown
  PCR primer
aatttaatacgactcactatagggaagtatcat

SEQ ID NO: 862 - DNA, 22 bp, Unknown
  PCR primer
tcggaacgactgttcattgaac

SEQ ID NO: 863 - DNA, 19 bp, Unknown
  PCR primer
cccgattgcatcatggtga

SEQ ID NO: 864 - DNA, 19 bp, Unknown
  PCR primer
attgggacaactccagtga

SEQ ID NO: 865 - DNA, 14 bp, Unknown
  PCR Primer
gattgtcttgggat

(19) United States
(12) Patent Application Publication
(10) Pub. No.: US 2009/0307788 A1
(43) Pub. Date: Dec. 10, 2009

(54) RIBOZYME MEDIATED STABILIZATION OF POLYNUCLEOTIDES

(75) Inventor: Henrik Nielsen, Bronshoj (DK)

Correspondence Address: WEINGARTEN, SCHURGIN, GAGNEBIN & LEBOVICI LLP, TEN POST OFFICE SQUARE, BOSTON, MA, 02109, US

(73) Assignee: KOBENHAVNS UNIVERSITET, COPENHAGEN N (DK)

(21) Appl. No.: 12/305,266

(22) PCT Filed: Jun. 19, 2007
(86) PCT No.: PCT/DK2007/000296

§ 371 Date: Dec. 17, 2008

Related U.S. Application Data
(60) Provisional application No. 60/814,564, filed on Jun. 19, 2006.

(30) Foreign Application Priority Data
Jun. 19, 2006 (DK) PA 2006 00833

(51) Int. Cl. A01K 67/027; A01H 5/00; C07H 21/04; C07H 21/02; C12N 15/63; C12N 5/10; C12N 5/08; C12P 21/06; A61K 35/12; C12N 5/02; C12N 1/21; C12N 1/19; C12N 1/15

(52) U.S. Cl. 800/13; 800/298; 536/23.5; 536/23.7; 536/23.74; 435/320.1; 435/325; 435/366; 435/419; 435/69.1; 424/93.7; 435/420; 435/252.3; 435/254.2; 435/254.11

(57) ABSTRACT

The present invention relates to the production of novel, recombinant polynucleotides comprising the GIR1 ribozyme, or a variant thereof, vectors comprising such polynucleotides and recombinant host cells comprising such polynucleotides and/or such vectors. The invention furthermore relates to the use of said polynucleotides in the treatment of an individual suffering from a disease associated with or caused by instability of a transcript of said second subsequence such as cancer, cachexia, α-Thallasemia or leukaemia.

All patent and non-patent references cited in this application are hereby incorporated by reference in their entirety.

### FIELD OF THE INVENTION

The present invention relates to ribozyme mediated stabilization of polynucleotides, such as ribonucleic acids (RNA). Stabilization of polynucleotides according to the present invention can be exploited in molecular biology, genetic engineering, genetics and disease treatment, prevention and/or alleviation. The invention exploits the fact that ribozyme GIR1 has been shown to stabilize polynucleotides.

### BACKGROUND OF THE INVENTION

RNA splicing is found in most prokaryotic and eukaryotic organisms and different RNA splicing mechanisms have evolved for different classes of genes (C. B. Burge, T. Tuschl, P. A. Sharp, in The RNA World, Second Edition, R. F. Gesteland, T. R. Cech, J. F. Atkins, Eds. [Cold Spring Harbor Laboratory (CSHL) Press, Cold Spring Harbor, N.Y. 1999] pp. 525-560; C. R. Trotta, J. Abelson, in The RNA World, Second Edition, R. F. Gesteland, T. R. Cech, J. F. Atkins, Eds. (CSHL Press, Cold Spring Harbor, N.Y., 1999) pp. 561-584B). Group I introns (T. R. Cech, in The RNA World, Second Edition, R. F. Gesteland, T. R. Cech, J. F. Atkins, Eds. (CSHL Press, Cold Spring Harbor, N.Y., 1999), pp. 321-349) carry out splicing in a structurally and chemically distinct way from that of group II introns and the spliceosomal introns found widespread in higher eukaryotes.

Group I introns are widespread in nature, but with a notable sporadic occurrence. Whereas organellar group I introns have been identified in rRNA, mRNA, and tRNA transcription units, the nuclear group I introns are confined to the rRNA transcription units (Johansen et al. 1996).

Group I introns have been studied from two different perspectives: (1) as a selfish genetic element, and (2) as a ribozyme responsible for its own splicing reaction.

Several observations support the notion of group I introns as selfish genetic elements. Group I introns catalyze their own excision, although secondarily recruited host factors are implicated in many instances (Lambowitz et al. 1999). The presence of a group I intron appears to have little effect on the host (see Nielsen and Engberg 1985 for an analysis of the Tetrahymena intron). Also, the mobility of group I introns within species is well documented and occurs by allelic homing, initiated by cleavage of the intron-lacking allele by an intron homing endonuclease (Lambowitz and Belfort 1993).

The GIR1 ribozyme is found in so-called “twin-ribozyme introns” in rDNA of isolates of the myxomycete Didymium and the amoebaflagellate Naegleria. It is structurally related to the group I splicing ribozymes. However, it catalyzes a cleavage reaction rather than splicing and is crucial in the formation of the 5′ end of an mRNA encoded within the intron.

### SUMMARY OF THE INVENTION

The present invention in one aspect is directed to novel, recombinant polynucleotides comprising the GIR1 ribozyme, or a variant thereof as defined herein, vectors comprising such polynucleotides and recombinant host cells comprising such polynucleotides and/or such vectors.

GIR1 is a naturally occurring ribozyme (RNA enzyme) isolated from myxomycetes and amoebaeflagellates. It catalyses cleavage at an internal position and generate a 5′ fragment with a 3′OH and a 5′-fragment with a lariat cap. The lariat cap is a unique structure in which the first and the third nucleotide of the chain are connected with a 2′, 5′-phosphodiester bond.

In its natural setting, the group I introns of the twin-ribozyme type, the cleavage results in the release of a 3′-fragment that acts as an mRNA encoding a homing endonuclease. The lariat cap protects the mRNA against 5′-3′ exonucleases. In addition, it is possible that the lariat cap is involved in translation of the message.

Specific examples of GIR1 molecules are disclosed in Table 1 below:

TABLE 1
Specific GIR1 molecules
according to the present invention

| Source | Sequence |
|---|---|
| Didymium iridis | ttttggttgggttgggaagtatcatggctaatcac |
| GIR1 (DGIR1) | catgatgcaatcgggttgaacacttaattgggttaa |
| (SEQ ID NO: 1) | aacggtgggggacgatcccgtaacatccgtcctaa |
| | cggcgacagactgcacggccctgcctcttaggtgtg |
| | ttcaatgaacagtcgttccgaaaggaagcatccggt |
| | atcccaagacaatcaaatctaaggataccaatctgt |
| | gcacttcaacaacaatggtga |
| Naegleria | ccgttgttgtgcgatggggttcataccttaatctgc |
| jamiesoni GIR1 | caaaacgggacctctgttgaggtataaccaatatt |
| (NGIR1) | ccgtactaaggatttcgatccagaacgtctagaga |
| (SEQ ID NO: 2) | ctacacggtagaccaattttggtggtatgaatggat |
| | agtccctagtaaccatctaggcatcccatacaaa |
| | atgg |

Below is provided a structure based alignment of DiGIR1 and NaGIR1 core sequences (excluding sequence originating from P2 and P2.1 as illustrated in FIG. 1, panel B):

| | | |
|---|---|---|
| DiGIR1 | aatcggg ttgaacac ttaat tgggtt aaa acggtg gggg- acga tccc- | (SEQ ID NO: 1A) |
| NaGIR1 | gatgggg ttcatacc ttaat ctgcc- aaa acggg- acctc tgtt gaggt | (SEQ ID NO: 2A) |
| Domain | P10′ P15′ J15/3 P3′ J3/4 P4′ P5′ L5 P5″ | |
| DiGIR1 | --- ----- --- gtaa catccgt cc----- taac gg--------- cga | |
| NaGIR1 | ata accaa tat ---- tccgtac taaggat ttcg atccagaacgt cta | |
| Domain | P5.1′ L5.1 P5.1″ J5/4 P4″ P6′ L6 P6″ J6/7 | |
| DiGIR1 | cagactg cac ggccct gcct ctt- aggt gtgttcaa tga acagtcg | |
| NaGIR1 | gagacta cac ggtag- acca attt tggt ggtatgaa tgg atagtcc | |
| Domain | P7′ J7/3 P3″ P8′ L8 P8″ P15″ J15/7 P7″ | |
| DiGIR1 | ttcc gaaa--- ggaa gcat ccggta | |
| NaGIR1 | ctag taaccat ctag gcat cccata | |
| Domain | P9′ L9 P9″ J9/10 P10″ | |

The alignment shown is between GIR1 from Didymium iridis and Naegleria jamiesoni with annotation derived from structure modelling of the two. As with the closely related splicing ribozymes, the structure is more conserved than the sequence. In vitro mutagenesis has revealed that most of the paired (P) sequences and several tertiary interactions that are not described in the figure are necessary for activity. However, very few residues are obligatory at the sequence level. These include the G-binding site in P7 (in particular the pair G174:C215), G229 at the cleavage site, A231, and A153 that is involved in recognition of the G-U pair at the branch point. The nucleotides involved in the characteristic 2′, 5′ phosphodiester bond (C230 and U232) are not critical at the sequence level (H. Nielsen, unpublished). Sequences in bold represent the “core” of the ribozyme. These sequences appear to be more conserved than the remainder of GIR1 (43 of 61 identical residues in the present comprison).

The above polynucleotides, vectors and host cells have utility e.g. in the fields of genetics, recombinant DNA technology and applications thereof in e.g. development of novel and innovative methods for treating diseases associated with or caused by ribonucleotide instability.

In one aspect the invention is directed to a polynucleotide comprising a first and a second subsequence,

wherein the first subsequence comprises or encodes
a) a GIRl ribozyme defined herein as SEQ ID NO:1, or a transcript thereof, or
b) a polynucleotide at least 80% identical, such as 85% identical, for example 90% identical, such as 91% identical, for example 92% identical, such as 93% identical, for example 94% identical, such as 95% identical to a), or
c) a fragment of a) or b) capable of cleaving the second subsequence, or the complementary strand thereof, or
d) a polynucleotide, the complementary strand of which hybridizes, under stringent conditions, with a polynucleotide as defined in any of a), b) and c),

wherein the first and second subsequences together are capable of forming a secondary and/or tertiary interaction resulting in modification and/or stabilization of the transcript of said second subsequence

wherein the first subsequence is not natively associated with the second subsequence.

In another aspect the present invention is directed to a polynucleotide comprising a first and a second subsequence,

wherein the first subsequence comprises or encodes
a) a GIR1 ribbzyme defined herein as SEQ ID NO:2, or a transcript thereof, or
b) a polynucleotide at least 80% identical, such as 85% identical, for example 90% identical, such as 91% identical, for example 92% identical, such as 93% identical, for example 94% identical, such as 95% identical to a), or
c) a fragment of a) or b) capable of cleaving the second subsequence, or the complementary strand thereof, or
d) a polynucleotide, the complementary strand of which hybridizes, under stringent conditions, with a polynucleotide as defined in any of a), b) and c),

wherein the first and second subsequences together are capable of forming a secondary and/or tertiary interaction resulting in modification and/or stabilization of the transcript of said second subsequence

wherein the first subsequence is not natively associated with the second subsequence.

In yet another aspect the present invention is directed to a polynucleotide comprising a first and a second subsequence,

wherein the first subsequence comprises or encodes
a) a GIRl ribozyme comprising SEQ ID NO:1A, or a transcript thereof, or
b) a polynucleotide at least 80% identical, such as 85% identical, for example 90% identical, such as 91% identical, for example 92% identical, such as 93% identical, for example 94% identical, such as 95% identical to a), or
c) a fragment of a) or b) capable of cleaving the second subsequence, or the complementary strand thereof, or
d) a polynucleotide, the complementary strand of which hybridizes, under stringent conditions, with a polynucleotide as defined in any of a), b) and c),

wherein the first and second subsequences together are capable of forming a secondary and/or tertiary interaction resulting in modification and/or stabilization of a transcript of said second subsequence

wherein the first subsequence is not natively associated with the second subsequence.

In another aspect the present invention is directed to a polynucleotide comprising a first and a second subsequence,

wherein the first subsequence comprises or encodes
a) a GIR1 ribozyme comprising SEQ ID NO:2A, or a transcript thereof, or
b) a polynucleotide at least 80% identical, such as 85% identical, for example 90% identical, such as 91% identical, for example 92% identical, such as 93% identical, for example 94% identical, such as 95% identical to a), or
c) a fragment of a) or b) capable of cleaving the second subsequence, or the complementary strand thereof, or
d) a polynucleotide, the complementary strand of which hybridizes, under stringent conditions, with a polynucleotide as defined in any of a), b) and c),

wherein the first and second subsequences together are capable of forming a secondary and/or tertiary interaction resulting in modification and/or stabilization of a transcript of said second subsequence,

wherein the first subsequence is not natively associated with the second subsequence.

Stringent conditions as used herein shall denote stringency as normally applied in connection with Southern blotting and hybridization as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denaturated salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

The above polynucleotides can be DNA (deoxyribonucleic acids) or RNA (ribonucleic acids) and the nucleotide residues can be natural and/or non-natural nucleotide residues preferably capable of being incorporated into a polynucleotide by polymerase mediated incorporation.

The second subsequence can be a DNA coding for an RNA (such as a coding RNA or non-coding RNA). The transcript can thus be in the form of mRNA; tRNA or rRNA (coding RNA), or the transcript can be in the form of a non-coding RNA having a (further) regulatory function in a biological cell. Examples of non-coding (regulatory) RNAs are cited herein below.

First and second subsequences are listed herein interchangably in both RNA and DNA annotation as is usual in the art.

The following table of features illustrates consensus sequences and constraints of GIR1 ribozymes and variants thereof. Reference is made to FIG. 1, panel B.

TABLE 2
Sequence constraints and structural motifs of GIR1 and
variants thereof

| Structure | Consensus and constraints |
|---|---|
| P10 | 5 bp; possible tertiary interaction |
| P15 | 9 bp; includes critical GU pair at active site |
| J9/10 | Consensus 5′-GYAU; G and A are critical (Y = C or U) |
| J15/7 | Consensus 5′-UGR (R = A or G) |
| P5 | Highly variable |
| J5/4 | Highly variable. Includes 5′-AA critically involved in |
| | recognition of GU pair at active site |
| P4 | Conserved for unknown reasons. Consensus 5′-strand: 5′- |
| | ACGGNN/3′-strand: 5′-NNUCCGU (N = A, C, G or U) |
| P6 | Variable; tertiary contact with P3 |
| J6/7 | Consensus 5′-CAN (N = A, C, G or U) |
| J3/4 | Consensus 5′-AAA |
| P9 | 4 bp stem, highly variable loop. Involved in tertiary |
| | interaction |
| P7 | Conserved G-binding architecture involving critical |
| | G174:C215 pair |
| J7/3 | Consensus 5′-CAC |
| P3 | Consensus 5′-strand: 5′-GGCNN/3′-strand: 5′-NNGNN. |
| | Tertiary interaction with P6. (N = A, C, G or U) |
| P8 | Variable. Interacts with J15/3 |
| J15/3 | Consensus 5′-UUAAUU; forms 3 way-junction (WJ) of |
| | family C. Interacts with P8 |
| P2 | Highly variable. A short base paired segment is required for |
| | minimal ribozyme. Involved in tertiary contacts |

Variants of GIR1 include ribozymes comprising the above-mentioned consensus sequences in combination with critical nucleotide residues and conserved sequences as indicated in Tables 1 and/or 2.

Further preferred GIR1 molecules according to the present invention are nucleotide sequences having greater than 80 percent sequence identity, and preferably greater than 90 percent sequence identity (such as greater than 91% sequence identity, for example greater than 92% sequence identity, such as greater than 93% sequence identity, for example greater than 94% sequence identity, such as greater than 95% sequence identity, for example greater than 96% sequence identity, such as greater than 97% sequence identity, for example greater than 98% sequence identity, such as greater than 99% sequence identity, for example greater than 99.5% sequence identity), to any of SEQ ID NO:1, SEQ ID NO:2, SEQ ID NO:1A and SEQ ID NO:2A.

The present invention further includes the use of recombinant or synthetically or transgenically produced GIR1 molecules. In one embodiment, the GIR1 molecule is a homologue of GIR1.

There is also provided a recombinant polynucleotide molecule in the form of an expression vector comprising a recombinant polynucleotide according to the present invention. The vector can further comprise a replicon capable of directing extrachromosomal replication. The vector can also comprise an expression signal capable of directing the expression of the first and/or second subsequence either in vitro under suitable conditions, or in vivo in a host cell, The vector can also comprise a selection marker for suitable selection when transformed or transfected into a host cell.

In a further aspect there is provided a host organism or host cell transfected or transformed with the polynucleotide according to the invention or the vector according to the invention.

In one aspect there is provided a host cell or host organism transfected or transformed with
i) a first polynucleotide comprising a first subsequence comprising
   wherein the first subsequence comprises or encodes
   a) a GIR1 ribozyme defined herein as SEQ ID NO:1 or SEQ ID:2, or a ribozyme comprising SEQ ID NO:1A or SEQ ID NO:2A, or a transcript thereof, or
   b) a polynucleotide at least 80% identical, such as 85% identical, for example 90% identical, such as 91% identical, for example 92% identical, such as 93% identical, for example 94% identical, such as 95% identical to any polynucleotide of a), or
   c) a fragment of a) or b) capable of cleaving the second subsequence, or the complementary strand thereof, or
   d) a polynucleotide, the complementary strand of which hybridizes, under stringent conditions, with a polynucleotide as defined in any of a), b) and c), and
ii) a second polynucleotide comprising a second subsequence not natively associated with the first subsequence.
wherein the first and second subsequences together are capable of forming a secondary and/or tertiary interaction resulting in modification and/or stabilization of a transcript of said second subsequence,
wherein the first subsequence is not natively associated with the second subsequence, or wherein the host cell does not natively comprise said first and second subsequences.

There is also provided a composition comprising the recombinant polynucleotide according to the invention, a composition comprising the vector according to the invention claim, and a host organism according to the invention, said composition further comprising a physiologically acceptable carrier.

The present invention also provides a method for stabilizing polynucleotides, such as RNAs, for example non-coding, regulatory RNAs having an affinity for the GIR1 ribozyme, or a variant thereof as defined herein. There is also provided a method for improving the production of polypeptides as a result of mRNA stabilization.

The invention in a further aspect is directed to a method for manipulating the phenotype of a biological cell, wherein said manipulation is achieved by modulation of GIR1 mediated polynucleotide stability in said biological cell. The.modulation can also be achieved by a GIR1 variant as defined herein below.

The types of cells which can be targeted includes mammalian cells, such as animal and human cells, higher eucaryotes, fungal calls, yeasts, as well as bacteria. Plant cells are also contemplated. Methods for introducing into the afore-mentioned cells a recombinant polynucleotide according to the present invention, a vector comprising such a polynucleotide and a recombinant host cell comprising such a polynucleotide and/or such a vector are well known in the art. Also, expression signals capable of directing consitutive or inducible expression of GIR1, or a GIR1 variant, are well known in the art.

In a further interesting aspect there is provided a method of treatment of an individual suffering from a disease caused by or associated with increased polynucleotide degradation, such as increased RNA degradation.

Non-Coding (Regulatory) RNA

Examples of further second subsequences according to the present invention are provided herein below.

A variety of RNAs do not function as mRNA, tRNAs or rRNAs. The latter class of RNAs can collectively be termed non-coding RNAs or regulatory RNAs. Such non-coding (regulatory) RNAs are present in many different biological cells and it is one object of the invention to stabilise such non-coding (regulatory) RNAs either in vivo or in vitro. ncRNAs may target RNA or DNA by direct base pairing, by mimicking the structure of other nucleic acids or as part of a larger RNA-protein complex. Non-coding RNAs (ncRNAs) have been referred to as small RNAs in bacteria (see Storz et al., 2002).

Second subsequences in the form of non-coding (regulatory) RNAs control and regulate a wide range of developmental and physiological pathways in animals, including hematopoietic differentiation, adipocyte differentiation and insulin secretion in mammals, and have been shown to be perturbed in cancer and other diseases. The extent of transcription -of non-coding sequences and the abundance of small RNAs suggests the existence of an extensive regulatory network on the basis of RNA signaling which may underpin the development and much of the phenotypic variation in mammals and other complex organisms and which may have different genetic signatures from sequences encoding proteins.

The sizes of ncRNAs varies depending on their function. For example, those associated with development in the nematode Caenorrhabditis elegans, Drosophila and mammals have been found to be 21 to 25 nucleotides in length. The translational regulators in bacteria are from 100 to 200 nucleotides in length and those e.g. involved in gene silencing in eukaryotes are larger than 10,000 nucleotides. All of the above are contemplated as second subsequences.

An example of second subsequences in the form of macro ncRNAs (i.e. larger than 10,000 nucleotides) include Xist and Air, which in mouse are approximately 18 and 108 Kb, respectively. Xist plays an essential role in mammals by associating with chromatin and causing widespread gene silencing on the inactive X chromosome, while Air is required for paternal silencing of the Igf2r/Slc22a2/Slc22a3 gene cluster. Apart from their extreme length, Xist and Air share two other important features: genomic imprinting and antisense transcription.

Genomic imprinting is a process by which certain genes are expressed differently according to whether they have been inherited from the maternal or paternal allele. Imprinting is critical for normal development, and loss of imprinting has been implicated in a variety of human diseases. ncRNAs have been discovered at many different imprinted loci and appear to be important in the imprinting process itself.

The other feature that Xist and Air have in common is that both are members of naturally occurring cis-antisense transcript pairs. Previous studies have indicated the existence of thousands of mammalian cis-antisense transcripts. These transcripts may regulate gene expression in a variety of ways including RNA interference, translational regulation; RNA editing, alternative splicing, and alternative polyadenylation, although the exact mechanisms by which antisense RNAs function are unknown. Mammalian cis-antisense transcripts constitute one example of second subsequences.

Mammalian cells harbor numerous small non-protein-coding RNAs. Examples of second subsequences of mammalian origin include small nucleolar RNAs (snoRNAs), microRNAs (miRNAs), short interfering RNAs (siRNAs), small nuclear RNAs (snRNAs) and small double-stranded RNAs, which regulate gene expression at many levels including chromatin architecture, RNA editing, RNA stability, translation, and quite possibly transcription and splicing.

ncRNAs have also been found to have a role in protein degradation and translocation. For example tRNAs in combination with spliceosomal snRNAs are housekeeping RNAs involved in mRNA splicing and translation. These RNAs are processed by multistep pathways from the introns and exons of longer primary transcripts, including protein-coding transcripts. Most show distinctive temporal- and tissue-specific expression patterns in different tissues, including embryonal stem cells and the brain, and some are imprinted.

mRNA Instability

It is one objective of the present invention to improve mRNA instability in cells and in vitro when such a stabilisation is desirabel. Messenger RNA (mRNA) expression in mammalian cells is highly regulated. Traditionally, emphasis has been placed on elucidating mechanisms by which genes are regulated at the transcriptional level; however, steady-state levels of mRNA is also dependent on its half-life or degradation rate.

Changes in mRNA stability play an important role in modulating the level of expression of many eukaryotic genes and different mechanisms have been proposed for the regulation of mRNA turnover (Cleveland and Yen, 1989, New Biol. 1:121; Mitchell and Tollervey, 2000, Curr. Opin. Genet. Dev. 10:193; Mitchell and Tollervey, 2001, Curr. Opin. Cell. Biol. 13:320; Ross, J. 1995, Microbiol. Rev. 59:423; Sachs, A. B., 1993, Cell 74:413; Staton et al. 2000, J. Mol. Endocrinology 25:17; Wilusz et al. 2001, Nat. Rev. Mol. Cell Biol. 2:237).

Regulation of mRNA stability is complex and the regulation can involve sequence elements in the mRNA itself, activation of nucleases, as well as the involvement of complex signal transduction pathway(s) that ultimately influence trans-acting factors' interaction with mRNA stability sequence determinants.

Recently, it has become increasingly apparent that the regulation of RNA half-life plays a critical role in the tight control of gene expression and that mRNA degradation is a highly controlled process. RNA instability allows for rapid up- or down-regulation of mRNA transcript levels upon changes in transcription rates.

A number of critical cellular factors, e.g. transcription factors such as c-myc, or gene products which are involved in the host immune response such as cytokines, are required to be present only transiently to perform their normal functions. Transient stabilization of the mRNAs which code for these factors permits accumulation and translation of these messages to express the desired cellular factors when required; whereas, under nonstabilized, normal conditions the rapid turnover rates of these mRNAs effectively limit and “switch off” expression of the cellular factors. Thus, aberrant mRNA turnover usually leads to altered protein levels, which can dramatically modify cellular properties. Dysregulation of mRNA stability has been associated with human diseases including cancer, inflammatory disease, and Alzheimer's disease.

The stabilization of mRNA appears to be a major regulatory mechanism involved in the expression of inflammatory cytokines, growth factors, and certain protooncogenes. In the diseased state, mRNA half-life and levels of disease-related factors are significantly increased due to mRNA stabilization (Ross, J. 1995, Microbiol. Rev. 59:423; Sachs, A. B., 1993, Cell 74:413; Staton et al. 2000, J. Mol. Endocrinology 25:17; Wilusz et al. 2001, Nat. Rev. Mol. Cell Biol. 2:237).

Transcription rates and mRNA stability are often tightly and coordinately regulated for transiently expressed genes such as c-myc and c-fos, and cytokines such as IL-1, IL-2, IL-3, TNF.alpha., and GM-CSF. In addition, abnormal regulation of mRNA stabilization can lead to unwanted build up of cellular factors leading to undesirable cell transformation, e.g. tumour formation, or inappropriate and tissue damaging inflammatory responses.

### DEFINITIONS

mRNA: Messenger RNA

rRNA(s): Ribosomal RNA

tRNA: Transfer RNA

miRNA(s): MicroRNA—putative translational regulatory gene family

ncRNA(s): Non-coding RNA—all RNAs other than mRNA

siRNA(s): Small interfering RNA—active molecules in RNA interference

snRNA(s): Small nuclear RNA—includes spliceosomal RNAs

snmRNA(s): Small non-mRNA—essentially synonymous with small ncRNAs

snoRNA(s): Small nucleolar RNA—most known snoRNAs are involved in rRNA modification

stRNA: Small temporal RNA—for example, lin-4 and let-7 in Caenorhabditis elegans tRNA Transfer RNA

Natural nucleotide: Any of the four deoxyribonucleotides, dA, dG, dT, and dC (constituents of DNA), and the four ribonucleotides, A, G, U, and C (constituents of RNA) are the natural nucleotides. Each natural nucleotide comprises or essentially consists of a sugar moiety (ribose or deoxyribose), a phosphate moiety, and a natural/standard base moiety. Natural nucleotides hybridize to complementary nucleotides in a number of ways. One way of hybridization is by means of the well-known rules of base pairing (Watson and Crick), where adenine (A) pairs with thymine (T) or uracil (U); and where guanine (G) pairs with cytosine (C), wherein corresponding base-pairs are part of complementary, anti-parallel nucleotide strands. The base pairing results in a specific hybridization between predetermined and complementary nucleotides. In nature, the specific interactibins leading to base pairing are governed by the size of the bases and the pattern of hydrogen bond donors and acceptors of the bases. A large purine base (A or G) pairs with a small pyrimidine base (T, U or C). Additionally, base pair recognition between bases is influenced by hydrogen bonds formed between the bases. In the geometry of the Watson-Crick base pair, a six membered ring (a-pyrimidine in natural oligonucleotides) is juxtaposed to a ring system composed of a fused, six membered ring and a five membered ring (a purine in natural oligonucleotides), with a middle hydrogen bond linking two ring atoms, and hydrogen bonds on either side joining functional groups appended to each of the rings, with donor groups paired with acceptor groups.

Base moiety: Nitrogeneous base moiety of a natural or non-natural nucleotide, or a derivative of such a nucleotide comprising alternative sugar or phosphate moieties. Base moieties include any moiety that is different from a naturally occurring moiety and capable of complementing one or more bases of the opposite nucleotide strad of a double helix.

Polynucleotide: A molecule comprising consecutively linked natural and/or non-natural nucleic acid residues. The polynucleotide can e.g. be an RNA or DNA molecule.

Isolated polynucleotide: Either (1) a DNA or RNA molecule that is separated from sequences with which it is immediately contiguous (in the 5′ and 3′ directions) in the naturally occurring genome of the organism from which it was derived or (2) a DNA or RNA molecule with an indicated sequence, but which has undergone some degree of purification relative to the genome and may retains some number of immediately contiguous genomic sequences. For example, such molecules include those present on an isolated restriction fragment or such molecules obtained by PCR amplification. DNA or RNA can be isolated and purified to any degree using methods well known in the art.

In accordance with the invention, the “isolated polynucleotide” may be inserted into or itself comprise a vector, such as a plasmid or virus vector, or be integrated into the genomic DNA of a prokaryote or eukaryote. With respect to RNA molecules of the invention, the term “isolated nucleic acid” primarily refers to an RNA molecule encoded by an isolated DNA molecule as defined above. But also includes RNA that has been isolated from a cellular source or RNA that has been chemically synthesized (and obtained at any level of purity). In these cases, the RNA molecule has been sufficiently separated from RNA molecules with which it would be associated in its natural state (i.e., in cells or tissues), such that it exists in a purified pure form, e.g., that the RNA is enriched in the mixture relative to its abundance as naturally produced.

RNA polynucLeotide: RNA molecule, such as mRNA, pre-mRNA, mature messenger RNA molecule, mRNA which was produced due to splicing of the pre-mRNA, ncRNA, small nucleolar RNAs (snoRNAs), microRNAs (miRNAs), short interfering RNAs (siRNAs), small nuclear RNAs (snRNAs) and small double-stranded RNAs that contains the same sequence information as the corresponding DNA molecule (albeit that U nucleotides replace T nucleotides) as the DNA molecule.

Ribose derivative: Non-natural ribose moiety forming part of a nucleoside capable of being enzymatically incorporated into a template or complementing template. Examples include e.g. derivatives distinguishing the ribose derivative from the riboses of natural ribonucleosides, including adenosine (A), guanosine (G), uridine (U) and cytidine (C). Further examples of ribose derivatives are described in e.g. U.S. Pat. No. 5,786,461.

Transcriptional product of a gene: A pre-messenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

Translational product of a gene: A protein, which is encoded by a gene.

Polypeptide: A molecule comprising amino acid residues which do not contain linkages other than amide linkages between adjacent amino acid residues.

### DESCRIPTION OF THE FIGURES

FIG. 1. (A) Schematic drawing of the structure of the Dir S956-1 intron and the GIR1 RNAs described in the text. (166)22 RNA refers to a 22-nt fragment isolated from-cleavage of a 166.22 RNA precursor. (B) Structure diagram of Didymium GIR1. (C) Primer extension analysis of RNA from an experiment parallel to that shown in FIG. 4. A sequencing ladder is shown to the left. (D) Cleavage analysis performed as in FIG. 4A, but by using precursor RNA that was labeled at its 3′ end with [32P]pCp instead of body-labeling with [a-32P]UTP. (E) Primer extension analysis of gel-isolated and reincubated (157)22 RNA alone, with 157 RNA, and with 166 RNA. The time points are 0, 1, 4, and 8 hours. (F) Ligation of a 22-nt 3′ fragment to a 166-nt 5′ fragment. The 3′ fragment was labeled at its 3′ end with [32P]pCp. The 5′ fragment was unlabeled. The time points are 0 and 20 min, and 1, 2, 3, and 4 hours. M1 and M2: 166.22 and 157.22, respectively, cleaved and labeled with [32P]pCp.

FIG. 2. (A) Characterization of the 5′ end of the 22-nt 3′ fragment. [32P]pCplabeled 3′ fragment was isolated from 157.22 and 166.22 and subjected to treatment with alkaline phosphatase (AP), AP followed by rephosphorylation with T4 polynucleotide kinase (APxPNK), or treatment with PNK alone (PNK). The sample denoted (157)22_{—}166 was preincubated at reaction conditions for 30 min before the analysis. OH and T1: Alkaline ladder and T1 digest of [32P]pCp-labeled precursor 157.22. (B) Diagram of the 22 nt lariat used for experiments in (C) and (D). The RNA was body-labeled at the phosphates in bold by incorporation of 32P. Arrows indicate potential cleavage sites for mung bean nuclease (MB) and snake venom phosphodiesterase (SV). Cleavage of the 22-nt fragment at sites labeled 1 with SV results in a protected lariat circle (LC). Cleavage at sites labeled 1 and 2 with MB results in a protected branched nucleotide (BR). Subsequent cleavage of BR with SV at sites labeled 3 releases the nucleotides involved in the branch. (C) Characterization of the lariat circle by gel purification and subsequent digestion with MB (LCxMB). The 22-nt fragment and digests with MB or SV serves as markers. (D) Characterization of the branched nucleotide by purification of its phosphorylated and dephosphorylated form, and subsequent TLC analysis of nucleotides liberated by digestion with SV. The first two runs show digests of the 22-nt fragment. The following show the isolated branch (BR), and dephosphorylated branch (BRAP), respectively. Finally, the last two-runs show the subsequent digests of these with SV (BRxSV and BRAPXSV).

FIG. 3. (A) Outline of the reaction catalyzed by GIR1. The 2′OH of the internal residue U232 makes a nucleophilic attack at the IPS. Bond lengths are not drawn to scale. (B) Cleavage experiment using 157.-7 ribozyme combined with four different deoxy-substituted substrates each containing 7 nucleotides upstream and 22 nucleotides downstream of IPS. Numbering of nucleotides is according to their position in the intron. (C) Diagram showing the structure of the fully processed I-Dir I mRNA that encodes the homing endonuclease.

FIG. 4 (A) Kinetic analysis of the two length variants 166.22 (filled circles) and 157.22 (open circles) performed as described (C. Einvik, H. Nielsen, R. Nour, S. Johansen, Nucl. Acids Res. 28, 2194 (2000)). (B) Gel electrophoretic analysis of the cleavage products of the two length variants. The time points are 0, 1, 2, 5, 10, 30, 60, 120, and 240 min. Pre: Precursor RNA. 5′-prod: 5′-product. The 3′-product was run out of the gel. The experiment shows that the two RNAs have similar cleavage kinetics.

FIG. 5. Inhibition of ligation by β-elimination. 32P-labeled 166.22 RNA was cleaved and the 166 fragment gel-purified. One aliquot was subjected to β-elimination and gel-purified a second time. The two aliquots of 166 were reacted with labeled 3′-fragment for 45 min. M: 166.22 cleaved and labeled with [32P]pCp. The experiment shows that G229 is critical for the ligation reaction.

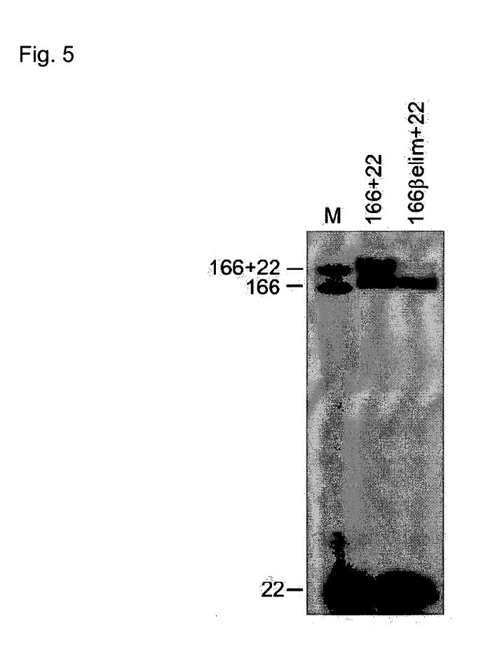

FIG. 6. Alkaline hydrolysis of [32P]pC-labeled 3′-fragment isolated from 157.22 and 166.22. The samples were incubated in a carbonate-buffer at pH 9.0 for 0, 4, 8, and 12 min, respectively. Two signals (corresponding to A231 and U232) are missing from the ladder of (157)22 RNA compared with (166)22. This indicates that the 2′-OH of U232 is blocked by the formation of a 2′, 5′ bond with C230. A complete ladder when (157)22 is preincubated with 166 because of ligation and recleavage by hydrolysis.

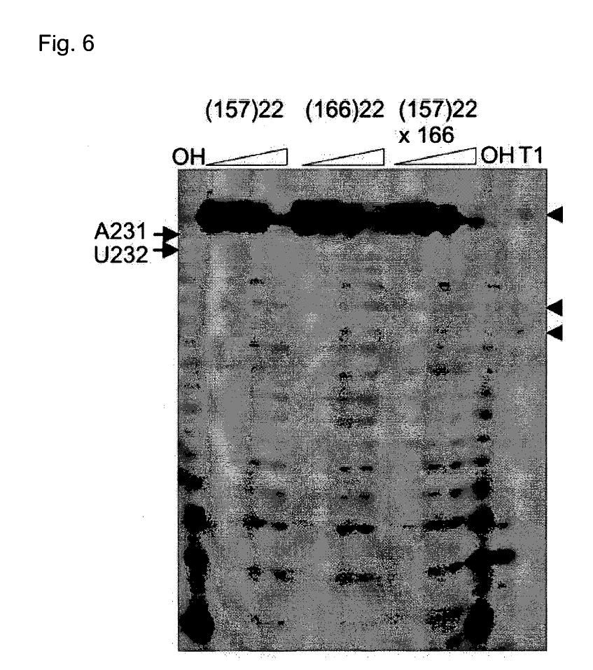

FIG. 7. (A) Diagram showing the proposed structure of the branch with labeled phosphate in bold face (top diagram). (B) Gel electrophoretic analysis of 22 nt 3′-fragments treated with mung bean nuclease (MB) and MB followed by alkaline phosphatase (AP). OH: Alkaline hydrolysis of [32P]pCp-labeled 3′-fragment isolated from 166.22. pN: free nucleotides. Pi: phosphate. The resistant fragments are marked with an asterisk. The resistant fragment is only observed with (157)22 RNA and not with (166)22 RNA. The position of labeled fragments in the gel is consistent with the structures in (A).

FIG. 8. Primer extension analysis of all RNA and site-specifically deoxy substituted 29 nt oligos after incubation with ribozyme at standard cleavage conditions for 2 hours. The ribozyme was of the 157.-7 format and the oligos 7.22 (indicating number of nucleotides included, upstream and downstream of IPS, respectively). A primer extension stop at IPS2 indicates that the cleavage occurs by transesterification.

FIG. 9 In the basic construct (pBAD-GFP (Guzman L M et al. J. Bacteriol. 177, 4121-4130 (95) pBAD-GFP is a modified construct ); top line, a GFP (Green Fluorescent Protein) open reading frame is transcribed from the arabinose inducible promoter pBAD. An Ndel restriction site for insertion is placed at the initiation codon. In GIR1wtGFP, a wild-type GIR1 fused to a synthetic 3′-part that contain a 22 nt duplication of sequence immediately upstream of the initiation codon is cloned into the Ndel-site. The GIR1 used in this study is in the 157.22 format (Nielsen H et al. Science 309, 1584-1587 (05)). GIR1invGFP has the same insert in the opposite orientation. As a result, there is no RBS (Ribosome Binding Site) in the vicinity of the initiation codon. GIR1P7⁻GFP is different from GIR1wtGFP in that it has an inactivating mutation ((G174C) at the G-binding site in P7. In the P7⁻mutant, no cleavage at the IPS (Internal Processing Site) is expected. All cloning procedures and other basic procedures were according to Sambrook J et al. “Molecular Cloning” 2nd ed. Cold Spring Harbor Laboratory Press (89).

FIG. 10 The constructs described in FIG. 1 were transformed into competent E. coli DH5α. Cells were grown on LB medium and analysed in the absence or presence of the inducer arabinose. RNA was extracted by the hot phenol method (Aiba H et al. J. Biol. Chem., 256, 11905-11910 (81)) and analysed by primer extension using primers complementary to GIR1 (A) (C473: 5′-CCC GAT TGC ATC ATG GTG A) or GFP (B) (C474: 5′-ATT GGG ACA ACT CCA GTG A). The products were run on 6% denaturing (urea) acylamide gels along with sequencing ladders made with the same primers and plasmid preps of the constructs as templates. pBAD-GFP shows the expected inducibility by arabinose. No transcript is detected in GIR1invGFP. This is expected because the lack of a RBS positioned in front of the initiation codon results in very rapid turn-over of the transcript. In GIR1wtGFP and GIR1P7⁻GFP, the same arabinose inducibility is found as in the starting construct pBAD-GFP. The difference between the two is the presence of a primer extension stop signal in GIR1wtGFP, but not in GIR1 P7⁻GFP corresponding to GIR1 catalysed cleavage at IPS. Notably, a primer extension product at this position is also found in the uninduced state where no primer extension stop signal corresponding to the 5′-end of the primary transcript is detected in any of the constructs. This signal is taken to represent low level transcription in the culture that is stabilized by the action of GIR1. The absence of a signal with either of the two primers in uninduced GIR1P7⁻GFP cells makes an effect on transcription of the GIR1 insert unlikely. In other experiments it was shown that the half-life of the 5′-end of the transcripts from the pBAD-GFP and GIR1wtGFP constructs were of the same order (ca. 1 min).

FIG. 11 Cells containing the different constructs were plated on LB/Amp plates without or with the inducer arabinose. On the ara⁺ plate, bright fluorescence is observed with the pBAD-GFP construct, medium fluorescence with the GIR1wtGFP and GIR1P7⁻GFP constructs, and no fluorescence with the GIR1invGFP construct, as expected. In line with the above interpretation of the primer extension analysis, the only construct that result in GFP production in the absence of arabinose is GIR1wtGFP.

### DETAILED DESCRIPTION OF THE INVENTION

In a preferred embodiment, the isolated nucleic acid is a polynucleotide or an expression vector and comprises a SNA or RNA sequence operably linked to a promoter or other regulatory sequences to control expression thereof. Expression vectors can encode one or more DNAs or RNAs and these can be coordinately or individually expressed, e.g., using one promoter or multiple promoters. Useful promoters and regulatory sequences for any of the expression vectors are well known to those of skill in the art.

Expression vectors are useful for any one of the following purposes: propagation of the DNA or RNA, purification of the DNA or RNA, or delivery and expression or transcription of the DNA or RNA in a subject. Expression vectors can be used for any cell type, including bacterial, yeast, fungi and mammalian systems, and include all types of vectors including viral vectors. Methods of making and using expression vectors, as well as selecting the appropriate host cell system are well known to those of skill in the art. Well-known promoters can be present, such as the lactose promoter system, a tryptophan (Trp) promoter system, a beta-lactamase promoter system, an arabinose-inducible promoter or a promoter system from phage lambda. The promoters typically control expression, optionally with an operator sequence and have ribosome binding site sequences for example, for initiating and completing transcription and translation. Among vectors preferred for use in bacteria include pQE70, pQE60 and pQE9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Any expression vector is contemplated.

In one embodiment there are provided methods for stabilising polynucleotides and methods for improving the production of polypeptides as a result of said stabilisation. In a preferred embodiment the polynucleotide to be stabilized is an RNA polynucleotide.

The RNA polynucleotide to be stabilized can be any RNA, in particular a messenger RNA (mRNA), a ribosomal RNA (rRNA), a transfer RNA (tRNA), a nuclear RNA, a RNA-ribozyme, a small nucleolar RNA (snoRNA), a microRNA (miRNA), a short interfering RNA (siRNA), a small nuclear RNA (snRNA) and a small double-stranded RNA, an in vitro transcribed RNA or a chemically synthesised RNA, in which respect all said RNA molecules can be chemically modified. In that respect the RNA may have between 10 and 100,000 nucleotides, such as from 15 to 2500 nucleotides, for example from 20 to 1000 nucleotides.

Selected, but non-limited examples of second subsequences in the form of RNA polynucleotides of the invention are given in Table 3 below. It will be understood that such sequences, or a complementary strand thereof, can be operably linked to a first subsequence as defined herein elsewhere:

The below second subsequences can be accessed through the miRBASE: (http://microrna.sanger.ac.uk/sequences/)

TABLE 3

| SEQ | | | |
|---|---|---|---|
| ID | | Accesion | |
| NO | ID | NO. miRBASE | SEQUENCE |
| 3 | hsa-miR- | MIMAT0002872 | AAUCCUUUGUCCCUGGGUGAGA |
| | 501 | | |
| 4 | hsa-miR- | MIMAT0003274 | AAACUACUGAAAAUCAAAGAU |
| | 606 | | |
| 5 | hsa-miR- | MIMAT0002171 | AAUAUAACACAGAUGGCCUGU |
| | 410 | | |
| 6 | hsa-miR- | MIMAT0002809 | UGAGAACUGAAUUCCAUAGGCU |
| | 146b | | |
| 7 | hsa-miR- | MIMAT0003339 | AUCAACAGACAUUAAUUGGGCGC |
| | 421 | | |
| 8 | hsa-miR- | MIMAT0003311 | AAAGACAUAGGAUAGAGUCACCUC |
| | 641 | | |
| 9 | hsa-miR- | MIMAT0002854 | AACGCACUUCCCUUUAGAGUGU |
| | 521 | | |
| 10 | hsa-miR- | MIMAT0000269 | UAACAGUCUCCAGUCACGGCC |
| | 212 | | |
| 11 | hsa-miR- | MIMAT0000088 | CUUUCAGUCGGAUGUUUGCAGC |
| | 30a-3p | | |
| 12 | hsa-miR- | MIMAT0003242 | UAGAUAAAAUAUUGGUACCUG |
| | 577 | | |
| 13 | hsa-miR- | MIMAT0000462 | UGGAAUGUAAGGAAGUGUGUGG |
| | 206 | | |
| 14 | hsa-miR- | MIMAT0003316 | AAGCAGCUGCCUCUGAGGC |
| | 646 | | |
| 15 | hsa-miR- | MIMAT0001629 | AACACACCUGGUUAACCUCUUU |
| | 329 | | |
| 16 | hsa-miR- | MIMAT0000079 | GUGCCUACUGAGCUGAUAUCAGU |
| | 189 | | |
| 17 | hsa-miR- | MIMAT0000439 | UUGCAUAGUCACAAAAGUGA |
| | 153 | | |
| 18 | hsa-miR- | MIMAT0000226 | UAGGUAGUUUCAUGUUGUUGG |
| | 196a | | |
| 19 | hsa-miR- | MIMAT0002816 | UGAAACAUACACGGGAAACCUCUU |
| | 494 | | |
| 20 | hsa-miR- | MIMAT0002839 | GAAGGCGCUUCCCUUUAGAGC |
| | 525* | | |
| 21 | hsa-miR- | MIMAT0000073 | UGUGCAAAUCUAUGCAAAACUGA |
| | 19a | | |
| 22 | hsa-miR- | MIMAT0000104 | AGCAGCAUUGUACAGGGCUAUCA |
| | 107 | | |
| 23 | hsa-miR- | MIMAT0000676 | UCACAGUGAACCGGUCUCUUUC |
| | 128b | | |
| 24 | hsa-miR- | MIMAT0002863 | AAAGCGCUUCCCUUUGCUGGA |
| | 518a | | |
| 25 | hsa-miR- | MIMAT0000068 | UAGCAGCACAUAAUGGUUUGUG |
| | 15a | | |
| 26 | hsa-miR- | MIMAT0003329 | UAGUAGACCGUAUAGCGUACG |
| | 411 | | |
| 27 | hsa-miR- | MIMAT0002176 | GUCAUACACGGCUCUCCUCUCU |
| | 485-3p | | |
| 28 | hsa-miR- | MIMAT0003302 | GUGUCUGCUUCCUGUGGGA |
| | 632 | | |
| 29 | hsa-miR- | MIMAT0003322 | AAUGGCGCCACUAGGGUUGUGCA |
| | 652 | | |
| 30 | hsa-miR- | MIMAT0000755 | GCACAUUACACGGUCGACCUCU |
| | 323 | | |
| 31 | hsa-miR- | MIMAT0003224 | GCGUGCGCCGGCCGGCCGCC |
| | 560 | | |
| 32 | hsa-miR- | MIMAT0000242 | CUUUUUGCGGUCUGGGCUUGC |
| | 129 | | |
| 33 | hsa-miR- | MIMAT0000459 | AACUGGCCUACAAAGUCCCAG |
| | 193a | | |
| 34 | hsa-miR- | MIMAT0002805 | AGUGACAUCACAUAUACGGCAGC |
| | 489 | | |
| 35 | hsa-miR- | MIMAT0003228 | AGGCACGGUGUCAGCAGGC |
| | 564 | | |
| 36 | hsa-miR- | MIMAT0000443 | UCCCUGAGACCCUUUAACCUGUG |
| | 125a | | |
| 37 | hsa-miR- | MIMAT0000424 | UCACAGUGAACCGGUCUCUUUU |
| | 128a | | |
| 38 | hsa-miR- | MIMAT0003269 | UGGUCUAGGAUUGUUGGAGGAG |
| | 601 | | |
| 39 | hsa-miR- | MIMAT0000075 | UAAAGUGCUUAUAGUGCAGGUAG |
| | 20a | | |
| 40 | hsa-miR- | MIMAT0002846 | AAAGUGCUUCCUUUUAGAGGGUU |
| | 520c | | |
| 41 | hsa-miR- | MIMAT0003296 | GUGAGUCUCUAAGAAAAGAGGA |
| | 627 | | |
| 42 | hsa-miR- | MIMAT0003291 | ACAGUCUGCUGAGGUUGGAGC |
| | 622 | | |
| 43 | hsa-miR- | MIMAT0000762 | CCACUGCCCCAGGUGCUGCUGG |
| | 324-3p | | |
| 44 | hsa-miR- | MIMAT0001639 | CGAAUGUUGCUCGGUGAACCCCU |
| | 409-3p | | |
| 45 | hsa-miR- | MIMAT0000261 | UAUGGCACUGGUAGAAUUCACUG |
| | 183 | | |
| 46 | hsa- | MIMAT0000416 | UGGAAUGUAAAGAAGUAUGUA |
| miR-1 | | | |
| 47 | hsa-miR- | MIMAT0002806 | CAACCUGGAGGACUCCAUGCUG |
| | 490 | | |
| 48 | hsa-miR- | MIMAT0000729 | AUCAUAGAGGAAAAUCCACGU |
| | 376a | | |
| 49 | hsa-miR- | MIMAT0000726 | GAAGUGCUUCGAUUUUGGGGUGU |
| | 373 | | |
| 50 | hsa-miR- | MIMAT0003150 | UAUGUGCCUUUGGACUACAUCG |
| | 455 | | |
| 51 | hsa-miR- | MIMAT0000617 | UAAUACUGCCGGGUAAUGAUGG |
| | 200c | | |
| 52 | hsa-miR- | MIMAT0000720 | ACAUAGAGGAAAUUCCACGUUU |
| | 368 | | |
| 53 | hsa-miR- | MIMAT0003393 | AAUGACACGAUCACUCCCGUUGA |
| | 425-5p | | |
| 54 | hsa-miR- | MIMAT0003246 | UCUUGUGUUCUCUAGAUCAGU |
| | 581 | | |
| 55 | hsa-miR- | MIMAT0000444 | CAUUAUUACUUUUGGUACGCG |
| | 126* | | |
| 56 | hsa-miR- | MIMAT0001080 | UAGGUAGUUUCCUGUUGUUGG |
| | 196b | | |
| 57 | hsa-miR- | MIMAT0000754 | UCCAGCUCCUAUAUGAUGCCUUU |
| | 337 | | |
| 58 | hsa-miR- | MIMAT0000707 | AAUUGCACGGUAUCCAUCUGUA |
| | 363 | | |
| 59 | hsa-miR- | MIMAT0000437 | GUCCAGUUUUCCCAGGAAUCCCUU |
| | 145 | | |
| 60 | hsa-miR- | MIMAT0000425 | CAGUGCAAUGUUAAAAGGGCAU |
| | 130a | | |
| 61 | hsa-miR- | MIMAT0003215 | AACAGGUGACUGGUUAGACAA |
| | 552 | | |
| 62 | hsa-miR- | MIMAT0002849 | CUACAAAGGGAAGCACUUUCUC |
| | 524* | | |
| 63 | hsa-miR- | MIMAT0000759 | UCAGUGCAUCACAGAACUUUGU |
| | 148b | | |
| 64 | hsa-miR- | MIMAT0001545 | UUUUUGCGAUGUGUUCCUAAUA |
| | 450 | | |
| 65 | hsa-miR- | MIMAT0000750 | UCCGUCUCAGUUACUUUAUAGCC |
| | 340 | | |
| 66 | hsa-miR- | MIMAT0000725 | ACUCAAAAUGGGGGCGCUUUCC |
| | 373* | | |
| 67 | hsa-miR- | MIMAT0002827 | GAGUGCCUUCUUUUGGAGCGU |
| | 515-3p | | |
| 68 | hsa-miR- | MIMAT0000083 | UUCAAGUAAUUCAGGAUAGGUU |
| | 26b | | |
| 69 | hsa-miR- | MIMAT0003234 | AGUUAAUGAAUCCUGGAAAGU |
| | 569 | | |
| 70 | hsa-miR- | MIMAT0000680 | UAAAGUGCUGACAGUGCAGAU |
| | 106b | | |
| 71 | hsa-miR- | MIMAT0002844 | CAAAGCGCUCCCCUUUAGAGGU |
| | 518b | | |
| 72 | hsa-miR- | MIMAT0002820 | CAGCAGCACACUGUGGUUUGU |
| | 497 | | |
| 73 | hsa-miR- | MIMAT0002174 | UCAGGCUCAGUCCCCUCCCGAU |
| | 484 | | |
| 74 | hsa-let- | MIMAT0000067 | UGAGGUAGUAGAUUGUAUAGUU |
| | 7f | | |
| 75 | hsa-miR- | MIMAT0000721 | AAUAAUACAUGGUUGAUCUUU |
| | 369-3p | | |
| 76 | hsa-miR- | MIMAT0003238 | CUGAAGUGAUGUGUAACUGAUCAG |
| | 573 | | |
| 77 | hsa-miR- | MIMAT0002850 | GAAGGCGCUUCCCUUUGGAGU |
| | 524 | | |
| 78 | hsa-miR- | MIMAT0002881 | UGAUUGGUACGUCUGUGGGUAGA |
| | 509 | | |
| 79 | hsa-miR- | MIMAT0000718 | UAAGUGCUUCCAUGUUUGAGUGU |
| | 302d | | |
| 80 | hsa-miR- | MIMAT0002866 | AUCGUGCAUCCUUUUAGAGUGU |
| | 517c | | |
| 81 | hsa-miR- | MIMAT0003293 | UAGUACCAGUACCUUGUGUUCA |
| | 624 | | |
| 82 | hsa-miR- | MIMAT0000773 | UGUCUGCCCGCAUGCCUGCCUCU |
| | 346 | | |
| 83 | hsa-miR- | MIMAT0003309 | AUCGCUGCGGUUGCGAGCGCUGU |
| | 639 | | |
| 84 | hsa-miR- | MIMAT0000275 | UUGUGCUUGAUCUAACCAUGU |
| | 218 | | |
| 85 | hsa-miR- | MIMAT0000714 | ACUUUAACAUGGAAGUGCUUUCU |
| | 302b* | | |
| 86 | hsa-miR- | MIMAT0003233 | GCGACCCAUACUUGGUUUCAG |
| | 551b | | |
| 87 | hsa-miR- | MIMAT0003319 | AAACCUGUGUUGUUCAAGAGUC |
| | 649 | | |
| 88 | hsa-miR- | MIMAT0000724 | AAAGUGCUGCGACAUUUGAGCGU |
| | 372 | | |
| 89 | hsa-miR- | MIMAT0000756 | CCUCUGGGCCCUUCCUCCAG |
| | 326 | | |
| 90 | hsa-miR- | MIMAT0000705 | AAUCCUUGGAACCUAGGUGUGAGU |
| | 362 | | |
| 91 | hsa-miR- | MIMAT0000417 | UAGCAGCACAUCAUGGUUUACA |
| | 15b | | |
| 92 | hsa-miR- | MIMAT0002876 | GUCAACACUUGCUGGUUUCCUC |
| | 505 | | |
| 93 | hsa-miR- | MIMAT0001627 | AUCAUGAUGGGCUCCUCGGUGU |
| | 433 | | |
| 94 | hsa-miR- | MIMAT0003263 | GAAGUGUGCCGUGGUGUGUCU |
| | 595 | | |
| 95 | hsa-miR- | MIMAT0000452 | UAGGUUAUCCGUGUUGCCUUCG |
| | 154 | | |
| 96 | hsa-miR- | MIMAT0003270 | GACACGGGCGACAGCUGCGGCCC |
| | 602 | | |
| 97 | hsa-miR- | MIMAT0003325 | UCCCACGUUGUGGCCCAGCAG |
| | 662 | | |
| 98 | hsa-miR- | MIMAT0003286 | AGACUUCCCAUUUGAAGGUGGC |
| | 617 | | |
| 99 | hsa-miR- | MIMAT0003267 | GUUGUGUCAGUUUAUCAAAC |
| | 599 | | |
| 100 | hsa-miR- | MIMAT0000082 | UUCAAGUAAUCCAGGAUAGGC |
| | 26a | | |
| 101 | hsa-miR- | MIMAT0000434 | UGUAGUGUUUCCUACUUUAUGGA |
| | 142-3p | | |
| 102 | hsa-miR- | MIMAT0000421 | UGGAGUGUGACAAUGGUGUUUGU |
| | 122a | | |
| 103 | hsa-miR- | MIMAT0002817 | AAACAAACAUGGUGCACUUCUUU |
| | 495 | | |
| 104 | hsa-miR- | MIMAT0001620 | CAUCUUACCGGACAGUGCUGGA |
| | 200a* | | |
| 105 | hsa-miR- | MIMAT0000681 | UAGCACCAUUUGAAAUCGGU |
| | 29c | | |
| 106 | hsa-miR- | MIMAT0000426 | UAACAGUCUACAGCCAUGGUCG |
| | 132 | | |
| 107 | hsa-miR- | MIMAT0003259 | AGACCAUGGGUUCUCAUUGU |
| | 591 | | |
| 108 | hsa-miR- | MIMAT0003222 | UGAGCUGCUGUACCAAAAU |
| | 558 | | |
| 109 | hsa-miR- | MIMAT0003281 | AGGAAUGUUCCUUCUUUGCC |
| | 613 | | |
| 110 | hsa-miR- | MIMAT0003256 | UCAGAACAAAUGCCGGUUCCCAGA |
| | 589 | | |
| 111 | hsa-miR- | MIMAT0000272 | AUGACCUAUGAAUUGACAGAC |
| | 215 | | |
| 112 | hsa-miR- | MIMAT0003305 | ACUUGGGCACUGAAACAAUGUCC |
| | 635 | | |
| 113 | hsa-miR- | MIMAT0000094 | UUCAACGGGUAUUUAUUGAGCA |
| | 95 | | |
| 114 | hsa-miR- | MIMAT0000453 | AAUCAUACACGGUUGACCUAUU |
| | 154* | | |
| 115 | hsa-miR- | MIMAT0002822 | CACUCAGCCUUGAGGGCACUUUC |
| | 512-5p | | |
| 116 | hsa-miR- | MIMAT0000255 | UGGCAGUGUCUUAGCUGGUUGUU |
| | 34a | | |
| 117 | hsa-miR- | MIMAT0003313 | ACUUGUAUGCUAGCUCAGGUAG |
| | 643 | | |
| 118 | hsa-miR- | MIMAT0003249 | UUAUGGUUUGCCUGGGACUGAG |
| | 584 | | |
| 119 | hsa-miR- | MIMAT0000428 | UAUGGCUUUUUAUUCCUAUGUGA |
| | 135a | | |
| 120 | hsa-miR- | MIMAT0000100 | UAGCACCAUUUGAAAUCAGUGUU |
| | 29b | | |

Further non-limited examples of second subsequences in the form of RNA polynucleotides according to the present invention are listed in Table 4 below. It will be understood that such sequences, or a complementary strand thereof, can be operably linked to a first subsequence as defined herein elsewhere:

The sequences can be accessed through the miRBASE: (http://microrna.sanger.ac.uk/sequences/)

TABLE 4

| SEQ ID | | Accesion | |
|---|---|---|---|
| NO | ID | NO. miRBASE | SEQUENCE |
| 121. | hsa-mir- | MI0003157 | CUCAGGCUGUGACCCU- |
| | 526a-1 | | CUAGAGGGAAGCACUUU- |
| | | | CUGUUGCUUGAAAGAAGA- |
| | | | GAAAGCGCUUCCUUUUA- |
| | | | GAGGAUUACUCUUUGAG |
| 122. | hsa-mir- | MI0000293 | AGUAUAAUUAUUACAUA- |
| | 217 | | GUUUUUGAUGUCGCA- |
| | | | GAUACUGCAUCAGGAACU- |
| | | | GAUUGGAUAAGAAUCAGU- |
| | | | CACCAUCAGUUCCUAAUG- |
| | | | CAUUGCCUUCAGCAU- |
| | | | CUAAACAAG |
| 123. | hsa-mir- | MI0000456 | UGUGUCUCUCUCUGUGUC- |
| | 140 | | CUGCCAGUGGUUUUACC- |
| | | | CUAUGGUAGGUUACGU- |
| | | | CAUGCUGUUCUACCA- |
| | | | CAGGGUAGAACCACGGA- |
| | | | CAGGAUACCGGGGCACC |
| 124. | hsa-mir- | MI0003175 | UCCCAUGCUGUGACCCU- |
| | 520h | | CUAGAGGAAGCACUUUCU- |
| | | | GUUUGUUGUCUGA- |
| | | | GAAAAAACAAAGUG- |
| | | | CUUCCCUUUAGAGUUACU- |
| | | | GUUUGGGA |
| 125. | hsa-mir- | MI0003142 | AACCCUCCUUGGGAAGU- |
| | 498 | | GAAGCUCAGGCUGU- |
| | | | GAUUUCAAGCCAGGGGGC- |
| | | | GUUUUUCUAUAACUGGAU- |
| | | | GAAAAGCACCUCCAGAG- |
| | | | CUUGAAGCUCACAGUUU- |
| | | | GAGAGCAAUCGUCUAAG- |
| | | | GAAGUU |
| 126. | hsa-mir- | MI0000478 | GCCGGCGCCCGAGCU- |
| | 149 | | CUGGCUCCGUGUCUUCA- |
| | | | CUCCCGUGCUUGUCCGAG- |
| | | | GAGGGAGGGAGGGAC- |
| | | | GGGGGCUGUGCUGGGG- |
| | | | CAGCUGGA |
| 127. | hsa-mir- | MI0000743 | AGUCUAGUUACUAGGCA- |
| | 34c | | GUGUAGUUAGCUGAUUG- |
| | | | CUAAUAGUACCAAUCA- |
| | | | CUAACCACACGGCCAG- |
| | | | GUAAAAAGAUU |
| 128. | hsa-mir- | MI0001723 | CCGGGGAGAAGUACGGU- |
| | 433 | | GAGCCUGUCAUUAUUCA- |
| | | | GAGAGGCUAGAUCCUCU- |
| | | | GUGUUGAGAAGGAUCAU- |
| | | | GAUGGGCUCCUCGGUGUU- |
| | | | CUCCAGG |
| 129. | hsa-mir- | MI0000078 | GGCUGAGCCGCAGUAGUU- |
| | 22 | | CUUCAGUGGCAAG- |
| | | | CUUUAUGUCCUGACCCAG- |
| | | | CUAAAGCUGCCAGUUGAA- |
| | | | GAACUGUUGCCCUCUGCC |
| 130. | hsa-mir- | MI0003625 | UCCCAUCUGGACCCUG- |
| | 612 | | CUGGGCAGGGCUUCUGAG- |
| | | | CUCCUUAGCACUAGCAG- |
| | | | GAGGGGCUCCAGGGGCC- |
| | | | CUCCCUCCAUGGCAGC- |
| | | | CAGGACAGGACUCUCA |
| 131. | hsa-mir- | MI0000787 | AGAGAUGGUAGACUAUG- |
| | 379 | | GAACGUAGGCGUUAU- |
| | | | GAUUUCUGACCUAUGUAA- |
| | | | CAUGGUCCACUAACUCU |
| 132. | hsa-mir- | MI0000490 | UGCUUCCCGAGGCCA- |
| | 206 | | CAUGCUUCUUUAUAUCCC- |
| | | | CAUAUGGAUUACUUUG- |
| | | | CUAUGGAAUGUAAGGAA- |
| | | | GUGUGUGGUUUCGGCAA- |
| | | | GUG |
| 133. | hsa-mir- | MI0000443 | AGGCCUCUCUCUCCGU- |
| | 124a-1 | | GUUCACAGCGGACCUU- |
| | | | GAUUUAAAUGUCCAUA- |
| | | | CAAUUAAGGCACGCGGU- |
| | | | GAAUGCCAAGAAUGGGG- |
| | | | CUG |
| 134. | hsa-mir- | MI0003577 | CUAGAUAAGUUAUUAG- |
| | 570 | | GUGGGUGCAAAG- |
| | | | GUAAUUGCAGUUUUUCC- |
| | | | CAUUAUUUUAAUUGC- |
| | | | GAAAACAGCAAUUAC- |
| | | | CUUUGCACCAACCUGAUG- |
| | | | GAGU |
| 135. | hsa-mir- | MI0003588 | GUUAUGUGAAGGUAUU- |
| | 581 | | CUUGUGUUCUCUAGAUCA- |
| | | | GUGCUUUUAGAAAAUUU- |
| | | | GUGUGAUCUAAAGAACA- |
| | | | CAAAGAAUACCUACACA- |
| | | | GAACCACCUGC |
| 136. | hsa-mir- | MI0003572 | GCUAGGCGUGGUGGC- |
| | 566 | | GGGCGCCUGUGAUCCCAA- |
| | | | CUACUCAGGAGGCUGGGG- |
| | | | CAGCAGAAUCGCUU- |
| | | | GAACCCGGGAGGCGAAG- |
| | | | GUUGCAGUGAGC |
| 137. | hsa-mir- | MI0000790 | UACUUGAAGAGAAGUU- |
| | 382 | | GUUCGUGGUGGAUUC- |
| | | | GCUUUACUUAUGACGAAU- |
| | | | CAUUCACGGACAACA- |
| | | | CUUUUUUCAGUA |
| 138. | hsa-mir- | MI0003137 | GUGGUCUCAGAAUC- |
| | 193b | | GGGGUUUUGAGGGCGA- |
| | | | GAUGAGUUUAU- |
| | | | GUUUUAUCCAACUGGCC- |
| | | | CUCAAAGUCCC- |
| | | | GCUUUUGGGGUCAU |
| 139. | hsa-mir- | MI0000484 | UGCUCCCUCUCUCA- |
| | 188 | | CAUCCCUUGCAUGGUG- |
| | | | GAGGGUGAGCUUUCU- |
| | | | GAAAACCCCUCCCACAUG- |
| | | | CAGGGUUUGCAGGAUGGC- |
| | | | GAGCC |
| 140. | hsa-mir- | MI0001448 | GAAAGCGCUUUGGAAUGA- |
| | 425 | | CACGAUCACUCCCGUUGA- |
| | | | GUGGGCACCCGAGAAGC- |
| | | | CAUCGGGAAUGUCGU- |
| | | | GUCCGCCCAGUGCUCUUUC |
| 141. | hsa-mir- | MI0000089 | GGAGAGGAGGCAAGAUG- |
| | 31 | | CUGGCAUAGCUGUUGAA- |
| | | | CUGGGAACCUGCUAUGC- |
| | | | CAACAUAUUGCCAU- |
| | | | CUUUCC |
| 142. | hsa-mir- | MI0003188 | UGCCCUAGCAGCGGGAA- |
| | 503 | | CAGUUCUGCAGUGAGC- |
| | | | GAUCGGUGCUCUGGG- |
| | | | GUAUUGUUUCCGCUGC- |
| | | | CAGGGUA |
| 143. | hsa-mir- | MI0000098 | UGGCCGAUUUUGGCA- |
| | 96 | | CUAGCACAUUUUUGCUU- |
| | | | GUGUCUCUCCGCUCUGAG- |
| | | | CAAUCAUGUGCAGUGC- |
| | | | CAAUAUGGGAAA |
| 144. | hsa-mir- | MI0000441 | ACCAAGUUUCAGUUCAU- |
| | 30b | | GUAAACAUCCUACACU- |
| | | | CAGCUGUAAUACAUG- |
| | | | GAUUGGCUGGGAGGUG- |
| | | | GAUGUUUACUUCAGCUGA- |
| | | | CUUGGA |
| 145. | hsa-mir- | MI0003139 | GUCCCCUCCCCUAGGCCA- |
| | 181d | | CAGCCGAGGUCACAAU- |
| | | | CAACAUUCAUUGUUGUC- |
| | | | GGUGGGUUGUGAGGACU- |
| | | | GAGGCCAGACCCACC- |
| | | | GGGGGAUGAAUGUCACU- |
| | | | GUGGCUGGGCCAGACAC- |
| | | | GGCUUAAGGGGAAUGGG- |
| | | | GAC |
| 146. | hsa-mir- | MI0000270 | CCUGUGCAGA- |
| | 181b-1 | | GAUUAUUUUUUAAAAGGU- |
| | | | CACAAUCAACAUUCAUUG- |
| | | | CUGUCGGUGGGUUGAACU- |
| | | | GUGUGGACAAGCUCACU- |
| | | | GAACAAUGAAUGCAACU- |
| | | | GUGGCCCCGCUU |
| 147. | hsa-mir- | MI0003161 | UCUCAGGCAGUGACCCU- |
| | 517a | | CUAGAUGGAAGCACUGU- |
| | | | CUGUUGUAUAAAAGAAAA- |
| | | | GAUCGUGCAUCCCUUUA- |
| | | | GAGUGUUACUGUUUGAGA |
| 148. | hsa-mir- | MI0003183 | GCCCUGUCCCCUGUGC- |
| | 499 | | CUUGGGCGGGCGGCU- |
| | | | GUUAAGACUUGCAGUGAU- |
| | | | GUUUAACUCCUCUCCAC- |
| | | | GUGAACAUCACAGCAAGU- |
| | | | CUGUGCUGCUUCCCGUCC- |
| | | | CUACGCUGCCUGGGCAGG- |
| | | | GU |
| 149. | hsa-mir- | MI0000457 | CGGCCGGCCCUGGGUC- |
| | 141 | | CAUCUUCCAGUACAGU- |
| | | | GUUGGAUGGUCUAAUUGU- |
| | | | GAAGCUCCUAACACUGU- |
| | | | CUGGUAAAGAUGGCUCCC- |
| | | | GGGUGGGUUC |
| 150. | hsa-mir- | MI0003666 | AAUCUAUCACUG- |
| | 651 | | CUUUUUAGGAUAAGCUU- |
| | | | GACUUUUGUU- |
| | | | CAAAUAAAAAUGCAAAAG- |
| | | | GAAAGUGUAUC- |
| | | | CUAAAAGGCAAUGACA- |
| | | | GUUUAAUGUGUUU |
| 151. | hsa-mir- | MI0000805 | GAAACUGGGCUCAAGGU- |
| | 342 | | GAGGGGUGCUAUCUGU- |
| | | | GAUUGAGGGACAUG- |
| | | | GUUAAUGGAAUUGUCUCA- |
| | | | CACAGAAAUCGCACCCGU- |
| | | | CACCUUGGCCUACUUA |
| 152. | hsa-mir- | MI0003609 | UACUUACUCUACGUGUGU- |
| | 597 | | GUCACUCGAUGACCACU- |
| | | | GUGAAGACAGUAAAAU- |
| | | | GUACAGUGGUUCUCUU- |
| | | | GUGGCUCAAGCGUAAU- |
| | | | GUAGAGUACUGGUC |
| 153. | hsa-mir- | MI0000252 | GGAUCUUUUUGCGGU- |
| | 129-1 | | CUGGGCUUGCUGUUCCU- |
| | | | CUCAACAGUAGUCAG- |
| | | | GAAGCCCUUACCC- |
| | | | CAAAAAGUAUCU |
| 154. | hsa-mir- | MI0000109 | UACUGCCCUCGGCUU- |
| | 103-1 | | CUUUACAGUGCUGCCUU- |
| | | | GUUGCAUAUGGAUCAAG- |
| | | | CAGCAUUGUACAGGG- |
| | | | CUAUGAAGGCAUUG |
| 155. | hsa-mir- | MI0000472 | UGUGAUCACUGUCUC- |
| | 127 | | CAGCCUGCUGAAGCUCA- |
| | | | GAGGGCUCUGAUUCA- |
| | | | GAAAGAUCAUCGGAUCC- |
| | | | GUCUGAGCUUGGCUGGUC- |
| | | | GGAAGUCUCAUCAUC |
| 156. | hsa-mir- | MI0000824 | AUACAGUGCUUGGUUC- |
| | 325 | | CUAGUAGGUGUCCAGUAA- |
| | | | GUGUUUGUGACAUAAUUU- |
| | | | GUUUAUUGAGGACCUC- |
| | | | CUAUCAAUCAAGCACU- |
| | | | GUGCUAGGCUCUGG |
| 157. | hsa-mir- | MI0003177 | UCUCAGGCUGUGUCCCU- |
| | 522 | | CUAGAGGGAAGCGCUUU- |
| | | | CUGUUGUCUGAAAGAAAA- |
| | | | GAAAAUGGUUCCCUUUA- |
| | | | GAGUGUUACGCUUUGAGA |
| 158. | hsa-mir- | MI0003148 | UCUCAGCCUGUGACCCU- |
| | 519c | | CUAGAGGGAAGCGCUUU- |
| | | | CUGUUGUCUGAAAGAAAA- |
| | | | GAAAGUGCAUCUUUUUA- |
| | | | GAGGAUUACAGUUUGAGA |
| 159. | hsa-mir- | MI0000076 | GUAGCACUAAAGUG- |
| | 20a | | CUUAUAGUGCAGGUAGU- |
| | | | GUUUAGUUAUCUACUG- |
| | | | CAUUAUGAGCACUUAAA- |
| | | | GUACUGC |
| 160. | hsa-mir- | MI0002466 | CAGUCCUUCUUUG- |
| | 376b | | GUAUUUAAAACGUG- |
| | | | GAUAUUCCUUCUAU- |
| | | | GUUUACGUGAUUCCUG- |
| | | | GUUAAUCAUAGAG- |
| | | | GAAAAUCCAUGUUUUCA- |
| | | | GUAUCAAAUGCUG |
| 161. | hsa-mir- | MI0000812 | GAGUUUGGUUUUGUUUGG- |
| | 331 | | GUUUGUUCUAGGUAUG- |
| | | | GUCCCAGGGAUCCCAGAU- |
| | | | CAAACCAGGCCCCUGGGC- |
| | | | CUAUCCUAGAACCAAC- |
| | | | CUAAGCUC |
| 162. | hsa-mir- | MI0003613 | AAGUCACGUGCUGUGG- |
| | 600 | | CUCCAGCUUCAUAG- |
| | | | GAAGGCUCUUGUCUGU- |
| | | | CAGGCAGUGGAGUUA- |
| | | | CUUACAGACAAGAGC- |
| | | | CUUGCUCAGGCCAGCC- |
| | | | CUGCCC |
| 163. | hsa-mir- | MI0000301 | GGGCUUUCAAGUCACUA- |
| | 224 | | GUGGUUCCGUUUAGUA- |
| | | | GAUGAUUGUGCAUUGUUU- |
| | | | CAAAAUGGUGCCCUAGU- |
| | | | GACUACAAAGCCC |
| 164. | hsa-mir- | MI0000084 | CCGGGACCCAGUUCAA- |
| | 26b | | GUAAUUCAGGAUAGGUU- |
| | | | GUGUGCUGUCCAGCCU- |
| | | | GUUCUCCAUUACUUGG- |
| | | | CUCGGGGACCGG |
| 165. | hsa-mir- | MI0003600 | UGAUGCUUUGCUGGCUG- |
| | 550-1 | | GUGCAGUGCCUGAGGGA- |
| | | | GUAAGAGCCCUGUUGUU- |
| | | | GUAAGAUAGUGUCUUA- |
| | | | CUCCCUCAGGCACAUCUC- |
| | | | CAACAAGUCUCU |
| 166. | hsa-mir- | MI0003673 | UGACCUGAAUCAGGUAGG- |
| | 449b | | CAGUGUAUUGUUAGCUGG- |
| | | | CUGCUUGGGUCAAGUCAG- |
| | | | CAGCCACAACUACCCUGC- |
| | | | CACUUGCUUCUG- |
| | | | GAUAAAUUCUUCU |
| 167. | hsa-mir- | MI0003658 | ACCAAGUGAUAUUCAUU- |
| | 643 | | GUCUACCUGAGCUA- |
| | | | GAAUACAAGUAGUUGGC- |
| | | | GUCUUCAGAGACACUU- |
| | | | GUAUGCUAGCUCAGGUA- |
| | | | GAUAUUGAAUGAAAAA |
| 168. | hsa-mir- | MI0003558 | CUU- |
| | 553 | | CAAUUUUAUUUUAAAAC- |
| | | | GGUGAGAUUUUGUUUUGU- |
| | | | CUGAGAAAAUCUCGCU- |
| | | | GUUUUAGACUGAGG |
| 169. | hsa-mir- | MI0003566 | UCCCCUCUGGCGGCUGC- |
| | 560 | | GCACGGGCCGUGUGAG- |
| | | | CUAUUGCGGUGGG- |
| | | | CUGGGGCAGAUGAC- |
| | | | GCGUGC- |
| | | | GCCGGCCGGCCGCCGAGGG |
| | | | GCUACCGUUC |
| 170. | hsa-mir- | MI0000542 | GCUUCGCUCCCCUCC- |
| | 320 | | GCCUUCUCUUCCCGGUU- |
| | | | CUUCCCGGAGUC- |
| | | | GGGAAAAGCUGGGUUGA- |
| | | | GAGGGCGAAAAAGGAU- |
| | | | GAGGU |
| 171. | hsa-mir- | MI0003163 | UCUCGGGCUGUGACUCUC- |
| | 521-2 | | CAAAGGGAAGAAUUUUCU- |
| | | | CUUGUCUAAAAGAAAA- |
| | | | GAACGCACUUCCCUUUA- |
| | | | GAGUGUUACCGUGUGAGA |
| 172. | hsa-mir- | MI0000072 | UGUUCUAAGGUGCAUCUA- |
| | 18a | | GUGCAGAUAGUGAAGUA- |
| | | | GAUUAGCAUCUACUGCC- |
| | | | CUAAGUGCUCCUUCUGGCA |
| 173. | hsa-mir- | MI0003643 | UCCCUUUCCCAGGG- |
| | 629 | | GAGGGGCUGGGUUUAC- |
| | | | GUUGGGAGAACUUUUAC- |
| | | | GGUGAACCAGGAGGUU- |
| | | | CUCCCAACGUAAGCC- |
| | | | CAGCCCCUCCCCUCUGCCU |
| 174. | hsa-mir- | MI0000764 | UGUUGUCGGGUGGAUCAC- |
| | 363 | | GAUGCAAUUUUGAUGA- |
| | | | GUAUCAUAGGA- |
| | | | GAAAAAUUGCACGGUAUC- |
| | | | CAUCUGUAAACC |
| 175. | hsa-mir- | MI0003636 | AGAGAAGCUGGACAAGUA- |
| | 622 | | CUGGUCUCAGCAGAUU- |
| | | | GAGGAGAGCACCACAGUG- |
| | | | GUCAUCACACAGUCUGCU- |
| | | | GAGGUUGGAGCUGCUGA- |
| | | | GAUGACACU |
| 176. | hsa-mir- | MI0003602 | UAGCCAGUCAGAAAUGAG- |
| | 590 | | CUUAUUCAUAAAAGUGCA- |
| | | | GUAUGGUGAAGUCAAUCU- |
| | | | GUAAUUUUAUGUAUAAG- |
| | | | CUAGUCUCUGAUUGAAA- |
| | | | CAUGCAGCA |
| 177. | hsa-mir- | MI0003513 | UCCCUGGCGUGAGGGUAU- |
| | 455 | | GUGCCUUUGGACUACAUC- |
| | | | GUGGAAGCCAGCACCAUG- |
| | | | CAGUCCAUGGGCAUAUA- |
| | | | CACUUGCCUCAAGGC- |
| | | | CUAUGUCAUC |
| 178. | hsa-mir- | MI0003135 | UGGUACCUGAAAAGAA- |
| | 495 | | GUUGCCCAUGUUAUUUUC- |
| | | | GCUUUAUAUGUGACGAAA- |
| | | | CAAACAUGGUGCACUU- |
| | | | CUUUUUCGGUAUCA |
| 179. | hsa-mir- | MI0003124 | GUGGCAGCUUGGUGGUC- |
| | 489 | | GUAUGUGUGAC- |
| | | | GCCAUUUACUUGAAC- |
| | | | CUUUAGGAGUGACAUCA- |
| | | | CAUAUACGGCAGCUAAA- |
| | | | CUGCUAC |
| 180. | hsa-mir- | MI0000470 | ACCAGACUUUUCCUA- |
| | 125b-2 | | GUCCCUGAGACCCUAA- |
| | | | CUUGUGAGGUAUUUUA- |
| | | | GUAACAUCACAAGUCAGG- |
| | | | CUCUUGGGACCUAGGC- |
| | | | GGAGGGGA |
| 181. | hsa-mir- | MI0000094 | UCAUCCCUGGGUGGG- |
| | 92-2 | | GAUUUGUUGCAUUACUU- |
| | | | GUGUUCUAUAUAAA- |
| | | | GUAUUGCACUUGUCCC- |
| | | | GGCCUGUGGAAGA |
| 182. | hsa-mir- | MI0003156 | UCAUGCUGUGGCCCUCCA- |
| | 518b | | GAGGGAAGCGCUUUCU- |
| | | | GUUGUCUGAAAGAAAA- |
| | | | CAAAGCGCUCCCCUUUA- |
| | | | GAGGUUUACGGUUUGA |
| 183. | hsa-mir- | MI0003158 | UCUCAGGCUGUCGUCCU- |
| | 520c | | CUAGAGGGAAGCACUUU- |
| | | | CUGUUGUCUGAAAGAAAA- |
| | | | GAAAGUGCUUCCUUUUA- |
| | | | GAGGGUUACCGUUUGAGA |
| 184. | hsa-let- | MI0000065 | CCUAGGAAGAGGUAGUAG- |
| | 7d | | GUUGCAUAGUUUUAGGG- |
| | | | CAGGGAUUUUGCCCA- |
| | | | CAAGGAGGUAACUAUAC- |
| | | | GACCUGCUGCCUUU- |
| | | | CUUAGG |
| 185. | hsa-let- | MI0000061 | AGGUUGAGGUAGUAGGUU- |
| | 7a-2 | | GUAUAGUUUAGAAUUA- |
| | | | CAUCAAGGGAGAUAACU- |
| | | | GUACAGCCUCCUAG- |
| | | | CUUUCCU |
| 186. | hsa-mir- | MI0003153 | UCUCAUGCUGUGACCCU- |
| | 523 | | CUAGAGGGAAGCGCUUU- |
| | | | CUGUUGUCUGAAAGAAAA- |
| | | | GAACGCGCUUCCCUAUA- |
| | | | GAGGGUUACCCUUUGAGA |
| 187. | hsa-mir- | MI0003684 | CUGCUCCUUCUCC- |
| | 660 | | CAUACCCAUUGCAUAUC- |
| | | | GGAGUUGUGAAUUCU- |
| | | | CAAAACACCUCCUGUGUG- |
| | | | CAUGGAUUACAGGAGGGU- |
| | | | GAGCCUUGUCAUCGUG |
| 188. | hsa-mir- | MI0003567 | CUUCAUCCACCAGUCCUC- |
| | 561 | | CAGGAACAUCAAGGAU- |
| | | | CUUAAACUUUGCCAGAG- |
| | | | CUACAAAGGCAAA- |
| | | | GUUUAAGAUCCUUGAA- |
| | | | GUUCCUGGGGGAACCAU |
| 189. | hsa-mir- | MI0003182 | UCUCAGGCUGUGUCCCU- |
| | 519a-2 | | CUACAGGGAAGCGCUUU- |
| | | | CUGUUGUCUGAAAGAAAG- |
| | | | GAAAGUGCAUCCUUUUA- |
| | | | GAGUGUUACUGUUUGAGA |
| 190. | hsa-mir- | MI0000342 | CCAGCUCGGGCAGCC- |
| | 200b | | GUGGCCAUCUUACUGGG- |
| | | | CAGCAUUGGAUGGAGU- |
| | | | CAGGUCUCUAAUACUGC- |
| | | | CUGGUAAUGAUGAC- |
| | | | GGCGGAGCCCUGCACG |
| 191. | hsa-mir- | MI0000239 | GGCUGUGCCGGGUAGA- |
| | 197 | | GAGGGCAGUGGGAGGUAA- |
| | | | GAGCUCUUCACCCUUCAC- |
| | | | CACCUUCUCCACCCAG- |
| | | | CAUGGCC |
| 192. | hsa-mir- | MI0000269 | AGAAGGGCUAUCAGGC- |
| | 181a-2 | | CAGCCUUCAGAGGACUC- |
| | | | CAAGGAACAUUCAACGCU- |
| | | | GUCGGUGAGUUUGG- |
| | | | GAUUUGAAAAAACCACU- |
| | | | GACCGUUGACUGUAC- |
| | | | CUUGGGGUCCUUA |
| 193. | hsa-mir- | MI0003126 | UUGACUUAGCUGGGUA- |
| | 491 | | GUGGGGAACCCUUCCAU- |
| | | | GAGGAGUAGAACACUC- |
| | | | CUUAUGCAAGAUUCCCUU- |
| | | | CUACCUGGCUGGGUUGG |
| 194. | hsa-let- | MI0000433 | AGGCUGAGGUAGUAGUUU- |
| | 7g | | GUACAGUUUGAGGGU- |
| | | | CUAUGAUACCACCCGGUA- |
| | | | CAGGAGAUAACUGUA- |
| | | | CAGGCCACUGCCUUGCCA |
| 195. | hsa-mir- | MI0000087 | AUGACUGAUUUCUUUUG- |
| | 29a | | GUGUUCAGAGU- |
| | | | CAAUAUAAUUUUCUAG- |
| | | | CACCAUCUGAAAUC- |
| | | | GGUUAU |
| 196. | hsa-mir- | MI0003583 | UACAAUCCAACGAGGAUU- |
| | 576 | | CUAAUUUCUCCACGU- |
| | | | CUUUGGUAAUAAG- |
| | | | GUUUGGCAAAGAUGUG- |
| | | | GAAAAAUUGGAAUCCU- |
| | | | CAUUCGAUUGGUUAUAAC- |
| | | | CA |
| 197. | hsa-mir- | MI0000283 | GUGUUGGGGACUC- |
| | 203 | | GCGCGCUGGGUCCAGUG- |
| | | | GUUCUUAACAGUUCAACA- |
| | | | GUUCUGUAGCGCAAUUGU- |
| | | | GAAAUGUUUAGGACCA- |
| | | | CUAGACCC- |
| | | | GGCGGGCGCGGCGACAGC- |
| | | | GA |
| 198. | hsa-mir- | MI0000261 | GUGUAUUCUACAGUGCAC- |
| | 139 | | GUGUCUCCAGUGUGGCUC- |
| | | | GGAGGCUGGAGAC- |
| | | | GCGGCCCUGUUGGAGUAAC |
| 199. | hsa-mir- | MI0003662 | AGGAAGUGUUGGCCU- |
| | 647 | | GUGGCUGCACUCACUUC- |
| | | | CUUCAGCCCCAGGAAGC- |
| | | | CUUGGUCGGGGGCAG- |
| | | | GAGGGAGGGUCAGG- |
| | | | CAGGGCUGGGGGCCUGAC |
| 200. | hsa-mir- | MI0003667 | ACGAAUGGCUAUGCACUG- |
| | 652 | | CACAACCCUAGGAGAGG- |
| | | | GUGCCAUUCACAUAGA- |
| | | | CUAUAAUUGAAUGGC- |
| | | | GCCACUAGGGUUGUGCA- |
| | | | GUGCACAACCUACAC |
| 201. | hsa-mir- | MI0000486 | UGCAGGCCUCUGUGU- |
| | 190 | | GAUAUGUUU- |
| | | | GAUAUAUUAGGUU- |
| | | | GUUAUUUAAUCCAA- |
| | | | CUAUAUAUCAAA- |
| | | | CAUAUUCCUACAGUGU- |
| | | | CUUGCC |
| 202. | hsa-mir- | MI0003685 | GCACAUUGUAGGCCU- |
| | 421 | | CAUUAAAUGUUUGUU- |
| | | | GAAUGAAAAAAUGAAU- |
| | | | CAUCAACAGA- |
| | | | CAUUAAUUGGGCGCCUG- |
| | | | CUCUGUGAUCUC |
| 203. | hsa-mir- | MI0003599 | UCCAGCCUGUGCCCAG- |
| | 589 | | CAGCCCCUGAGAACCAC- |
| | | | GUCUGCUCUGAGCUGG- |
| | | | GUACUGCCUGUUCAGAA- |
| | | | CAAAUGCCGGUUCCCA- |
| | | | GACGCUGCCAGCUGGCC |
| 204. | hsa-mir- | MI0000298 | UGAACAUCCAGGU- |
| | 221 | | CUGGGGCAUGAACCUGG- |
| | | | CAUACAAUGUAGAUUUCU- |
| | | | GUGUUCGUUAGGCAACAG- |
| | | | CUACAUUGUCUGCUGG- |
| | | | GUUUCAGGCUACCUG- |
| | | | GAAACAUGUUCUC |
| 205. | hsa-mir- | MI0003653 | GUGAGCGGGCGCGGCAGG- |
| | 638 | | GAUCGCGGGCGGGUGGC- |
| | | | GGCCUAGGGC- |
| | | | GCGGAGGGCGGACC- |
| | | | GGGAAUGGCGCGCCGUGC- |
| | | | GCCGCCGGCGUAACUGC- |
| | | | GGCGCU |
| 206. | hsa-mir- | MI0003630 | CAUUGGCAUCUAUUAG- |
| | 548c | | GUUGGUGCAAAA- |
| | | | GUAAUUGCGGUUUUUGC- |
| | | | CAUUACUUUCAGUAG- |
| | | | CAAAAAUCUCAAUUA- |
| | | | CUUUUGCACCAA- |
| | | | CUUAAUACUU |
| 207. | hsa-mir- | MI0000449 | CCGCCCCCGCGUCUC- |
| | 132 | | CAGGGCAACCGUGG- |
| | | | CUUUCGAUUGUUACU- |
| | | | GUGGGAACUGGAGGUAA- |
| | | | CAGUCUACAGCCAUGGUC- |
| | | | GCCCCGCAGCAC- |
| | | | GCCCACGCGC |
| 208. | hsa-mir- | MI0000746 | GGCACCCACCCGUA- |
| | 99b | | GAACCGACCUUGC- |
| | | | GGGGCCUUCGCCGCACA- |
| | | | CAAGCUCGUGUCUGUGG- |
| | | | GUCCGUGUC |
| 209. | hsa-mir- | MI0003581 | GGGACCUGCGUGGGUGC- |
| | 574 | | GGGCGUGUGAGUGUGUGU- |
| | | | GUGUGAGUGUGUGUC- |
| | | | GCUCCGGGUCCACGCU- |
| | | | CAUGCACACACCCACAC- |
| | | | GCCCACACUCAGG |
| 210. | hsa-mir- | MI0000073 | GCAGUCCUCUGUUA- |
| | 19a | | GUUUUGCAUAGUUGCA- |
| | | | CUACAAGAAGAAUGUA- |
| | | | GUUGUGCAAAUCUAUG- |
| | | | CAAAACUGAUGGUGGC- |
| | | | CUGC |
| 211. | hsa-mir- | MI0003172 | UCUCAGGCUGUGACCAU- |
| | 516-4 | | CUGGAGGUAAGAAGCA- |
| | | | CUUUCUGUUUUGUGAAA- |
| | | | GAAAAGAAAGUGCUUC- |
| | | | CUUUCAGAGGGUUACU- |
| | | | CUUUGAGA |
| 212. | hsa-mir- | MI0002464 | CUGGGGUACGGGGAUG- |
| | 412 | | GAUGGUCGACCAGUUG- |
| | | | GAAAGUAAUUGUUU- |
| | | | CUAAUGUACUUCACCUG- |
| | | | GUCCACUAGCCGUCC- |
| | | | GUAUCCGCUGCAG |
| 213. | hsa-mir- | MI0000774 | CCUCUACUUUAACAUG- |
| | 302d | | GAGGCACUUGCUGUGA- |
| | | | CAUGACAAAAAUAAGUG- |
| | | | CUUCCAUGUUUGAGUGUGG |
| 214. | hsa-mir- | MI0000463 | CUCACAGCUGCCAGUGU- |
| | 153-1 | | CAUUUUUGUGAUCUGCAG- |
| | | | CUAGUAUUCUCACUCCA- |
| | | | GUUGCAUAGUCACAAAA- |
| | | | GUGAUCAUUGGCAGGU- |
| | | | GUGGC |
| 215. | hsa-mir- | MI0003131 | CAACUACAGCCACUACUA- |
| | 492 | | CAGGACCAUCGAGGAC- |
| | | | CUGCGGGACAAGAUU- |
| | | | CUUGGUGCCACCAUUGA- |
| | | | GAACGCCAGGAUUGUC- |
| | | | CUGCAGAUCAACAAUGCU- |
| | | | CAACUGGCUGCAGAUG |
| 216. | hsa-mir- | MI0000444 | AUCAAGAUUAGAGGCU- |
| | 124a-2 | | CUGCUCUCCGUGUUCA- |
| | | | CAGCGGACCUU- |
| | | | GAUUUAAUGUCAUA- |
| | | | CAAUUAAGGCACGCGGU- |
| | | | GAAUGCCAAGAGCGGAGC- |
| | | | CUACGGCUGCACUUGAA |
| 217. | hsa-mir- | MI0003140 | UCUCAGUCUGUGGCACU- |
| | 512-1 | | CAGCCUUGAGGGCACUUU- |
| | | | CUGGUGCCAGAAUGAAA- |
| | | | GUGCUGUCAUAGCUGAG- |
| | | | GUCCAAUGACUGAGG |
| 218. | hsa-mir- | MI0000681 | CUGUUAAUGCUAAUCGU- |
| | 155 | | GAUAGGGGUUUUUGCCUC- |
| | | | CAACUGACUCCUA- |
| | | | CAUAUUAGCAUUAACAG |
| 219. | hsa-mir- | MI0000781 | GGGAUACU- |
| | 373 | | CAAAAUGGGGGCGCUUUC- |
| | | | CUUUUUGUCUGUACUGG- |
| | | | GAAGUGCUUC- |
| | | | GAUUUUGGGGUGUCCC |
| 220. | hsa-mir- | MI0003557 | AACCAUUCAAAUAUACCA- |
| | 552 | | CAGUUUGUUUAAC- |
| | | | CUUUUGCCUGUUGGUU- |
| | | | GAAGAUGCCUUUCAACAG- |
| | | | GUGACUGGUUAGACAAA- |
| | | | CUGUGGUAUAUACA |
| 221. | hsa-mir- | MI0000750 | GGCUGUGGCUGGAUUCAA- |
| | 26a-2 | | GUAAUCCAGGAUAGGCU- |
| | | | GUUUCCAUCUGUGAGGC- |
| | | | CUAUUCUUGAUUACUU- |
| | | | GUUUCUGGAGGCAGCU |
| 222. | hsa-mir- | MI0000292 | GAUGGCUGUGAGUUGG- |
| | 216 | | CUUAAUCUCAGCUGGCAA- |
| | | | CUGUGAGAUGUUCAUA- |
| | | | CAAUCCCUCACAGUGGU- |
| | | | CUCUGGGAUUAUGCUAAA- |
| | | | CAGAGCAAUUUCCUAGCC- |
| | | | CUCACGA |
| 223. | hsa-mir- | MI0003605 | CCCCCAGAAUCUGUCAGG- |
| | 593 | | CACCAGCCAGGCAUUGCU- |
| | | | CAGCCCGUUUCCCU- |
| | | | CUGGGGGAGCAAGGAGUG- |
| | | | GUGCUGGGUUUGUCUCUG- |
| | | | CUGGGGUUUCUCCU |
| 224. | hsa-mir- | MI0003152 | CUCAAGCUGUGACUCUC- |
| | 525 | | CAGAGGGAUGCACUUUCU- |
| | | | CUUAUGUGAAAAAAAA- |
| | | | GAAGGCGCUUCCCUUUA- |
| | | | GAGCGUUACGGUUUGGG |
| 225. | hsa-mir- | MI0000452 | AGGCCUCGCUGUUCU- |
| | 135a-1 | | CUAUGGCUUUUUAUUC- |
| | | | CUAUGUGAUUCUACUGCU- |
| | | | CACUCAUAUAGGGAUUG- |
| | | | GAGCCGUGGCGCAC- |
| | | | GGCGGGGACA |
| 226. | hsa-mir- | MI0003635 | UAGAUUGAGGAAGGGGCU- |
| | 621 | | GAGUGGUAGGCGGUGCUG- |
| | | | CUGUGCUCUGAUGAA- |
| | | | GACCCAUGUGGCUAGCAA- |
| | | | CAGCGCUUACCUUUUGU- |
| | | | CUCUGGGUCC |
| 227. | hsa-mir- | MI0003598 | UGUGAUGUGUAUUAG- |
| | 548a-2 | | GUUUGUGCAAAA- |
| | | | GUAAUUGGG- |
| | | | GUUUUUUGCCGUUAAAA- |
| | | | GUAAUGGCAAAACUGG- |
| | | | CAAUUACUUUUGCAC- |
| | | | CAAACUAAUAUAA |
| 228. | hsa-mir- | MI0000082 | GGCCAGUGUUGAGAGGC- |
| | 25 | | GGAGACUUGGGCAAUUG- |
| | | | CUGGACGCUGCCCUGGG- |
| | | | CAUUGCACUUGUCUCGGU- |
| | | | CUGACAGUGCCGGCC |
| 229. | hsa-mir- | MI0001729 | CUUGGGAAUGGCAAG- |
| | 451 | | GAAACCGUUACCAUUACU- |
| | | | GAGUUUAGUAAUG- |
| | | | GUAAUGGUUCUCUUG- |
| | | | CUAUACCCAGA |
| 230. | hsa-mir- | MI0000461 | CACCUUGUCCUCACGGUC- |
| | 145 | | CAGUUUUCCCAGGAAUCC- |
| | | | CUUAGAUGCUAAGAUGGG- |
| | | | GAUUCCUGGAAAUACU- |
| | | | GUUCUUGAGGUCAUGGUU |
| 231. | hsa-mir- | MI0000738 | CCACCACUUAAACGUG- |
| | 302a | | GAUGUACUUGCUUUGAAA- |
| | | | CUAAAGAAGUAAGUG- |
| | | | CUUCCAUGUUUUGGU- |
| | | | GAUGG |
| 232. | hsa-mir- | MI0003668 | AAACAAGUUAUAUUAG- |
| | 548d-1 | | GUUGGUGCAAAAGUAAUU- |
| | | | GUGGUUUUUGCCU- |
| | | | GUAAAAGUAAUGG- |
| | | | CAAAAACCACAGUUU- |
| | | | CUUUUGCACCAGA- |
| | | | CUAAUAAAG |
| 233. | hsa-mir- | MI0000264 | CUGGAUACAGAGUGGACC |
| | 7-2 | | GGCUGGCCCCAUCUGGAA- |
| | | | GACUAGUGAUUUUGUU- |
| | | | GUUGUCUUACUGCGCU- |
| | | | CAACAACAAAUCCCAGU- |
| | | | CUACCUAAUGGUGCCAGC- |
| | | | CAUCGCA |
| 234. | hsa-mir- | MI0003194 | GUGCUGUGUGUAGUGCUU- |
| | 507 | | CACUUCAAGAAGUGC- |
| | | | CAUGCAUGUGUCUA- |
| | | | GAAAUAUGUUUUGCAC- |
| | | | CUUUUGGAGU- |
| | | | GAAAUAAUGCACAACA- |
| | | | GAUAC |
| 235. | hsa-mir- | MI0000826 | GUCUGUCUGCCCGCAUGC- |
| | 346 | | CUGCCUCUCUGUUGCUCU- |
| | | | GAAGGAGGCAGGGG- |
| | | | CUGGGCCUGCAGCUGC- |
| | | | CUGGGCAGAGCGGCUC- |
| | | | CUGC |
| 236. | hsa-mir- | MI0003682 | GCUCGGUUGCCGUG- |
| | 658 | | GUUGCGGGCCCUGCCC- |
| | | | GCCCGCCAGCUCGCUGA- |
| | | | CAGCACGACUCAGGGC- |
| | | | GGAGGGAAGUAGGUCC- |
| | | | GUUGGUCGGUCGGGAAC- |
| | | | GAGG |
| 237. | hsa-mir- | MI0003141 | GGUACUUCUCAGUCU- |
| | 512-2 | | GUGGCACUCAGCCUU- |
| | | | GAGGGCACUUUCUGGUGC- |
| | | | CAGAAUGAAAGUGCUGU- |
| | | | CAUAGCUGAGGUCCAAU- |
| | | | GACUGAGGCGAGCACC |
| 238. | hsa-mir- | MI0000287 | UCACCUGGCCAUGUGA- |
| | 211 | | CUUGUGGGCUUCCCUUU- |
| | | | GUCAUCCUUCGCCUAGGG- |
| | | | CUCUGAGCAGGGCAGGGA- |
| | | | CAGCAAAGGGGUGCUCA- |
| | | | GUUGUCACUUCCCACAG- |
| | | | CACGGAG |
| 239. | hsa-mir- | MI0000075 | ACAUUGCUACUUA- |
| | 19b-2 | | CAAUUAGUUUUGCAG- |
| | | | GUUUGCAUUUCAGC- |
| | | | GUAUAUAUGUAUAUGUGG- |
| | | | CUGUGCAAAUCCAUG- |
| | | | CAAAACUGAUUGU- |
| | | | GAUAAUGU |
| 240. | hsa-mir- | MI0003165 | GUGACCCUCUAGAUG- |
| | 517b | | GAAGCACUGUCUGUUGU- |
| | | | CUAAGAAAAGAUCGUG- |
| | | | CAUCCCUUUAGAGUGUUAC |
| 241. | hsa-mir- | MI0000458 | GACAGUGCAGUCACC- |
| | 142 | | CAUAAAGUAGAAAGCA- |
| | | | CUACUAACAGCACUG- |
| | | | GAGGGUGUAGUGUUUC- |
| | | | CUACUUUAUGGAUGAGU- |
| | | | GUACUGUG |
| 242. | hsa-mir- | MI0000777 | UUGAAGGGAGAUCGACC- |
| | 369 | | GUGUUAUAUUC- |
| | | | GCUUUAUUGACUUC- |
| | | | GAAUAAUACAUGGUUGAU- |
| | | | CUUUUCUCAG |
| 243. | hsa-mir- | MI0003607 | ACGGAAGCCUGCAC- |
| | 595 | | GCAUUUAACACCAGCAC- |
| | | | GCUCAAUGUAGUCUU- |
| | | | GUAAGGAACAGGUUGAA- |
| | | | GUGUGCCGUGGUGUGU- |
| | | | CUGGAGGAAGCGCCUGU |
| 244. | hsa-mir- | MI0003604 | UAUUAUGCCAUGACAUU- |
| | 592 | | GUGUCAAUAUGCGAUGAU- |
| | | | GUGUUGUGAUGGCACAGC- |
| | | | GUCAUCACGUGGUGAC- |
| | | | GCAACAUCAUGACGUAA- |
| | | | GACGUCACAAC |
| 245. | hsa-mir- | MI0003171 | UCCCAUGCUGUGACCCU- |
| | 518d | | CUAGAGGGAAGCACUUU- |
| | | | CUGUUGUCUGAAAGAAAC- |
| | | | CAAAGCGCUUCCCUUUG- |
| | | | GAGCGUUACGGUUUGAGA |
| 246. | hsa-mir- | MI0002470 | GUAUCCUGUACUGAG- |
| | 486 | | CUGCCCCGAGCUGGGCAG- |
| | | | CAUGAAGGGCCUC- |
| | | | GGGGCAGCUCAGUACAG- |
| | | | GAUGC |
| 247. | hsa-mir- | MI0000477 | CCGAUGUGUAUCCUCAG- |
| | 146a | | CUUUGAGAACUGAAUUC- |
| | | | CAUGGGUUGUGUCAGUGU- |
| | | | CAGACCUCUGAAAUUCA- |
| | | | GUUCUUCAGCUGGGAUAU- |
| | | | CUCUGUCAUCGU |
| 248. | hsa-mir- | MI0003514 | AUACUUGAGGA- |
| | 539 | | GAAAUUAUCCUUGGUGU- |
| | | | GUUCGCUUUAUUUAUGAU- |
| | | | GAAUCAUACAAGGA- |
| | | | CAAUUUCUUUUUGAGUAU |
| 249. | hsa-mir- | MI0003147 | UCUCAUGCAGUCAUUCUC- |
| | 515-2 | | CAAAAGAAAGCACUUUCU- |
| | | | GUUGUCUGAAAGCAGA- |
| | | | GUGCCUUCUUUUGGAGC- |
| | | | GUUACUGUUUGAGA |
| 250. | hsa-mir- | MI0000095 | CUGGGGGCUCCAAAGUG- |
| | 93 | | CUGUUCGUGCAGGUAGU- |
| | | | GUGAUUACCCAACCUA- |
| | | | CUGCUGAGCUAGCA- |
| | | | CUUCCCGAGCCCCCGG |
| 251. | hsa-mir- | MI0003565 | GCUCCAGUAACAU- |
| | 559 | | CUUAAAGUAAAUAUGCAC- |
| | | | CAAAAUUACUUUUG- |
| | | | GUAAAUACAGUUUUGGUG- |
| | | | CAUAUUUACUUUAGGAU- |
| | | | GUUACUGGAGCUCCCA |
| 252. | hsa-mir- | MI0003619 | UGUAUCCUUGGUUUUUA- |
| | 606 | | GUAGUUUUACUAUGAU- |
| | | | GAGGUGUGCCAUCCACCC- |
| | | | CAUCAUAGUAAACUACU- |
| | | | GAAAAUCAAAGAUACAA- |
| | | | GUGCCUGACCA |
| 253. | hsa-mir- | MI0001519 | AGUACCAAAGUGCUCAUA- |
| | 20b | | GUGCAGGUAGUUUUGG- |
| | | | CAUGACUCUACUGUA- |
| | | | GUAUGGGCACUUCCAGUA- |
| | | | CU |
| 254. | hsa-mir- | MI0003608 | AGCACGGCCUCUCC- |
| | 596 | | GAAGCCUGCCCGGCUC- |
| | | | CUCGGGAACCUGCCUCCC- |
| | | | GCAUGGCAGCUGCUGCC- |
| | | | CUUCGGAGGCCG |
| 255. | hsa-let- | MI0000434 | CUGGCUGAGGUAGUA- |
| | 7i | | GUUUGUGCUGUUGGUC- |
| | | | GGGUUGUGACAUUGCCC- |
| | | | GCUGUGGAGAUAACUGC- |
| | | | GCAAGCUACUGCCUUGCUA |
| 256. | hsa-mir- | MI0003186 | UGCUCCCCCUCUCUAAUC- |
| | 502 | | CUUGCUAUCUGGGUGCUA- |
| | | | GUGCUGGCUCAAUG- |
| | | | CAAUGCACCUGGGCAAG- |
| | | | GAUUCAGAGAGGGGGAGCU |
| 257. | hsa-mir- | MI0003563 | AGAAUGGGCAAAUGAACA- |
| | 557 | | GUAAAUUUGGAGGC- |
| | | | CUGGGGCCCUCCCUGCUG- |
| | | | CUGGAGAAGUGUUUGCAC- |
| | | | GGGUGGGCCUUGUCUUU- |
| | | | GAAAGGAGGUGGA |
| 258. | hsa-mir- | MI0000740 | ACUCAGGGGCUUCGCCA- |
| | 219-2 | | CUGAUUGUCCAAAC- |
| | | | GCAAUUCUUGUACGAGU- |
| | | | CUGCGGCCAACCGA- |
| | | | GAAUUGUGGCUGGACAU- |
| | | | CUGUGGCUGAGCUCCGGG |
| 259. | hsa-mir- | MI0003649 | AAACCCACACCACUG- |
| | 634 | | CAUUUUGGCCAUCGAGG- |
| | | | GUUGGGGCUUGGUGU- |
| | | | CAUGCCCCAAGAUAAC- |
| | | | CAGCACCCCAACUUUGGA- |
| | | | CAGCAUGGAUUAGUCU |
| 260. | hsa-mir- | MI0003134 | GAUACUCGAAGGAGAG- |
| | 494 | | GUUGUCCGUGUUGUCUU- |
| | | | CUCUUUAUUUAUGAU- |
| | | | GAAACAUACACGGGAAAC- |
| | | | CUCUUUUUUAGUAUC |
| 261. | hsa-mir- | MI0000809 | UUUCCUGCCCUCGAGGAG- |
| | 151 | | CUCACAGUCUAGUAUGU- |
| | | | CUCAUCCCCUACUAGACU- |
| | | | GAAGCUCCUUGAGGA- |
| | | | CAGGGAUGGUCAUACU- |
| | | | CACCUC |
| 262. | hsa-mir- | MI0003128 | CAAUAGACACCCAUCGU- |
| | 511-2 | | GUCUUUUGCUCUGCAGU- |
| | | | CAGUAAAUAUUUUUUUGU- |
| | | | GAAUGUGUAGCAAAAGA- |
| | | | CAGAAUGGUGGUCCAUUG |
| 263. | hsa-mir- | MI0001721 | UCCUGCUUGUCCUGCGAG- |
| | 431 | | GUGUCUUGCAGGCCGU- |
| | | | CAUGCAGGCCACACUGAC- |
| | | | GGUAACGUUGCAGGUCGU- |
| | | | CUUGCAGGGCUUCUC- |
| | | | GCAAGACGACAUCCUCAU- |
| | | | CACCAACGACG |
| 264. | hsa-mir- | MI0000779 | GUGGCACUCAAACU- |
| | 371 | | GUGGGGGCACUUUCUGCU- |
| | | | CUCUGGUGAAAGUGCC- |
| | | | GCCAUCUUUUGAGUGUUAC |
| 265. | hsa-mir- | MI0000773 | CCUUUGCUUUAA- |
| | 302c | | CAUGGGGGUACCUGCUGU- |
| | | | GUGAAACAAAAGUAAGUG- |
| | | | CUUCCAUGUUUCAGUG- |
| | | | GAGG |
| 266. | hsa-mir- | MI0003587 | AUAAAAUUUCCAAUUG- |
| | 580 | | GAACCUAAUGAUUCAUCA- |
| | | | GACUCAGAUAUUUAA- |
| | | | GUUAACAGUAUUUGA- |
| | | | GAAUGAUGAAUCAUUAG- |
| | | | GUUCCGGUCAGAAAUU |
| 267. | hsa-mir- | MI0000271 | CGGAAAAUUUGCCAAGG- |
| | 181c | | GUUUGGGGGAACAUU- |
| | | | CAACCUGUCGGUGA- |
| | | | GUUUGGGCAGCUCAGG- |
| | | | CAAACCAUCGACCGUUGA- |
| | | | GUGGACCCUGAGGCCUG- |
| | | | GAAUUGCCAUCCU |
| 268. | hsa-mir- | MI0001641 | CGCCGGCCGAUGGGCGU- |
| | 429 | | CUUACCAGACAUGGUUA- |
| | | | GACCUGGCCCUCUGU- |
| | | | CUAAUACUGUCUG- |
| | | | GUAAAACCGUCCAUCC- |
| | | | GCUGC |
| 269. | hsa-mir- | MI0000789 | UACUUAAAGCGAG- |
| | 381 | | GUUGCCCUUUGUAUAUUC- |
| | | | GGUUUAUUGACAUG- |
| | | | GAAUAUACAAGGGCAAG- |
| | | | CUCUCUGUGAGUA |
| 270. | hsa-mir- | MI0003657 | AUCUGAGUUGGGAGG- |
| | 642 | | GUCCCUCUCCAAAUGUGU- |
| | | | CUUGGGGUGGGGGAUCAA- |
| | | | GACACAUUUGGAGAGG- |
| | | | GAACCUCCCAACUC- |
| | | | GGCCUCUGCCAUCAUU |
| 271. | hsa-mir- | MI0003571 | CCAGUGGCGCAAUG- |
| | 565 | | GAUAACGCGUCUGACUAC- |
| | | | GGAUCAGAAGAUUCUAG- |
| | | | GUUCGACUCCUGGCUGG- |
| | | | CUCGCGAUGUCU- |
| | | | GUUUUGCCACACUUGACCC |
| 272. | hsa-mir- | MI0000266 | GAUCUGUCUGUCUUCU- |
| | 10a | | GUAUAUACCCUGUA- |
| | | | GAUCCGAAUUUGUGUAAG- |
| | | | GAAUUUUGUGGUCA- |
| | | | CAAAUUCGUAUCUAGGG- |
| | | | GAAUAUGUAGUUGA- |
| | | | CAUAAACACUCCGCUCU |
| 273. | hsa-mir- | MI0000808 | CUCAUCUGUCUGUUGGG- |
| | 326 | | CUGGAGGCAGGGCCUUU- |
| | | | GUGAAGGCGGGUGGUGCU- |
| | | | CAGAUCGCCUCUGGGCC- |
| | | | CUUCCUCCAGCCCC- |
| | | | GAGGCGGAUUCA |
| 274. | hsa-mir- | MI0003159 | GCGAGAAGAUCUCAUGCU- |
| | 518c | | GUGACUCUCUGGAGG- |
| | | | GAAGCACUUUCUGUUGU- |
| | | | CUGAAAGAAAACAAAGC- |
| | | | GCUUCUCUUUAGAGU- |
| | | | GUUACGGUUUGAGAAAAGC |
| 275. | hsa-mir- | MI0003127 | CAAUAGACACCCAUCGU- |
| | 511-1 | | GUCUUUUGCUCUGCAGU- |
| | | | CAGUAAAUAUUUUUUUGU- |
| | | | GAAUGUGUAGCAAAAGA- |
| | | | CAGAAUGGUGGUCCAUUG |
| 276. | hsa-mir- | MI0000825 | ACCCAAACCCUAGGUCUG- |
| | 345 | | CUGACUCCUAGUCCAGGG- |
| | | | CUCGUGAUGGCUG- |
| | | | GUGGGCCCUGAACGAGGG- |
| | | | GUCUGGAGGCCUGGGUUU- |
| | | | GAAUAUCGACAGC |
| 277. | hsa-mir- | MI0000742 | GUGCUCGGUUUGUAGGCA- |
| | 34b | | GUGUCAUUAGCUGAUU- |
| | | | GUACUGUGGUGGUUA- |
| | | | CAAUCACUAACUCCA- |
| | | | CUGCCAUCAAAACAAGG- |
| | | | CAC |
| 278. | hsa-mir- | MI0000080 | CUCCGGUGCCUACUGAG- |
| | 24-1 | | CUGAUAUCAGUUCU- |
| | | | CAUUUUACACACUGGCU- |
| | | | CAGUUCAGCAGGAACAG- |
| | | | GAG |
| 279. | hsa-mir- | MI0003638 | AAUGCUGUUUCAAGGUA- |
| | 624 | | GUACCAGUACCUUGUGUU- |
| | | | CAGUGGAACCAAGGUAAA- |
| | | | CACAAGGUAUUG- |
| | | | GUAUUACCUUGAGAUAG- |
| | | | CAUUACACCUAAGUG |
| 280. | hsa-mir- | MI0003575 | AGAUGUGCUCUCCUGGCC- |
| | 551b | | CAUGAAAUCAAGCGUGG- |
| | | | GUGAGACCUGGUGCA- |
| | | | GAACGGGAAGGCGACC- |
| | | | CAUACUUGGUUUCAGAGG- |
| | | | CUGUGAGAAUAA |
| 281. | hsa-mir- | MI0000475 | UGAGCCCUCGGAGGACUC- |
| | 136 | | CAUUUGUUUUGAUGAUG- |
| | | | GAUUCUUAUGCUCCAU- |
| | | | CAUCGUCUCAAAUGAGU- |
| | | | CUUCAGAGGGUUCU |
| 282. | hsa-mir- | MI0000263 | UUGGAUGUUGGCCUAGUU- |
| | 7-1 | | CUGUGUGGAAGACUAGU- |
| | | | GAUUUUGUUGUUUUUA- |
| | | | GAUAACUAAAUCGACAA- |
| | | | CAAAUCACAGUCUGC- |
| | | | CAUAUGGCACAGGC- |
| | | | CAUGCCUCUACAG |
| 283. | hsa-mir- | MI0003189 | GCUGCUGUUGGGAGACC- |
| | 504 | | CUGGUCUGCACUCUAUCU- |
| | | | GUAUUCUUACUGAAGGGA- |
| | | | GUGCAGGGCAGGGUUUCC- |
| | | | CAUACAGAGGGC |
| 284. | hsa-mir- | MI0003125 | UGGAGGCCUUGCUG- |
| | 490 | | GUUUGGAAAGUUCAUU- |
| | | | GUUCGACACCAUGGAU- |
| | | | CUCCAGGUGGGUCAA- |
| | | | GUUUAGAGAUGCACCAAC- |
| | | | CUGGAGGACUCCAUGCU- |
| | | | GUUGAGCUGUUCACAAG- |
| | | | CAGCGGACACUUCCA |
| 285. | hsa-let- | MI0000060 | UGGGAUGAGGUAGUAG- |
| | 7a-1 | | GUUGUAUAGUUUUAGGGU- |
| | | | CACACCCACCACUGGGA- |
| | | | GAUAACUAUACAAUCUA- |
| | | | CUGUCUUUCCUA |
| 286. | hsa-mir- | MI0000299 | GCUGCUGGAAGGUGUAG- |
| | 222 | | GUACCCUCAAUGGCUCA- |
| | | | GUAGCCAGUGUAGAUCCU- |
| | | | GUCUUUCGUAAUCAGCAG- |
| | | | CUACAUCUGGCUACUGG- |
| | | | GUCUCUGAUGGCAUCUU- |
| | | | CUAGCU |
| 287. | hsa-let- | MI0000063 | CGGGGUGAGGUAGUAG- |
| | 7b | | GUUGUGUGGUUUCAGGG- |
| | | | CAGUGAUGUUGCCCCUC- |
| | | | GGAAGAUAACUAUACAAC- |
| | | | CUACUGCCUUCCCUG |
| 288. | hsa-mir- | MI0003631 | CAUCAUAAGGAGCCUAGA- |
| | 617 | | CUUCCCAUUUGAAG- |
| | | | GUGGCCAUUUCCUACCAC- |
| | | | CUUCAAAUGGUAAGUC- |
| | | | CAGGCUCCUUCUGAUU- |
| | | | CAAUAAAUGAGGAGC |
| 289. | hsa-mir- | MI0000077 | UGUCGGGUAGCUUAUCA- |
| | 21 | | GACUGAUGUUGACUGUU- |
| | | | GAAUCUCAUGGCAACAC- |
| | | | CAGUCGAUGGGCUGUCU- |
| | | | GACA |
| 290. | hsa-mir- | MI0003642 | AUAGCUGUUGUGUCA- |
| | 628 | | CUUCCUCAUGCUGA- |
| | | | CAUAUUUACUAGAGG- |
| | | | GUAAAAUUAAUAACCUU- |
| | | | CUAGUAAGAGUGGCAGUC- |
| | | | GAAGGGAAGGGCUCAU |
| 291. | hsa-mir- | MI0003656 | UGGGUGAAAGGAAGGAAA- |
| | 641 | | GACAUAGGAUAGAGUCAC- |
| | | | CUCUGUCCUCUGUCCU- |
| | | | CUACCUAUAGAGGUGACU- |
| | | | GUCCUAUGUCUUUCCUUC- |
| | | | CUCUUACCCCU |
| 292. | hsa-mir- | MI0000785 | UUGAGCAGAGGUUGCC- |
| | 377 | | CUUGGUGAAUUC- |
| | | | GCUUUAUUUAUGUUGAAU- |
| | | | CACACAAAGGCAACUUUU- |
| | | | GUUUG |
| 293. | hsa-mir- | MI0003198 | AACAUGUUGUCUGUG- |
| | 514-1 | | GUACCCUACUCUGGAGA- |
| | | | GUGACAAUCAU- |
| | | | GUAUAAUUAAAUUUGAUU- |
| | | | GACACUUCUGUGAGUAGA- |
| | | | GUAACGCAUGACACGUACG |
| 294. | hsa-mir- | MI0000481 | CCAGUCACGUCCCCUUAU- |
| | 184 | | CACUUUUCCAGCCCAG- |
| | | | CUUUGUGACUGUAAGU- |
| | | | GUUGGACGGAGAACU- |
| | | | GAUAAGGGUAGGUGAUUGA |
| 295. | hsa-mir- | MI0000079 | GGCCGGCUGGGGUUC- |
| | 23a | | CUGGGGAUGGGAUUUG- |
| | | | CUUCCUGUCACAAAUCA- |
| | | | CAUUGCCAGGGAUUUC- |
| | | | CAACCGACC |
| 296. | hsa-mir- | MI0000267 | CCAGAGGUUGUAACGUU- |
| | 10b | | GUCUAUAUAUACCCUGUA- |
| | | | GAACCGAAUUUGUGUG- |
| | | | GUAUCCGUAUAGUCACA- |
| | | | GAUUCGAUUCUAGGG- |
| | | | GAAUAUAUGGUCGAUG- |
| | | | CAAAAACUUCA |
| 297. | hsa-mir- | MI0003677 | AACUAUGCAAGGAUAUUU- |
| | 655 | | GAGGAGAGGUUAUCCGU- |
| | | | GUUAUGUUCGCUUCAUU- |
| | | | CAUCAUGAAUAAUACAUG- |
| | | | GUUAACCUCUUUUU- |
| | | | GAAUAUCAGACUC |
| 298. | hsa-mir- | MI0003597 | AGCUUAGGUAC- |
| | 588 | | CAAUUUGGCCACAAUGG- |
| | | | GUUAGAACACUAUUC- |
| | | | CAUUGUGUUCUUACCCAC- |
| | | | CAUGGCCAAAAUUGGGC- |
| | | | CUAAG |
| 299. | hsa-mir- | MI0000439 | CUCAGGUGCUCUGGCUG- |
| | 23b | | CUUGGGUUCCUGGCAUG- |
| | | | CUGAUUUGUGACUUAA- |
| | | | GAUUAAAAUCACAUUGC- |
| | | | CAGGGAUUACCACGCAAC- |
| | | | CACGACCUUGGC |
| 300. | hsa-mir- | MI0003661 | GAUCAGGAGUCUGCCA- |
| | 646 | | GUGGAGUCAGCACACCUG- |
| | | | CUUUUCACCUGUGAUCC- |
| | | | CAGGAGAGGAAGCAG- |
| | | | CUGCCUCUGAGGCCU- |
| | | | CAGGCUCAGUGGC |
| 301. | hsa-mir- | MI0003570 | CGGGCAGCGGGUGCCAGG- |
| | 564 | | CACGGUGUCAGCAGGCAA- |
| | | | CAUGGCCGAGAGGCC- |
| | | | GGGGCCUCC- |
| | | | GGGCGGCGCCGUGUCC- |
| | | | GCGACCGCGUACCCUGAC |
| 302. | hsa-mir- | MI0003646 | GCGGGCGGCCCCGCGGUG- |
| | 33b | | CAUUGCUGUUGCAUUG- |
| | | | CACGUGUGUGAGGC- |
| | | | GGGUGCAGUGCCUCGGCA- |
| | | | GUGCAGCCCGGAGCC- |
| | | | GGCCCCUGGCACCAC |
| 303. | hsa-mir- | MI0003180 | UCUCAGGCUGUGACCUU- |
| | 516-1 | | CUCGAGGAAAGAAGCA- |
| | | | CUUUCUGUUGUCUGAAA- |
| | | | GAAAAGAAAGUGCUUC- |
| | | | CUUUCAGAGGGUUAC- |
| | | | GGUUUGAGA |
| 304. | hsa-mir- | MI0003591 | UAGGGUGACCAGC- |
| | 584 | | CAUUAUGGUUUGCCUGG- |
| | | | GACUGAGGAAUUUGCUGG- |
| | | | GAUAUGUCAGUUCCAGGC- |
| | | | CAACCAGGCUGGUUGGU- |
| | | | CUCCCUGAAGCAAC |
| 305. | hsa-mir- | MI0000103 | UGCCCUGGCUCAGUUAU- |
| | 101-1 | | CACAGUGCUGAUGCUGU- |
| | | | CUAUUCUAAAGGUACA- |
| | | | GUACUGUGAUAACUGAAG- |
| | | | GAUGGCA |
| 306. | hsa-mir- | MI0000464 | AGCGGUGGCCAGUGU- |
| | 153-2 | | CAUUUUUGUGAUGUUG- |
| | | | CAGCUAGUAAUAUGAGCC- |
| | | | CAGUUGCAUAGUCA- |
| | | | CAAAAGUGAUCAUUG- |
| | | | GAAACUGUG |
| 307. | hsa-mir- | MI0000775 | CCAUUACUGUUG- |
| | 367 | | CUAAUAUGCAACUCUGUU- |
| | | | GAAUAUAAAUUGGAAUUG- |
| | | | CACUUUAGCAAUGGU- |
| | | | GAUGG |
| 308. | hsa-mir- | MI0000254 | AGAUACUGUAAACAUC- |
| | 30c-2 | | CUACACUCUCAGCUGUG- |
| | | | GAAAGUAAGAAAGCUGG- |
| | | | GAGAAGGCUGUUUACU- |
| | | | CUUUCU |
| 309. | hsa-mir- | MI0003618 | GCCCUAGCUUGGUU- |
| | 605 | | CUAAAUCCCAUGGUGC- |
| | | | CUUCUCCUUGGGAAAAA- |
| | | | CAGAGAAGGCACUAUGA- |
| | | | GAUUUAGAAUCAAGUUAGG |
| 310. | hsa-mir- | MI0000767 | ACCGCAGGGAAAAUGAGG- |
| | 365-1 | | GACUUUUGGGGGCAGAU- |
| | | | GUGUUUCCAUUCCACUAU- |
| | | | CAUAAUGCCC- |
| | | | CUAAAAAUCCUUAUUGCU- |
| | | | CUUGCA |
| 311. | hsa-mir- | MI0000265 | AGAUUAGAGUGGCUGUG- |
| | 7-3 | | GUCUAGUGCUGUGUGGAA- |
| | | | GACUAGUGAUUUUGUU- |
| | | | GUUCUGAUGUACUACGA- |
| | | | CAACAAGUCACAGCC- |
| | | | GGCCUCAUAGCGCAGA- |
| | | | CUCCCUUCGAC |
| 312. | hsa-mir- | MI0003576 | GGUAUUGUUA- |
| | 569 | | GAUUAAUUUUGUGGGA- |
| | | | CAUUAACAACAGCAUCA- |
| | | | GAAGCAACAUCAGCUUUA- |
| | | | GUUAAUGAAUCCUGGAAA- |
| | | | GUUAAGUGACUUUAUUU |
| 313. | hsa-mir- | MI0000284 | GGCUACAGUCUUUCUU- |
| | 204 | | CAUGUGACUCGUGGA- |
| | | | CUUCCCUUUGUCAUC- |
| | | | CUAUGCCUGAGAAUAUAU- |
| | | | GAAGGAGGCUGGGAAGG- |
| | | | CAAAGGGACGUUCAAUU- |
| | | | GUCAUCACUGGC |
| 314. | hsa-mir- | MI0000480 | GUGGUACUUGAAGAUAG- |
| | 154 | | GUUAUCCGUGUUGCCUUC- |
| | | | GCUUUAUUUGUGACGAAU- |
| | | | CAUACACGGUUGAC- |
| | | | CUAUUUUUCAGUACCAA |
| 315. | hsa-mir- | MI0003650 | CAGAGAGGAGCUGCCA- |
| | 635 | | CUUGGGCACUGAAACAAU- |
| | | | GUCCAUUAGGCUUU- |
| | | | GUUAUGGAAACUUCUCCU- |
| | | | GAUCAUUGUUUUGUGUC- |
| | | | CAUUGAGCUUCCAAU |
| 316. | hsa-mir- | MI0003579 | GUCGAGGCCGUGGCCC- |
| | 572 | | GGAAGUGGUC- |
| | | | GGGGCCGCUGC- |
| | | | GGGCGGAAGGGCGCCU- |
| | | | GUGCUUCGUCCGCUC- |
| | | | GGCGGUGGCCCAGC- |
| | | | CAGGCCCGCGGGA |
| 317. | hsa-mir- | MI0000451 | GGGAGCCAAAUGCUUUG- |
| | 133a-2 | | CUAGAGCUGGUAAAAUG- |
| | | | GAACCAAAUCGACUGUC- |
| | | | CAAUGGAUUUGGUCCC- |
| | | | CUUCAACCAGCUGUAGCU- |
| | | | GUGCAUUGAUGGCGCCG |
| 318. | hsa-mir- | MI0000734 | CCUGCCGGGGCUAAAGUG- |
| | 106b | | CUGACAGUGCAGAUAGUG- |
| | | | GUCCUCUCCGUGCUACC- |
| | | | GCACUGUGGGUACUUG- |
| | | | CUGCUCCAGCAGG |
| 319. | hsa-mir- | MI0003564 | GUGUGUGUGUGUGUGU- |
| | 558 | | GUGGUUAUUUUGGUAUA- |
| | | | GUAGCUCUAGACU- |
| | | | CUAUUAUAGUUUCCUGAG- |
| | | | CUGCUGUACCAAAAUAC- |
| | | | CACAAACGGGCUG |
| 320. | hsa-mir- | MI0003195 | CCACCUUCAGCUGAGU- |
| | 508 | | GUAGUGCCCUACUCCA- |
| | | | GAGGGCGUCACUCAU- |
| | | | GUAAACUAAAACAUGAUU- |
| | | | GUAGCCUUUUGGAGUAGA- |
| | | | GUAAUACACAUCAC- |
| | | | GUAACGCAUAUUUGGUGG |
| 321. | hsa-mir- | MI0003637 | GUACACAGUAGAAG- |
| | 623 | | CAUCCCUUGCAGGGGCU- |
| | | | GUUGGGUUGCAUCCUAAG- |
| | | | CUGUGCUGGAGCUUCCC- |
| | | | GAUGUACUCUGUAGAUGU- |
| | | | CUUUGCACCUUCUG |
| 322. | hsa-mir- | MI0003164 | UCUCAAGCUGUGAGUCUA- |
| | 520d | | CAAAGGGAAGCCCUUUCU- |
| | | | GUUGUCUAAAAGAAAA- |
| | | | GAAAGUGCUUCUCUUUG- |
| | | | GUGGGUUACGGUUUGAGA |
| 323. | hsa-mir- | MI0000727 | UGUGCAGUGG- |
| | 128b | | GAAGGGGGGCCGAUACA- |
| | | | CUGUACGAGAGUGAGUAG- |
| | | | CAGGUCUCACAGUGAACC- |
| | | | GGUCUCUUUCCCUACUGU- |
| | | | GUC |
| 324. | hsa-let- | MI0000067 | UCAGAGUGAGGUAGUA- |
| | 7f-1 | | GAUUGUAUAGUUGUGGG- |
| | | | GUAGUGAUUUUACCCU- |
| | | | GUUCAGGAGAUAACUAUA- |
| | | | CAAUCUAUUGCCUUCCCU- |
| | | | GA |
| 325. | hsa-mir- | MI0003593 | UGCAGGGAGGUAUUAA- |
| | 548a-1 | | GUUGGUGCAAAAGUAAUU- |
| | | | GUGAUUUUUGC- |
| | | | CAUUAAAAGUAACGA- |
| | | | CAAAACUGGCAAUUA- |
| | | | CUUUUGCACCAAACCUG- |
| | | | GUAUU |
| 326. | hsa-mir- | MI0003155 | CCCUCUACAGGGAAGC- |
| | 520b | | GCUUUCUGUUGUCUGAAA- |
| | | | GAAAAGAAAGUGCUUC- |
| | | | CUUUUAGAGGG |
| 327. | hsa-mir- | MI0003515 | AUUUUCAUCACCUAGG- |
| | 544 | | GAUCUUGUUAAAAAGCA- |
| | | | GAUUCUGAUUCAGGGAC- |
| | | | CAAGAUUCUG- |
| | | | CAUUUUUAGCAAGUUCU- |
| | | | CAAGUGAUGCUAAU |
| 328. | hsa-mir- | MI0000479 | CUCCCCAUGGCCCUGU- |
| | 150 | | CUCCCAACCCUUGUACCA- |
| | | | GUGCUGGGCUCAGACC- |
| | | | CUGGUACAGGCCUGGGG- |
| | | | GACAGGGACCUGGGGAC |
| 329. | hsa-mir- | MI0000806 | GUAGUCAGUAGUUGGGGG- |
| | 337 | | GUGGGAACGGCUUCAUA- |
| | | | CAGGAGUUGAUGCACA- |
| | | | GUUAUCCAGCUCCUAUAU- |
| | | | GAUGCCUUUCUUCAUCCC- |
| | | | CUUCAA |
| 330. | hsa-mir- | MI0003143 | UCUCCUGCUGUGACCCU- |
| | 520e | | CAAGAUGGAAGCAGUUU- |
| | | | CUGUUGUCUGAAAGGAAA- |
| | | | GAAAGUGCUUCCUUUUU- |
| | | | GAGGGUUACUGUUUGAGA |
| 331. | hsa-mir- | MI0003648 | AACCUCUCUUAGCCUCU- |
| | 633 | | GUUUCUUUAUUGCGGUA- |
| | | | GAUACUAUUAAC- |
| | | | CUAAAAUGAGAAGG- |
| | | | CUAAUAGUAUCUACCA- |
| | | | CAAUAAAAUUGUUGUGAG- |
| | | | GAUA |
| 332. | hsa-mir- | MI0003623 | UCUAUUUGUCUUAGGU- |
| | 610 | | GAGCUAAAUGUGUGCUGG- |
| | | | GACACAUUUGAGCCAAAU- |
| | | | GUCCCAGCACACAUUUAG- |
| | | | CUCACAUAAGAAAAAUG- |
| | | | GACUCUAGU |
| 333. | hsa-mir- | MI0003530 | UUGGUACUUGGAGAGUG- |
| | 487b | | GUUAUCCCUGUCCUGUUC- |
| | | | GUUUUGCUCAUGUC- |
| | | | GAAUCGUACAGGGUCAUC- |
| | | | CACUUUUUCAGUAUCAA |
| 334. | hsa-mir- | MI0000291 | AUCAUUCAGAAAUG- |
| | 215 | | GUAUACAGGAAAAUGAC- |
| | | | CUAUGAAUUGACAGA- |
| | | | CAAUAUAGCUGAGUUUGU- |
| | | | CUGUCAUUUCUUUAGGC- |
| | | | CAAUAUUCUGUAUGACU- |
| | | | GUGCUACUUCAA |
| 335. | hsa-mir- | MI0003671 | GAGAGGGAAGAUUUAG- |
| | 548d-2 | | GUUGGUGCAAAAGUAAUU- |
| | | | GUGGUUUUUGCCAUU- |
| | | | GAAAGUAAUGGCAAAAAC- |
| | | | CACAGUUUCUUUUGCAC- |
| | | | CAACCUAAUAAAA |
| 336. | hsa-mir- | MI0003665 | CAGUGCUGGGGUCUCAG- |
| | 650 | | GAGGCAGCGCUCUCAG- |
| | | | GACGUCACCACCAUGGC- |
| | | | CUGGGCUCUGCUCCUCCU- |
| | | | CACCCUCCUCACUCAGGG- |
| | | | CACAGGUGAU |
| 337. | hsa-mir- | MI0003629 | UUAGGUAAUUCCUCCACU- |
| | 616 | | CAAAACCCUUCAGUGA- |
| | | | CUUCCAUGACAU- |
| | | | GAAAUAGGAAGUCAUUG- |
| | | | GAGGGUUUGAGCAGAG- |
| | | | GAAUGACCUGUUUUAAAA |
| 338. | hsa-mir- | MI0000288 | CGGGGCACCCCGCCCGGA- |
| | 212 | | CAGCGCGCCGGCAC- |
| | | | CUUGGCUCUAGACUG- |
| | | | CUUACUGCCC- |
| | | | GGGCCGCCCUCAGUAACA- |
| | | | GUCUCCAGUCAC- |
| | | | GGCCACCGAC- |
| | | | GCCUGGCCCCGCC |
| 339. | hsa-mir- | MI0001733 | GCUAAGCACUUACAACU- |
| | 452 | | GUUUGCAGAGGAAACUGA- |
| | | | GACUUUGUAACUAUGUCU- |
| | | | CAGUCUCAUCUGCAAA- |
| | | | GAAGUAAGUGCUUUGC |
| 340. | hsa-mir- | MI0001637 | GCCGGGAGGUUGAACAUC- |
| | 448 | | CUGCAUAGUGCUGCCAG- |
| | | | GAAAUCCCUAUUU- |
| | | | CAUAUAAGAGGGGGCUGG- |
| | | | CUGGUUGCAUAUGUAG- |
| | | | GAUGUCCCAUCUCC- |
| | | | CAGCCCACUUCGUCA |
| 341. | hsa-mir- | MI0000069 | CCUUGGAGUAAAGUAG- |
| | 15a | | CAGCACAUAAUGGUUU- |
| | | | GUGGAUUUUGAAAAGGUG- |
| | | | CAGGCCAUAUUGUGCUGC- |
| | | | CUCAAAAAUACAAGG |
| 342. | hsa-mir- | MI0003197 | GUGGUGUCCUACUCAGGA- |
| | 510 | | GAGUGGCAAUCACAU- |
| | | | GUAAUUAGGUGUGAUU- |
| | | | GAAACCUCUAAGAGUGGA- |
| | | | GUAACAC |
| 343. | hsa-mir- | MI0003654 | UGGCCGAC- |
| | 639 | | GGGGCGCGCGCGGCCUG- |
| | | | GAGGGGCGGGGCGGAC- |
| | | | GCAGAGCCGCGUUUAGU- |
| | | | CUAUCGCUGCGGUUGC- |
| | | | GAGCGCUGUAGGGAGCCU- |
| | | | GUGCUG |
| 344. | hsa-mir- | MI0003641 | UACUUAUUACUGGUAGU- |
| | 627 | | GAGUCUCUAAGAAAAGAG- |
| | | | GAGGUGGUUGUUUUCCUC- |
| | | | CUCUUUUCUUUGAGACU- |
| | | | CACUACCAAUAAUAA- |
| | | | GAAAUACUACUA |
| 345. | hsa-mir- | MI0003634 | AUAUAUAUCUAUAUCUAG- |
| | 620 | | CUCC- |
| | | | GUAUAUAUAUAUAUAUAUA |
| | | | UAUAGAUAUCUC- |
| | | | CAUAUAUAUGGAGAUA- |
| | | | GAUAUAGAAAUAAAA- |
| | | | CAAGCAAAGAA |
| 346. | hsa-mir- | MI0000083 | GUGGCCUCGUUCAA- |
| | 26a-1 | | GUAAUCCAGGAUAGGCU- |
| | | | GUGCAGGUCCCAAUGGGC- |
| | | | CUAUUCUUGGUUACUUG- |
| | | | CACGGGGACGC |
| 347. | hsa-mir- | MI0000784 | UAAAAGGUAGAUUCUC- |
| | 376a-1 | | CUUCUAUGAGUA- |
| | | | CAUUAUUUAUGAUUAAU- |
| | | | CAUAGAGGAAAAUCCAC- |
| | | | GUUUUC |
| 348. | hsa-mir- | MI0003683 | UACCGACCCUCGAUUUG- |
| | 659 | | GUUCAGGACCUUCCCU- |
| | | | GAACCAAGGAAGAGUCA- |
| | | | CAGUCUCUUCCUUGGUU- |
| | | | CAGGGAGGGUCCCCAA- |
| | | | CAAUGUCCUCAUGG |
| 349. | hsa-mir- | MI0003149 | CUCAGGCUGUGACCCUC- |
| | 520a | | CAGAGGGAAGUACUUUCU- |
| | | | GUUGUCUGAGAGAAAA- |
| | | | GAAAGUGCUUCCCUUUG- |
| | | | GACUGUUUCGGUUUGAG |
| 350. | hsa-let- | MI0000062 | GGGUGAGGUAGUAGGUU- |
| | 7a-3 | | GUAUAGUUUGGGGCU- |
| | | | CUGCCCUGCUAUGG- |
| | | | GAUAACUAUACAAUCUA- |
| | | | CUGUCUUUCCU |
| 351. | hsa-mir- | MI0000455 | CGUUGCUGCAGCUGGU- |
| | 138-2 | | GUUGUGAAUCAGGCCGAC- |
| | | | GAGCAGCGCAUCCU- |
| | | | CUUACCCGGCUAUUUCAC- |
| | | | GACACCAGGGUUGCAUCA |
| 352. | hsa-mir- | MI0003627 | UCUAAGAAACGCAGUGGU- |
| | 614 | | CUCUGAAGCCUGCAGGGG- |
| | | | CAGGCCAGCCCUGCACU- |
| | | | GAACGCCUGUUCUUGC- |
| | | | CAGGUGGCAGAAGGUUG- |
| | | | CUGC |
| 353. | hsa-mir- | MI0003138 | CCACCCCGGUCCUG- |
| | 497 | | CUCCCGCCCCAGCAGCA- |
| | | | CACUGUGGUUUGUAC- |
| | | | GGCACUGUGGCCACGUC- |
| | | | CAAACCACACUGUGGU- |
| | | | GUUAGAGCGAGGGUGGGG- |
| | | | GAGGCACCGCCGAGG |
| 354. | hsa-mir- | MI0003193 | GCCACCACCAUCAGC- |
| | 506 | | CAUACUAUGUGUAGUGC- |
| | | | CUUAUUCAGGAAGGU- |
| | | | GUUACUUAAUA- |
| | | | GAUUAAUAUUUGUAAGG- |
| | | | CACCCUUCUGAGUAGA- |
| | | | GUAAUGUGCAACAUGGA- |
| | | | CAACAUUUGUGGUGGC |
| 355. | hsa-mir- | MI0002471 | GGUACUUGAAGAGUG- |
| | 487a | | GUUAUCCCUGCUGUGUUC- |
| | | | GCUUAAUUUAUGACGAAU- |
| | | | CAUACAGGGACAUCCA- |
| | | | GUUUUUCAGUAUC |
| 356. | hsa-mir- | MI0001445 | AUAAAGGAAGUUAGGCU- |
| | 423 | | GAGGGGCAGAGAGCGAGA- |
| | | | CUUUUCUAUUUUC- |
| | | | CAAAAGCUCGGUCU- |
| | | | GAGGCCCCUCAGUCUUG- |
| | | | CUUCCUAACCCGCGC |
| 357. | hsa-mir- | MI0003622 | UGCUCGGCUGUUCCUAGG- |
| | 609 | | GUGUUUCUCUCAUCUCUG- |
| | | | GUCUAUAAUGG- |
| | | | GUUAAAUAGUAGAGAU- |
| | | | GAGGGCAACACCCUAG- |
| | | | GAACAGCAGAGGAACC |
| 358. | hsa-mir- | MI0000466 | CGGGGUUGGUUGUUAU- |
| | 9-1 | | CUUUGGUUAUCUAGCU- |
| | | | GUAUGAGUGGUGUGGAGU- |
| | | | CUUCAUAAAGCUA- |
| | | | GAUAACCGAAA- |
| | | | GUAAAAAUAACCCCA |
| 359. | hsa-mir- | MI0000459 | GCGCAGCGCCCUGUCUCC- |
| | 143 | | CAGCCUGAGGUGCAGUG- |
| | | | CUGCAUCUCUGGUCA- |
| | | | GUUGGGAGUCUGAGAU- |
| | | | GAAGCACUGUAGCUCAG- |
| | | | GAAGAGAGAAGUUGUU- |
| | | | CUGCAGC |
| 360. | hsa-mir- | MI0000807 | UUGGUACUUGGAGAGAG- |
| | 323 | | GUGGUCCGUGGCGCGUUC- |
| | | | GCUUUAUUUAUGGCGCA- |
| | | | CAUUACACGGUCGACCU- |
| | | | CUUUGCAGUAUCUAAUC |
| 361. | hsa-mir- | MI0000462 | UGUCCCCCCCGGCCCAG- |
| | 152 | | GUUCUGUGAUACACUCC- |
| | | | GACUCGGGCUCUGGAGCA- |
| | | | GUCAGUGCAUGACAGAA- |
| | | | CUUGGGCCCGGAAGGACC |
| 362. | hsa-mir- | MI0000453 | AGAUAAAUUCACUCUA- |
| | 135a-2 | | GUGCUUUAUGG- |
| | | | CUUUUUAUUCCUAUGU- |
| | | | GAUAGUAAUAAAGUCU- |
| | | | CAUGUAGGGAUGGAAGC- |
| | | | CAUGAAAUACAUUGU- |
| | | | GAAAAAUCA |
| 363. | hsa-mir- | MI0001726 | GUGGUACCUGAAGAGAG- |
| | 329-2 | | GUUUUCUGGGUUUCU- |
| | | | GUUUCUUUAUUGAGGAC- |
| | | | GAAACACACCUGGUUAAC- |
| | | | CUCUUUUCCAGUAUCAA |
| 364. | hsa-mir- | MI0003144 | UCUCAUGCAGUCAUUCUC- |
| | 515-1 | | CAAAAGAAAGCACUUUCU- |
| | | | GUUGUCUGAAAGCAGA- |
| | | | GUGCCUUCUUUUGGAGC- |
| | | | GUUACUGUUUGAGA |
| 365. | hsa-mir- | MI0000782 | UACAUC- |
| | 374 | | GGCCAUUAUAAUACAAC- |
| | | | CUGAUAAGUGUUAUAGCA- |
| | | | CUUAUCAGAUUGUAUU- |
| | | | GUAAUUGUCUGUGUA |
| 366. | hsa-mir- | MI0003603 | UCUUAUCAAUGAGGUA- |
| | 591 | | GACCAUGGGUUCUCAUU- |
| | | | GUAAUAGUGUAGAAU- |
| | | | GUUGGUUAACUGUGGA- |
| | | | CUCCCUGGCUCUGUCU- |
| | | | CAAAUCUACUGAUUC |
| 367. | hsa-mir- | MI0003179 | UCUCAAGCUGUGACUG- |
| | 527 | | CAAAGGGAAGCCCUUUCU- |
| | | | GUUGUCUAAAAGAAAA- |
| | | | GAAAGUGCUUCCCUUUG- |
| | | | GUGAAUUACGGUUUGAGA |
| 368. | hsa-mir- | MI0000070 | GUCAGCAGUGCCUUAG- |
| | 16-1 | | CAGCACGUAAAUAUUGGC- |
| | | | GUUAAGAUU- |
| | | | CUAAAAUUAUCUCCA- |
| | | | GUAUUAACUGUGCUGCU- |
| | | | GAAGUAAGGUUGAC |
| 369. | hsa-mir- | MI0000803 | CUUUGGCGAUCACUGCCU- |
| | 330 | | CUCUGGGCCUGUGU- |
| | | | CUUAGGCUCUGCAAGAU- |
| | | | CAACCGAGCAAAGCACAC- |
| | | | GGCCUGCAGAGAGGCAGC- |
| | | | GCUCUGCCC |
| 370. | hsa-let- | MI0000066 | CCCGGGCUGAGGUAGGAG- |
| | 7e | | GUUGUAUAGUUGAGGAG- |
| | | | GACACCCAAGGAGAUCA- |
| | | | CUAUACGGCCUCCUAG- |
| | | | CUUUCCCCAGG |
| 371. | hsa-mir- | MI0000454 | GGUCCUCUGACUCUCUUC- |
| | 137 | | GGUGACGGGUAUUCUUGG- |
| | | | GUGGAUAAUACGGAUUAC- |
| | | | GUUGUUAUUGCUUAA- |
| | | | GAAUACGCGUAGUCGAG- |
| | | | GAGAGUACCAGCGGCA |
| 372. | hsa-mir- | MI0003586 | CAUAUUAGGUUAAUG- |
| | 579 | | CAAAAGUAAUCGCGGUUU- |
| | | | GUGCCAGAUGACGAUUU- |
| | | | GAAUUAAUAAAUU- |
| | | | CAUUUGGUAUAAACC- |
| | | | GCGAUUAUUUUUGCAU- |
| | | | CAAC |
| 373. | hsa-mir- | MI0000300 | CCUGGCCUCCUGCAGUGC- |
| | 223 | | CACGCUCCGUGUAUUUGA- |
| | | | CAAGCUGAGUUGGACA- |
| | | | CUCCAUGUGGUAGAGUGU- |
| | | | CAGUUUGUCAAAUACCC- |
| | | | CAAGUGCGGCACAUG- |
| | | | CUUACCAG |
| 374. | hsa-mir- | MI0000268 | GGCCAGCUGUGAGUGUUU- |
| | 34a | | CUUUGGCAGUGUCUUAG- |
| | | | CUGGUUGUUGUGAG- |
| | | | CAAUAGUAAGGAAGCAAU- |
| | | | CAGCAAGUAUACUGCC- |
| | | | CUAGAAGUGCUGCACGUU- |
| | | | GUGGGGCCC |
| 375. | hsa-mir- | MI0003664 | GGCCUAGCCAAAUACU- |
| | 649 | | GUAUUUUUGAUCGA- |
| | | | CAUUUGGUUGAAAAAUAU- |
| | | | CUAUGUAUUAGUAAACCU- |
| | | | GUGUUGUUCAAGAGUCCA- |
| | | | CUGUGUUUUGCUG |
| 376. | hsa-mir- | MI0000081 | CUCUGCCUCCCGUGCCUA- |
| | 24-2 | | CUGAGCUGAAACACA- |
| | | | GUUGGUUUGUGUACA- |
| | | | CUGGCUCAGUUCAGCAG- |
| | | | GAACAGGG |
| 377. | hsa-mir- | MI0000111 | UGUGCAUCGUGGU- |
| | 105-1 | | CAAAUGCUCAGACUCCU- |
| | | | GUGGUGGCUGCUCAUG- |
| | | | CACCACGGAUGUUUGAG- |
| | | | CAUGUGCUACGGUGUCUA |
| 378. | hsa-mir- | MI0000242 | GCCAACCCAGUGUUCAGA- |
| | 199a-1 | | CUACCUGUUCAGGAGGCU- |
| | | | CUCAAUGUGUACAGUAGU- |
| | | | CUGCACAUUGGUUAGGC |
| 379. | hsa-mir- | MI0003178 | CUCAGGCUGUGACACU- |
| | 519a-1 | | CUAGAGGGAAGCGCUUU- |
| | | | CUGUUGUCUGAAAGAAAG- |
| | | | GAAAGUGCAUCCUUUUA- |
| | | | GAGUGUUACUGUUUGAG |
| 380. | hsa-mir- | MI0000487 | CGAGGAUGGGAGCU- |
| | 193a | | GAGGGCUGGGUCUUUGC- |
| | | | GGGCGAGAUGAGGGUGUC- |
| | | | GGAUCAACUGGCCUA- |
| | | | CAAAGUCCCAGUU- |
| | | | CUCGGCCCCCG |
| 381. | hsa-let- | MI0000064 | GCAUCCGGGUUGAGGUA- |
| | 7c | | GUAGGUUGUAUGGUUUA- |
| | | | GAGUUACACCCUGGGA- |
| | | | GUUAACUGUACAACCUU- |
| | | | CUAGCUUUCCUUGGAGC |
| 382. | hsa-mir- | MI0000445 | UGAGGGCCCCUCUGCGU- |
| | 124a-3 | | GUUCACAGCGGACCUU- |
| | | | GAUUUAAUGUCUAUA- |
| | | | CAAUUAAGGCACGCGGU- |
| | | | GAAUGCCAAGAGAGGC- |
| | | | GCCUCC |
| 383. | hsa-mir- | MI0003574 | GAUAUACACUAUAUUAU- |
| | 568 | | GUAUAAAUGUAUACACA- |
| | | | CUUCCUAUAUGUAUCCA- |
| | | | CAUAUAUAUAGU- |
| | | | GUAUAUAUUAUACAU- |
| | | | GUAUAGGUGUGUAUAUG |
| 384. | hsa-mir- | MI0000071 | GUCAGAAUAAUGUCAAA- |
| | 17 | | GUGCUUACAGUGCAGGUA- |
| | | | GUGAUAUGUGCAUCUA- |
| | | | CUGCAGUGAAGGCACUU- |
| | | | GUAGCAUUAUGGUGAC |
| 385. | hsa-mir- | MI0000822 | CCUCAGAAGAAA- |
| | 133b | | GAUGCCCCCUGCUCUGG- |
| | | | CUGGUCAAACGGAACCAA- |
| | | | GUCCGUCUUCCUGAGAG- |
| | | | GUUUGGUCCCCUUCAAC- |
| | | | CAGCUACAGCAGGGCUGG- |
| | | | CAAUGCCCAGUCCUUGGA- |
| | | | GA |
| 386. | hsa-mir- | MI0003595 | CUCCUAUGCACCCU- |
| | 587 | | CUUUCCAUAGGUGAUGA- |
| | | | GUCACAGGGCUCAGG- |
| | | | GAAUGUGUCUGCACCUGU- |
| | | | GACUCAUCACCAGUG- |
| | | | GAAAGCCCAUCCCAUAU |
| 387. | hsa-mir- | MI0000788 | AAGAUGGUUGACCAUA- |
| | 380 | | GAACAUGCGCUAUCUCU- |
| | | | GUGUCGUAUGUAAUAUG- |
| | | | GUCCACAUCUU |
| 388. | hsa-mir- | MI0003169 | UCUCAGGCUGUGACCCU- |
| | 518e | | CUAGAGGGAAGCGCUUU- |
| | | | CUGUUGGCUAAAAGAAAA- |
| | | | GAAAGCGCUUCCCUUCA- |
| | | | GAGUGUUAACGCUUUGAGA |
| 389. | hsa-mir- | MI0000093 | CUUUCUACACAGGUUGG- |
| | 92-1 | | GAUCGGUUGCAAUGCUGU- |
| | | | GUUUCUGUAUGGUAUUG- |
| | | | CACUUGUCCCGGCCUGUU- |
| | | | GAGUUUGG |
| 390. | hsa-mir- | MI0003615 | UUCUCACCCCCGCCUGA- |
| | 602 | | CACGGGCGACAGCUGC- |
| | | | GGCCCGCUGUGUUCACUC- |
| | | | GGGCCGAGUGCGUCUCCU- |
| | | | GUCAGGCAAGGGAGAGCA- |
| | | | GAGCCCCCCUG |
| 391. | hsa-mir- | MI0003160 | UCUCAUGCUGUGACCCUA- |
| | 524 | | CAAAGGGAAGCACUUUCU- |
| | | | CUUGUCCAAAGGAAAA- |
| | | | GAAGGCGCUUCCCUUUG- |
| | | | GAGUGUUACGGUUUGAGA |
| 392. | hsa-mir- | MI0003660 | CAGUUCCUAACAGGCCU- |
| | 645 | | CAGACCAGUACCGGUCU- |
| | | | GUGGCCUGGGGGUUGAG- |
| | | | GACCCCUGCUCUAGGCUG- |
| | | | GUACUGCUGAUG- |
| | | | CUUAAAAAGAGAG |
| 393. | hsa-mir- | MI0003568 | AGUGAAAUUGCUAGGU- |
| | 562 | | CAUAUGGUCAGUCUA- |
| | | | CUUUUAGAGUAAUUGU- |
| | | | GAAACUGUUUUUCAAA- |
| | | | GUAGCUGUACCAUUUGCA- |
| | | | CUCCCUGUGGCAAU |
| 394. | hsa-mir- | MI0000279 | UGCUCGCUCAGCUGAUCU- |
| | 196a-2 | | GUGGCUUAGGUAGUUU- |
| | | | CAUGUUGUUGGGAUUGA- |
| | | | GUUUUGAACUCGGCAA- |
| | | | CAAGAAACUGCCUGA- |
| | | | GUUACAUCAGUC- |
| | | | GGUUUUCGUCGAGGGC |
| 395. | hsa-mir- | MI0003672 | CCUUCCGGCGUCCCAGGC- |
| | 663 | | GGGGCGCCGCGGGACC- |
| | | | GCCCUCGUGUCUGUGGC- |
| | | | GGUGGGAUCCC- |
| | | | GCGGCCGUGUUUUCCUG- |
| | | | GUGGCCCGGCCAUG |
| 396. | hsa-mir- | MI0003185 | GCUCUUCCUCUCUAAUC- |
| | 501 | | CUUUGUCCCUGGGUGAGA- |
| | | | GUGCUUUCUGAAUG- |
| | | | CAAUGCACCCGGGCAAG- |
| | | | GAUUCUGAGAGGGUGAGC |
| 397. | hsa-mir- | MI0003129 | CCUGGCACUGAGAACU- |
| | 146b | | GAAUUCCAUAGGCUGU- |
| | | | GAGCUCUAGCAAUGCCCU- |
| | | | GUGGACUCAGUUCUG- |
| | | | GUGCCCGG |
| 398. | hsa-mir- | MI0003174 | GAAGAUCUCAGGCAGU- |
| | 517c | | GACCCUCUAGAUGGAAG- |
| | | | CACUGUCUGUUGUCUAA- |
| | | | GAAAAGAUCGUGCAUC- |
| | | | CUUUUAGAGUGUUACU- |
| | | | GUUUGAGAAAAUC |
| 399. | hsa-mir- | MI0003632 | CUCUUGUUCACAGCCAAA- |
| | 618 | | CUCUACUUGUCCUUCUGA- |
| | | | GUGUAAUUACGUACAUG- |
| | | | CAGUAGCUCAGGAGA- |
| | | | CAAGCAGGUUUACCCU- |
| | | | GUGGAUGAGUCUGA |
| 400. | hsa-mir- | MI0003582 | AAUUCAGCCCUGCCA- |
| | 575 | | CUGGCUUAUGUCAUGAC- |
| | | | CUUGGGCUACUCAGGCU- |
| | | | GUCUGCACAAUGAGCCA- |
| | | | GUUGGACAGGAGCAGUGC- |
| | | | CACUCAACUC |
| 401. | hsa-mir- | MI0003620 | UUGCCUAAAGUCACACAG- |
| | 607 | | GUUAUAGAUCUGGAUUG- |
| | | | GAACCCAGGGAGCCAGA- |
| | | | CUGCCUGGGUUCAAAUC- |
| | | | CAGAUCUAUAACUUGUGU- |
| | | | GACUUUGGG |
| 402. | hsa-mir- | MI0000747 | AGGACCCUUCCA- |
| | 296 | | GAGGGCCCCCCCUCAAUC- |
| | | | CUGUUGUGCCUAAUUCA- |
| | | | GAGGGUUGGGUGGAGGCU- |
| | | | CUCCUGAAGGGCUCU |
| 403. | hsa-mir- | MI0000651 | UGGGAAACAUACUU- |
| | 1-1 | | CUUUAUAUGCCCAUAUG- |
| | | | GACCUGCUAAGCUAUG- |
| | | | GAAUGUAAAGAAGUAU- |
| | | | GUAUCUCA |
| 404. | hsa-mir- | MI0000483 | UGCUUGUAACUUUCCAAA- |
| | 186 | | GAAUUCUCCUUUUGGG- |
| | | | CUUUCUG- |
| | | | GUUUUAUUUUAAGCC- |
| | | | CAAAGGUGAAUUUUUUGG- |
| | | | GAAGUUUGAGCU |
| 405. | hsa-mir- | MI0000778 | AGACAGAGAAGCCAGGU- |
| | 370 | | CACGUCUCUGCAGUUACA- |
| | | | CAGCUCACGAGUGCCUG- |
| | | | CUGGGGUGGAACCUGGU- |
| | | | CUGUCU |
| 406. | hsa-mir- | MI0000469 | UGCCAGUCUCUAGGUCC- |
| | 125a | | CUGAGACCCUUUAACCU- |
| | | | GUGAGGACAUCCAGGGU- |
| | | | CACAGGUGAGGUUCUUGG- |
| | | | GAGCCUGGCGUCUGGCC |
| 407. | hsa-mir- | MI0003123 | GAGAAUCAUCUCUCCCA- |
| | 488 | | GAUAAUGGCACUCUCAAA- |
| | | | CAAGUUCCAAAUUGUUU- |
| | | | GAAAGGCUAUUUCUUGGU- |
| | | | CAGAUGACUCUC |
| 408. | hsa-mir- | MI0003559 | ACCUGAGUAACCUUUG- |
| | 554 | | CUAGUCCUGACUCAGCCA- |
| | | | GUACUGGUCUUAGACUG- |
| | | | GUGAUGGGUCAGGGUU- |
| | | | CAUAUUUUGGCAUCUCU- |
| | | | CUCUGGGCAUCU |
| 409. | hsa-mir- | MI0003196 | CAUGCUGUGUGUGGUACC- |
| | 509 | | CUACUGCAGACAGUGG- |
| | | | CAAUCAU- |
| | | | GUAUAAUUAAAAAU- |
| | | | GAUUGGUACGUCUGUGG- |
| | | | GUAGAGUACUGCAUGACA- |
| | | | CAUG |
| 410. | hsa-mir- | MI0000086 | GGUCCUUGCCCUCAAG- |
| | 28 | | GAGCUCACAGUCUAUUGA- |
| | | | GUUACCUUUCUGA- |
| | | | CUUUCCCACUAGAUUGU- |
| | | | GAGCUCCUGGAGGGCAGG- |
| | | | CACU |
| 411. | hsa-mir- | MI0000273 | CCGCAGAGUGUGACUCCU- |
| | 183 | | GUUCUGUGUAUGGCACUG- |
| | | | GUAGAAUUCACUGUGAA- |
| | | | CAGUCUCAGUCAGU- |
| | | | GAAUUACCGAAGGGC- |
| | | | CAUAAACAGAGCAGAGA- |
| | | | CAGAUCCACGA |
| 412. | hsa-mir- | MI0002469 | ACUUGGAGAGAGG- |
| | 485 | | CUGGCCGUGAUGAAUUC- |
| | | | GAUUCAUCAAAGCGAGU- |
| | | | CAUACACGGCUCUCCUCU- |
| | | | CUUUUAGU |
| 413. | hsa-mir- | MI0000488 | AUGGUGUUAUCAAGU- |
| | 194-1 | | GUAACAGCAACUCCAU- |
| | | | GUGGACUGUGUAC- |
| | | | CAAUUUCCAGUGGAGAUG- |
| | | | CUGUUACUUUUGAUG- |
| | | | GUUACCAA |
| 414. | hsa-mir- | MI0000769 | AGAGUGUUCAAGGACAG- |
| | 365-2 | | CAAGAAAAAUGAGGGA- |
| | | | CUUUCAGGGGCAGCUGU- |
| | | | GUUUUCUGACUCAGU- |
| | | | CAUAAUGCCC- |
| | | | CUAAAAAUCCUUAUUGUU- |
| | | | CUUGCAGUGUGCAUCGGG |
| 415. | hsa-mir- | MI0000238 | GUGAAUUAGGUAGUUU- |
| | 196a-1 | | CAUGUUGUUGGGCCUGG- |
| | | | GUUUCUGAACACAACAA- |
| | | | CAUUAAACCACCCGAUU- |
| | | | CAC |
| 416. | hsa-mir- | MI0003611 | AAAGACAUGCUGUCCACA- |
| | 599 | | GUGUGUUUGAUAAGCUGA- |
| | | | CAUGGGACAGGGAUU- |
| | | | CUUUUCACUGUUGUGUCA- |
| | | | GUUUAUCAAACCCAUA- |
| | | | CUUGGAUGAC |
| 417. | hsa-mir- | MI0003146 | UCUCAGGCUGUGACCCU- |
| | 520f | | CUAAAGGGAAGCGCUUU- |
| | | | CUGUGGUCAGAAA- |
| | | | GAAAAGCAAGUGCUUC- |
| | | | CUUUUAGAGGGUUACC- |
| | | | GUUUGGGA |
| 418. | hsa-mir- | MI0001150 | ACUGGUCGGUGAUUUAG- |
| | 196b | | GUAGUUUCCUGUUGUUGG- |
| | | | GAUCCACCUUUCUCUCGA- |
| | | | CAGCACGACACUGCCUU- |
| | | | CAUUACUUCAGUUG |
| 419. | hsa-mir- | MI0003624 | AAAAUGGUGAGAGCGUU- |
| | 611 | | GAGGGGAGUUCCAGAC- |
| | | | GGAGAUGCGAGGACCC- |
| | | | CUCGGGGUCUGACCCACA |
| 420. | hsa-mir- | MI0000114 | CUCUCUGCUUUCAGCUU- |
| | 107 | | CUUUACAGUGUUGCCUU- |
| | | | GUGGCAUGGAGUUCAAG- |
| | | | CAGCAUUGUACAGGG- |
| | | | CUAUCAAAGCACAGA |
| 421. | hsa-mir- | MI0000489 | AGCUUCCCUGGCUCUAG- |
| | 195 | | CAGCACAGAAAUAUUGG- |
| | | | CACAGGGAAGCGAGU- |
| | | | CUGCCAAUAUUGGCUGUG- |
| | | | CUGCUCCAGGCAGGGUG- |
| | | | GUG |
| 422. | hsa-mir- | MI0000234 | GCCGAGACCGAGUGCA- |
| | 192 | | CAGGGCUCUGACCUAU- |
| | | | GAAUUGACAGCCAGUGCU- |
| | | | CUCGUCUCCCCUCUGG- |
| | | | CUGCCAAUUCCAUAGGU- |
| | | | CACAGGUAUGUUCGCCU- |
| | | | CAAUGCCAGC |
| 423. | hsa-mir- | MI0000442 | CCUUAGCAGAGCUGUGGA- |
| | 122a | | GUGUGACAAUGGUGUUU- |
| | | | GUGUCUAAACUAUCAAAC- |
| | | | GCCAUUAUCACA- |
| | | | CUAAAUAGCUACUG- |
| | | | CUAGGC |
| 424. | hsa-mir- | MI0003562 | GAUAGUAAUAAGAAAGAU- |
| | 556 | | GAGCUCAUUGUAAUAU- |
| | | | GAGCUUCAUUUAUA- |
| | | | CAUUUCAUAUUAC- |
| | | | CAUUAGCUCAU- |
| | | | CUUUUUUAUUACUACCUU- |
| | | | CAACA |
| 425. | hsa-mir- | MI0003556 | GGGGACUGCCGGGUGACC- |
| | 551a | | CUGGAAAUCCAGAGUGG- |
| | | | GUGGGGCCAGUCUGACC- |
| | | | GUUUCUAGGCGACCCACU- |
| | | | CUUGGUUUCCAGG- |
| | | | GUUGCCCUGGAAA |
| 426. | hsa-mir- | MI0000736 | ACCAUGCUGUAGUGUGU- |
| | 30c-1 | | GUAAACAUCCUACACUCU- |
| | | | CAGCUGUGAGCUCAAG- |
| | | | GUGGCUGGGAGAGGGUU- |
| | | | GUUUACUCCUUCUGC- |
| | | | CAUGGA |
| 427. | hsa-mir- | MI0003644 | AACUUAACAUCAUGCUAC- |
| | 630 | | CUCUUUGUAUCAUAUUUU- |
| | | | GUUAUUCUGGUCACA- |
| | | | GAAUGACCUAGUAUUCU- |
| | | | GUACCAGGGAAGGUAGUU- |
| | | | CUUAACUAUAU |
| 428. | hsa-mir- | MI0003162 | UCCCAUGCUGUGACCCUC- |
| | 519d | | CAAAGGGAAGCGCUUUCU- |
| | | | GUUUGUUUUCUCUUAAA- |
| | | | CAAAGUGCCUCCCUUUA- |
| | | | GAGUGUUACCGUUUGGGA |
| 429. | hsa-mir- | MI0003191 | GGGAUGCCACAUUCAGC- |
| | 513-1 | | CAUUCAGCGUACAGUGC- |
| | | | CUUUCACAGGGAGGUGU- |
| | | | CAUUUAUGUGAA- |
| | | | CUAAAAUAUAAAUUUCAC- |
| | | | CUUUCUGAGAAGGGUAAU- |
| | | | GUACAGCAUGCACUG- |
| | | | CAUAUGUGGUGUCCC |
| 430. | hsa-mir- | MI0000251 | UGACGGGCGAG- |
| | 208 | | CUUUUGGCCC- |
| | | | GGGUUAUACCUGAUGCU- |
| | | | CACGUAUAAGACGAG- |
| | | | CAAAAAGCUUGUUGGUCA |
| 431. | hsa-mir- | MI0000295 | GACCAGUCGCUGC- |
| | 218-2 | | GGGGCUUUCCUUUGUG- |
| | | | CUUGAUCUAACCAUGUG- |
| | | | GUGGAACGAUGGAAAC- |
| | | | GGAACAUGGUUCUGU- |
| | | | CAAGCACCGCGGAAAG- |
| | | | CACCGUGCUCUCCUGCA |
| 432. | hsa-mir- | MI0001735 | UGGUACUCGGGGAGAG- |
| | 409 | | GUUACCCGAGCAACUUUG- |
| | | | CAUCUGGACGACGAAU- |
| | | | GUUGCUCGGUGAACCC- |
| | | | CUUUUCGGUAUCA |
| 433. | hsa-mir- | MI0002465 | GGUACCUGAGAAGAGGUU- |
| | 410 | | GUCUGUGAUGAGUUC- |
| | | | GCUUUUAUUAAUGAC- |
| | | | GAAUAUAACACAGAUGGC- |
| | | | CUGUUUUCAGUACC |
| 434. | hsa-mir- | MI0001727 | GCAGGAAUGCUGCGAGCA- |
| | 453 | | GUGCCACCUCAUGGUA- |
| | | | CUCGGAGGGAGGUUGUCC- |
| | | | GUGGUGAGUUC- |
| | | | GCAUUAUUUAAUGAUGC |
| 435. | hsa-mir- | MI0000091 | CUGUGGUGCAUUGUA- |
| | 33 | | GUUGCAUUGCAUGUUCUG- |
| | | | GUGGUACCCAUGCAAU- |
| | | | GUUUCCACAGUGCAUCA- |
| | | | CAG |
| 436. | hsa-mir- | MI0000482 | AGGGGGCGAGGGAUUGGA- |
| | 185 | | GAGAAAGGCAGUUCCU- |
| | | | GAUGGUCCCCUCCC- |
| | | | CAGGGGCUGGCUUUCCU- |
| | | | CUGGUCCUUCCCUCCCA |
| 437. | hsa-mir- | MI0000786 | AGGGCUCCUGACUCCAG- |
| | 378 | | GUCCUGUGUGUUACCUA- |
| | | | GAAAUAGCACUGGACUUG- |
| | | | GAGUCAGAAGGCCU |
| 438. | hsa-mir- | MI0003686 | CAGAUCUCAGACAUCUC- |
| | 542 | | GGGGAUCAUCAUGUCAC- |
| | | | GAGAUACCAGUGUGCA- |
| | | | CUUGUGACAGAUUGAUAA- |
| | | | CUGAAAGGUCUGGGAGC- |
| | | | CACUCAUCUUCA |
| 439. | hsa-mir- | MI0003173 | UCUCAAGCUGUGGGUCUG- |
| | 518a-2 | | CAAAGGGAAGCCCUUUCU- |
| | | | GUUGUCUAAAAGAAGA- |
| | | | GAAAGCGCUUCCCUUUG- |
| | | | CUGGAUUACGGUUUGAGA |
| 440. | hsa-mir- | MI0000074 | CACUGUUCUAUGGUUA- |
| | 19b-1 | | GUUUUGCAGGUUUGCAUC- |
| | | | CAGCUGUGUGAUAUUCUG- |
| | | | CUGUGCAAAUCCAUG- |
| | | | CAAAACUGACUGUGGUA- |
| | | | GUG |
| 441. | hsa-mir- | MI0000748 | GGCCUGCCCGACACU- |
| | 130b | | CUUUCCCUGUUGCACUA- |
| | | | CUAUAGGCCGCUGGGAAG- |
| | | | CAGUGCAAUGAU- |
| | | | GAAAGGGCAUCGGUCAG- |
| | | | GUC |
| 442. | hsa-mir- | MI0000772 | GCUCCCUUCAACUUUAA- |
| | 302b | | CAUGGAAGUGCUUUCUGU- |
| | | | GACUUUAAAAGUAAGUG- |
| | | | CUUCCAUGUUUUAGUAG- |
| | | | GAGU |
| 443. | hsa-mir- | MI0003181 | UCUCAGGUUGUGACCUU- |
| | 516-2 | | CUCGAGGAAAGAAGCA- |
| | | | CUUUCUGUUGUCUGAAA- |
| | | | GAAAAGAAAGUGCUUC- |
| | | | CUUUCAGAGGGUUAC- |
| | | | GGUUUGAGA |
| 444. | hsa-mir- | MI0001648 | CUGUGUGUGAUGAGCUGG- |
| | 449 | | CAGUGUAUUGUUAGCUG- |
| | | | GUUGAAUAUGUGAAUGG- |
| | | | CAUCGGCUAACAUGCAA- |
| | | | CUGCUGUCUUAUUG- |
| | | | CAUAUACA |
| 445. | hsa-mir- | MI0003578 | CCUCAGUAAGACCAAGCU- |
| | 571 | | CAGUGUGCCAUUUCCUU- |
| | | | GUCUGUAGCCAUGU- |
| | | | CUAUGGGCUCUUGA- |
| | | | GUUGGCCAUCUGAGU- |
| | | | GAGGGCCUGCUUAUUCUA |
| 446. | hsa-mir- | MI0003176 | UCUCAGGCUGUGACCCUC- |
| | 521-1 | | CAAAGGGAAGAACUUUCU- |
| | | | GUUGUCUAAAAGAAAA- |
| | | | GAACGCACUUCCCUUUA- |
| | | | GAGUGUUACCGUGUGAGA |
| 447. | hsa-mir- | MI0003590 | AACUCACACAUUAAC- |
| | 583 | | CAAAGAGGAAGGUCC- |
| | | | CAUUACUGCAGGGAU- |
| | | | CUUAGCAGUACUGGGAC- |
| | | | CUACCUCUUUGGU |
| 448. | hsa-mir- | MI0000262 | AAUCUAAAGACAACAUUU- |
| | 147 | | CUGCACACACACCAGA- |
| | | | CUAUGGAAGCCAGUGU- |
| | | | GUGGAAAUGCUUCUGCUA- |
| | | | GAUU |
| 449. | hsa-mir- | MI0003569 | AGCAAAGAAGUGU- |
| | 563 | | GUUGCCCUCUAGGAAAU- |
| | | | GUGUGUUGCUCUGAU- |
| | | | GUAAUUAGGUUGACAUAC- |
| | | | GUUUCCCUGGUAGCCA |
| 450. | hsa-mir- | MI0003651 | UGGCGGCCUGGGCGGGAGC |
| | 636 | | GCGCGGGCGGGGCCGGCCC |
| | | | CGCUGCCUG- |
| | | | GAAUUAACCCCGCUGUG- |
| | | | CUUGCUCGUCCC- |
| | | | GCCCGCAGCCCUAGGC- |
| | | | GGCGUCG |
| 451. | hsa-mir- | MI0000810 | CACUCUGCUGUGGC- |
| | 135b | | CUAUGGCUUUUCAUUC- |
| | | | CUAUGUGAUUGCUGUCC- |
| | | | CAAACUCAUGUAGGG- |
| | | | CUAAAAGCCAUGGGCUA- |
| | | | CAGUGAGGGGCGAGCUCC |
| 452. | hsa-mir- | MI0000113 | CCUUGGCCAUGUAAAA- |
| | 106a | | GUGCUUACAGUGCAG- |
| | | | GUAGCUUUUUGAGAUCUA- |
| | | | CUGCAAUGUAAGCACUU- |
| | | | CUUACAUUACCAUGG |
| 453. | hsa-mir- | MI0001652 | AAACGAUACUAAACU- |
| | 450-1 | | GUUUUUGCGAUGUGUUC- |
| | | | CUAAUAUGCA- |
| | | | CUAUAAAUAUAUUGGGAA- |
| | | | CAUUUUGCAUGUAUA- |
| | | | GUUUUGUAUCAAUAUA |
| 454. | hsa-mir- | MI0000450 | ACAAUGCUUUGCUAGAG- |
| | 133a-1 | | CUGGUAAAAUGGAAC- |
| | | | CAAAUCGCCUCUUCAAUG- |
| | | | GAUUUGGUCCCCUUCAAC- |
| | | | CAGCUGUAGCUAUGCAUU- |
| | | | GA |
| 455. | hsa-mir- | MI0000253 | GAGGCAAAGUUCUGAGA- |
| | 148a | | CACUCCGACUCUGAGUAU- |
| | | | GAUAGAAGUCAGUGCA- |
| | | | CUACAGAACUUUGUCUC |
| 456. | hsa-mir- | MI0000802 | UUGUACCUGGUGU- |
| | 340 | | GAUUAUAAAGCAAUGAGA- |
| | | | CUGAUUGUCAUAUGUC- |
| | | | GUUUGUGGGAUCCGUCU- |
| | | | CAGUUACUUUAUAGC- |
| | | | CAUACCUGGUAUCUUA |
| 457. | hsa-mir- | MI0003678 | CUGAAAUAGGUUGCCUGU- |
| | 656 | | GAGGUGUUCACUUU- |
| | | | CUAUAUGAU- |
| | | | GAAUAUUAUACAGUCAAC- |
| | | | CUCUUUCCGAUAUCGAAUC |
| 458. | hsa-mir- | MI0003628 | CUCGGGAGGGGC- |
| | 615 | | GGGAGGGGGGUCCCC- |
| | | | GGUGCUCGGAUCUCGAGG- |
| | | | GUGCUUAUUGUUCGGUCC- |
| | | | GAGCCUGGGUCUCCCU- |
| | | | CUUCCCCCCAACCCCCC |
| 459. | hsa-mir- | MI0003676 | GGGUAAGUGGAAAGAUG- |
| | 654 | | GUGGGCCGCAGAACAU- |
| | | | GUGCUGAGUUCGUGC- |
| | | | CAUAUGUCUGCUGACCAU- |
| | | | CACCUUUAGAAGCCC |
| 460. | hsa-mir- | MI0000107 | CUUCUGGAAGCUGGUUU- |
| | 29b-2 | | CACAUGGUGGCUUA- |
| | | | GAUUUUUCCAUCUUU- |
| | | | GUAUCUAGCACCAUUU- |
| | | | GAAAUCAGUGUUUUAGGAG |
| 461. | hsa-mir- | MI0000650 | CCCUCGUCUUACCCAGCA- |
| | 200c | | GUGUUUGGGUGCGGUUGG- |
| | | | GAGUCUCUAAUACUGCC- |
| | | | GGGUAAUGAUGGAGG |
| 462. | hsa-mir- | MI0003592 | UGGGGUGUCUGUG- |
| | 585 | | CUAUGGCAGCCCUAGCA- |
| | | | CACAGAUACGCCCAGA- |
| | | | GAAAGCCUGAAC- |
| | | | GUUGGGCGUAUCUGUAUG- |
| | | | CUAGGGCUGCUGUAACAA |
| 463. | hsa-mir- | MI0003670 | GCUGUUGAGGCUGC- |
| | 662 | | GCAGCCAGGCCCUGAC- |
| | | | GGUGGGGUGGCUGC- |
| | | | GGGCCUUCUGAAGGU- |
| | | | CUCCCACGUUGUGGCC- |
| | | | CAGCAGCGCAGUCAC- |
| | | | GUUGC |
| 464. | hsa-mir- | MI0003614 | UGCAUGAGUUCGUCUUG- |
| | 601 | | GUCUAGGAUUGUUGGAG- |
| | | | GAGUCAGAAAAACUACCC- |
| | | | CAGGGAUCCUGAAGUC- |
| | | | CUUUGGGUGGA |
| 465. | hsa-mir- | MI0003154 | UCUCAUGCUGUGACCCU- |
| | 518f | | CUAGAGGGAAGCACUUU- |
| | | | CUCUUGUCUAAAAGAAAA- |
| | | | GAAAGCGCUUCUCUUUA- |
| | | | GAGGAUUACUCUUUGAGA |
| 466. | hsa-mir- | MI0003647 | CGCCUCCUACCGCAGUG- |
| | 632 | | CUUGACGGGAGGCGGAGC- |
| | | | GGGGAACGAGGCCGUC- |
| | | | GGCCAUUUUGUGUCUG- |
| | | | CUUCCUGUGGGACGUG- |
| | | | GUGGUAGCCGU |
| 467. | hsa-mir- | MI0003516 | CCCAGCCUGGCACAUUA- |
| | 545 | | GUAGGCCUCAGUAAAU- |
| | | | GUUUAUUAGAU- |
| | | | GAAUAAAUGAAUGACU- |
| | | | CAUCAGCAAACAUUUAUU- |
| | | | GUGUGCCUGCUAAAGU- |
| | | | GAGCUCCACAGG |
| 468. | hsa-mir- | MI0000811 | CAAGCACGAUUAGCAUUU- |
| | 148b | | GAGGUGAAGUUCU- |
| | | | GUUAUACACUCAGGCU- |
| | | | GUGGCUCUCUGAAAGUCA- |
| | | | GUGCAUCACAGAACUUU- |
| | | | GUCUCGAAAGCUUUCUA |
| 469. | hsa-mir- | MI0000437 | ACCUACUCAGAGUACAUA- |
| | 1-2 | | CUUCUUUAUGUACC- |
| | | | CAUAUGAACAUACAAUG- |
| | | | CUAUGGAAUGUAAAGAA- |
| | | | GUAUGUAUUUUUGGUAGGC |
| 470. | hsa-mir- | MI0003199 | GUUGUCUGUGGUACCCUA- |
| | 514-2 | | CUCUGGAGAGUGACAAU- |
| | | | CAUGUAUAACUAAAUUU- |
| | | | GAUUGACACUUCUGUGA- |
| | | | GUAGAGUAACGCAUGACAC |
| 471. | hsa-mir- | MI0000088 | GCGACUGUAAACAUCCUC- |
| | 30a | | GACUGGAAGCUGUGAAGC- |
| | | | CACAGAUGGGCUUUCA- |
| | | | GUCGGAUGUUUGCAGCUGC |
| 472. | hsa-mir- | MI0000813 | CUGACUAUGCCUCCCC- |
| | 324 | | GCAUCCCCUAGGGCAUUG- |
| | | | GUGUAAAGCUGGAGACC- |
| | | | CACUGCCCCAGGUGCUG- |
| | | | CUGGGGGUUGUAGUC |
| 473. | hsa-mir- | MI0000115 | GUUCCACUCUAGCAGCAC- |
| | 16-2 | | GUAAAUAUUGGCGUAGU- |
| | | | GAAAUAUAUAUUAAACAC- |
| | | | CAAUAUUACUGUGCUG- |
| | | | CUUUAGUGUGAC |
| 474. | hsa-mir- | MI0003645 | GUGGGGAGCCUGGUUA- |
| | 631 | | GACCUGGCCCAGACCU- |
| | | | CAGCUACACAAGCUGAUG- |
| | | | GACUGAGUCAGGGGCCA- |
| | | | CACUCUCC |
| 475. | hsa-mir- | MI0003610 | GCUUGAUGAUGCUGCU- |
| | 598 | | GAUGCUGGCGGUGAUCCC- |
| | | | GAUGGUGUGAGCUG- |
| | | | GAAAUGGGGUGCUACGU- |
| | | | CAUCGUUGUCAUCGUCAU- |
| | | | CAUCAUCAUCCGAG |
| 476. | hsa-mir- | MI0000102 | CCUGUUGCCACAAACCC- |
| | 100 | | GUAGAUCCGAACUUGUG- |
| | | | GUAUUAGUCCGCACAAG- |
| | | | CUUGUAUCUAUAGGUAU- |
| | | | GUGUCUGUUAGG |
| 477. | hsa-mir- | MI0000783 | CCCCGCGACGAGCCCCUC- |
| | 375 | | GCACAAACCGGACCU- |
| | | | GAGCGUUUUGUUCGUUC- |
| | | | GGCUCGCGUGAGGC |
| 478. | hsa-mir- | MI0003589 | AUCUGUGCUCUUUGAUUA- |
| | 582 | | CAGUUGUUCAACCAGUUA- |
| | | | CUAAUCUAACUAAUU- |
| | | | GUAACUGGUUGAACAACU- |
| | | | GAACCCAAAGGGUGCAAA- |
| | | | GUAGAAACAUU |
| 479. | hsa-mir- | MI0003166 | UCCCAUGCUGUGACCCU- |
| | 520g | | CUAGAGGAAGCACUUUCU- |
| | | | GUUUGUUGUCUGA- |
| | | | GAAAAAACAAAGUG- |
| | | | CUUCCCUUUAGAGU- |
| | | | GUUACCGUUUGGGA |
| 480. | hsa-mir- | MI0003626 | GGUGAGUGCGUUUCCAA- |
| | 613 | | GUGUGAAGGGACCCUUC- |
| | | | CUGUAGUGUCUUAUAUA- |
| | | | CAAUACAGUAGGAAU- |
| | | | GUUCCUUCUUUGCCACU- |
| | | | CAUACACCUUUA |
| 481. | hsa-mir- | MI0000744 | AAGAAAUGGUUUACC- |
| | 299 | | GUCCCACAUACAUUUU- |
| | | | GAAUAUGUAUGUGGGAUG- |
| | | | GUAAACCGCUUCUU |
| 482. | hsa-mir- | MI0000814 | UCUCCAACAAUAUCCUG- |
| | 338 | | GUGCUGAGUGAUGACU- |
| | | | CAGGCGACUCCAGCAUCA- |
| | | | GUGAUUUUGUUGAAGA |
| 483. | hsa-mir- | MI0000760 | GGAGCUUAUCAGAAUCUC- |
| | 361 | | CAGGGGUA- |
| | | | CUUUAUAAUUUCAAAAA- |
| | | | GUCCCCCAGGUGUGAUU- |
| | | | CUGAUUUGCUUC |
| 484. | hsa-mir- | MI0000438 | UUGAGGCCUUAAAGUACU- |
| | 15b | | GUAGCAGCACAUCAUG- |
| | | | GUUUACAUGCUACAGU- |
| | | | CAAGAUGCGAAU- |
| | | | CAUUAUUUGCUGCUCUA- |
| | | | GAAAUUUAAGGAAAUUCAU |
| 485. | hsa-mir- | MI0000476 | CCCUGGCAUGGUGUG- |
| | 138-1 | | GUGGGGCAGCUGGUGUU- |
| | | | GUGAAUCAGGCCGUUGC- |
| | | | CAAUCAGAGAACGGCUA- |
| | | | CUUCACAACACCAGGGC- |
| | | | CACACCACACUACAGG |
| 486. | hsa-let- | MI0000068 | UGUGGGAUGAGGUAGUA- |
| | 7f-2 | | GAUUGUAUAGUUUUAGG- |
| | | | GUCAUACCCCAUCUUGGA- |
| | | | GAUAACUAUACAGUCUA- |
| | | | CUGUCUUUCCCACG |
| 487. | hsa-mir- | MI0000447 | UGAGCUGUUGGAUUC- |
| | 128a | | GGGGCCGUAGCACUGUCU- |
| | | | GAGAGGUUUACAUUUCU- |
| | | | CACAGUGAACCGGUCU- |
| | | | CUUUUUCAGCUGCUUC |
| 488. | hsa-mir- | MI0000749 | GGGCAGUCUUUGCUACU- |
| | 30e | | GUAAACAUCCUUGACUG- |
| | | | GAAGCUGUAAGGUGUUCA- |
| | | | GAGGAGCUUUCAGUC- |
| | | | GGAUGUUUACAGC- |
| | | | GGCAGGCUGCCA |
| 489. | hsa-mir- | MI0003187 | CCAAAGAAAGAUGCUAAA- |
| | 450-2 | | CUAUUUUUGCGAUGU- |
| | | | GUUCCUAAUAU- |
| | | | GUAAUAUAAAU- |
| | | | GUAUUGGGGACAUUUUG- |
| | | | CAUUCAUAGUUUUGUAU- |
| | | | CAAUAAUAUGG |
| 490. | hsa-mir- | MI0000815 | CGGGGCGGCCGCUCUCC- |
| | 339 | | CUGUCCUCCAGGAGCU- |
| | | | CACGUGUGCCUGCCUGU- |
| | | | GAGCGCCUCGACGACA- |
| | | | GAGCCGGCGCCUGCCCCA- |
| | | | GUGUCUGCGC |
| 491. | hsa-mir- | MI0003529 | GGUAUUUAAAAGGUA- |
| | 376a-2 | | GAUUUUCCUUCUAUG- |
| | | | GUUACGUGUUUGAUG- |
| | | | GUUAAUCAUAGAG- |
| | | | GAAAAUCCACGUUUUCA- |
| | | | GUAUC |
| 492. | hsa-mir- | MI0003145 | UCUCAUGCAGUCAUUCUC- |
| | 519e | | CAAAAGGGAGCACUUUCU- |
| | | | GUUUGAAAGAAAACAAA- |
| | | | GUGCCUCCUUUUAGAGU- |
| | | | GUUACUGUUUGAGA |
| 493. | hsa-mir- | MI0000446 | UGCGCUCCUCUCAGUCC- |
| | 125b-1 | | CUGAGACCCUAACUUGU- |
| | | | GAUGUUUACC- |
| | | | GUUUAAAUCCAC- |
| | | | GGGUUAGGCUCUUGGGAG- |
| | | | CUGCGAGUCGUGCU |
| 494. | hsa-mir- | MI0003633 | CGCCCACCUCAGCCUCC- |
| | 619 | | CAAAAUGCUGGGAUUA- |
| | | | CAGGCAUGAGCCACUGC- |
| | | | GGUCGACCAUGACCUGGA- |
| | | | CAUGUUUGUGCCCAGUA- |
| | | | CUGUCAGUUUGCAG |
| 495. | hsa-mir- | MI0003184 | GCUCCCCCUCUCUAAUC- |
| | 500 | | CUUGCUACCUGGGUGAGA- |
| | | | GUGCUGUCUGAAUG- |
| | | | CAAUGCACCUGGGCAAG- |
| | | | GAUUCUGAGAGCGAGAGC |
| 496. | hsa-mir- | MI0003168 | GUGACCCUCUAGAGG- |
| | 526a-2 | | GAAGCACUUUCUGUU- |
| | | | GAAAGAAAAGAACAUG- |
| | | | CAUCCUUUCAGAGGGUUAC |
| 497. | hsa-mir- | MI0000473 | UGCCCUUCGCGAAU- |
| | 129-2 | | CUUUUUGCGGUCUGGG- |
| | | | CUUGCUGUACAUAACU- |
| | | | CAAUAGCCGGAAGCC- |
| | | | CUUACCCCAAAAAG- |
| | | | CAUUUGCGGAGGGCG |
| 498. | hsa-mir- | MI0000090 | GGAGAUAUUGCACAUUA- |
| | 32 | | CUAAGUUGCAUGUUGU- |
| | | | CACGGCCUCAAUG- |
| | | | CAAUUUAGUGUGUGU- |
| | | | GAUAUUUUC |
| 499. | hsa-mir- | MI0000297 | GACAGUGUGGCAUU- |
| | 220 | | GUAGGGCUCCACACC- |
| | | | GUAUCUGACACUUUGGGC- |
| | | | GAGGGCACCAUGCUGAAG- |
| | | | GUGUUCAUGAUGCGGU- |
| | | | CUGGGAACUCCUCAC- |
| | | | GGAUCUUACUGAUG |
| 500. | hsa-mir- | MI0003601 | UGAUGCUUUGCUGGCUG- |
| | 550-2 | | GUGCAGUGCCUGAGGGA- |
| | | | GUAAGAGCCCUGUUGUU- |
| | | | GUCAGAUAGUGUCUUA- |
| | | | CUCCCUCAGGCACAUCUC- |
| | | | CAGCGAGUCUCU |
| 501. | hsa-mir- | MI0001446 | CGAGGGGAUACAGCAG- |
| | 424 | | CAAUUCAUGUUUUGAAGU- |
| | | | GUUCUAAAUGGUU- |
| | | | CAAAACGUGAGGCGCUG- |
| | | | CUAUACCCCCUCGUGGG- |
| | | | GAAGGUAGAAGGUGGGG |
| 502. | hsa-mir- | MI0000474 | CAGGGUGUGUGACUGGUU- |
| | 134 | | GACCAGAGGGGCAUGCA- |
| | | | CUGUGUUCACCCU- |
| | | | GUGGGCCACCUAGUCAC- |
| | | | CAACCCUC |
| 503. | hsa-mir- | MI0000737 | CCGGGCCCCUGUGAGCAU- |
| | 200a | | CUUACCGGACAGUGCUG- |
| | | | GAUUUCCCAGCUUGACU- |
| | | | CUAACACUGUCUGGUAAC- |
| | | | GAUGUUCAAAGGU- |
| | | | GACCCGC |
| 504. | hsa-mir- | MI0003621 | GGGCCAAGGUGGGC- |
| | 608 | | CAGGGGUGGUGUUGGGA- |
| | | | CAGCUCCGUUUAAAAAGG- |
| | | | CAUCUCCAAGAGCUUC- |
| | | | CAUCAAAGGCUGCCU- |
| | | | CUUGGUGCAGCACAGGUA- |
| | | | GA |
| 505. | hsa-mir- | MI0003606 | CUAAUGGAUAAGG- |
| | 594 | | CAUUGGCCUCCUAAGC- |
| | | | CAGGGAUUGUGGGUUCGA- |
| | | | GUCCCAUCUGGGGUGGC- |
| | | | CUGUGACUUUUGUC- |
| | | | CUUUUUUCCCC |
| 506. | hsa-mir- | MI0003612 | CCUAGAAUGUUAUUAG- |
| | 548a-3 | | GUCGGUGCAAAA- |
| | | | GUAAUUGCGAGUUUUAC- |
| | | | CAUUACUUUCAAUGG- |
| | | | CAAAACUGGCAAUUA- |
| | | | CUUUUGCACCAAC- |
| | | | GUAAUACUU |
| 507. | hsa-mir- | MI0000739 | ACUGUCCUUUUUC- |
| | 101-2 | | GGUUAUCAUGGUACC- |
| | | | GAUGCUGUAUAUCU- |
| | | | GAAAGGUACAGUACUGU- |
| | | | GAUAACUGAAGAAUGGUG- |
| | | | GU |
| 508. | hsa-mir- | MI0001518 | UGUGUUAAGGUGCAUCUA- |
| | 18b | | GUGCAGUUAGUGAAGCAG- |
| | | | CUUAGAAUCUACUGCC- |
| | | | CUAAAUGCCCCUUCUGGCA |
| 509. | hsa-mir- | MI0003151 | CAUGCUGUGACCCUCUA- |
| | 519b | | GAGGGAAGCGCUUUCU- |
| | | | GUUGUCUGAAAGAAAA- |
| | | | GAAAGUGCAUCCUUUUA- |
| | | | GAGGUUUACUGUUUG |
| 510. | hsa-mir- | MI0003167 | UCUCAUGAUGUGACCAU- |
| | 516-3 | | CUGGAGGUAAGAAGCA- |
| | | | CUUUGUGUUUUGUGAAA- |
| | | | GAAAGUGCUUCCUUUCA- |
| | | | GAGGGUUACUCUUUGAGA |
| 511. | hsa-mir- | MI0000804 | UGGAGUGGGGGGGCAG- |
| | 328 | | GAGGGGCUCAGGGAGAAA- |
| | | | GUGCAUACAGCCC- |
| | | | CUGGCCCUCUCUGCC- |
| | | | CUUCCGUCCCCUG |
| 512. | hsa-mir- | MI0001725 | GGUACCUGAAGAGAG- |
| | 329-1 | | GUUUUCUGGGUUUCU- |
| | | | GUUUCUUUAAUGAGGAC- |
| | | | GAAACACACCUGGUUAAC- |
| | | | CUCUUUUCCAGUAUC |
| 513. | hsa-mir- | MI0003655 | GUGACCCUGGGCAAGUUC- |
| | 640 | | CUGAAGAUCAGACACAU- |
| | | | CAGAUCCCUUAUCU- |
| | | | GUAAAAUGGGCAUGAUC- |
| | | | CAGGAACCUGCCUCUAC- |
| | | | GGUUGCCUUGGGG |
| 514. | hsa-mir- | MI0003640 | ACUGAUAUAUUUGU- |
| | 626 | | CUUAUUUGAGAGCUGAG- |
| | | | GAGUAUUUUUAUGCAAU- |
| | | | CUGAAUGAUCUCAGCUGU- |
| | | | CUGAAAAUGUCUU- |
| | | | CAAUUUUAAAGGCUU |
| 515. | hsa-mir- | MI0003663 | AUCACAGACACCUCCAA- |
| | 648 | | GUGUGCAGGGCACUG- |
| | | | GUGGGGGCC- |
| | | | GGGGCAGGCCCAGCGAAA- |
| | | | GUGCAGGACCUGGCA- |
| | | | CUUAGUCGGAAGUGAGG- |
| | | | GUG |
| 516. | hsa-mir- | MI0003205 | CGACUUGCUUUCUCUC- |
| | 532 | | CUCCAUGCCUUGAGU- |
| | | | GUAGGACCGUUGGCAU- |
| | | | CUUAAUUACCCUCCCA- |
| | | | CACCCAAGGCUUG- |
| | | | CAAAAAAGCGAGCCU |
| 517. | hsa-mir- | MI0000097 | AACACAGUGGGCACU- |
| | 95 | | CAAUAAAUGUCUGUU- |
| | | | GAAUUGAAAUGCGUUA- |
| | | | CAUUCAAC- |
| | | | GGGUAUUUAUUGAGCACC- |
| | | | CACUCUGUG |
| 518. | hsa-mir- | MI0000286 | ACCCGGCAGUGCCUC- |
| | 210 | | CAGGCGCAGGGCAGCCC- |
| | | | CUGCCCACCGCACACUGC- |
| | | | GCUGCCCCAGACCCACU- |
| | | | GUGCGUGUGACAGCGGCU- |
| | | | GAUCUGUGCCUGGGCAGC- |
| | | | GCGACCC |
| 519. | hsa-mir- | MI0000290 | GGCCUGGCUGGACAGA- |
| | 214 | | GUUGUCAUGUGUCUGCCU- |
| | | | GUCUACACUUGCUGUGCA- |
| | | | GAACAUCCGCUCACCU- |
| | | | GUACAGCAGGCACAGA- |
| | | | CAGGCAGUCACAUGA- |
| | | | CAACCCAGCCU |
| 520. | hsa-mir- | MI0000281 | AGGAAGCUUCUGGAGAUC- |
| | 199a-2 | | CUGCUCCGUCGCCCCAGU- |
| | | | GUUCAGACUACCUGUU- |
| | | | CAGGACAAUGCCGUUGUA- |
| | | | CAGUAGUCUGCACAUUG- |
| | | | GUUAGACUGGGCAAGGGA- |
| | | | GAGCA |
| 521. | hsa-mir- | MI0000460 | UGGGGCCCUGGCUGG- |
| | 144 | | GAUAUCAUCAUAUACU- |
| | | | GUAAGUUUGCGAUGAGA- |
| | | | CACUACAGUAUAGAUGAU- |
| | | | GUACUAGUCC- |
| | | | GGGCACCCCC |
| 522. | hsa-mir- | MI0000285 | AAAGAUCCUCAGACAAUC- |
| | 205 | | CAUGUGCUUCUCUUGUC- |
| | | | CUUCAUUCCACCGGAGU- |
| | | | CUGUCUCAUACCCAACCA- |
| | | | GAUUUCAGUGGAGUGAA- |
| | | | GUUCAGGAGGCAUGGAG- |
| | | | CUGACA |
| 523. | hsa-mir- | MI0003659 | UUUUUUUUUA- |
| | 644 | | GUAUUUUUCCAUCAGU- |
| | | | GUUCAUAAGGAAUGUUG- |
| | | | CUCUGUAGUUUUCUUAUA- |
| | | | GUGUGGCUUUCUUAGAG- |
| | | | CAAAGAUGGUUCCCUA |
| 524. | hsa-mir- | MI0003679 | AGACAUGCAACUCAA- |
| | 549 | | GAAUAUAUUGAGAGCU- |
| | | | CAUCCAUAGUUGUCACU- |
| | | | GUCUCAAAUCAGUGACAA- |
| | | | CUAUGGAUGAGCU- |
| | | | CUUAAUAUAUCCCAGGC |
| 525. | hsa-mir- | MI0003617 | AGAGCAUCGUGCUUGAC- |
| | 604 | | CUUCCACGCUCUCGUGUC- |
| | | | CACUAGCAGGCAGGUUUU- |
| | | | CUGACACAGGCUGC- |
| | | | GGAAUUCAGGACAGUG- |
| | | | CAUCAUGGAGA |
| 526. | hsa-mir- | MI0000105 | CUUCAGGAAGCUGGUUU- |
| | 29b-1 | | CAUAUGGUGGUUUA- |
| | | | GAUUUAAAUAGUGAUUGU- |
| | | | CUAGCACCAUUUGAAAU- |
| | | | CAGUGUUCUUGGGGG |
| 527. | hsa-mir- | MI0003580 | UUUAGCGGUUUCUCCCU- |
| | 573 | | GAAGUGAUGUGUAACU- |
| | | | GAUCAGGAUCUACUCAU- |
| | | | GUCGUCUUUGGUAAA- |
| | | | GUUAUGUCGCUUGUCAGG- |
| | | | GUGAGGAGAGUUUUUG |
| 528. | hsa-mir- | MI0002468 | AGCCUCGUCAGGCUCA- |
| | 484 | | GUCCCCUCCC- |
| | | | GAUAAACCCCUAAAUAGG- |
| | | | GACUUUCCCGGGGGGU- |
| | | | GACCCUGGCUUUUUUGGCG |
| 529. | hsa-mir- | MI0000732 | UGGUUCCCGCCCCCU- |
| | 194-2 | | GUAACAGCAACUCCAU- |
| | | | GUGGAAGUGCCCACUG- |
| | | | GUUCCAGUGGGGCUGCU- |
| | | | GUUAUCUGGGGCGAGGGC- |
| | | | CAG |
| 530. | hsa-mir- | MI0000776 | AAAAGGUGGAUAUUCCUU- |
| | 368 | | CUAUGUUUAU- |
| | | | GUUAUUUAUGGUUAAA- |
| | | | CAUAGAGGAAAUUCCAC- |
| | | | GUUUU |
| 531. | hsa-mir- | MI0003561 | GGAGUGAACUCAGAUGUG- |
| | 555 | | GAGCACUACCUUUGUGAG- |
| | | | CAGUGUGACCCAAGGCCU- |
| | | | GUGGACAGGGUAAGCU- |
| | | | GAACCUCUGAUAAAACU- |
| | | | CUGAUCUAU |
| 532. | hsa-mir- | MI0003616 | GAUUGAUGCUGUUG- |
| | 603 | | GUUUGGUGCAAAA- |
| | | | GUAAUUGCAGUGCUUCC- |
| | | | CAUUUAAAAGUAAUGGCA- |
| | | | CACACUGCAAUUA- |
| | | | CUUUUGCUCCAA- |
| | | | CUUAAUACUU |
| 533. | hsa-mir- | MI0003170 | UCUCAAGCUGUGACUG- |
| | 518a-1 | | CAAAGGGAAGCCCUUUCU- |
| | | | GUUGUCUGAAAGAAGA- |
| | | | GAAAGCGCUUCCCUUUG- |
| | | | CUGGAUUACGGUUUGAGA |
| 534. | hsa-mir- | MI0000101 | CCCAUUGGCAUAAACCC- |
| | 99a | | GUAGAUCCGAUCUUGUG- |
| | | | GUGAAGUGGACCGCA- |
| | | | CAAGCUCGCUUCUAUGG- |
| | | | GUCUGUGUCAGUGUG |
| 535. | hsa-mir- | MI0003132 | CUGGCCUCCAGGGCUUU- |
| | 493 | | GUACAUGGUAGGCUUU- |
| | | | CAUUCAUUCGUUUGCA- |
| | | | CAUUCGGUGAAGGUCUA- |
| | | | CUGUGUGCCAGGCCCU- |
| | | | GUGCCAG |
| 536. | hsa-mir- | MI0000448 | UGCUGCUGGCCAGAGCU- |
| | 130a | | CUUUUCACAUUGUGCUA- |
| | | | CUGUCUGCACCUGUCA- |
| | | | CUAGCAGUGCAAU- |
| | | | GUUAAAAGGGCAUUGGCC- |
| | | | GUGUAGUG |
| 537. | hsa-mir- | MI0000108 | UUGUGCUUUCAGCUU- |
| | 103-2 | | CUUUACAGUGCUGCCUU- |
| | | | GUAGCAUUCAGGUCAAG- |
| | | | CAGCAUUGUACAGGG- |
| | | | CUAUGAAAGAACCA |
| 538. | hsa-mir- | MI0000085 | CUGAGGAGCAGGGCUUAG- |
| | 27a | | CUGCUUGUGAGCAGGGUC- |
| | | | CACACCAAGUCGUGUUCA- |
| | | | CAGUGGCUAAGUUCC- |
| | | | GCCCCCCAG |
| 539. | hsa-mir- | MI0001444 | GAGAGAAGCACUGGA- |
| | 422a | | CUUAGGGUCAGAAGGCCU- |
| | | | GAGUCUCUCUGCUGCA- |
| | | | GAUGGGCUCUCUGUCCCU- |
| | | | GAGCCAAGCUUUGUC- |
| | | | CUCCCUGG |
| 540. | hsa-mir- | MI0003669 | GGAGAGGCUGUGCU- |
| | 661 | | GUGGGGCAGGCGCAGGC- |
| | | | CUGAGCCCUGGUUUC- |
| | | | GGGCUGCCUGGGUCU- |
| | | | CUGGCCUGCGCGUGA- |
| | | | CUUUGGGGUGGCU |
| 541. | hsa-mir- | MI0003150 | UCAGGCUGUGACCCUCUU- |
| | 526b | | GAGGGAAGCACUUUCU- |
| | | | GUUGUCUGAAAGAAGA- |
| | | | GAAAGUGCUUCCUUUUA- |
| | | | GAGGCUUACUGUCUGA |
| 542. | hsa-mir- | MI0000440 | ACCUCUCUAACAAGGUG- |
| | 27b | | CAGAGCUUAGCUGAUUG- |
| | | | GUGAACAGUGAUUG- |
| | | | GUUUCCGCUUUGUUCACA- |
| | | | GUGGCUAAGUUCUGCAC- |
| | | | CUGAAGAGAAGGUG |
| 543. | hsa-mir- | MI0003573 | GGAUUCUUAUAGGACA- |
| | 567 | | GUAUGUUCUUCCAGGACA- |
| | | | GAACAUUCUUUG- |
| | | | CUAUUUUGUACUGGAA- |
| | | | GAACAUGCAAAA- |
| | | | CUAAAAAAAAAAAAA- |
| | | | GUUAUUGCU |
| 544. | hsa-mir- | MI0000683 | CUGAUGGCUGCACUCAA- |
| | 181b-2 | | CAUUCAUUGCUGUC- |
| | | | GGUGGGUUUGAGUCU- |
| | | | GAAUCAACUCACUGAU- |
| | | | CAAUGAAUGCAAACUGC- |
| | | | GGACCAAACA |
| 545. | hsa-mir- | MI0000762 | CUUGAAUCCUUGGAAC- |
| | 362 | | CUAGGUGUGAGUG- |
| | | | CUAUUUCAGUGCAACA- |
| | | | CACCUAUUCAAGGAUU- |
| | | | CAAA |
| 546. | hsa-mir- | MI0000780 | GUGGGCCUCAAAUGUG- |
| | 372 | | GAGCACUAUUCUGAUGUC- |
| | | | CAAGUGGAAAGUGCUGC- |
| | | | GACAUUUGAGCGUCAC |
| 547. | hsa-mir- | MI0000272 | GAGCUGCUUGC- |
| | 182 | | CUCCCCCCGUUUUUGG- |
| | | | CAAUGGUAGAACUCACA- |
| | | | CUGGUGAGGUAACAG- |
| | | | GAUCCGGUGGUUCUAGA- |
| | | | CUUGCCAACUAUGGGGC- |
| | | | GAGGACUCAGCCGGCAC |
| 548. | hsa-mir- | MI0000240 | UCAUUGGUCCAGAGGGGA- |
| | 198 | | GAUAGGUUCCUGU- |
| | | | GAUUUUUCCUUCUUCU- |
| | | | CUAUAGAAUAAAUGA |
| 549. | hsa-mir- | MI0003130 | CGCCUCAGAGCC- |
| | 202 | | GCCCGCCGUUCCUUUUUC- |
| | | | CUAUGCAUAUACUUCUUU- |
| | | | GAGGAUCUGGCCUAAA- |
| | | | GAGGUAUAGGGCAUGG- |
| | | | GAAAACGGGGCGGUC- |
| | | | GGGUCCUCCCCAGCG |
| 550. | hsa-mir- | MI0000468 | GGAGGCCCGUUUCUCU- |
| | 9-3 | | CUUUGGUUAUCUAGCU- |
| | | | GUAUGAGUGCCACA- |
| | | | GAGCCGUCAUAAAGCUA- |
| | | | GAUAACCGAAAGUA- |
| | | | GAAAUGAUUCUCA |
| 551. | hsa-mir- | MI0000294 | GUGAUAAUGUAGCGA- |
| | 218-1 | | GAUUUUCUGUUGUGCUU- |
| | | | GAUCUAACCAUGUG- |
| | | | GUUGCGAGGUAUGA- |
| | | | GUAAAACAUGGUUCCGU- |
| | | | CAAGCACCAUGGAACGU- |
| | | | CACGCAGCUUUCUACA |
| 552. | hsa-mir- | MI0000791 | CUCCUCAGAUCAGAAGGU- |
| | 383 | | GAUUGUGGCUUUGGGUG- |
| | | | GAUAUUAAUCAGCCACAG- |
| | | | CACUGCCUGGUCAGAAA- |
| | | | GAG |
| 553. | hsa-mir- | MI0000112 | UGUGCAUCGUGGU- |
| | 105-2 | | CAAAUGCUCAGACUCCU- |
| | | | GUGGUGGCUGCUUAUG- |
| | | | CACCACGGAUGUUUGAG- |
| | | | CAUGUGCUAUGGUGUCUA |
| 554. | hsa-mir- | MI0000100 | AGGAUUCUGCUCAUGC- |
| | 98 | | CAGGGUGAGGUAGUAA- |
| | | | GUUGUAUUGUUGUGGG- |
| | | | GUAGGGAUAUUAGGCCC- |
| | | | CAAUUAGAAGAUAA- |
| | | | CUAUACAACUUACUA- |
| | | | CUUUCCCUGGUGUGUGG- |
| | | | CAUAUUCA |
| 555. | hsa-mir- | MI0003585 | AGAUAAAUCUAUAGA- |
| | 578 | | CAAAAUACAAUCCCGGA- |
| | | | CAACAAGAAGCUC- |
| | | | CUAUAGCUCCUGUAGCUU- |
| | | | CUUGUGCUCUAGGAUU- |
| | | | GUAUUUUGUUUAUAUAU |
| 556. | hsa-mir- | MI0003594 | AUGGGGUAAAAC- |
| | 586 | | CAUUAUGCAUU- |
| | | | GUAUUUUUAGGUCC- |
| | | | CAAUACAUGUGGGCC- |
| | | | CUAAAAAUACAAUG- |
| | | | CAUAAUGGUUUUUCACU- |
| | | | CUUUAUCUUCUUAU |
| 557. | hsa-mir- | MI0003560 | CGGGCCCCGGGCGGGCGGG |
| | 92b | | AGGGACGGGACGCGGUG- |
| | | | CAGUGUUGUUUUUUCCCCC |
| | | | GCCAAUAUUGCACUC- |
| | | | GUCCC- |
| | | | GGCCUCCGGCCCCCCCGGC |
| | | | CC |
| 558. | hsa-mir- | MI0000296 | CCGCCCCGGGCCGCGGCUC |
| | 219-1 | | CUGAUUGUCCAAAC- |
| | | | GCAAUUCUCGAGU- |
| | | | CUAUGGCUCCGGCCGAGA- |
| | | | GUUGAGUCUGGACGUCCC- |
| | | | GAGCCGCCGCCCCCAAAC- |
| | | | CUCGAGCGGG |
| 559. | hsa-mir- | MI0003639 | AGGGUAGAGGGAU- |
| | 625 | | GAGGGGGAAAGUUCUAUA- |
| | | | GUCCUGUAAUUAGAUCU- |
| | | | CAGGACUAUAGAA- |
| | | | CUUUCCCCCUCAUCCCU- |
| | | | CUGCCCU |
| 560. | hsa-mir- | MI0003190 | GAUGCACCCAGUGGGG- |
| | 505 | | GAGCCAGGAAGUAUUGAU- |
| | | | GUUUCUGCCAGUUUAGC- |
| | | | GUCAACACUUGCUG- |
| | | | GUUUCCUCUCUGGAGCAUC |
| 561. | hsa-mir- | MI0003584 | UGGGGGAGUGAAGAGUA- |
| | 577 | | GAUAAAAUAUUGGUACCU- |
| | | | GAUGAAUCUGAGGCCAG- |
| | | | GUUUCAAUACUUUAUCUG- |
| | | | CUCUUCAUUUCCCCAUAU- |
| | | | CUACUUAC |
| 562. | hsa-mir- | MI0000467 | GGAAGCGAGUUGUUAU- |
| | 9-2 | | CUUUGGUUAUCUAGCU- |
| | | | GUAUGAGUGUAUUGGU- |
| | | | CUUCAUAAAGCUA- |
| | | | GAUAACCGAAAGUAAAAA- |
| | | | CUCCUUCA |
| 563. | hsa-mir- | MI0002467 | GAGGGGGAAGACGGGAG- |
| | 483 | | GAAAGAAGGGAGUGGUUC- |
| | | | CAUCACGCCUCCUCACUC- |
| | | | CUCUCCUCCCGUCUUCUC- |
| | | | CUCUC |
| 564. | hsa-mir- | MI0003200 | GUUGUCUGUGGUACCCUA- |
| | 514-3 | | CUCUGGAGAGUGACAAU- |
| | | | CAUGUAUAACUAAAUUU- |
| | | | GAUUGACACUUCUGUGA- |
| | | | GUAGAGUAACGCAUGACAC |
| 565. | hsa-mir- | MI0003133 | UGACUCCUCCAGGUCUUG- |
| | 432 | | GAGUAGGUCAUUGGGUG- |
| | | | GAUCCUCUAUUUCCUUAC- |
| | | | GUGGGCCACUGGAUGG- |
| | | | CUCCUCCAUGUCUUGGA- |
| | | | GUAGAUCA |
| 566. | hsa-mir- | MI0003674 | UUCAUUCCUUCAGUGUU- |
| | 653 | | GAAACAAUCUCUACU- |
| | | | GAACCAGCUUCAAACAA- |
| | | | GUUCACUGGAGUUUGUUU- |
| | | | CAAUAUUGCAAGAAU- |
| | | | GAUAAGAUGGAAGC |
| 567. | hsa-mir- | MI0003652 | UGGCUAAGGUGUUGGCUC- |
| | 637 | | GGGCUCCCCACUGCA- |
| | | | GUUACCCUCCCCUC- |
| | | | GGCGUUACUGAGCA- |
| | | | CUGGGGGCUUUCGGGCU- |
| | | | CUGCGUCUGCACAGAUA- |
| | | | CUUC |
| 568. | hsa-mir- | MI0003192 | GGAUGCCACAUUCAGC- |
| | 513-2 | | CAUUCAGUGUGCAGUGC- |
| | | | CUUUCACAGGGAGGUGU- |
| | | | CAUUUAUGUGAA- |
| | | | CUAAAAUAUAAAUUUCAC- |
| | | | CUUUCUGAGAAGGGUAAU- |
| | | | GUACAGCAUGCACUG- |
| | | | CAUAUGUGGUGUCC |
| 569. | hsa-mir- | MI0003681 | GUGUAGUAGAGCUAGGAG- |
| | 657 | | GAGAGGGUCCUGGA- |
| | | | GAAGCGUGGACCGGUCC- |
| | | | GGGUGGGUUCCGGCAG- |
| | | | GUUCUCACCCUCU- |
| | | | CUAGGCCCCAUUCUCCU- |
| | | | CUG |
| 570. | hsa-mir- | MI0000816 | UGUUUUGAGCGGGGGU- |
| | 335 | | CAAGAGCAAUAAC- |
| | | | GAAAAAUGUUUGU- |
| | | | CAUAAACCGUUUUU- |
| | | | CAUUAUUGCUCCUGAC- |
| | | | CUCCUCUCAUUUG- |
| | | | CUAUAUUCA |
| 571. | hsa-mir- | MI0003675 | UGGUACUUGGAGAGAUA- |
| | 411 | | GUAGACCGUAUAGCGUAC- |
| | | | GCUUUAUCUGUGACGUAU- |
| | | | GUAACACGGUCCA- |
| | | | CUAACCCUCAGUAU- |
| | | | CAAAUCCAUCCCCGAG |
| 572. | hsa-mir- | MI0003136 | CCCAAGUCAGGUACUC- |
| | 496 | | GAAUGGAGGUUGUCCAUG- |
| | | | GUGUGUU- |
| | | | CAUUUUAUUUAUGAUGA- |
| | | | GUAUUACAUGGCCAAU- |
| | | | CUCCUUUCGGUACU- |
| | | | CAAUUCUUCUUGGG |
| 573. | hsa-mir- | MI0000735 | AUCUCUUACACAGGCU- |
| | 29c | | GACCGAUUUCUCCUGGU- |
| | | | GUUCAGAGUCUGUUUUU- |
| | | | GUCUAGCACCAUUU- |
| | | | GAAAUCGGUUAUGAU- |
| | | | GUAGGGGGA |
| 574. | hsa-mir- | MI0003596 | CAGACUAUAUAUUUAG- |
| | 548b | | GUUGGCGCAAAAGUAAUU- |
| | | | GUGGUUUUGGC- |
| | | | CUUUAUUUUCAAUGGCAA- |
| | | | GAACCUCAGUUGCUUUU- |
| | | | GUGCCAACCUAAUACUU |
| 575. | hsa-mir- | MI0001145 | UGUUAAAUCAG- |
| | 384 | | GAAUUUUAAACAAUUC- |
| | | | CUAGACAAUAUGUAUAAU- |
| | | | GUUCAUAAGUCAUUCCUA- |
| | | | GAAAUUGUUCAUAAUGC- |
| | | | CUGUAACA |
| 576. | hsa-mir- | MI0000745 | ACUGCUAACGAAUGCUCU- |
| | 301 | | GACUUUAUUGCACUACU- |
| | | | GUACUUUACAGCUAGCA- |
| | | | GUGCAAUAGUAUUGU- |
| | | | CAAAGCAUCUGAAAGCAGG |
| 577. | hsa-mir- | MI0000289 | UGAGUUUUGAGGUUGCUU- |
| | 181a-1 | | CAGUGAACAUUCAACGCU- |
| | | | GUCGGUGAGUUUG- |
| | | | GAAUUAAAAUCAAAAC- |
| | | | CAUCGACCGUUGAUUGU |
| | | | CCCUAUGGCUAACCAU- |
| | | | CAUCUACUCCA |
| 578. | hsa-mir- | MI0000255 | GUUGUUGUAAACAUCCCC- |
| | 30d | | GACUGGAAGCUGUAAGA- |
| | | | CACAGCUAAGCUUUCAGU- |
| | | | CAGAUGUUUGCUGCUAC |
| 579. | hsa-mir- | MI0000274 | GGUCGGGCUCACCAUGA- |
| | 187 | | CACAGUGUGAGACCUC- |
| | | | GGGCUACAACACAG- |
| | | | GACCCGGGCGCUGCUCU- |
| | | | GACCCCUCGUGUCUUGU- |
| | | | GUUGCAGCCGGAGGGAC- |
| | | | GCAGGUCCGCA |
| 580. | hsa-mir- | MI0000282 | CCAGAGGACACCUCCA- |
| | 199b | | CUCCGUCUACCCAGU- |
| | | | GUUUAGACUAUCUGUU- |
| | | | CAGGACUCCCAAAUUGUA- |
| | | | CAGUAGUCUGCACAUUG- |
| | | | GUUAGGCUGGGCUGG- |
| | | | GUUAGACCCUCGG |
| 581. | hsa-mir- | MI0000471 | CGCUGGCGACGGGA- |
| | 126 | | CAUUAUUACUUUUGGUAC- |
| | | | GCGCUGUGACACUUCAAA- |
| | | | CUCGUACCGUGA- |
| | | | GUAAUAAUGCGCCGUC- |
| | | | CACGGCA |
| 582. | hsa-mir- | MI0000465 | CGGCUGGACAGC- |
| | 191 | | GGGCAACGGAAUCC- |
| | | | CAAAAGCAGCUGUUGU- |
| | | | CUCCAGAGCAUUCCAG- |
| | | | CUGCGCUUGGAUUUC- |
| | | | GUCCCCUGCUCUCCUGCCU |

Further non-limited examples of second subsequences in the form of RNA polynucleotides according to the present invention are listed in Table 5 below. It will be understood that such sequences, or a complementary strand thereof, can be operably linked to a first subsequence as defined herein elsewhere.

The sequences can also be accessed through the mammalian noncoding RNA database (RNAdb):

(http://jsm-research.imb.uq.edu.au/rnadb/Database/default.aspx)

TABLE 5

| | SEQ | | | |
|---|---|---|---|---|
| SEQ | Name | | | |
| ID | in | | Genbank | |
| NO | RNAdb | Description | accession | Species |
| 583. | LIT1110 | Homo sapiens PAR5 gene, complete | AF019618 | Homo |
| | | sequence. | | sapiens |
| 584. | LIT1227 | H. sapiens predicted non coding | X91348 | Homo |
| | | cDNA (DGCR5) | | sapiens |
| 585. | LIT1233 | Elephantidae gen. sp. H19 RNA | AF190054 | Elephantidae |
| | | gene, partial sequence | | gen. sp. |
| 586. | LIT1234 | Felis catus H19 RNA gene, partial | AF190057 | Felis catus |
| | | sequence | | |
| 587. | LIT1235 | Lynx lynx H19 RNA gene, partial | AF190056 | Lynx lynx |
| | | sequence | | |
| 588. | LIT1236 | Pongo pygmaeus H19 gene, partial | AF190058 | Pongo pygmaeus |
| | | sequence | | |
| 589. | LIT1242 | Thomomys monticola H19 RNA | AF190055 | Thomomys |
| | | gene, partial sequence | | monticola |
| 590. | LIT1245 | Homo sapiens steroid receptor RNA | XR_000132 | Homo |
| | | activator 1 (SRA1), misc RNA | | sapiens |
| 591. | LIT1246 | Homo sapiens steroid receptor RNA | AF293024 | Homo |
| | | activator isoform 1 mRNA, complete | | sapiens |
| | | cds | | |
| 592. | LIT1250 | Homo sapiens steroid receptor RNA | AF293025 | Homo |
| | | activator isoform 2 mRNA, complete | | sapiens |
| | | cds | | |
| 593. | LIT1251 | Homo sapiens steroid receptor RNA | AF293026 | Homo |
| | | activator isoform 3 mRNA, complete | | sapiens |
| | | cds | | |
| 594. | LIT1266 | Homo sapiens miR-16-1 stem-loop | | Homo |
| | | | | sapiens |
| 595. | LIT1275 | Homo sapiens DLEU1 noncoding | AF279660 | Homo |
| | | transcript (BCMS) | | sapiens |
| 596. | LIT1276 | Homo sapiens DLEU2 noncoding | NM_006021 | Homo |
| | | transcript | | sapiens |
| 597. | LIT1345 | Mus musculus makorin 1 pseudogene | AF494488 | Mus musculus |
| | | mRNA, partial sequence | | |
| 598. | LIT1545 | Homo sapiens testis-specific Testis | AF000990 | Homo |
| | | Transcript Y 1 (TTY1) mRNA, partial | | sapiens |
| | | cds | | |
| 599. | LIT1549 | Homo sapiens partial mRNA for | AJ297963 | Homo |
| | | TTY2 gene, clone TTY2L12A | | sapiens |
| 600. | LIT1550 | Homo sapiens partial mRNA for | AJ297964 | Homo |
| | | TTY2 gene, clone TTY2L2A | | sapiens |
| 601. | LIT1551 | Homo sapiens testis-specific Testis | AF000991 | Homo |
| | | Transcript Y 2 (TTY2) mRNA, partial | | sapiens |
| | | cds | | |
| 602. | LIT1552 | Homo sapiens non-coding RNA | AF103907 | Homo |
| | | DD3 sequence | | sapiens |
| 603. | LIT1553 | Homo sapiens non-coding RNA | AF103908 | Homo |
| | | DD3 gene, exons 2, 3, and 4 | | sapiens |
| 604. | LIT1554 | Homo sapiens non-coding RNA | AF103908 | Homo |
| | | DD3, transcript III | | sapiens |
| 605. | LIT1556 | Homo sapiens non-coding RNA | AF103908 | Homo |
| | | DD3, transcript (major) II | | sapiens |
| 606. | LIT1561 | Homo sapiens non-coding RNA | AF103908 | Homo |
| | | DD3, transcript I | | sapiens |
| 607. | LIT1562 | Homo sapiens PCGEM1 gene, non- | AF223389 | Homo |
| | | coding mRNA. | | sapiens |
| 608. | LIT1584 | Homo sapiens RNA for differentiation | D43770 | Homo |
| | | or sex determination (CMPD) | | sapiens |
| 609. | LIT1586 | Homo sapiens BIC noncoding | AF402776 | Homo |
| | | mRNA, complete sequence | | sapiens |
| 610. | LIT1587 | Mus musculus BIC noncoding | AY096003 | Mus musculus |
| | | mRNA, complete sequence | | |
| 611. | LIT1609 | Homo sapiens H19 gene, complete | AF087017 | Homo |
| | | sequence | | sapiens |
| 612. | LIT1610 | Homo sapiens H19 gene, complete | AF125183 | Homo |
| | | sequence | | sapiens |
| 613. | LIT1611 | Human H19 RNA gene, complete | M32053 | Homo |
| | | cds | | sapiens |
| 614. | LIT1615 | Mus musculus H19 fetal liver mRNA | NM_023123 | Mus musculus |
| | | (H19), mRNA | | |
| 615. | LIT1617 | Ovis aries H19 gene, partial sequence | AF105430 | Ovis aries |
| 616. | LIT1618 | Ovis aries H19 mRNA, partial sequence | AF105429 | Ovis aries |
| 617. | LIT1619 | Ovis aries H19 gene, complete sequence | AY091484 | Ovis aries |
| 618. | LIT1620 | Oryctolagus cuniculus H19/myoH | M97348 | Oryctolagus |
| | | mRNA sequence | | cuniculus |
| 619. | LIT1621 | Peromyscus maniculatus bairdii H19 | AF214115 | Peromyscus |
| | | mRNA, complete cds | | maniculatus |
| 620. | LIT1622 | Sus scrofa H19 gene, complete sequence | AY044827 | Sus scrofa |
| 621. | LIT1652 | Homo sapiens LIT1 transcript | AA359588 | Homo |
| | | | | sapiens |
| 622. | LIT1653 | Homo sapiens LIT1 transcript | AA155639 | Homo |
| | | | | sapiens |
| 623. | LIT1654 | Homo sapiens LIT1 transcript | AA701413 | Homo |
| | | | | sapiens |
| 624. | LIT1655 | Homo sapiens LIT1 transcript | AA331124 | Homo |
| | | | | sapiens |
| 625. | LIT1656 | Homo sapiens LIT1 transcript | AA889050 | Homo |
| | | | | sapiens |
| 626. | LIT1657 | Homo sapiens LIT1 transcript | AA693940 | Homo |
| | | | | sapiens |
| 627. | LIT1658 | Homo sapiens LIT1 transcript | H88273 | Homo |
| | | | | sapiens |
| 628. | LIT1659 | Homo sapiens LIT1 transcript | AA329719 | Homo |
| | | | | sapiens |
| 629. | LIT1660 | Homo sapiens LIT1 transcript | AA622687 | Homo |
| | | | | sapiens |
| 630. | LIT1661 | Homo sapiens LIT1 transcript | AA602136 | Homo |
| | | | | sapiens |
| 631. | LIT1673 | Mus musculus Peg8/lgf2as mRNA, | AB030734 | Mus musculus |
| | | imprinting gene | | |
| 632. | LIT1674 | Homo sapiens IPW mRNA sequence | U12897 | Homo |
| | | | | sapiens |
| 633. | LIT1702 | Homo sapiens hypoxia inducible | U85044 | Homo |
| | | factor (aHIF) antisense RNA sequence | | sapiens |
| 634. | LIT1710 | Rat neural specific BC1 RNA and ID | M16113 | Rattus |
| | | repetitive sequence | | norvegicus |
| 635. | LIT1711 | Mus musculus C57/Black6 BC1 | U01310 | Mus musculus |
| | | scRNA | | |
| 636. | LIT1712 | Mesocricetus auratus BC1 scRNA | U01309 | Mesocricetus |
| | | | | auratus |
| 637. | LIT1713 | Cavia porcellus Hartley BC1 scRNA | U01304 | Cavia porcellus |
| 638. | LIT1714 | Peromyscus maniculatus snRNA | U33851 | Peromyscus |
| | | (BC1 RNA) gene, partial sequence | | maniculatus |
| 639. | LIT1715 | Peromyscus californicus snRNA | U33850 | Peromyscus |
| | | (BC1 RNA) gene, partial sequence | | californicus |
| 640. | LIT1716 | Meriones unguiculatus snRNA (BC1 | U33852 | Meriones |
| | | RNA) gene, partial sequence | | unguiculatus |
| 641. | LIT1717 | Aotus trivirgatus BC200 alpha | AF067786 | Aotus trivirgatus |
| | | scRNA gene, complete sequence | | |
| 642. | LIT1718 | Chlorocebus aethiops BC200 alpha | AF067783 | Cercopithecus |
| | | scRNA gene, complete sequence | | aethiops |
| 643. | LIT1719 | Gorilla gorilla BC200 alpha scRNA | AF067779 | Gorilla gorilla |
| | | gene, complete sequence | | |
| 644. | LIT1721 | Human BC200 scRNA | U01305 | Homo |
| | | | | sapiens |
| 645. | LIT1724 | Hylobates lar BC200 alpha scRNA | AF067781 | Hylobates lar |
| | | gene, complete sequence. | | |
| 646. | LIT1725 | Macaca fascicularis BC200 alpha | AF067785 | Macaca fascicularis |
| | | scRNA gene, complete sequence | | |
| 647. | LIT1726 | Macaca mulatta BC200 alpha | AF067784 | Macaca mulatta |
| | | scRNA gene, complete sequence | | |
| 648. | LIT1727 | Pan paniscus BC200 alpha scRNA | AF067778 | Pan paniscus |
| | | gene, complete sequence | | |
| 649. | LIT1728 | Papio hamadryas BC200 alpha | AF067782 | Papio hamadryas |
| | | scRNA gene, complete sequence | | |
| 650. | LIT1729 | Pongo pygmaeus BC200 alpha | AF067780 | Pongo pygmaeus |
| | | scRNA gene, complete sequence | | |
| 651. | LIT1730 | Saguinus imperator BC200 alpha | AF067787 | Saguinus |
| | | scRNA gene, complete sequence | | imperator |
| 652. | LIT1731 | Saguinus oedipus BC200 alpha | AF067788 | Saguinus |
| | | scRNA gene, complete sequence | | oedipus |
| 653. | LIT1751 | Homo sapiens 1 DISC2 gene, complete | AF222981 | Homo |
| | | sequence | | sapiens |
| 654. | LIT1753 | Homo sapiens mitochondrial RNA- | AF334829 | Homo |
| | | processing endoribonuclease RNA | | sapiens |
| | | (RMRP) gene, complete sequence | | |
| 655. | LIT1757 | Homo sapiens RNase MRP RNA | AF458223 | Homo |
| | | component, complete sequence | | sapiens |
| 656. | LIT1758 | H. sapiens MRP RNA gene encoding | X51867 | Homo |
| | | the RNA component of RNase MRP | | sapiens |
| | | (RMRP) | | |
| 657. | LIT1759 | B. taurus RNase MRP (RMRP) gene, | Z25280 | Bos taurus |
| | | complete CDS | | |
| 658. | LIT1765 | Homo sapiens UBE3A antisense | AF400502 | Homo |
| | | RNA from clone R19540 SNURF- | | sapiens |
| | | SNRPN mRNA | | |
| 659. | LIT1766 | Mus musculus SJL/j viral integration | U09772 | Mus musculus |
| | | site (His-1) RNA transcript, exons 1, | | |
| | | 2b and 3, alternatively spliced | | |
| 660. | LIT1767 | Mus musculus SJL/j viral integration | U10269 | Mus musculus |
| | | site (His-1) RNA transcript, exons 1, | | |
| | | 2a and 3, alternatively spliced | | |
| 661. | LIT1768 | Mus musculus His-1 gene, exons 1, | U56439 | Mus musculus |
| | | 2a, 2b and 3 | | |
| 662. | LIT1836 | Mus musculus Tmevpg1, mRNA | AI592225 | Mus musculus |
| | | sequence | | |
| 663. | LIT1870 | Homo sapiens mRNA for B-cell | AJ412063 | Homo |
| | | neoplasia associated transcript, | | sapiens |
| | | (BCMS gene), splice variant AO, | | |
| | | non coding transcript | | |
| 664. | LIT1871 | Homo sapiens mRNA for B-cell | AJ412062 | Homo |
| | | neoplasia associated transcript, | | sapiens |
| | | (BCMS gene), splice variant AN, | | |
| | | non coding transcript | | |
| 665. | LIT1872 | Homo sapiens mRNA for B-cell | AJ412061 | Homo |
| | | neoplasia associated transcript, | | sapiens |
| | | (BCMS gene), splice variant AM, | | |
| | | non coding transcript | | |
| 666. | LIT1873 | Homo sapiens mRNA for B-cell | AJ412060 | Homo |
| | | neoplasia associated transcript, | | sapiens |
| | | (BCMS gene), splice variant AL, non | | |
| | | coding transcript | | |
| 667. | LIT1874 | Homo sapiens mRNA for B-cell | AJ412059 | Homo |
| | | neoplasia associated transcript, | | sapiens |
| | | (BCMS gene), splice variant AK, | | |
| | | non coding transcript | | |
| 668. | LIT1875 | Homo sapiens mRNA for B-cell | AJ412058 | Homo |
| | | neoplasia associated transcript, | | sapiens |
| | | (BCMS gene), splice variant AJ, non | | |
| | | coding transcript | | |
| 669. | LIT1876 | Homo sapiens mRNA for B-cell | AJ412057 | Homo |
| | | neoplasia associated transcript, | | sapiens |
| | | (BCMS gene), splice variant AI, non | | |
| | | coding transcript | | |
| 670. | LIT1884 | Homo sapiens mRNA for B-cell | AJ412056 | Homo |
| | | neoplasia associated transcript, | | sapiens |
| | | (BCMS gene), splice variant AH, | | |
| | | non coding transcript | | |
| 671. | LIT1885 | Homo sapiens mRNA for B-cell | AJ412055 | Homo |
| | | neoplasia associated transcript, | | sapiens |
| | | (BCMS gene), splice variant AG, | | |
| | | non coding transcript | | |
| 672. | LIT1886 | Homo sapiens mRNA for B-cell | AJ412054 | Homo |
| | | neoplasia associated transcript, | | sapiens |
| | | (BCMS gene), splice variant AF, non | | |
| | | coding transcript | | |
| 673. | LIT1887 | Homo sapiens mRNA for B-cell | AJ412053 | Homo |
| | | neoplasia associated transcript, | | sapiens |
| | | (BCMS gene), splice variant AE, | | |
| | | non coding transcript | | |
| 674. | LIT1888 | Homo sapiens mRNA for B-cell | AJ412052 | Homo |
| | | neoplasia associated transcript, | | sapiens |
| | | (BCMS gene), splice variant AD, | | |
| | | non coding transcript | | |
| 675. | LIT1889 | Homo sapiens mRNA for B-cell | AJ412051 | Homo |
| | | neoplasia associated transcript, | | sapiens |
| | | (BCMS gene), splice variant AC, | | |
| | | non coding transcript | | |
| 676. | LIT1890 | Homo sapiens mRNA for B-cell | AJ412050 | Homo |
| | | neoplasia associated transcript, | | sapiens |
| | | (BCMS gene), splice variant AB, | | |
| | | non coding transcript | | |
| 677. | LIT1891 | Homo sapiens mRNA for B-cell | AJ412049 | Homo |
| | | neoplasia associated transcript, | | sapiens |
| | | (BCMS gene), splice variant AA, | | |
| | | non coding transcript | | |
| 678. | LIT1892 | Homo sapiens mRNA for B-cell | AJ412048 | Homo |
| | | neoplasia associated transcript, | | sapiens |
| | | (BCMS gene), splice variant Z, non | | |
| | | coding transcript | | |
| 679. | LIT1893 | Homo sapiens mRNA for B-cell | AJ412047 | Homo |
| | | neoplasia associated transcript, | | sapiens |
| | | (BCMS gene), splice variant Y, non | | |
| | | coding transcript | | |
| 680. | LIT1894 | Homo sapiens mRNA for B-cell | AJ412046 | Homo |
| | | neoplasia associated transcript, | | sapiens |
| | | (BCMS gene), splice variant X, non | | |
| | | coding transcript | | |
| 681. | LIT1897 | Homo sapiens mRNA for B-cell | AJ412045 | Homo |
| | | neoplasia associated transcript, | | sapiens |
| | | (BCMS gene), splice variant W, non | | |
| | | coding transcript | | |
| 682. | LIT1898 | Homo sapiens mRNA for B-cell | AJ412044 | Homo |
| | | neoplasia associated transcript, | | sapiens |
| | | (BCMS gene), splice variant V, non | | |
| | | coding transcript | | |
| 683. | LIT1899 | Homo sapiens clone IMAGE: | AF400045 | Homo |
| | | 1409652 ST7OT2 mRNA, non- | | sapiens |
| | | coding transcript | | |
| 684. | LIT1900 | Homo sapiens clone IMAGE: | AF400044 | Homo |
| | | 1628386 ST7OT3 mRNA, non- | | sapiens |
| | | coding transcript | | |
| 685. | LIT1901 | Homo sapiens ST7 overlapping | NM_021908 | Homo |
| | | transcript 3 (non-coding RNA) taken | | sapiens |
| | | from suppression of tumorigenicity 7 | | |
| | | (ST7), transcript variant b, mRNA | | |
| 686. | LIT1902 | Homo sapiens mRNA for B-cell | AJ412043 | Homo |
| | | neoplasia associated transcript, | | sapiens |
| | | (BCMS gene), splice variant U, non | | |
| | | coding transcript | | |
| 687. | LIT1903 | Homo sapiens mRNA for B-cell | AJ412042 | Homo |
| | | neoplasia associated transcript, | | sapiens |
| | | (BCMS gene), splice variant T, non | | |
| | | coding transcript | | |
| 688. | LIT1904 | Homo sapiens mRNA for B-cell | AJ412041 | Homo |
| | | neoplasia associated transcript, | | sapiens |
| | | (BCMS gene), splice variant S, non | | |
| | | coding transcript | | |
| 689. | LIT1905 | Homo sapiens mRNA for B-cell | AJ412040 | Homo |
| | | neoplasia associated transcript, | | sapiens |
| | | (BCMS gene), splice variant R, non | | |
| | | coding transcript | | |
| 690. | LIT1906 | Homo sapiens mRNA for B-cell | AJ412039 | Homo |
| | | neoplasia associated transcript, | | sapiens |
| | | (BCMS gene), splice variant Q, non | | |
| | | coding transcript | | |
| 691. | LIT1907 | Homo sapiens mRNA for B-cell | AJ412038 | Homo |
| | | neoplasia associated transcript, | | sapiens |
| | | (BCMS gene), splice variant P, non | | |
| | | coding transcript | | |
| 692. | LIT1908 | Homo sapiens mRNA for B-cell | AJ412037 | Homo |
| | | neoplasia associated transcript, | | sapiens |
| | | (BCMS gene), splice variant O, non | | |
| | | coding transcript | | |
| 693. | LIT1909 | Homo sapiens mRNA for B-cell | AJ412036 | Homo |
| | | neoplasia associated transcript, | | sapiens |
| | | (BCMS gene), splice variant N, non | | |
| | | coding transcript | | |
| 694. | LIT1910 | Homo sapiens mRNA for B-cell | AJ412035 | Homo |
| | | neoplasia associated transcript, | | sapiens |
| | | (BCMS gene), splice variant M, non | | |
| | | coding transcript | | |
| 695. | LIT1911 | Homo sapiens mRNA for B-cell | AJ412034 | Homo |
| | | neoplasia associated transcript, | | sapiens |
| | | (BCMS gene), splice variant L, non | | |
| | | coding transcript | | |
| 696. | LIT1912 | Homo sapiens mRNA for B-cell | AJ412033 | Homo |
| | | neoplasia associated transcript, | | sapiens |
| | | (BCMS gene), splice variant K, non | | |
| | | coding transcript | | |
| 697. | LIT1916 | Homo sapiens mRNA for B-cell | AJ412032 | Homo |
| | | neoplasia associated transcript, | | sapiens |
| | | (BCMS gene), splice variant J, non | | |
| | | coding transcript | | |
| 698. | LIT1917 | Homo sapiens mRNA for B-cell | AJ412031 | Homo |
| | | neoplasia associated transcript, | | sapiens |
| | | (BCMS gene), splice variant I, non | | |
| | | coding transcript | | |
| 699. | LIT1921 | Homo sapiens miR-15a mature | | Homo |
| | | | | sapiens |
| 700. | LIT1922 | Homo sapiens mRNA for B-cell | AJ412030 | Homo |
| | | neoplasia associated transcript, | | sapiens |
| | | (BCMS gene), splice variant H, non | | |
| | | coding transcript | | |
| 701. | LIT1923 | Homo sapiens mRNA for B-cell | AJ412029 | Homo |
| | | neoplasia associated transcript, | | sapiens |
| | | (BCMS gene), splice variant G, non | | |
| | | coding transcript | | |
| 702. | LIT1924 | Homo sapiens mRNA for B-cell | AJ412028 | Homo |
| | | neoplasia associated transcript, | | sapiens |
| | | (BCMS gene), splice variant F, non | | |
| | | coding transcript | | |
| 703. | LIT1925 | Homo sapiens mRNA for B-cell | AJ412027 | Homo |
| | | neoplasia associated transcript, | | sapiens |
| | | (BCMS gene), splice variant E, non | | |
| | | coding transcript | | |
| 704. | LIT1926 | Homo sapiens mRNA for B-cell | AJ412026 | Homo |
| | | neoplasia associated transcript, | | sapiens |
| | | (BCMS gene), splice variant D, non | | |
| | | coding transcript | | |
| 705. | LIT1927 | Homo sapiens mRNA for B-cell | AJ412025 | Homo |
| | | neoplasia associated transcript, | | sapiens |
| | | (BCMS gene), splice variant C, non | | |
| | | coding transcript | | |
| 706. | LIT1928 | Homo sapiens mRNA for B-cell | AJ412024 | Homo |
| | | neoplasia associated transcript, | | sapiens |
| | | (BCMS gene), splice variant B, non | | |
| | | coding transcript | | |
| 707. | LIT1929 | Homo sapiens mRNA for B-cell | AJ412023 | Homo |
| | | neoplasia associated transcript, | | sapiens |
| | | (BCMS gene), splice variant A, non | | |
| | | coding transcript | | |
| 708. | LIT1930 | Homo sapiens partial BCMS gene | AJ412022 | Homo |
| | | for B-cell neoplasia associated transcript, | | sapiens |
| | | exon 47 | | |
| 709. | LIT1934 | Homo sapiens partial BCMS gene | AJ412021 | Homo |
| | | for B-cell neoplasia associated transcript, | | sapiens |
| | | exon 46 | | |
| 710. | LIT1935 | Homo sapiens partial BCMS gene | AJ412020 | Homo |
| | | for B-cell neoplasia associated transcript, | | sapiens |
| | | exon 45 | | |
| 711. | LIT1936 | Homo sapiens partial BCMS gene | AJ412019 | Homo |
| | | for B-cell neoplasia associated transcript, | | sapiens |
| | | exon 44 | | |
| 712. | LIT1937 | Homo sapiens partial BCMS gene | AJ412018 | Homo |
| | | for B-cell neoplasia associated transcript, | | sapiens |
| | | exon 43 | | |
| 713. | LIT1938 | Homo sapiens clone IMAGE: 782833 | AF400043 | Homo |
| | | ST7OT2 mRNA, non-coding transcript | | sapiens |
| 714. | LIT1942 | Homo sapiens partial BCMS gene | AJ412017 | Homo |
| | | for B-cell neoplasia associated transcript, | | sapiens |
| | | exon 42 | | |
| 715. | LIT1943 | Homo sapiens partial BCMS gene | AJ412016 | Homo |
| | | for B-cell neoplasia associated transcript, | | sapiens |
| | | exon 41 | | |
| 716. | LIT1944 | Homo sapiens partial BCMS gene | AJ412015 | Homo |
| | | for B-cell neoplasia associated transcript, | | sapiens |
| | | exon 40 | | |
| 717. | LIT1945 | Homo sapiens partial BCMS gene | AJ412014 | Homo |
| | | for B-cell neoplasia associated transcript, | | sapiens |
| | | exon 39 | | |
| 718. | LIT1946 | Homo sapiens partial BCMS gene | AJ412013 | Homo |
| | | for B-cell neoplasia associated transcript, | | sapiens |
| | | exon 38 | | |
| 719. | LIT1947 | Homo sapiens partial BCMS gene | AJ412012 | Homo |
| | | for B-cell neoplasia associated transcript, | | sapiens |
| | | exon 37 | | |
| 720. | LIT1948 | Homo sapiens partial BCMS gene | AJ412011 | Homo |
| | | for B-cell neoplasia associated transcript, | | sapiens |
| | | exon 36a | | |
| 721. | LIT1949 | Homo sapiens partial BCMS gene | AJ412010 | Homo |
| | | for B-cell neoplasia associated transcript, | | sapiens |
| | | exon 36 | | |
| 722. | LIT1950 | Homo sapiens partial BCMS gene | AJ412009 | Homo |
| | | for B-cell neoplasia associated transcript, | | sapiens |
| | | exon 35 | | |
| 723. | LIT1951 | Homo sapiens partial BCMS gene | AJ412008 | Homo |
| | | for B-cell neoplasia associated transcript, | | sapiens |
| | | exon 34 | | |
| 724. | LIT1955 | Homo sapiens partial BCMS gene | AJ412007 | Homo |
| | | for B-cell neoplasia associated transcript, | | sapiens |
| | | exon 33 | | |
| 725. | LIT1956 | Homo sapiens partial BCMS gene | AJ412006 | Homo |
| | | for B-cell neoplasia associated transcript, | | sapiens |
| | | exon 32 | | |
| 726. | LIT1957 | Homo sapiens partial BCMS gene | AJ412005 | Homo |
| | | for B-cell neoplasia associated transcript, | | sapiens |
| | | exon 31 | | |
| 727. | LIT1958 | Homo sapiens partial BCMS gene | AJ412004 | Homo |
| | | for B-cell neoplasia associated transcript, | | sapiens |
| | | exon 30 | | |
| 728. | LIT1962 | Homo sapiens partial BCMS gene | AJ412003 | Homo |
| | | for B-cell neoplasia associated transcript, | | sapiens |
| | | exon 29 | | |
| 729. | LIT1963 | Homo sapiens partial BCMS gene | AJ412002 | Homo |
| | | for B-cell neoplasia associated transcript, | | sapiens |
| | | exon 28 | | |
| 730. | LIT1964 | Homo sapiens partial BCMS gene | AJ412001 | Homo |
| | | for B-cell neoplasia associated transcript, | | sapiens |
| | | exon 27 | | |
| 731. | LIT1965 | Homo sapiens partial BCMS gene | AJ412000 | Homo |
| | | for B-cell neoplasia associated transcript, | | sapiens |
| | | exon 26 | | |
| 732. | LIT1966 | Homo sapiens partial BCMS gene | AJ411999 | Homo |
| | | for B-cell neoplasia associated transcript, | | sapiens |
| | | exon 25 | | |
| 733. | LIT1967 | Homo sapiens partial BCMS gene | AJ411998 | Homo |
| | | for B-cell neoplasia associated transcript, | | sapiens |
| | | exon 24 | | |
| 734. | LIT1968 | Homo sapiens partial BCMS gene | AJ411997 | Homo |
| | | for B-cell neoplasia associated transcript, | | sapiens |
| | | exon 23 | | |
| 735. | LIT1969 | Homo sapiens partial BCMS gene | AJ411996 | Homo |
| | | for B-cell neoplasia associated transcript, | | sapiens |
| | | exon 22 | | |
| 736. | LIT1970 | Homo sapiens partial BCMS gene | AJ411995 | Homo |
| | | for B-cell neoplasia associated transcript, | | sapiens |
| | | exon 21 | | |
| 737. | LIT1971 | Homo sapiens partial BCMS gene | AJ411994 | Homo |
| | | for B-cell neoplasia associated transcript, | | sapiens |
| | | exon 20 | | |
| 738. | LIT1972 | Homo sapiens partial BCMS gene | AJ411993 | Homo |
| | | for B-cell neoplasia associated transcript, | | sapiens |
| | | exon 19 | | |
| 739. | LIT1973 | Homo sapiens partial BCMS gene | AJ411992 | Homo |
| | | for B-cell neoplasia associated transcript, | | sapiens |
| | | exon 18 | | |
| 740. | LIT1974 | Homo sapiens partial BCMS gene | AJ411991 | Homo |
| | | for B-cell neoplasia associated transcript, | | sapiens |
| | | exon 17 | | |
| 741. | LIT1975 | Homo sapiens partial BCMS gene | AJ411990 | Homo |
| | | for B-cell neoplasia associated transcript, | | sapiens |
| | | exon 16 | | |
| 742. | LIT1976 | Homo sapiens partial BCMS gene | AJ411989 | Homo |
| | | for B-cell neoplasia associated transcript, | | sapiens |
| | | exon 15 | | |
| 743. | LIT1977 | Homo sapiens partial BCMS gene | AJ411988 | Homo |
| | | for B-cell neoplasia associated transcript, | | sapiens |
| | | exon 14 | | |
| 744. | LIT1978 | Homo sapiens ST7 overlapping | BM413623 | Homo |
| | | transcript 4, mRNA sequence | | sapiens |
| 745. | LIT1979 | Homo sapiens ST7 overlapping | BM413624 | Homo |
| | | transcript 4, mRNA sequence | | sapiens |
| 746. | LIT1980 | Homo sapiens ST7 overlapping | BM413625 | Homo |
| | | transcript 4, mRNA sequence | | sapiens |
| 747. | LIT1981 | Homo sapiens partial BCMS gene | AJ411987 | Homo |
| | | for B-cell neoplasia associated transcript, | | sapiens |
| | | exon 13 | | |
| 748. | LIT1982 | Homo sapiens partial BCMS gene | AJ411986 | Homo |
| | | for B-cell neoplasia associated transcript, | | sapiens |
| | | exon 12 | | |
| 749. | LIT1983 | Homo sapiens partial BCMS gene | AJ411985 | Homo |
| | | for B-cell neoplasia associated transcript, | | sapiens |
| | | exon 11a | | |
| 750. | LIT1984 | Homo sapiens partial BCMS gene | AJ411984 | Homo |
| | | for B-cell neoplasia associated transcript, | | sapiens |
| | | exon 11 | | |
| 751. | LIT1985 | Homo sapiens partial BCMS gene | AJ411983 | Homo |
| | | for B-cell neoplasia associated transcript, | | sapiens |
| | | exon 10 | | |
| 752. | LIT1989 | Homo sapiens partial BCMS gene | AJ411982 | Homo |
| | | for B-cell neoplasia associated transcript, | | sapiens |
| | | exon 9 | | |
| 753. | LIT1994 | Homo sapiens partial BCMS gene | AJ411981 | Homo |
| | | for B-cell neoplasia associated transcript, | | sapiens |
| | | exon 8 | | |
| 754. | LIT1995 | Homo sapiens partial BCMS gene | AJ411980 | Homo |
| | | for B-cell neoplasia associated transcript, | | sapiens |
| | | exon 7 | | |
| 755. | LIT1996 | Homo sapiens partial BCMS gene | AJ411979 | Homo |
| | | for B-cell neoplasia associated transcript, | | sapiens |
| | | exon 6 | | |
| 756. | LIT1997 | Homo sapiens partial BCMS gene | AJ411978 | Homo |
| | | for B-cell neoplasia associated transcript, | | sapiens |
| | | exon 5 | | |
| 757. | LIT1998 | Homo sapiens partial BCMS gene | AJ411977 | Homo |
| | | for B-cell neoplasia associated transcript, | | sapiens |
| | | exon 4a | | |
| 758. | LIT1999 | Homo sapiens clone IMAGE: | AF400040 | Homo |
| | | 1645555 ST7OT2 mRNA, non- | | sapiens |
| | | coding transcript | | |
| 759. | LIT2000 | Homo sapiens clone IMAGE: | AF400041 | Homo |
| | | 1642027 ST7OT2 mRNA, non- | | sapiens |
| | | coding transcript | | |
| 760. | LIT2001 | Homo sapiens clone IMAGE: | AF400042 | Homo |
| | | 2097781 ST7OT2 mRNA, non- | | sapiens |
| | | coding transcript | | |
| 761. | LIT2002 | Homo sapiens partial BCMS gene | AJ411976 | Homo |
| | | for B-cell neoplasia associated transcript, | | sapiens |
| | | exon 4 | | |
| 762. | LIT2003 | Homo sapiens partial BCMS gene | AJ411975 | Homo |
| | | for B-cell neoplasia associated transcript, | | sapiens |
| | | exon 3 | | |
| 763. | LIT2004 | Homo sapiens partial BCMS gene | AJ411974 | Homo |
| | | for B-cell neoplasia associated transcript, | | sapiens |
| | | exon 2 | | |
| 764. | LIT2005 | Homo sapiens partial BCMS gene | AJ411973 | Homo |
| | | for B-cell neoplasia associated transcript, | | sapiens |
| | | exon 1 | | |
| 765. | LIT2006 | Homo sapiens ST7OT1 mRNA, non- | AF400039 | Homo |
| | | coding transcript | | sapiens |
| 766. | LIT2007 | Homo sapiens ST7 overlapping | NM_018412 | Homo |
| | | transcript 3 (non-coding RNA) taken | | sapiens |
| | | from suppression of tumorigenicity 7 | | |
| | | (ST7), transcript variant a, mRNA | | |
| 767. | LIT2008 | Homo sapiens ST7 overlapping | BM413626 | Homo |
| | | transcript 4, mRNA sequence | | sapiens |
| 768. | LIT2012 | Homo sapiens metastasis associated | BK001418 | Homo |
| | | in lung adenocarcinoma transcript, | | sapiens |
| | | 1 long isoform, transcribed | | |
| | | non-coding RNA, complete sequence. | | |
| 769. | LIT2013 | Homo sapiens metastasis associated | BK001411 | Homo |
| | | in lung adenocarcinoma transcript, | | sapiens |
| | | 1 short isoform, transcribed | | |
| | | non-coding RNA, complete sequence | | |
| 770. | LIT2014 | Human gene hY1 encoding a cytoplasmic | V00584 | Homo |
| | | Ro RNA. | | sapiens |
| 771. | LIT2019 | Human Ro RNA (scRNA) hY3 from | K01563 | Homo |
| | | small cytoplasmic ribonucleoprotein | | sapiens |
| | | particles. | | |
| 772. | LIT2021 | Human hy4 Ro RNA (associated | X57566 | Homo |
| | | with erythrocyte Ro RNPs). | | sapiens |
| 773. | LIT2023 | Y RNA {clone Y5-125, small RNA | S76546 | Homo |
| | | known as Ro RNA} | | sapiens |
| 774. | LIT2024 | Human Ro RNA (scRNA) hY5 from | K01564 | Homo |
| | | small cytoplasmic ribonucleoprotein | | sapiens |
| | | particles. | | |
| 775. | LIT2055 | Homo sapiens PAR1 gene, complete | AF019616 | Homo |
| | | sequence. | | sapiens |
| 776. | LIT2116 | Homo sapiens SZ-1 mRNA | AF525782 | Homo |
| | | (PSZA11q14), complete sequence | | sapiens |
| 777. | LIT2117 | Homo sapiens telomerase RNA | NR_001566 | Homo |
| | | component (TERC) on chromosome 3 | | sapiens |
| 778. | LIT2121 | Homo sapiens noncoding RNA | CB338058 | Homo |
| | | GA3824 implicated in autism | | sapiens |
| 779. | LIT3143 | Homo sapiens miR-16 mature | AJ421734 | Homo |
| | | | | sapiens |
| 780. | LIT3317 | Homo sapiens AAA1 variant IB | AY312365 | Homo |
| | | mRNA, complete sequence; alternatively | | sapiens |
| | | spliced | | |
| 781. | LIT3319 | Homo sapiens non-coding RNA in | XR_000219 | Homo |
| | | rhabdomyosarcoma (RMS) | | sapiens |
| | | (NCRMS), misc RNA | | |
| 782. | LIT3320 | Homo sapiens SCA8 mRNA, repeat | AF126749 | Homo |
| | | region. | | sapiens |
| 783. | LIT3321 | Homo sapiens maternally expressed | AY314975 | Homo |
| | | gene 3 (MEG3) mRNA, complete | | sapiens |
| | | sequence. | | |
| 784. | LIT3323 | Mus musculus RNA component of | NR_001460 | Mus musculus |
| | | mitochondrial RNAase P (Rmrp) on | | |
| | | chromosome 4. | | |
| 785. | LIT3326 | Homo sapiens AAA1 variant II | AY312366 | Homo |
| | | mRNA, complete cds; alternatively | | sapiens |
| | | spliced | | |
| 786. | LIT3327 | Homo sapiens AAA1 variant III | AY312367 | Homo |
| | | mRNA, complete cds; alternatively | | sapiens |
| | | spliced | | |
| 787. | LIT3328 | Homo sapiens AAA1 variant IV | AY312368 | Homo |
| | | mRNA, complete cds; alternatively | | sapiens |
| | | spliced | | |
| 788. | LIT3331 | Homo sapiens AAA1 variant IX | AY312373 | Homo |
| | | mRNA, complete cds; alternatively | | sapiens |
| | | spliced | | |
| 789. | LIT3332 | Homo sapiens AAA1 variant V | AY312369 | Homo |
| | | mRNA, complete cds; alternatively | | sapiens |
| | | spliced | | |
| 790. | LIT3333 | Homo sapiens AAA1 variant VI | AY312370 | Homo |
| | | mRNA, complete cds; alternatively | | sapiens |
| | | spliced | | |
| 791. | LIT3334 | Homo sapiens AAA1 variant VII | AY312371 | Homo |
| | | mRNA, complete cds; alternatively | | sapiens |
| | | spliced | | |
| 792. | LIT3335 | Homo sapiens AAA1 variant VIII | AY312372 | Homo |
| | | mRNA, complete cds; alternatively | | sapiens |
| | | spliced | | |

Further non-limited examples of second subsequences in the form of bacterial RNA polynucleotides according to the present invention are listed in Table 6 below. It will be understood that such sequences, or a complementary strand thereof, can be operably linked to a first subsequence as defined herein elsewhere.

TABLE 6

| SEQ ID | | | | |
|---|---|---|---|---|
| NO | ID | Function | Sequence | Species |
| 793 | dsrA | translational | ACAUCAGAUUUCCUGGUGUA | Salmonella typhe [symbol] |
| | | regulator | ACGAAUUUU- | |
| | | | CAAGUGCUUCUUGCAUAAG- | |
| | | | CAAGUUUGAUCCCGACCCGU | |
| | | | AGGGCCGGGAUUUU | |
| 794 | | | AACACAUCAGAUUUCCUG- | Escherichia coli |
| | | | GUGUAACGAAUUUUUUAAGUGC | |
| | | | UUCUUGCUUAAGCAAGUUUC | |
| | | | AUCCCGACCCCCU- | |
| | | | CAGGGUCGGGAUU | |
| 795 | | | CACAUCAGAUUUCCUGGU- | Salmonella enter |
| | | | GUAACGAAUUUUCAAGUGCUU- | |
| | | | CUUGCAUAAGCAAGUUUGAUC | |
| | | | CCGACCCGUAGGGCCGGGAUU | |
| 796 | 6S RNA | transcriptional | UCCGCUCCCUGGUGUGUUGGC- | Pseudomonas aer [symbol] |
| | | regulator | CAGUCGGUGAUGUCCCU- | |
| | | | GAGCCGAUAACUGCAACAACGG | |
| | | | AGGUUGCCAGUUGGACCGGU- | |
| | | | GUGCAUGUCCGCACGAC- | |
| | | | GGAAAGCCUUAAGGUCUACUG- | |
| | | | CA | |
| | | | ACCGCCACCUUGAACUUUC- | |
| | | | GGGUUCAAGGGCUAACCCGA- | |
| | | | CAGCGGCACGACCGGGGAGCU | |
| | | | AUUUCUCUGAGAUGUUC- | Escherichia coli |
| | | | GCAAGCGGGCCAGUCCCCU- | |
| | | | GAGCCGAUAUUUCAUACCA- | |
| | | | CAAGA | |
| | | | AUGUGGCGCUCCGCGGUUG- | |
| | | | GUGAGCAUGCUCGGUCCGUCC- | |
| | | | GAGAAGCCUUAAAACUGCGA | |
| | | | CGACACAUUCACCUUGAAC- | |
| | | | CAAGGGUUCAAGGGUUACAGC- | |
| | | | CUGCGGCGGCAUCUCGGAGA | |
| | | | UUC | |
| 797 | rprA | transcriptional | ACGGUUAUAAAUCAACAUAUU- | Escherichia coli |
| | | regulator | GAUUUAUAAGCAUG- | |
| | | | GAAAUCCCCUGAGUGAAA- | |
| | | | CAACGAA | |
| | | | UUGCUGUGUGUAGUCUUUGCC- | |
| | | | CAUCUCCCACGAUGGG- | |
| | | | CUUUUUUUU | |
| | | | CGGUUAUAAAUCAACACAUU- | Salmonella typheri [symbol] |
| | | | GAUUUAUAAGCAUG- | |
| | | | GAAAUCCCCUGAGUGAAA- | |
| | | | CAACGAAU | |
| | | | UGCUGUGUGUAGUCUUUGCCC- | |
| | | | GUCUCCUACGAUGGG- | |
| | | | CUUUUUUUUUA | |
| 798 | micF | post- | UAAAAUCAAUAACUUAUU- | Escherichia coli |
| | | transcriptional | CUUAAGUAUUUGACAGCACU- | |
| | | regulator of | GAAUGUCAAAACAAAACCUUCA | |
| | | ompF expression | CUCGCAACUAGAAUAACUCCC- | |
| | | | GCUAUCAUCAUUAA- | |
| | | | CUUUAUUUAUUACCGUCAUU- | |
| | | | CAUUU | |
| | | | CUGAAUGUCUGUUUACCC- | |
| | | | CUAUUUCAACCGGAUGCCUC- | |
| | | | GCAUUCGGUUUUUUUU | |
| | | | GCUAUCAUCAUUAA- | Salmonella typhe [symbol] |
| | | | CUUUAUUUAUUACCGUCAUU- | |
| | | | CACUUCUGAAUGUCU- | |
| | | | GUUUACCCCUA | |
| | | | UUUCAACCGGAUGCUUC- | |
| | | | GCAUUCGGUUUUUUUU | |
| | | | GCUAUCAUCAUUAA- | Klebsiella pneum [symbol] |
| | | | CUUUAUUUAUUACCGUCAUU- | |
| | | | CAGUUCUGAAUGUCU- | |
| | | | GUUUACCCCUA | |
| | | | UUUCGACCGGAUGCUUC- | |
| | | | GCAUCCGGUUUUUUUU | |
| | | | AAAAUCAUGUAGUUAUACAAAU- | Serratia marcesc [symbol] |
| | | | CUUUAAGAAAAAAAAGCCAAC- | |
| | | | CAUACAAUUGUACUGGA | |
| | | | CAAUAAGCACAUUGUGC- | |
| | | | CAAAACGCCGCCUGCAC- | |
| | | | GCAGCCGCUAUAAUCACCUC- | |
| | | | GCUAUC | |
| | | | AUCAUUAUUUUCAUUAUUAC- | |
| | | | CUUCAUUAUCCGAA- | |
| | | | GAUAAUUUCUGCAUAC- | |
| | | | CUUUAACCGG | |
| | | | CUUCUGGCCGGUUUUUUAU | |
| | | | ACCAGUCGGCAAGUCCAUU- | Salmonella enter [symbol] |
| | | | CUCCGCAAAAAUACA- | |
| | | | GAAUAAUCCAACACGAAUAU- | |
| | | | GAUACU | |
| | | | AAAACUUUUAAGAUGUUA- | |
| | | | CAGUUAUCUAUAUAGAUGUUU- | |
| | | | CAAAAUAUGAAUUUUACGGAA | |
| | | | CUUUUUUAAAGCAAAAAU- | |
| | | | CAAGUAAAAAUAAGCACAAAUA- | |
| | | | GACAAAAUAUAUUCACGAAA | |
| | | | CUUUUAAAAU- | |
| | | | CAACGGGUUAAAUUGAU- | |
| | | | GAAAUUCAUAGCACUGAAU- | |
| | | | GAUAAAACAGAAUC | |
| | | | UUCAUUCG- | |
| | | | CAACUAAAAUAGUGACCGCUAU | |
| | | | CAUCAUUAACUUUAUUUAUUAC- | |
| | | | CGUCAUUC | |
| | | | ACUUCUGAAUGU- | |
| | | | CUGUUUACCCCUAUUU- | |
| | | | CAACCGGAUGCUUCGCAUUCG- | |
| | | | GUUUUUUU | |
| 799 | rtT | scRNA with | CAAAAGUCCCUGAACUUCC- | Escherichia coli |
| | | unknown function | CAACGAAUCC- | |
| | | | GCAAUUAAAUAUUCUGCC- | |
| | | | CAUGCGGGGAAGG | |
| | | | AUGAGAAGCUUCGACCAAG- | |
| | | | GUUCGACUCGAGCGCCAGCGA- | |
| | | | GAGAGCGUUGCCGCAGGCAA | |
| | | | CGACCCGAAGGGCGAAGC- | |
| | | | GCGCAGCGCUGAGUAAUC- | |
| | | | CUUCCCCCACCACCA | |
| 800 | ryhB | translational | GCGAUCAGGAAGACCCUC- | Escherichia coli |
| | | repressor in | GCGGAGAACCUGAAAGCACGA- | |
| | | iron utilization | CAUUGCUCACAUUGCUUCCAG | |
| | | pathway | UAUUACUUAGCCAGCCGGGUG- | |
| | | | CUGGCUUUU | |
| 801 | csrB | protein function | GAGUCA- | Escherichia coli |
| | | regulator | GACAACGAAGUGAACAUCAG- | |
| | | | GAUGAUGACACUUCUGCAG- | |
| | | | GACACACCAGGAUGG | |
| | | | UGUUUCAGGGAAAGGCUUCUG- | |
| | | | GAUGAAGCGAAGAGGAUGACG- | |
| | | | CAGGACGCGUUAAAGGAC | |
| | | | ACCUCCAGGAUGGAGAAUGA- | |
| | | | GAACCGGUCAGGAUGAUUCG- | |
| | | | GUGGGUCAGGAAGGCCAGGG | |
| | | | ACACUUCAGGAUGAAGUAUCA- | |
| | | | CAUCGGGGUGGUGUGAGCAG- | |
| | | | GAAGCAAUAGUUCAGGAUG | |
| | | | AACGAUUGGCCGCAAGGCCA- | |
| | | | GAGGAAAAGUUGUCAAGGAU- | |
| | | | GAGCAGGGAGCAACAAAAGU | |
| | | | AGCUGGAAUGCUG- | |
| | | | CGAAACGAACCGGGAGCGCUGU | |
| | | | GAAUACAGUG- | |
| | | | CUCCCUUUUUUUAUU | |
| | | | GUCGACAGGGAGUCGUA- | Salmonella typhe [symbol] |
| | | | CAACGAAGCGAACGUCAGGAU- | |
| | | | GAUGACGCUUCAGCAGGACACG | |
| | | | CCAGGAUGGUGUUACAAG- | |
| | | | GAAAGGCUUCAGGAUGAAG- | |
| | | | CAAAGUGGAAAGCGCAG- | |
| | | | GAUGCG | |
| | | | UUAAAGGACACCUCCAGGACG- | |
| | | | GAGAACGAGAGCCGAUCAG- | |
| | | | GAUGUUCGGCGGGUCUGGAU | |
| | | | GACCAGGGACGCUUCAGGAA- | |
| | | | GAAGCUAUCACAUCGGGCGAU- | |
| | | | GUGCGCAGGAUGCAAACGU | |
| | | | UCAGGAUGAACAGGCCGUAAG- | |
| | | | GUCACAGGAAAAGUUGUCACG- | |
| | | | GAUGAGCAGGGAGCACGA | |
| | | | AAAGUAGCUGGAAUGCUG- | |
| | | | CGAAACGAACCGGGAGCA- | |
| | | | CUGUUUAUACAGUG- | |
| | | | CUCCCUUUUU | |
| | | | UUU | |
| | | | GAGUCGUACAACGAAG- | Salmonella enteric [symbol] |
| | | | CGAACGUCAGGAUGAU- | |
| | | | GACGCUUCAGCAG- | |
| | | | GACACGCCAGGAUGG | |
| | | | UGUUACAAGGAAAGGCUUCAG- | |
| | | | GAUGAAGCAAAGUG- | |
| | | | GAAAGCGCAGGAUG- | |
| | | | CGUUAAAGGAC | |
| | | | ACCUCCAGGACGGAGAACGA- | |
| | | | GAGCCGAUCAGGAU- | |
| | | | GUUCGGCGGAUCUGGAUAAC- | |
| | | | CAGGGA | |
| | | | CGCUUCAGGAUGAAGCUAUCA- | |
| | | | CAUCGGGCGAUGUGCGCAG- | |
| | | | GAUGUAAACGUUCAGGAUGA | |
| | | | ACAGGCCGUAAGGUCACAG- | |
| | | | GAAAAGUUGUCACGGAUGAG- | |
| | | | CAGGGAGCACGAAAAGUAGCU | |
| | | | GGAAUGCUG- | |
| | | | CGAAACGAACCGGGAGCA- | |
| | | | CUGUUUAUACAGUG- | |
| | | | CUCCCUUUUUUUGUU | |
| 802 | dicF | translational | UUUCUGGUGACGUUUGGCGGUAUCA- | Escherichia coli |
| | | repressor | GUUUUACUCCGUGACUGCU- | |
| | | | CUGCCGCCC | |
| 803 | oxyS | translational | GAAACGGAGCGGCACCUCUUUUAACC- | Escherichia coli |
| | | repressor | CUUGAAGUCACUGCCCGUUUCGAGA- | |
| | | | GUUUCUCAA | |
| | | | CUCGAAUAACUAAAGCCAACGUGAA- | |
| | | | CUUUUGCGGAUCUCCAGGAUCCGCU | |
| | | | AGCAUAGCAACGAACGAUUAUCC- | Salmonella enteric [symbol] |
| | | | CUAUCAACCUUUCUGAUUAAUAAUA- | |
| | | | CAUCACAGAACG | |
| | | | GAGCGGUUUCUCGUUUAACCCUUGAA- | |
| | | | GACACCGCCCGUUCAGAGGGUAUCU- | |
| | | | CUCGAACCC | |
| | | | GAAAUAACUAAAGCCAACGUGAA- | |
| | | | CUUUUGCGGACCUCUGGUCC- | |
| | | | GCUUUUUUUUGCGUAAA | |
| | | | AAA | |
| 804 | uptR | extracytoplasmic | GCUGAAUAUGAUUCAAUAUCGCAC- | Escherichia coli |
| | | toxicity | GCUACUCAUCCAUCCAAGGAUAAUGA- | |
| | | suppressor | GUACAUAGGU | |
| | | | UGAAGUUUCAACACCCCCACUAC- | |
| | | | GGGGGUGUUUUUU | |

[symbol]

indicates data missing or illegible when filed

Further non-limited examples of second subsequences in the form of plant RNA polynucleotides according to the present invention are listed in Table 7 below. It will be understood that such sequences, or a complementary strand thereof, can be operably linked to a first subsequence as defined herein elsewhere.

TABLE 7

| | | accession | |
|---|---|---|---|
| SEQ ID NO | ID | number | species |
| 805 | AtGUT15 | U84973 | Arabidopsis thaliana |
| 806 | GUT15 | U84972 | Nicotiana tabacum |
| 807 | SRE1a | U75693 | Solanum tuberosum |
| 808 | SRE1b | U75694 | Solanum tuberosum |
| 809 | SRE1c | U75695 | Solanum tuberosum |
| 810 | AtCR20-1 | D79218 | Arabidopsis thaliana |
| 811 | CR20 | D79216 | Cucumis sativus |
| 812 | Gm-c1025-1333 | AW317238 | Glycine max |
| 813 | pGVN-47L6 | AW573678 | Medicago truncatula |
| 814 | LP148-26-h10 | BE122467 | Lotus japonicus |
| 815 | A034p17u | AI163153 | Hybrid aspen |
| 816 | EST00587 | AI563463 | Citrullus lanatus |
| 817 | GF-FV-P1D2 | BE205699 | Grapefruit |
| 818 | cLEN7C4 | AW222192 | Lycopersicon esculentum |
| 819 | BNLGHi9947 | AW187098 | Gossypium hirsutum |
| 820 | 603030H12.x1 | AI947916 | Zea mays |
| 821 | S20758_1A | AU056647 | Oryza sativa |
| 822 | At4 | AF055372 | Arabidopsis thaliana |
| 823 | Mt4 | U76742 | Medicago truncatula |
| 824 | AtIPS1 | AF236376 | Arabidopsis thaliana |
| 825 | TPSI1 | U34808 | Lycopersicon esculentum |
| 826 | LP169-27-c1 | BE122482 | Lotus japonicus |
| 827 | su32a08.y1 | BF325311 | Glycine max |
| 828 | 179K9T7 | H37319 | Arabidopsis thaliana |
| 829 | 248G6T7 | W43209 | Arabidopsis thaliana |
| 830 | E6G11T7 | AA042352 | Arabidopsis thaliana |
| 831 | ZCF120 | AB028200 | Arabidopsis thaliana |
| 832 | ZCF112 | AB028193 | Arabidopsis thaliana |
| 833 | ZF2 | AB028197 | Arabidopsis thaliana |
| 834 | RXF6 | AB008026 | Arabidopsis thaliana |
| 835 | RXW18 | AB008024 | Arabidopsis thaliana |
| 836 | ZCF44 | AB028227 | Arabidopsis thaliana |
| 837 | ZCF58 | AB028192 | Arabidopsis thaliana |
| 838 | ATH132404 | AJ132404 | Arabidopsis thaliana |
| 839 | ZCF83 | note | Arabidopsis thaliana |
| 840 | SRK | — | Brassica oleracea |
| 841 | AS-ZmSLR | AJ001485 | Zea mays |
| 842 | SLA2 | L43495 | Brassica oleracea |
| 843 | Bz2 | — | Zea mays |

Further non-limited examples of second subsequences in the form of yeast RNA polynucleotides according to the present invention are listed in Table 8 below. It will be understood that such sequences, or a complementary strand thereof, can be operably linked to a first subsequence as defined herein elsewhere.

TABLE 8

| SEQ ID NO | ID | sequence | species |
|---|---|---|---|
| 844 | RUF5-1 | AACAAAGTATCTAAA- | Saccharomyces |
| | | CAAAATACATAAGT- | cerevisiae |
| | | GTACTCAAACTGAGTA- | |
| | | GAATCGTCGATTAAA | |
| | | CTTCCTTCTCCTTTTAA | |
| | | AAATTAAAAACAG- | |
| | | CAAATAGTTAGATGAA- | |
| | | TATATTAAAGACTA | |
| | | TTCGTTTCATTTCCCA- | |
| | | GAGCAGCATGACTTCTT | |
| | | GGTTTCTTCAGACTT- | |
| | | GTTACCGCAGGG | |
| | | GCATTT- | |
| | | GTCGTCGCTGTTA- | |
| | | CACCCCGTTGGGCAGC- | |
| | | TACATGATTTTT- | |
| | | GGCATTGTTCATT | |
| | | ATTTTTGCAGCTACCA- | |
| | | CATTGGCATTGGCACT- | |
| | | CATGACCTTCATTTT- | |
| | | GGAAGTTAATTAA | |
| | | TTCGCTGAACATTT- | |
| | | TATGTGATGATTGATT- | |
| | | GATTGATTGTACAGTTT | |
| | | GTTTTTCTTAATA | |
| | | TCTATTTCGAT- | |
| | | GACTTCTATATGA- | |
| | | TATTGCACTAACAA- | |
| | | GAAGATATTATAAT- | |
| | | GCAATTGA | |
| | | TACAAGACAAGGAGT- | |
| | | TATTT- | |
| | | GCTTCTCTTTTATAT- | |
| | | GATTCTGACAATCCA- | |
| | | TATTGCGTTG | |
| | | GTAGTCTTTTTT- | |
| | | GCTGGAACGGTTCAGC- | |
| | | GGAAAAGACGCATC- | |
| | | GCTCTTTTTGCTTCTA- | |
| | | GA | |
| | | AGAAATGCCAGCAAAA- | |
| | | GAATCTCTTGACAGT- | |
| | | GACTGACAGCAAAAAT- | |
| | | GTCTTTTTCTAAC | |
| | | TAGTAACAAGGCTAA- | |
| | | GATATCAGCCTGAAA- | |
| | | TAAAGGGTGGTGAAG- | |
| | | TAATAATTAAATCAT | |
| | | CCGTATAAACCTATA- | |
| | | CACATATATGAG- | |
| | | GAAAAATAATA- | |
| | | CAAAAGTGTTTT | |
| 845 | RUF5-2 | AACAAAGTATCTAAA- | Saccharomyces |
| | | CAAAATACATAAGT- | cerevisiae |
| | | GTACTCAAACTGAGTA- | |
| | | GAATCGTCGATTAAA | |
| | | CTTCCTTCTCCTTTTAA | |
| | | AAATTAAAAACAG- | |
| | | CAAATAGTTAGATGAA- | |
| | | TATATTAAAGACTA | |
| | | TTCGTTTCATTTCCCA- | |
| | | GAGCAGCATGACTTCTT | |
| | | GGTTTCTTCAGACTT- | |
| | | GTTACCGCAGGG | |
| | | GCATTT- | |
| | | GTCGTCGCTGTTA- | |
| | | CACCCCGTTGGGCAGC- | |
| | | TACATGATTTTT- | |
| | | GGCATTGTTCATT | |
| | | ATTTTTGCAGCTACCA- | |
| | | CATTGGCATTGGCACT- | |
| | | CATGACCTTCATTTT- | |
| | | GGAAGTTAATTAA | |
| | | TTCGCTGAACATTT- | |
| | | TATGTGATGATTGATT- | |
| | | GATTGATTGTACAGTTT | |
| | | GTTTTTCTTAATA | |
| | | TCTATTTCGAT- | |
| | | GACTTCTATATGA- | |
| | | TATTGCACTAACAA- | |
| | | GAAGATATTATAAT- | |
| | | GCAATTGA | |
| | | TACAAGACAAGGAGT- | |
| | | TATTT- | |
| | | GCTTCTCTTTTATAT- | |
| | | GATTCTGACAATCCA- | |
| | | TATTGCGTTG | |
| | | GTAGTCTTTTTT- | |
| | | GCTGGAACGGTTCAGC- | |
| | | GGAAAAGACGCATC- | |
| | | GCTCTTTTTGCTTCTA- | |
| | | GA | |
| | | AGAAATGCCAGCAAAA- | |
| | | GAATCTCTTGACAGT- | |
| | | GACTGACAGCAAAAAT- | |
| | | GTCTTTTTCTAAC | |
| | | TAGTAACAAGGCTAA- | |
| | | GATATCAGCCTGAAA- | |
| | | TAAAGGGTGGTGAAG- | |
| | | TAATAATTAAATCAT | |
| | | CCGTATAAACCTATA- | |
| | | CACATATATGAG- | |
| | | GAAAAATAATA- | |
| | | CAAAAGTGTTTT | |
| 846 | SNR84 | ATTGCACAACT- | Saccharomyces |
| | | TAAGTTTGTCGAGGAT- | cerevisiae |
| | | CATTTTTTTGAACT- | |
| | | GAATCAT- | |
| | | GCTCTTTTTAAG | |
| | | TGCTTTGAAACCCTC- | |
| | | GATGAATGTGTCAAT- | |
| | | GTGCAAAGATAAAC- | |
| | | CATTGTTCTCTGTTGA | |
| | | TCAGTGACTTAAT- | |
| | | GTTTGCTTTGGAGAAT- | |
| | | GATATTTTCCCTTTCC- | |
| | | TATATTTGACTTTTG | |
| | | TTCTAAAAGTTATTT- | |
| | | GGAGAGAAAAGGCAT- | |
| | | GATTGAGGTT- | |
| | | GCGACTTTTTCGTTTTT | |
| | | GCT | |
| | | TTTGCATGGATAATT- | |
| | | CATCCATGCACATCT- | |
| | | CACTTTATTGGACCTT- | |
| | | CAAGATTGGTTTCC | |
| | | CATGTAATT- | |
| | | TAATTTTCTCTCCTC- | |
| | | TACATTTAATAT- | |
| | | GTTCTATATTAATTAA- | |
| | | TACCAATT | |
| | | GAGTTGTGCGTACTT- | |
| | | CATTGCAGATATTT- | |
| | | TACCAGACCT- | |
| | | GTCTGAGTTTTTC- | |
| | | GTTCAAGT | |
| | | TTGGTTGAAATC- | |
| | | GGCTTGAGGTATAT- | |
| | | GAACGTGGTTGGGA- | |
| | | TATGGAGATTGGGA- | |
| | | GATCAA | |
| | | AGAAGCGAAAATACCT- | |
| | | GAGACAGTTTTTT- | |
| | | TAAAAAAGAAGCTAAG- | |
| | | GAACATGACTCAAAG | |
| | | AGACACATTA | |
| 847 | SNR82 | ATGGCTCTTCAACA- | Saccharomyces |
| | | CATTTCAACAT- | cerevisiae |
| | | GTTCAAGTAATTT- | |
| | | GTGTTAGTGGATGAC- | |
| | | CATTTAG | |
| | | GGGCTGCTGGCCTGGTT | |
| | | ACCGGGAGTTTTTCTT- | |
| | | GGATCCAAGC- | |
| | | TAGCTTTTCCGTCTGAT | |
| | | TATCCTTAAGCTTCA- | |
| | | CAAATTA- | |
| | | CAATTTTTCCCAC- | |
| | | GCATTAAGAAA- | |
| | | TAAGCTCAAGATGC | |
| | | CTAAAATAAGTTC- | |
| | | TATCCC- | |
| | | GCCTTTTTTCGCTAA- | |
| | | CAATGACTGAG- | |
| | | TATTCCCACAGTCTA | |
| | | TAGTTTGATAGTAGAT- | |
| | | GGGCGGAAATTT | |
| 848 | SNR83 | ACCCAAAAACATCAA- | Saccharomyces |
| | | GAAAAGCCTTTCAA- | cerevisiae |
| | | TAAATT- | |
| | | GCTCTTCTCTTGGCGAA | |
| | | AGAAAGCG | |
| | | GGGGGCAAAAAGAAT- | |
| | | CACGGGACTTAT- | |
| | | GTTTCGGGATCTCTTTG | |
| | | TTTCTTCTTTTTTTCC | |
| | | CGGAGAATAATTTTT- | |
| | | TAGGACCAATTACC- | |
| | | GTAGTTGCGACTACAA- | |
| | | CAATTGTTGTTCATA | |
| | | CCCCCACGATT- | |
| | | TACTTTTTGAAAAC- | |
| | | TAGTTTTTGGAATAA- | |
| | | TAAT- | |
| | | GTTGTAAAATTTCCCT | |
| | | TTTTCCACCCCGATTT- | |
| | | GTATTTTATTTTTC- | |
| | | GTTACAAAATTGGGAC- | |
| | | TAATATTAAGGGCG | |
| | | ACAGTT | |

It will be understood that in preferred embodiments, mammalian second subsequences are expressed in mammalian cells, human second subsequences are expressed in human cells, fungal second subsequences are expressed in fungal cells, yeast second subsequences are expressed in yeast cells, and bacterial second subsequences are expressed in bacterial cells.

Also, It will be understood that in preferred embodiments, mammalian second subsequences are cloned in vectors capable of being transformed or transfected into mammalian cells prior to expression, human second subsequences are cloned in vectors capable of being transformed or transfected into human cells prior to expression, fungal second subsequences are cloned in vectors capable of being transformed or transfected into fungal cells prior to expression, yeast second subsequences are cloned in vectors capable of being transformed or transfected into yeast cells prior to expression, and bacterial second subsequences are cloned in vectors capable of being transformed or transfected into bacterial cells prior to expression.

In all of the above cases the expression of the first subsequence and the second subsequence is directed by an expression signal capable of directing said expression in the host cell in question under appropriate cultivation conditions.

Gene Therapy

Having identified RNA instability or a decrease in the RNA level, for example due to decreased transcription, as the cause of a disease it is also rendered possible in accordance with the present invention to provide a genetic therapy for subjects being diagnosed as having.a-predisposition for or suffering from a disease associated With RNA instability or a decrease in RNA level, said therapy comprising administering to said subject a therapeutically effective amount of a gene therapy vector.

The gene therapy vectors comprise a sequence coding for the RNA associated with the disease and/or a polynucleotide sequence comprising GIR1 or a variant thereof. In particular the invention relates to a gene therapy vector comprising i) a first DNA or RNA subsequence selected from the group consisting of SEQ ID NO 1, SEQ ID NO:2; SEQ ID NO:1A and SEQ ID NO:2A, or a variant or a fragment thereof, or the complementary strand thereof, and a second subsequence selected from the group consisting of second subsequences listed in Table 3, second subsequences listed in Table 4, second subsequences listed in Table 5, second subsequences listed in Table 6 and second subsequences listed in Table 7, or a variant or a fragment thereof, or the complementary strand of any of said sequences.

Various different methods of gene therapy can be used for treating subjects suffering from a disease as defined in the present invention. The person skilled in the art will be well aware of such methods.

Other types of gene therapy include the use of retrovirus (RNA-virus). Retrovirus can be used to target many cells and integrate stably into the genome. Adenovirus and adeno-associated virus can also be used. A suitable retrovirus or adenovirus for this purpose comprises an expression construct comprising a sequence coding for the RNA associated with the disease and/or a polynucleotide sequence comprising GIR1 or a variant thereof under the control of a constitutive promoter or a regulatable promoter such as a repressible and/or inducible promoter or a promoter comprising both repressible and inducible elements. The construct comprising a sequence coding for the RNA associated with the disease and/or a sequence comprising GIR1, or a variant thereof, may be inserted into the appropriate cells within a patient, using vectors that include, but are not limited to adenovirus, adeno-associated virus, and retrovirus vectors, in addition to other particles that introduce DNA into cells, such as liposomes.

Described below are- methods and compositions whereby a disorder associated with RNA instability may be treated. In particular diseases associated with RNA instability selected from the group consisting of but not limited to: Cancer, such as for example chronic lymphocytic leukemia, ovarian cancer, breast cancer and melanoma; Cachexia and a-thalessemia.

Gene replacement therapy techniques should be capable delivering a sequence coding for the RNA associated with the disease and/or GIR1 or a variant thereof to cells transcribing the corresponding RNA within patients. Thus, in one embodiment, techniques that are well known to those of skill in the art (see, e.g., PCT Publication No. WO89/10134, published Apr. 25, 1988) can be used to enable the sequence coding for the RNA associated with the disease and/or GIR1 or a variant thereof to be uptaken by the cells. Viral vectors may advantageously be used for the purpose. Also included are methods using liposomes either in vivo ex vivo or in vitro, wherein the sense or antisense DNA sequence coding for the RNA associated with the disease and/or GIR1 or a variant thereof is delivered to the cytoplasm and nucleus of target cells. Liposomes can deliver the sense or antisense DNA sequence coding for the RNA associated with the disease and/or GIR1 or a variant thereof to humans and the lungs or skin through intrathecal delivery either as part of a viral vector or as DNA conjugated with nuclear localizing proteins or other proteins that increase take up into the cell nucleus.

In another embodiment, techniques for delivery involve direct administration of such sense or antisense DNA sequence coding for the RNA associated with the disease and/or GIR1 or a variant thereof to the site of the cells in which the sense or antisense DNA sequence coding for the RNA associated with the disease and/or GIR1 or a variant thereof are to be expressed.

Treatment of Cachexia

Muscle wasting (cachexia) is a consequence of chronic diseases, such as cancer, and is associated with degradation of muscle proteins such as MyoD. Cachexia is a condition that leads to the alteration of several physiological and behavioral attributes, ranging from fatigue and fever to excessive weight loss. The detrimental effects of cachexia occur as a consequence of excessive wasting of skeletal muscle tissue. It is well established that muscle atrophy requires the activation of transcription factors such as NF-κB and Foxo-3, leading to the rapid decrease of MyoD mRNA. three highly conserved muscle-specific microRNAs, miR-1, miR-133 and miR-206, are robustly induced during the myoblast-myotube transition, both in primary human myoblasts and in the mouse mesenchymal C(2)C(12) stem cell line. MyoD binds to regions upstream of these microRNAs and, therefore, are likely to regulate their expression.

Thus in one embodiment the RNA to be stabilized is MyoD mRNA or a variant thereof.

Treatment of α-Thalassemia

Globin mRNA is particularly stable. Three C-rich elements located in the 39UTR of α-globin mRNA are targets for binding of the a-complex, a group of proteins predominantly containing the PCBPs, which maintain stability. An α-globin gene variant, a constant spring, or acs, is the most common cause of nondeletional a-thalassemia worldwide. This variant contains a stop codon. mutation that allows read through of translation into the 39UTR, and this is associated with a major decrease in mRNA half-life, which is associated with a-thalassemia.

Thus in one embodiment the polynucleotide to be stabilized is α-globin mRNA or a variant thereof.

Treatment of Cancer

A number of miRNAs are associated with cancer diseases. For example a high portion of miRNA containing genes exhibit copy number alterations in ovarian cancer, breast cancer, and melanoma and these copy changes correlate with miRNA expression. For example the miRNA mir-320 is located in regions with DNA copy number loss in all of the three cancer types. A notable mir-320 target predicted by two independent programs is methyl CpG binding protein 2 (MECP2), which is overexpressed in breast cancer and serves as an oncogene promoting cell proliferation. Also mir-218-1 is located within the tumor suppressor gene SLIT2 (human homologue of Drosophila Slit2), which is frequently inactivated in breast, lung, and colorectal cancer because of allelic loss. It has been shown that there is a copy number loss of the region containing mir-218-1 ovarian cancers, breast cancers, and melanoma lines.

Treatment of Chronic Lymphocytic Leukemia

Chronic lymphocytic leukemia is the most common form of adult leukemia in the Western world. To miRNAs miR15 and miR16 lie within a small regionof chromosome 13q14 that is deleted in more than 65% of CLL and that allelic loss in this region correlates with down-regulation of both miR-15 and miR-16 expression suggest that these genes represent the targets of inactivation by allelic loss in CLL.

Thus in one embodiment the polynucleotide to be stabilized is mir-15 miRNA or a variant thereof. In another embodiment the polynucleotide to be stabilized is mir-16 miRNA or a variant thereof.

Compositions

Compositions or pharmaceutical compositions or formulations for use in the present invention include a preparation of a recombinant polynucleotide or a vector or a host cell according to the invention in combination with, preferably dissolved in, a pharmaceutically acceptable carrier, preferably an aqueous carrier or diluent. The composition may be a solid, a liquid, a gel or an aerosol. A variety of aqueous carriers may be used, such as 0.9% saline, buffered saline, physiologically compatible buffers and the like. The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc. The formulations may contain pharmaceutically acceptable carriers and excipients including microspheres, liposomes, microcapsules, nanoparticles or the like. Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar Lipids, Inc, Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235;871, 4,501,728 and 4,837,028, all of which are incorporated herein by reference. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butano, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In some cases, it will be advantageous to include a compound, which promotes delivery of the active substance to its target. For example a ligand which is capable of binding to a receptor present on the target tissue(s) and/or the target cell(s) can be included.

Dosing Regimes

The preparations are administered in a manner compatible With the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred pg active ingredient per administration with a preferred range of from about 0.1 μg to 10000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. A preferred dosage would be from about 0.1 to about 5.0 mg, preferably from about 0.3 mg to about 3.0 mg, such as from about 0.5 to about 1.5 mg and especially in the range from 0.8 to 1.0 mg per administration. Administration may be performed once or may be followed by subsequent administrations. The dosage will also depend on the route of administration and will vary with the age, sex and weight of the subject to be treated. A preferred dosage of multimeric forms would be in the interval 1 mg to 70 mg per 70 kilo body weight.

Suitable daily dosage ranges are per kilo body weight per day normally of the order of several hundred pg active ingredient per day with a preferred range of from about 0.1 μg to 10000 μg per kilo body weight per day. Using monomeric forms of the compounds, the suitable dosages are often in the range of from 0.1 μg to 5000 μg per kilo body weight per day, such as in the range of from about 0.1 μg to 3000 μg per kilo body weight per day, and especially in the range of from about 0.1 μg to 1000 μg per kilo body weight per day. Using multimeric forms of the compounds, the suitable dosages are often in the range of from 0.1 μg to 1000 μg per kilo body weight per day, such as in the range of from about 0.1 μg to 750 μg per kilo body weight per day, and especially in the range of from about 0.1 μg to 500 μg per kilo body weight per day, such as in the range of from about 0.1 μg to 250 μg per kilo body weight per day. A preferred dosage would be from about 0.1 to about 100 μg, preferably from about 0.1 μg to about 50 μg, such as from about 0.3 to about 30 μg and especially in the range from 1.0 to 10 μg per kilo body weight per day. Administration may be performed once or may be followed by subsequent administrations. The dosage will also depend on the route of administration and will vary with the age, sex and weight of the subject to be treated. A preferred dosage of multimeric forms would be in the interval 1 mg to 70 mg per 70 kilo body weight per day.

Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a composition comprising a compound as defined above or a pharmaceutically acceptable salt thereof and pharmaceutically acceptable carriers, vehicles and/or excipients, said packaging comprising from 1 to 7 dosage units, thereby having dosage units for one or more days, or from 7 to 21 dosage units, or multiples thereof, thereby having dosage units for one week of administration or several weeks of administration.

The dosage units can be as defined above. The medical packaging may be in any suitable form for systemic or local administration. In a preferred embodiment the packaging is in the form of a vial, ampule, tube, blister pack, cartridge or capsule.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the composition.

### EXAMPLES

The following examples illustrate embodiments of the present invention and shall not be construed as a narrowing of the protection sought.

### Example 1

Reference is made to Science, vol. 309, September 2005.

Templates, In Vitro Transcription and Cleavage Analysis:

Templates for in vitro transcription were made by standard PCR using Pfu DNA polymerase (Stratagene) and pDi162SG1 (C. Einvik, H. Nielsen, E. Westhof, F. Michel, S. Johansen, RNA 4, 530 (1998)) as template. The oligonucleotide primers were C289 (5′-AAT TTA ATA CGA CTC ACT ATA GGT TGG GTT GGG MG TAT CAT) and OP233 (5′-GAT TGT CTT GGG ATA CCG) for 166.22, and C294 (5′-AAT TTA ATA CGA CTC ACT ATA GGG MG TAT CAT) and OP233 for 157.22. The PCR products were purified using a commercial kit (GenElute PCR Clean-up kit, Sigma) and transcribed by T7 RNA polymerase (Fermentas) in a 50-μl reaction according to the manufacturer's recommendations. For radioactive labeling of the RNA, 1 μl of [α-32P]UTP (3000 Ci/mmol; Amersham Biosciences) was included in the transcription reaction. Transcripts were purified by phenol:chloroform:isoamylalcohol (25:24:1) extraction and ethanol precipitated. Cleavage experiments were carried out as described in C. Einvik, H. Nielsen, R. Nour, S. Johansen, Nucl. Acids Res. 28, 2194 (2000).

Briefly, the RNA was renatured at 45° C. for 5 min in acetate buffer (pH=5.5) containing 1 M KCl and 25 mM MgCl2. Then the reaction was started by addition of 4 vols. of 47.5 mM Hepes-KOH (pH=7.5) containing 1 M KCl and 25 mM MgCl2 and time samples withdrawn at the appropriate times. The kinetic analysis was performed as described in C. Einvik, H. Nielsen, R. Nour, S. Johansen, Nucl. Acids Res. 28, 2194 (2000) except in that Sigmaplot 8.0 was used in data treatment.

RNA Purification From Gels, 3′-End Labeling, and Primer Extension Analysis:

RNA was purified from polyacrylamide gels by overnight elution at 4° C. in 250 mM sodium acetate (pH=5.2), 1 mM EDTA mixed with 1 vol. of phenol see J. Kjems, J. Egebjerg, J. Christiansen, Analysis of RNA-Protein Complexes in Vitro, (Elsevier Science Ltd, Amsterdam, 1998). pCp was made from Cp and [γ-32P]ATP (6000 Ci/mmol; Amersham Biosciences) using T4 polynucleotide kinase (Fermentas) see J. Kjems, J. Egebjerg, J. Christiansen, Analysis of RNA-Protein Complexes in Vitro, (Elsevier Science Ltd, Amsterdam, 1998). The [32P]pCp was used without further purification to 3′-end label RNA using T4 RNA ligase (Amersham Biosciences) see J. Kjems, J. Egebjerg, J. Christiansen, Analysis of RNA-Protein Complexes in Vitro, (Elsevier Science Ltd, Amsterdam, 1998). The 3′-end labeled RNA was gel-purified before use. Primer extension analysis of cleavage reactions were performed as described (C. Einvik, H. Nielsen, E. Westhof, F. Michel, S. Johansen, RNA 4, 530 (1998) using C291 (5′-GAT TGT CTT GGG AT) as primer.

Ligation Experiments and β-Elimination:

Ligation experiments were performed by mixing gel purified RNAs in dH2O followed by addition of 1 vol. of a 2×reaction buffer (2 M KCl, 50 mM MgCl2, 95 mM Hepes-KOH, pH=7.5) at 45° C. Time samples were withdrawn and stopped by pipetting into denaturing (7 M urea) loading buffer. β-elimination of gel purified 166 RNA was carried out by oxidation in 20 mM sodium periodate followed by aniline cleavage as described (N. K. Tanner, T. R. Cech, Biochemistry 26, 3330 (1987). The RNA was gel-purified before subsequent ligation experiments.

Enzymatic 5′-End Analysis and Alkaline Ladders:

For analysis of the 5′-end, RNAs were initially 3′-end labeled by [32P]pCp and gel purified. Aliquots of the RNA were then subjected to enzymatic analysis using shrimp alkaline phosphatase (SAP; Fermentas) and T4 polynucleotide kinase (Fermentas) or to partial alkaline hydrolysis by boiling in 50 mM NaHCO3/Na2CO3, pH 9.0 (3). The samples were analyzed on 10% denaturing (7 M urea) polyacrylamide gels. A partial RNase T1 (Sigma) digest was used as a size marker.

Analysis of Branch Nucleotides:

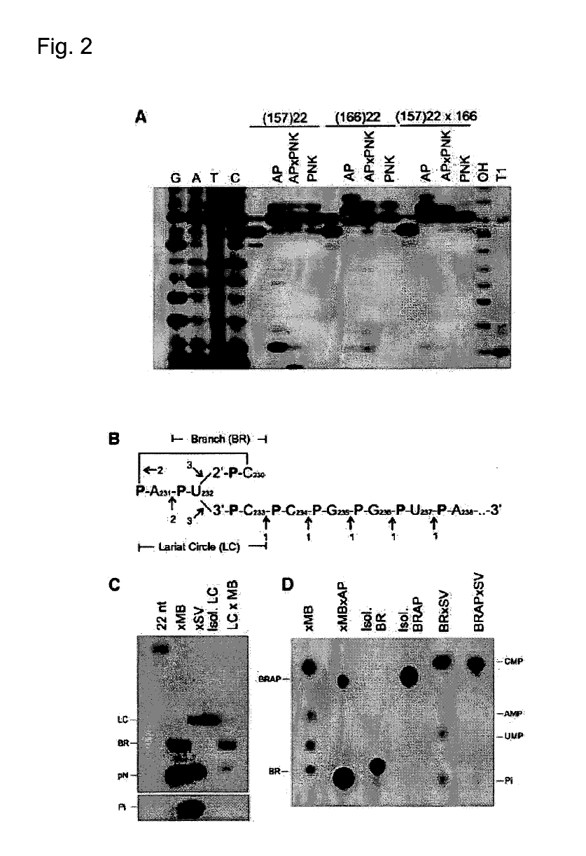

The structure analysis of the branch nucleotides were performed on gel purified 3′-fragments isolated from cleavage reactions with body-labeled RNA. Aliquots of the RNA were subjected to enzymatic analysis using mung bean nuclease (Stratagene) and calf intestinal phosphatase (New England Biolab) according to the manufacturers' recommendations. In double digestions, 1 vol. of a 2×reaction buffer (200 mM Tris (pH=9.0), 20 mM MgCl2, 1 mM ZnCl2, 10 mM spermidine) was added to the mung bean nuclease digest and incubation continued in the presence of CIP. The samples were analyzed on 20% denaturing (7 M urea) polyacrylamide gels. A partial alkaline hydrolysis reaction was used as a size marker. Digestion with snake venom phosphodiesterase (Crotalus atrox venom; Pharmacia) was in 100 mM Tris-HCl (pH=8.9), 100 mM NaCl, 14 mM MgCl2 at 25° C. for 30 min. TLC analyses were performed on PEI-cellulose plates using 0.9 M Acetic acid/0.3 M LiCl as running buffer. In preparative experiments, the material was scaped of the plate and the nucleotides eluted in 2 M NH4OH. In the experiments on characterization of the lariat circle (FIG. 2B) and branch nucleotide (FIG. 2C), the RNA was labeled with a combination of [α-32P]UTP, [α-32P]CTP, and [α-32P]ATP.

Cleavage Experiment With Deoxy-Substituted RNA Oligos:

The deoxy-substituted oligonucleotides were purchased from Dharmacon. The ribozyme version used in cleavage experiments with these oligos was made by PCR using C294 and C421 (5′-TCG GM CGA CTG TTC ATT GM C). The cleavage experiments were carried out as described above.

Individual RNA species described in the document are named according to the number of nucleotides included. For example, 166.22 refers to a GIR1 ribozyme including 166 nt upstream of the IPS (internal processing site), and 22 nt down-stream of the IPS. Parentheses are used to describe the origin of a particular RNA species. (166)22 means a 22-nt fragment isolated from cleavage of a 166.22 precursor RNA. Nucleotide numbering is according to the position in the full-length intron (The sequence of Dir.S956 intron has acc. no. X71792 in Genbank).

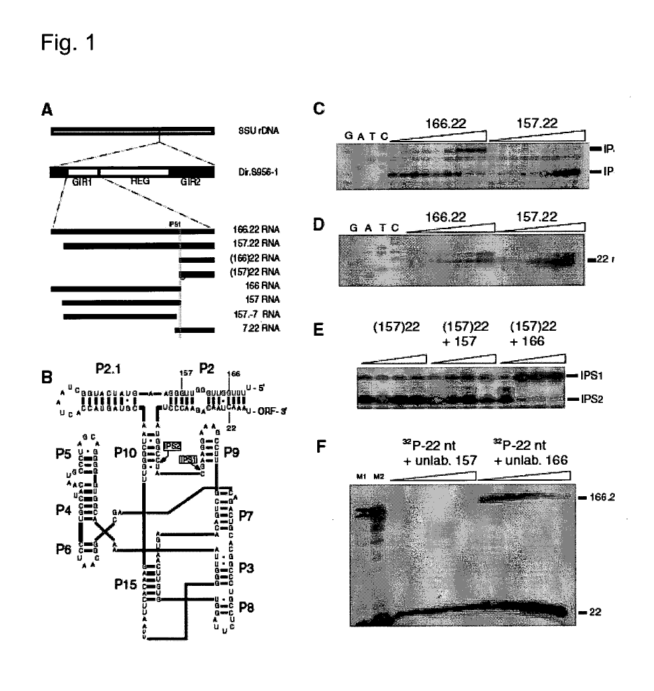

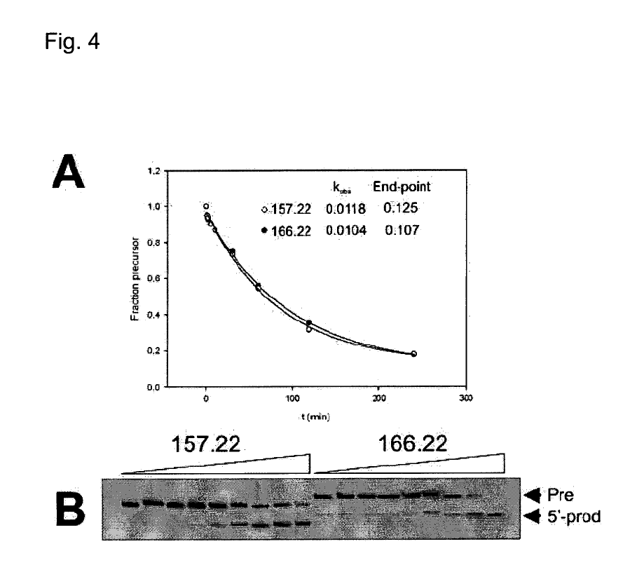

The cleavage analysis shown in FIG. 4 is complicated by the reversibility of the reaction. It is interpreted that the reaction of 166.22 to be the sum of a forward transesterification, an efficient reverse (ligation) reaction (as demonstrated in FIG. 1F), and a relatively slow forward hydrolytic reaction.

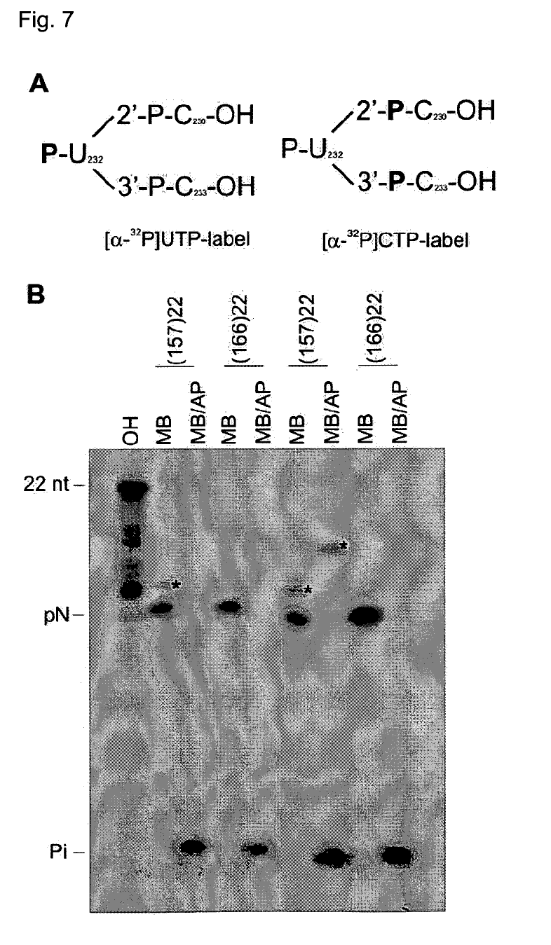

The reaction with 157.22 is dominated by the forward transesterification. In the mung bean nuclease analysis of branched nucleotides (FIG. 7), a parallel experiment [α-32P]ATP or [α-32P]GTP was performed. No mung bean-resistant fragments was observed with these labels in either (157.22) or (166)22 RNAs.

### Example 2

The group I twin-ribozyme intron found in the extrachromosomal ribosomal DNA (rDNA) of the myxomycete Didymium iridis (Dir.S956-1) consists of two self-catalytic units, a conventional group I splicing ribozyme (GIR2) and a group I-like cleavage ribozyme (GIR1) (FIG. 1A). A homing endonuclease gene (HEG) encoding the l-Dirl mRNA is found inserted downstream of GIR1 (4-6). The 5* end of the I-Dirl mRNA is formed by cleavage catalyzed by the GIR1 ribozyme (7).

Primer extension analyses have led to the suggestion of two cleavage sites located three nucleotides apart (5, 8) referred to as IPS1 (internal processing site 1), and IPS2, respectively (FIG. 1B).

A primer extension stop at IPS1 accumulates over time in 166.22 and a stop at IPS2 accumulates in 157.22 (FIG. 1C). In a parallel cleavage analysis with 3′ end-labeled RNA (FIG. 1D) the 3′ fragment that accumulates from cleavage of both 166.22 and 157.22 is of the same length (22 nt). This is inconsistent with cleavage at IPS2, and it was conclude that the observed primer extension stop at IPS2 is a structural stop. Incubation of a 22-nt 3′ fragment isolated from cleavage of 157.22 (IPS2) with the 166-nt 5′ fragment results in a complete conversion of the primer extension signal from IPS2 to IPS1 (FIG. 1E) because of ligation and recleavage by hydrolysis. Ligation of the 22-nt fragment onto the 3′ end of the 5′ fragment followed by recleavage is shown in FIG. 1F.

The 5′ ends of the two 22-nt RNAs were analyzed by treatment of 3′ end-labeled RNA with modifying enzymes (FIG. 2A). Incubation of the 3′ fragment carrying the IPS-2 modification E(157)22 RNA^{A} with AP (alkaline phosphatase) or AP and PNK (polynucleotide kinase), or PNK alone all shifted the mobility of the fragment one position upward in the gel, which was consistent with the removal of the 3′-phosphate of the pCp label. In contrast, a 3′ fragment that resulted from cleavage at IPS1 without the IPS2 modification E(166)22 RNA was shifted two positions upward with AP, one position when phosphorylated with PNK after AP treatment, and one position with PNK alone. This is consistent with removal of the 3′-phosphate (from the pCp) as well as an additional phosphate at the 5′ end left by IPSi cleavage. Thus, the phosphate at the 5′ end of the 22-nt 3′ fragment is accessible to phosphatase in the absence of the IPS2 modification but inaccessible when the IPS2 modification is present. This feature of the IPS2 modification could be removed by incubation of (157)22 RNA with 166 RNA before the analysis, as shown in the last panel in FIG. 2A. Thus, both the primer extension stop at IPS2 and blocking of the 5′ end are reversible. An explanation for these observations is that GIR1 cleavage occurs by a transesterification reaction in which cleavage at IPS1 is coupled to formation of a 2′,5′-phosphodiester bond between C230 and U232. This explains the primer extension stop at IPS2, the blocking of the 5′ end, the conservation of internal energy after cleavage, and the reversibility of the reaction.

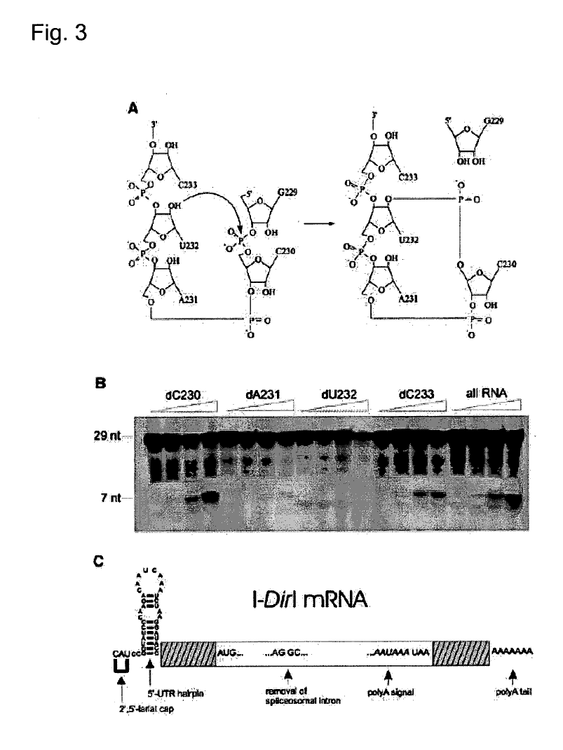

Branches in RNA are resistant to digestion with various RNases including mung bean nuclease (13). A resistant fragment was found in mung bean nuclease digests of bodylabeled (157)22 RNA but not (166)22 RNA (FIG. 7 and SOM text). Digestion of (157)22 RNA with the exonuclease snake venom phosphodiesterase resulted in a resistant fragment corresponding to the 4-nt lariat circle (FIG. 2B) that could subsequently be cleaved by the endonuclease mung bean nuclease to release the branched nucleotide and pA (FIG. 2C). These analyses are consistent with the presence of the proposed 2′,5′-phosphodiester bond between C230 and U232. The sequence of the branch was verified by thin-layer chromatography (TLC) analysis of the nucleotides liberated by snake venom phosphodiesterase cleavage of purified branch nucleotide (FIG. 2D). Formation of the branched nucleotide implies a reaction mechanism in which the 2′OH of U232 makes a nucleophilic attack at the phosphodiester bond at IPS (FIG. 3A). To test this mechanism, a cleavage analyses combining a ribozyme truncated in L9 (157.-7) and site-specifically deoxy-substituted substrates that complemented the truncated ribozyme (7.22) was made.

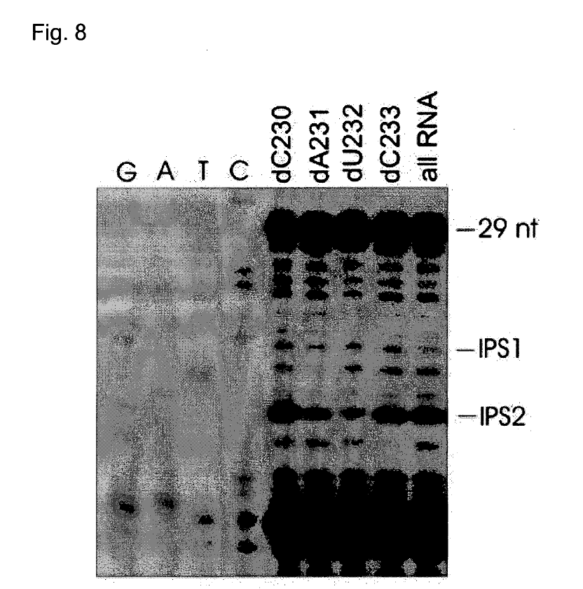

Only the dU232 substrate did not support cleavage (FIG. 3B). Weak cleavage with the dA231 substrate is ascribed to a critical structural role of this nucleotide. The cleavage in the all-RNA, dC230, dA231, and dC233 substrates was by transesterification as shown by primer extension analysis (FIG. 8). It previously has been shown that GIR1 cleaves by transesterification, not by hydrolysis as proposed previously. The reaction leaves a 5′ fragment containing a fully active ribozyme with a 3′OH, and a 3′ fragment in which the first and the third nucleotides are linked by a 2′,5′-phosphodiester bond. A 4-nt lariat was found by nuclear magnetic resonance (NMR) imaging to have an unusual structure with the sugars in the lariat ring locked in a rigid South-type conformation (14). The similarly sized lariat in Didymium is referred to as the lariat cap because it is found to cap the cellular I-Dir I mRNA (FIG. 3C).

Individual RNA species described are named according to the number of nucleotides included. For example, 166.22 refers to a GIR1 ribozyme including 166 nt up-stream of the IPS (internal processing site), and 22 nt downstream of the IPS. Parentheses are used to describe the origin of a particular RNA species. (166)22 means a 22-nt fragment isolated from cleavage of a 166.22 precursor RNA. Nucleotide numbering is according to the position in the full-length intron.

The cleavage analysis shown in FIG. 4 is complicated by the reversibility of the reaction. We interpret the reaction of 166.22 to be the sum of a forward transesterification, an efficient reverse (ligation) reaction (as demonstrated in FIG. 1F), and a relatively slow forward hydrolytic reaction. The reaction with 157.22 is dominated by the forward transesterification. In the mung bean nuclease analysis of branched nucleotides (FIG. 7), a parallel experiment [α-32P]ATP or [α-32P]GTP was performed. No mung bean-resistant fragments was observed with these labels in either (157.22) or (166)22 RNAs.

### Example 3

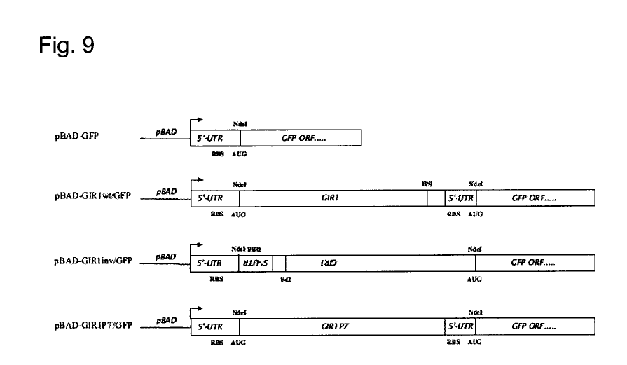

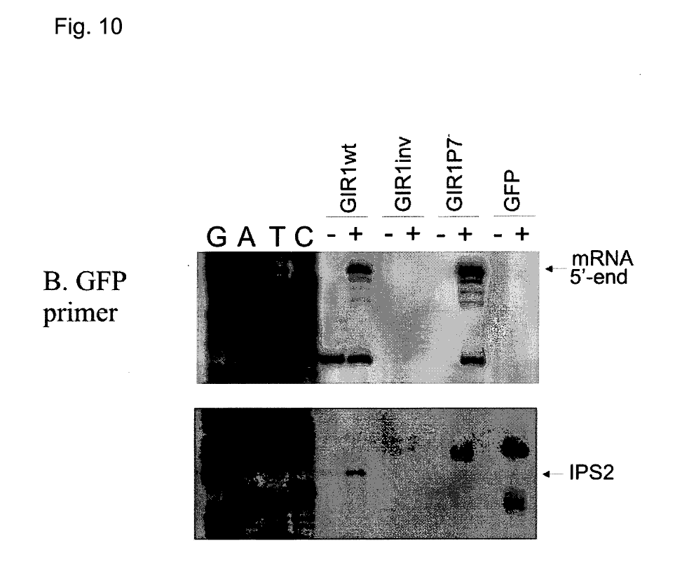

The constructs described in FIG. 9 were transformed into competent E. coli DH5α. Cells were grown on LB medium and analysed in the absence or presence of the inducer arabinose. RNA was extracted by the hot phenol method (Aiba H et al. J. Biol. Chem. 256, 11905-11910 (81)) and analysed by primer extension using primers complementary to GIR1 (A) (C473: 5′-CCC GAT TGC ATC ATG GTG A) or GFP (B) (C474: 5′-ATT GGG ACA ACT CCA GTG A). The products were run on 6% denaturing (urea) acylamide gels along with sequencing ladders made with the same primers and plasmid preps of the constructs as templates (FIG. 10). pBAD-GFP shows the expected inducibility by arabinose. No transcript is detected in GIR1invGFP. This is expected because the lack of a RBS positioned in front of the initiation codon results in very rapid turn-over of the transcript. In GIR1wtGFP and GIRlP7⁻GFP, the same arabinose inducibility is found as in the starting construct pBAD-GFP. The difference between the two is the presence of a primer extension stop signal in GIR1wtGFP, but not in GIR1P7⁻GFP corresponding to GIR1 catalysed cleavage at IPS. Notably, a primer extension product at this position is also found in the uninduced state where no primer extension stop signal corresponding to the 5′-end of the primary transcript is detected in any of the constructs. This signal is taken to represent low level transcription in the culture that is stabilized by the action of GIR1. The absence of a signal with either of the two primers in uninduced GIR1 P7⁻GFP cells makes an effect on transcription of the GIR1 insert unlikely. In other experiments it was shown that the half-life of the 5′-end of the transcripts from the pBAD-GFP and GIR1wtGFP constructs were of the same order (ca. 1 min).

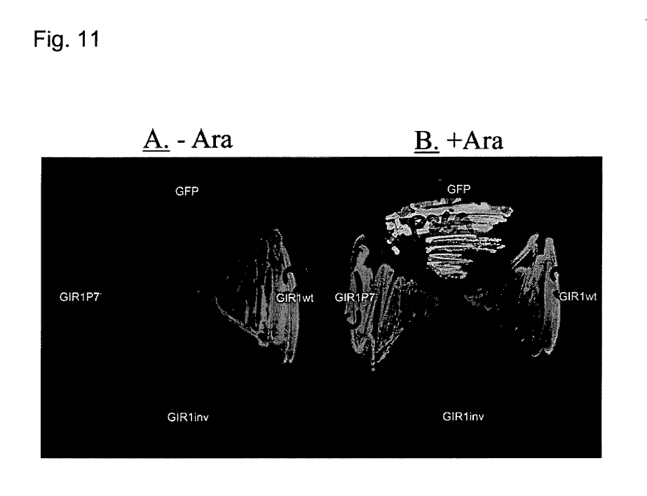

Cells containing the different constructs were plated on LB/Amp plates without or with the inducer arabinose. On the ara⁺ plate, bright fluorescence is observed with the pBAD-GFP construct, medium fluorescence with the GIR1wtGFP and GIR1 P7⁻GFP constructs, and no fluorescence with the GIR1 invGFP construct, as expected (FIG. 11). In line with the above interpretation of the primer extension analysis, the only construct that result in GFP production in the absence of arabinose is GIR1wtGFP.

1. An isolated polynucleotide comprising a first and a second subsequence operably linked to each other,
wherein the first subsequence comprises or encodes
a) a GIR1 ribozyme comprising or consisting of SEQ ID NO:1, or
a GIR1 ribozyme comprising or consisting of SEQ ID NO:2, or
a GIR1 ribozyme comprising or consisting of SEQ ID NO:849, or
a GIR1 ribozyme comprising or consisting of SEQ ID NO:850; or
a transcript of any of SEQ ID NO:1, SEQ ID NO:2, SEQ ID NO:849 and SEQ ID NO:850; or
b) a polynucleotide at least 80% identical to any polynucleotide of a); or
c) a fragment of a) or b) capable of cleaving the second subsequence, or the complementary strand thereof; or
d) a polynucleotide, the complementary strand of which hybridizes, under stringent conditions, with a polynucleotide as defined in any of a), b) and c);
wherein the first and second subsequences together are capable of forming a secondary and/or tertiary interaction resulting in stabilization of a transcript of said second subsequence;
wherein the first subsequence is not natively associated with the second subsequence; and
wherein the second subsequence originates from organisms other than Didymium iridis and/or Naegleria jamiesoni.

2. The polynucleotide according to claim 1 further comprising an expression signal capable of directing the expression of said polynucleotide in vitro or in vivo under suitable incubation or cultivation conditions.

3. The polynucleotide according to claim 1, wherein the second subsequence is a coding RNA selected from the group consisting of mRNA, tRNA and rRNA.

4. The polynucleotide according to claim 1, wherein the second subsequence is a non-coding RNA selected from the group consisting of miRNAs, ncRNAs, siRNAs, snRNA(s), snmRNA(s), snoRNA(s), and stRNA.

5. The polynucleotide according to claim 4, wherein the second subsequence originates from a mammal.

6. The polynucleotide according to claim 4, wherein the second subsequence originates from a fungal cell.

7. The polynucleotide according to claim 4, wherein the second subsequence originates from a yeast.

8. The polynucleotide according to claim 4, wherein the second subsequence originates from a bacteria.

9. The polynucleotide according to claim 1, wherein the second subsequence is selected from the group of sequences cited in Table 3.

10. The polynucleotide according to claim 1, wherein the second subsequence is selected from the group of sequences cited in Table 4.

11. The polynucleotide according to claim 1, wherein the second subsequence is selected from the group of sequences cited in Table 5.

12. The polynucleotide according to claim 1, wherein the second subsequence is selected from the group of sequences cited in Table 6.

13. The polynucleotide according to claim 1, wherein the second subsequence is selected from the group of sequences cited in Table 7.

14. The polynucleotide according to claim 1, wherein the second subsequence is human MyoD DNA or mRNA.

15. The polynucleotide according to claim 1, wherein the second subsequence is α-globin DNA or mRNA.

16. The polynucleotide according to claim 1, wherein the second subsequence is human mi RNA mir-218-1.

17. The polynucleotide according to claim 1, wherein the second subsequence is human mi RNA mir-320.

18. The polynucleotide according to claim 1, wherein the second subsequence is human miR15.

19. The polynucleotide according to claim 1, wherein the second subsequence is human miR16.

20. A recombinant polynucleotide molecule in the form of an expression vector comprising the polynucleotide according to claim 1.

21. A host cell transfected or transformed with the polynucleotide according to claim 1.

22. A host cell transfected or transformed with the vector according to claim 20.

23. The host cell according to claim 22, wherein said cell is mammalian.

24. The mammalian host cell according to claim 23, wherein the cell is a human cell.

25. A host cell transfected or transformed with
i) a first polynucleotide comprising a first subsequence comprising or encoding
a) a GIR1 ribozyme comprising or consisting of SEQ ID NO:1, or
a GIR1 ribozyme comprising or consisting of SEQ ID NO:2, or
a GIR1 ribozyme comprising or consisting of SEQ ID NO:849, or
a GIR1 ribozyme comprising or consisting of SEQ ID NO:850, or
a transcript of any of the above;
b) a polynucleotide at least 80% identical to any polynucleotide of a); or
c) a fragment of a) or b) capable of cleaving the second subsequence, or the complementary strand thereof; or
d) a polynucleotide, the complementary strand of which hybridizes, under stringent conditions, with a polynucleotide as defined in any of a), b) and c); and
ii) a second polynucleotide comprising a second subsequence not natively associated with the first subsequence;
wherein the first and second subsequences together are capable of forming a secondary and/or tertiary interaction resulting in stabilization of a transcript of said second subsequence;
wherein the first subsequence is not natively associated with the second subsequence;
wherein the second subsequence originates from organisms other than Didymium iridis and/or Naegleria jamiesoni; and
wherein the host cell does not natively comprise said first and second subsequences.

26. A transgenic organism comprising the polynucleotide according to claim 1.

27. The transgenic organism according to claim 26, wherein the transgenic organism is mammalian.

28. A plant seed comprising the polynucleotide according to claim 1.

29. A plant cell comprising the polynucleotide according to claim 1.

30. A transgenic plant comprising the plant cell according to claim 29.

31. A composition comprising the polynucleotide according to claim 1 in combination with a physiologically acceptable carrier.

32. A composition comprising the vector according to claim 20 in combination with a physiologically acceptable carrier.

33. A composition comprising the host cell according to claim 21 in combination with a physiologically acceptable carrier.

34. A kit-of-parts comprising the polynucleotide according to claim 1, suitable media for host cell transformation or transfection, and at least one host cell.

35. A kit-of-parts comprising the polynucleotide according to claim 1 and a polymerase capable of recognising the expression signal and expressing said first and/or second subsequences.

36. A method for stabilising a polynucleotide, said method comprising the steps of
a) providing the polynucleotide according to claim 1.
b) incubating said polynucleotide under conditions allowing said first and second subsequences to be transcribed and/or translated, and
c) stabilising a transcript of said second subsequence of said polynucleotide.

37. A method for stabilising a polynucleotide, said method comprising the steps of
a) providing the vector according to claim 20,
b) incubating said vector under conditions allowing said first and second subsequences to be transcribed and/or translated, and
c) stabilising a transcript of said second subsequence of said vector.

38. A method for stabilising a polynucleotide, said method comprising the steps of
a) providing the recombinant host cell according to claim 21,
b) incubating said recombinant host cell under conditions allowing said first and second subsequences to be transcribed and/or translated, and
c) stabilising a transcript of said second subsequence.

39. A method for improving the amount of polypeptide produced when expressing a polynucleotide, said method comprising the steps of
a) providing the polynucleotide according to claim 1, wherein said second subsequence encodes a polypeptide
b) incubating said polynucleotide under conditions allowing said first and second subsequences to be transcribed and/or translated, and
c) stabilising a transcript of the second subsequence of said polynucleotide, thereby improving the amount of polypeptide produced when expressing the second subsequence.

40. A method for improving the amount of polypeptide produced when expressing a polynucleotide, said method comprising the steps of
a) providing the vector according to claim 20, wherein said second subsequence encodes a polypeptide,
b) incubating said vector under conditions allowing said first and second subsequences to be transcribed and/or translated, and
c) stabilising a transcript of the second subsequence of said vector, thereby improving the amount of polypeptide produced when expressing the second subsequence.

41. A method for improving the amount of polypeptide produced when expressing a polynucleotide, said method comprising the steps of
a) providing the recombinant host cell according to claim 21, wherein said second subsequence of said host cell encodes a polypeptide,
b) incubating said recombinant host cell under conditions allowing said first and second subsequences to be transcribed and/or translated, and
c) stabilising a transcript of the second subsequence of said recombinant host cell, thereby improving the amount of polypeptide produced when expressing the second subsequence.

42. A method for treating an individual suffering from a disease associated with or caused by instability of a transcript of said second subsequence, said method comprising the steps of
a) providing a recombinant host cell comprising the polynucleotide according to claim 1,
b) transfecting or transforming said host cell into the individual to be treated,
c) expressing said first and second subsequences in said host cell transfected or transformed into said individual, thereby producing a transcript of said first and second subsequences, and
d) stabilising the transcript of said second subsequence to a degree which at least alleviates said disease.

43. The method of claim 42, wherein the disease is cancer.

44. The method of claim 42, wherein the disease is cachexia.

45. The method of claim 42, wherein the disease is α-Thallasemia.

46. The method of claim 42, wherein the disease is leukemia.

47. A method for controlling the phenotype of a biological cell, said method comprising the steps of
a) providing a biological cell comprising the polynucleotide according to claim 1,
b) expressing said first and second subsequences in said biological cell, thereby producing transcripts of said first and second subsequences, and
c) stabilising the transcript of said second subsequence to a degree which controls the phenotype of the biological cell.

48. The method of claim 47, wherein the biological cell is selected from bacteria, yeast cells, fungal cells and plants.

49. The method of claim 47, wherein the second subsequence encodes a non-coding RNA.

50. The method of claim 47, wherein the control of the phenotype allows the cell to adapt to one or more of: an alteration in the composition of the growth medium, including at least one of carbon source, nitrogen source including amino acids or precursors thereof, changes in oxygen content, changes in ionic strength, including NaCl content, changes in pH, presence or absence or changes in low molecular weight compounds, changes in cAMP, and the presence or absence of a cell constituent, or a precursor thereof.
